SEQUENCE LISTING (103 sequences)

SEQ ID NO: 1 - protein, 359 aa, Homo sapiens
  IL1RAP isoform1-ECD-C-terminal His
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATGSGSGSHHHHHH

SEQ ID NO: 2 - protein, 353 aa, Homo sapiens
  IL1RAP isoform2-ECD-N-terminal His
SHHHHHHGSLEVLFQGPSERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKGNRCGQ

SEQ ID NO: 3 - protein, 348 aa, Homo sapiens
  IL1RAP isoform2-ECD-C-terminal His
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKGNRCGQGSGSGSHHHHHH

SEQ ID NO: 4 - protein, 359 aa, Macaca fascicularis
  IL1RAP-ECD-C-terminal His
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIAFISNNGNYTCVVTYPENGRTFHLTRTLTVKVVGSPKNAVPPVIHSPNDHVVYEKEPGEELLIPCTVYFSFLMDSRNEVWWTIDGKKPDDIPIDVTINESISHSRTEDETRTQILSIKKVTSEDLKRSYVCHARSAKGEVAKAATVKQKVPAPRYTVELACGFGATGSGSGSHHHHHH

SEQ ID NO: 5 - protein, 350 aa, Homo sapiens
  IL1RAP isoform1-ECD-C-terminal His-no linker
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEAHHHHHHHHHH

SEQ ID NO: 6 - protein, 347 aa, Homo sapiens
  IL1RAP isoform1-ECD
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGAT

SEQ ID NO: 7 - protein, 347 aa, Macaca fascicularis
  IL1RAP- ECD
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIAFISNNGNYTCVVTYPENGRTFHLTRTLTVKVVGSPKNAVPPVIHSPNDHVVYEKEPGEELLIPCTVYFSFLMDSRNEVWWTIDGKKPDDIPIDVTINESISHSRTEDETRTQILSIKKVTSEDLKRSYVCHARSAKGEVAKAATVKQKVPAPRYTVELACGFGAT

SEQ ID NO: 8 - protein, 347 aa, Mus sp.
  IL1RAP-ECD
SERCDDWGLDTMRQIQVFEDEPARIKCPLFEHFLKYNYSTAHSSGLTLIWYWTRQDRDLEEPINFRLPENRISKEKDVLWFRPTLLNDTGNYTCMLRNTTYCSKVAFPLEVVQKDSCFNSAMRFPVHKMYIEHGIHKITCPNVDGYFPSSVKPSVTWYKGCTEIVDFHNVLPEGMNLSFFIPLVSNNGNYTCVVTYPENGRLFHLTRTVTVKVVGSPKDALPPQIYSPNDRVVYEKEPGEELVIPCKVYFSFIMDSHNEVWWTIDGKKPDDVTVDITINESVSYSSTEDETRTQILSIKKVTPEDLRRNYVCHARNTKGEAEQAAKVKQKVIPPRYTVELACGFGAT

SEQ ID NO: 9 - protein, 347 aa, Rattus sp.
  IL1RAP-ECD
SERCDDWGLDTMRQIQVFEDEPARIKCPLFEHFLKYNYSTAHSSGLTLIWYWTRQDRDLEEPINFRLPENRISKEKDVLWFRPTLLNDTGNYTCMLRNTTYCSKVAFPLEVVQKDSCFNSPMRLPVHRLYIEQGIHNITCPNVDGYFPSSVKPSVTWYKGCTEIVNFHNVQPKGMNLSFFIPLVSNNGNYTCVVTYLENGRLFHLTRTMTVKVVGSPKDAVPPHIYSPNDRVVYEKEPGEELVIPCKVYFSFIMDSHNEIWWTIDGKKPDDVPVDITIIESVSYSSTEDETRTQILSIKKVTPEDLKRNYVCHARNAEGEAEQAAKVKQKVIPPRYTVELACGFGAT

SEQ ID NO: 10 - protein, 8 aa, Homo sapiens
  IAPB47-HCDR1
GYSFTSYW

SEQ ID NO: 11 - protein, 8 aa, Homo sapiens
  IAPB47-HCDR2
IYPSDSYT

SEQ ID NO: 12 - protein, 14 aa, Homo sapiens
  IAPB47-HCDR3
ARRNSAENYADLDY

SEQ ID NO: 13 - protein, 8 aa, Homo sapiens
  IAPB38, and IAPB29-HCDR1
GFTFSNYA

SEQ ID NO: 14 - protein, 8 aa, Homo sapiens
  IAPB38-HCDR2
INYGGGSK

SEQ ID NO: 15 - protein, 11 aa, Homo sapiens
  IAPB38-HCDR3
AKDYGPFALDY

SEQ ID NO: 16 - protein, 10 aa, Homo sapiens
  IAPB57-HCDR1
GGSISSSTYY

SEQ ID NO: 17 - protein, 7 aa, Homo sapiens
  IAPB57-HCDR2
IYFTGST

SEQ ID NO: 18 - protein, 14 aa, Homo sapiens
  IAPB57-HCDR3
AKEDDSSGYYSFDY

SEQ ID NO: 19 - protein, 10 aa, Homo sapiens
  IAPB61 and IAPB55-HCDR1
GVSISSSTYY

SEQ ID NO: 20 - protein, 7 aa, Homo sapiens
  IAPB61 and IAPB55-HCDR2
IYFTGNT

SEQ ID NO: 21 - protein, 12 aa, Homo sapiens
  IAPB61 and IAPB55-HCDR3
GSLFGDYGYFDY

SEQ ID NO: 22 - protein, 8 aa, Homo sapiens
  IAPB62, IAPB63 and IAPB64-HCDR1
GYTFNTYA

SEQ ID NO: 23 - protein, 8 aa, Homo sapiens
  IAPB62, IAPB63 and IAPB64-HCDR2
INTNTGNP

SEQ ID NO: 24 - protein, 14 aa, Homo sapiens
  IAPB62, IAPB63 and IAPB64-HCDR3
ARRYFDWLLGAFDI

SEQ ID NO: 25 - protein, 8 aa, Homo sapiens
  IAPB3, IAPB17, IAPB9 and IAPB65-HCDR1
GGTFSSYA

SEQ ID NO: 26 - protein, 8 aa, Homo sapiens
  IAPB3 and IAPB65-HCDR2
ISAIFGTA

SEQ ID NO: 27 - protein, 16 aa, Homo sapiens
  IAPB3-HCDR3
ARGNSFHALWDYAFDY

SEQ ID NO: 28 - protein, 8 aa, Homo sapiens
  IAPB17-HCDR2
IIPIFGNA

SEQ ID NO: 29 - protein, 15 aa, Homo sapiens
  IAPB17-HCDR3
ARTIIYLDYVHILDY

SEQ ID NO: 30 - protein, 8 aa, Homo sapiens
  IAPB23-HCDR1
GFTFSNYW

SEQ ID NO: 31 - protein, 8 aa, Homo sapiens
  IAPB23-HCDR2
IRYDGGSK

SEQ ID NO: 32 - protein, 12 aa, Homo sapiens
  IAPB23-HCDR3
AKDAYPPYSFDY

SEQ ID NO: 33 - protein, 8 aa, Homo sapiens
  IAPB25-HCDR1
GFTFSSYA

SEQ ID NO: 34 - protein, 8 aa, Homo sapiens
  IAPB25 and IAPB29-HCDR2
ISGSGGST

SEQ ID NO: 35 - protein, 14 aa, Homo sapiens
  IAPB25-HCDR3
AKGDEYYYPDPLDY

SEQ ID NO: 36 - protein, 12 aa, Homo sapiens
  IAPB29-HCDR3
AKEWSSYFGLDY

SEQ ID NO: 37 - protein, 8 aa, Homo sapiens
  IAPB9-HCDR2
ISPIFGTA

SEQ ID NO: 38 - protein, 15 aa, Homo sapiens
  IAPB9-HCDR3
ARRYDNFARSGDLDY

SEQ ID NO: 39 - protein, 12 aa, Homo sapiens
  IAPB65-HCDR3
ARHLHNAIHLDY

SEQ ID NO: 40 - protein, 6 aa, Homo sapiens
  IAPB47-LCDR1
QSISND

SEQ ID NO: 41 - protein, 3 aa, Homo sapiens
  IAPB47-LCDR2
YAS

SEQ ID NO: 42 - protein, 9 aa, Homo sapiens
  IAPB47-LCDR3
QQSFTAPLT

SEQ ID NO: 43 - protein, 6 aa, Homo sapiens
  IAPB38-LCDR1
QSVDDW

SEQ ID NO: 44 - protein, 3 aa, Homo sapiens
  IAPB38-LCDR2
TAS

SEQ ID NO: 45 - protein, 9 aa, Homo sapiens
  IAPB38-LCDR3
QQYHHWPLT

SEQ ID NO: 46 - protein, 6 aa, Homo sapiens
  IAPB57-LCDR1
QGISSY

SEQ ID NO: 47 - protein, 3 aa, Homo sapiens
  IAPB57, IAPB62, IAPB25, IAPB29, and IAPB9-LCDR2
AAS

SEQ ID NO: 48 - protein, 9 aa, Homo sapiens
  IAPB25, IAPB29, and IAPB9-LCDR3
QQSYSTPLT

SEQ ID NO: 49 - protein, 6 aa, Homo sapiens
  IAPB61 and IAPB55-LCDR1
QFISSN

SEQ ID NO: 50 - protein, 3 aa, Homo sapiens
  IAPB61, IAPB55 and IAPB65-LCDR2
GAS

SEQ ID NO: 51 - protein, 9 aa, Homo sapiens
  IAPB61-LCDR3
QQYNNWPST

SEQ ID NO: 52 - protein, 6 aa, Homo sapiens
  IAPB62-LCDR1
QGISSW

SEQ ID NO: 53 - protein, 9 aa, Homo sapiens
  IAPB62-LCDR3
QQANSFPLT

SEQ ID NO: 54 - protein, 12 aa, Homo sapiens
  IAPB3 and IAPB17-LCDR1
QSVLYSSNNKNY

SEQ ID NO: 55 - protein, 3 aa, Homo sapiens
  IAPB3 and IAPB17-LCDR2
WAS

SEQ ID NO: 56 - protein, 9 aa, Homo sapiens
  IAPB3 and IAPB17-LCDR3
QQYYSTPLT

SEQ ID NO: 57 - protein, 6 aa, Homo sapiens
  IAPB23-LCDR1
QSVSSY

SEQ ID NO: 58 - protein, 3 aa, Homo sapiens
  IAPB23-LCDR2
DAS

SEQ ID NO: 59 - protein, 9 aa, Homo sapiens
  IAPB23-LCDR3
QQRSNWPLT

SEQ ID NO: 60 - protein, 6 aa, Homo sapiens
  IAPB25, IAPB29 and IAPB9-LCDR1
QSISSY

SEQ ID NO: 61 - protein, 9 aa, Homo sapiens
  IAPB55-LCDR3
QQYNNWPFT

SEQ ID NO: 62 - protein, 9 aa, Homo sapiens
  IAPB63 and IAPB64-LCDR1
SSDVGDYNY

SEQ ID NO: 63 - protein, 3 aa, Homo sapiens
  IAPB63 and IAPB64-LCDR2
DVS

SEQ ID NO: 64 - protein, 10 aa, Homo sapiens
  IAPB63-LCDR3
ASYAGNYNVV

SEQ ID NO: 65 - protein, 10 aa, Homo sapiens
  IAPB64-LCDR3
SSYAGNYNVV

SEQ ID NO: 66 - protein, 6 aa, Homo sapiens
  IAPB65-LCDR1
QSVSNF

SEQ ID NO: 67 - protein, 9 aa, Homo sapiens
  IAPB65-LCDR3
QQGKHWPWT

SEQ ID NO: 68 - protein, 448 aa, Homo sapiens
  IAPB47-VH
EVQLVQSGAEVKKPGESLKISCKGSGYSFTSYWIGWVRQMPGKGLEWMGIIYPSDSYTRYSPSFQGQVTISADKSISTAYLQWSSLKASDTAMYYCARRNSAENYADLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 214 aa, Homo sapiens
  IAPB47-VL
EIVLTQSPGTLSLSPGERATLSCRASQSISNDLNWYQQKPGKAPKLLIYYASSLQSGVPSRFSGSGSGTDFTLTINSLQPEDFATYYCQQSFTAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 445 aa, Homo sapiens
  IAPB38-VH
EVQLLESGGGLVQPGGSLRLSCAASGFTFSNYAMNWVRQAPGKGLEWVSGINYGGGSKYYADSVKGRFTISRDNSKNTLYLQMNSLRAEDTAVYYCAKDYGPFALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 214 aa, Homo sapiens
  IAPB38-VL
EIVLTQSPATLSLSPGERATLSCRASQSVDDWLAWYQQKPGQAPRLLIYTASNRATGIPARFSGSGSGTDFTLTISSLEPEDFAVYYCQQYHHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 449 aa, Homo sapiens
  IAPB57VH
QLQLQESGPGLVKPSETLSLTCTVSGGSISSSTYYWGWIRQPPGKGLEWIGSIYFTGSTDYNPSLKSRVSISVDTSKNQFSLKLSSVTAADTAVYYCAKEDDSSGYYSFDYWGQGNLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 214 aa, Homo sapiens
  IAPB57-VL
DIQLTQSPSFLSASVGDRVTITCRASQGISSYLAWYQQKPGKAPKLLIYAASTLQSGVPSRFSGSGSGTEFTLTISSLQPEDFATYYCQQVNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 447 aa, Homo sapiens
  IAPB61 and IAPB55-VH
QLQLQESGPGLVKPSETLSLTCTVSGVSISSSTYYWGWLRQPPGMGLEWTGSIYFTGNTYYNPSLKSRVTISVDTSRNQFSLKLSSVTAADTAVYYCGSLFGDYGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 214 aa, Homo sapiens
  IAPB61-VL
EIVMTQSPATLSVPPGERATLSCRASQFISSNLAWYQQKPGQAPRLLIYGASTRATGIPARFSGSGSGTDFTLTISSLQSEDFAVYYCQQYNNWPSTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 448 aa, Homo sapiens
  IAPB62, IAPB63 and IAPB64-VH
QVQLVQSGSELKKPGASVKVSCKASGYTFNTYAMNWVRQAPGQGLEWMGWINTNTGNPTYAQGFTGRFVFSLDTSVSTAYLQISSLKAEDTAVYYCARRYFDWLLGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 214 aa, Homo sapiens
  IAPB62-VL
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 450 aa, Homo sapiens
  IAPB3-VH
QVQLVQSGAEVKKPGSSVKVSCKASGGTFSSYAISWVRQAPGQGLEWMGGISAIFGTANYAQKFQGRVTITADESTSTAYMELSSLRSEDTAVYYCARGNSFHALWDYAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 220 aa, Homo sapiens
  IAPB3 and IAPB17-VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 449 aa, Homo sapiens
  IAPB17-VH
QVQLVQSGAEVKKPGSSVKVSCKASGGTFSSYAISWVRQAPGQGLEWMGGIIPIFGNANYAQKFQGRVTITADESTSTAYMELSSLRSEDTAVYYCARTIIYLDYVHILDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 446 aa, Homo sapiens
  IAPB23-VH
EVQLLESGGGLVQPGGSLRLSCAASGFTFSNYWMNWVRQAPGKGLEWVSAIRYDGGSKYYADSVKGRFTISRDNSKNTLYLQMNSLRAEDTAVYYCAKDAYPPYSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 214 aa, Homo sapiens
  IAPB23-VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 448 aa, Homo sapiens
  IAPB25-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGDEYYYPDPLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 214 aa, Homo sapiens
  IAPB25, IAPB29 and IAPB9-VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 446 aa, Homo sapiens
  IAPB29-VH
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKEWSSYFGLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 449 aa, Homo sapiens
  IAPB9-VH
QVQLVQSGAEVKKPGSSVKVSCKASGGTFSSYAISWVRQAPGQGLEWMGWISPIFGTANYAQKFQGRVTITADESTSTAYMELSSLRSEDTAVYYCARRYDNFARSGDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 214 aa, Homo sapiens
  IAPB55-VL
EIVMTQSPATLSVSPGERATLSCRASQFISSNLAWYQQKPGQAPRLLIYGASTRATGIPARFSGSGSGTDFTLTISSLQSEDFAVYYCQQYNNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 216 aa, Homo sapiens
  IAPB63-VL
QSALTQPRSVSGSPGHSVTISCTGTSSDVGDYNYVSWYQQRPGKVPKLLIYDVSKRPSGVPDRFSGSKSGNTASLTISGLQAEDEAIYFCASYAGNYNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 89 - protein, 216 aa, Homo sapiens
  IAPB64-VL
QSALTQPRSVSGSPGHSVTISCTGTSSDVGDYNYVSWYQQRPGKVPKLLIYDVSKRPSGVPDRFSGSKSGNTASLTISGLQAEDEAIYFCSSYAGNYNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 90 - protein, 446 aa, Homo sapiens
  IAPB65-VH
QVQLVQSGAEVKKPGSSVKVSCKASGGTFSSYAISWVRQAPGQGLEWMGGISAIFGTANYAQKFQGRVTITADESTSTAYMELSSLRSEDTAVYYCARHLHNAIHLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 214 aa, Homo sapiens
  IAPB65-VL
EIVLTQSPATLSLSPGERATLSCRASQSVSNFLAWYQQKPGQAPRLLIYGASNRATGIPARFSGSGSGTDFTLTISSLEPEDFAVYYCQQGKHWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 452 aa, Artificial Sequence
  Description of Artificial Sequence Synthetic polypeptide
  CD3B220-VH
EVQLVESGGGLVQPGGSLKLSCAASGFTFNTYAMNWVRQASGKGLEWVGRIRSKYNAYATYYAASVKGRFTISRDDSKNTAYLQMNSLKTEDTAVYYCTRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 93 - protein, 215 aa, Artificial Sequence
  Description of Artificial Sequence Synthetic polypeptide
  CD3B220-VL
QAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 94 - protein, 452 aa, Artificial Sequence
  Description of Artificial Sequence Synthetic polypeptide
  CD3B219-VH
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 95 - protein, 215 aa, Artificial Sequence
  Description of Artificial Sequence Synthetic polypeptide
  CD3B219-VL
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 96 - protein, 5 aa, Mus sp.
  CD3B219 and CD3B220-HCDR1
TYAMN

SEQ ID NO: 97 - protein, 19 aa, Mus sp.
  CD3B220-HCDR2
RIRSKYNAYATYYAASVKG

SEQ ID NO: 98 - protein, 14 aa, Mus sp.
  CD3B219 and CD3B220-HCDR3
HGNFGNSYVSWFAY

SEQ ID NO: 99 - protein, 14 aa, Mus sp.
  CD3B219 and CD3B220-LCDR1
RSSTGAVTTSNYAN

SEQ ID NO: 100 - protein, 7 aa, Mus sp.
  CD3B219 and CD3B220-LCDR2
GTNKRAP

SEQ ID NO: 101 - protein, 9 aa, Mus sp.
  CD3B219 and CD3B220-LCDR3
ALWYSNLWV

SEQ ID NO: 102 - protein, 19 aa, Artificial Sequence
  Description of Artificial Sequence Synthetic peptide
  CD3B219-HCDR2
RIRSKYNNYATYYAASVKG

SEQ ID NO: 103 - protein, 9 aa, Homo sapiens
  IAPB57-LCDR3
QQVNSYPLT

(19) United States
(12) Patent Application Publication
(10) Pub. No.: US 2019/0270826 A1
(43) Pub. Date: Sep. 5, 2019

(54) ANTI-IL1RAP ANTIBODIES, BISPECIFIC ANTIGEN BINDING MOLECULES THAT BIND IL1RAP AND CD3, AND USES THEREOF

(71) Applicant: JANSSEN PHARMACEUTICA NV, BEERSE (BE)

(72) Inventors: Bradley J. Heidrich, Spring House, PA (US); Jennifer F. Nemeth, Spring House, PA (US); Walter K. Nishioka, JR., San Diego, CA (US); Thai Dinh, San Diego, CA (US); Rosa Maria Fernandes Cardoso, Spring House, PA (US); Ricardo Marcos Attar, Spring House, PA (US); Francois Gaudet, Spring House, PA (US); Mark E. Salvati, Spring House, PA (US)

(73) Assignee: JANSSEN PHARMACEUTICA NV, BEERSE (BE)

(21) Appl. No.: 16/358,894

(22) Filed: Mar. 20, 2019

Related U.S. Application Data
(62) Division of application No. 15/340,149, filed on Nov. 1, 2016, now abandoned.
(60) Provisional application No. 62/249,466, filed on Nov. 2, 2015.

(51) Int. Cl. C07K 16/30; A61K 39/395; C07K 16/46; A61K 45/06; C07K 16/28

(52) U.S. Cl. CPC C07K 16/30; A61K 2039/572; A61K 39/39558; C07K 16/468; C07K 16/3069; C07K 16/3061; C07K 16/3053; C07K 16/3046; C07K 16/303; C07K 16/3023; C07K 16/3015; A61K 45/06; C07K 16/2866; C07K 16/2809; C07K 2317/76; C07K 2317/21; C07K 2317/34; C07K 2317/24; C07K 2317/565; C07K 16/3038

(57) ABSTRACT

Provided herein are antibodies that specifically bind to IL1RAP. Also described are related polynucleotides capable of encoding the provided IL1RAP-specific antibodies or antigen-binding fragments, cells expressing the provided antibodies or antigen-binding fragments, as well as associated vectors and detectably labeled antibodies or antigen-binding fragments. In addition, methods of using the provided antibodies are described. For example, the provided antibodies may be used to diagnose, treat, or monitor IL1RAP-expressing cancer progression, regression, or stability; to determine whether or not a patient should be treated for cancer; or to determine whether or not a subject is afflicted with IL1RAP-expressing cancer and thus may be amenable to treatment with an IL1RAP-specific anti-cancer therapeutic, such as the multi specific antibodies against IL1RAP and CD3 described herein.

This application claims the benefit of U.S. Provisional Patent Application Ser. No. 62/249,466, filed Nov. 2, 2015, which is hereby incorporated by reference in its entirety.

The instant application contains a Sequence Listing which has been submitted electronically in ASCII format and is hereby incorporated by reference in its entirety. Said ASCII copy, created on Oct. 27, 2016, is named PRD3394USNP SL.txt and is 121,828 bytes in size.

### TECHNICAL FIELD

The disclosure provided herein relates to monoclonal antibodies that specifically bind interleukin-1 receptor accessory protein (IL1RAP), multispecific antibodies that specifically bind IL1RAP and cluster determinant 3 (CD3), and methods of producing and using the described antibodies.

### BACKGROUND

Acute myeloid leukemia (AML) is a genetically heterogeneous disease characterized by clonal expansion of leukemic cells. Despite an increased understanding of the underlying disease biology in AML, the standard treatment with cytotoxic chemotherapy has remained largely unchanged over the last decades and the overall five year survival remains poor, being <30% (Cancer Genome Atlas Research Network (2013) N Engl J Med 368(22):2059-2074; Burnett A, Wetzler M, Lowenberg B (2011) J Clin Oncol 29(5):487-494.). Hence, there is a pressing need for novel therapies with increased efficacy and decreased toxicity, ideally targeting the AML stem cells because these cells are believed to be critical in the pathogenesis of AML, and their inadequate eradication by standard therapy is thought to contribute to the high incidence of relapse (Hope K J, Jin L, Dick J E (2004) Nat Immunol 5(7):738-743; Ishikawa F, et al. (2007) Nat Biotechnol 25(11):1315-1321.). Although therapeutic antibodies directed at cell-surface molecules have proven effective for the treatment of malignant disorders such as lymphomas and acute lymphoblastic leukemia, as well as solid tumors (Hoelzer D (2013) Curr Opin Oncol 25(6):701-706, Jackson S E, Chester J D (2015) Int J Cancer 137(2):262-266.), no antibody-based therapy is currently approved for AML.

The interleukin 1 receptor accessory protein (IL1RAP), also called IL1R3, is a coreceptor of type 1 interleukin 1 receptor (IL1R1), interleukin-33 receptor (IL-33R, also called ST2), and interleukin-36 receptor (IL-36R, also called IL-1RL2) and is indispensable for transmission of IL-1, IL-33, and IL-36 signaling (Subramaniam S, Stansberg C, Cunningham C (2004) Dev Comp Immunol 28(5):415-428.). IL1RAP has been reported as a biomarker for putative chronic myeloid leukemia stem cells (Järås M, et al. (2010) Proc Natl Acad Sci USA 107(37):16280-16285.). A recent study shows that IL1RAP is expressed on the cell surface in ˜80% of AML patients and that candidate CD34⁺CD38⁻ AML stem cells can be selectively killed in vitro by antibody-dependent cellular cytotoxicity (ADCC) (Askmyr M, et al. (2013) Blood 121(18):3709-3713.). Furthermore, IL1RAP is up-regulated on immature cells in high-risk AML with chromosome 7 aberrations, and increased IL1RAP expression correlates with poor prognosis (Barreyro L, et al. (2012) Blood 120(6):1290-1298.). These findings suggest that IL1RAP is a suitable target for an antibody-based therapy in AML.

The use of anti-IL1RAP antibodies for the treatment of AML is mentioned in WO2009120903 and WO2011021014. Antibodies against IL1RAP are described e.g. in WO2014100772. The described IL1RAP antibodies utilize ADCC as their mode of action. Unfortunately, the triggering of ADCC by therapeutic antibodies faces several limitations. First of all, the affinity between the Fc and its receptors plays a crucial role, and the fact that 80% of the population expresses a low affinity variant of the receptor is a major issue (Chames P, Van Regenmortel M, Weiss E, Baty D. (2009) British Journal of Pharmacology. 157(2):220-233.). Second, IgG1 molecules are glycosylated in the CH2 domain (Asn 297) of the Fc region. This modification has been shown to decrease ADCC efficiency (Shinkawa T, Nakamura K, Yamane N, Shoji-Hosaka E, Kanda Y, Sakurada M, et al. J Biol Chem. 2003; 278:3466-3473.). A third limitation lies in the fact that therapeutic antibodies have to compete with a high concentration of patient's IgGs for binding to FcγRIIIa (Preithner S, Elm S, Lippold S, Locher M, Wolf A, da Silva A J, et al. Mol Immunol. 2006; 43:1183-1193.). Finally, a fourth limitation of the use of therapeutic antibodies may be their affinity for inhibitory Fc receptors such as FcγRIIb, expressed by B-cells, macrophages, dendritic cells and neutrophils (Nimmerjahn F, Ravetch J V. Antibodies, Fc receptors and cancer. Curr Opin Immunol. 2007; 19:239-245.).

Thus, there is still a need for having available further options for the treatment of IL1RAP-expressing cancers.

### SUMMARY

Provided herein are antibodies that specifically bind to IL1RAP and antigen-binding fragments thereof. Also described are related polynucleotides capable of encoding the provided IL1RAP-specific antibodies and antigen-binding fragments, cells expressing the provided antibodies and antigen-binding fragments, as well as associated vectors and detectably labeled antibodies and antigen-binding fragments. In addition, methods of using the provided antibodies and antigen-binding fragments are described. For example, the IL1RAP-specific antibodies and antigen-binding fragments may be used to diagnose or monitor IL1RAP-expressing cancer progression, regression, or stability; to determine whether or not a patient should be treated for cancer; or to determine whether or not a subject is afflicted with IL1RAP-expressing cancer and thus may be amenable to treatment with an IL1RAP-specific anti-cancer therapeutic, such as the multispecific antibodies against IL1RAP and CD3 described herein.

Further provided herein are multispecific antibodies that specifically bind to IL1RAP and CD3 and multispecific antigen-binding fragments thereof. Also described are related polynucleotides capable of encoding the provided IL1RAP x CD3-multispecific antibodies, cells expressing the provided antibodies, as well as associated vectors and detectably labeled multispecific antibodies. In addition, methods of using the provided multispecific antibodies are described. For example, the IL1RAP x CD3-multispecific antibodies may be used to diagnose or monitor IL1RAP-expressing cancer progression, regression, or stability; to determine whether or not a patient should be treated for cancer; or to determine whether or not a subject is afflicted with IL1RAP-expressing cancer and thus may be amenable to treatment with an IL1RAP-specific anti-cancer therapeutic, such as the IL1RAP x CD3-multispecific antibodies described herein.

IL1RAP-Specific Antibodies

Described herein are recombinant antibodies and antigen-binding fragments specific for IL1RAP. In some embodiments, the IL1RAP-specific antibodies and antigen-binding fragments bind human IL1RAP. In some embodiments, the IL1RAP-specific antibodies and antigen-binding fragments bind human IL1RAP and cynomolgus monkey IL1RAP. In some embodiments, the IL1RAP-specific antibodies and antigen-binding fragments bind to an epitope including one or more residues from the IL1RAP extracellular domain (ECD). This IL1RAP-specific antibody or antigen-binding fragment may bind to IL1RAP with an affinity of 50 nM or less.

Table 1 provides a summary of examples of some IL1RAP-specific antibodies described herein:

TABLE 1
CDR sequences of antibodies generated against human IL1RAP. CDRs are
defined using IMGT.
(SEQ ID NO:)

| | | | | | LC- | |
|---|---|---|---|---|---|---|
| ID | HC-CDR1 | HC-CDR2 | HC-CDR3 | LC-CDR1 | CDR2 | LC-CDR3 |
| IAPB47 | GYSFTSYW | IYPSDSYT | ARRNSAENYADLDY (12) | (QSISND (40) | YAS | QQSFTAPLT |
| | (10) | (11) | | | (41) | (42) |
| IAPB38 | GFTFSNYA | INYGGGSK | AKDYGPFALDY (15) | QSVDDW (43) | TAS | QQYHHWPLT |
| | (13) | (14) | | | (44) | (45) |
| IAPB57 | GGSISSSTYY | IYFTGST | AKEDDSSGYYSFDY (18) | QGISSY (46) | AAS | QQVNSYPLT |
| | (16) | (17) | | | (47) | (103) |
| IAPB61 | GVSISSSTYY | IYFTGNT | GSLFGDYGYFDY (21) | QFISSN (49) | GAS | QQYNNWPST |
| | (19) | (20) | | | (50) | (51) |
| IAPB62 | GYTFNTYA | INTNTGNP | ARRYFDWLLGAFDI (24) | QGISSW (52) | AAS (47) | QQANSFPLT |
| | (22) | (23) | | | | (53) |
| IAPB3 | GGTFSSYA | ISAIFGTA | ARGNSFHALWDYAFDY (27) | QSVLYSSNNKNY | WAS (55) | QQYYSTPLT |
| | (25) | (26) | | (54) | | (56) |
| IAPB17 | GGTFSSYA | IIPIFGNA | ARTIIYLDYVHILDY (29) | QSVLYSSNNKNY | WAS (55) | QQYYSTPLT |
| | (25) | (28) | | (54) | | (56) |
| IAPB23 | GFTFSNYW | IRYDGGSK | AKDAYPPYSFDY (32) | QSVSSY (57) | DAS (58) | QQRSNWPLT |
| | (30) | (31) | | | | (59) |
| IAPB25 | GFTFSSYA | ISGSGGST | AKGDEYYYPDPLDY (35) | QSISSY (60) | AAS (47) | QQSYSTPLT |
| | (33) | (34) | | | | (48) |
| IAPB29 | GFTFSNYA | ISGSGGST | AKEWSSYFGLDY (36) | QSISSY (60) | AAS (47) | QQSYSTPLT |
| | (13) | (34) | | | | (48) |
| IAPB9 | GGTFSSYA | ISPIFGTA | ARRYDNFARSGDLDY (38) | QSISSY (60) | AAS (47) | QQSYSTPLT |
| | (25) | (37) | | | | (48) |
| IAPB55 | GVSISSSTYY | IYFTGNT | GSLFGDYGYFDY (21) | QFISSN (49) | GAS (50) | QQYNNWPFT |
| | (19) | (20) | | | | (61) |
| IAPB63 | GYTFNTYA | INTNTGNP | ARRYFDWLLGAFDI (24) | SSDVGDYNY (62) | DVS (63) | ASYAGNYNVV |
| | (22) | (23) | | | | (64) |
| IAPB64 | GYTFNTYA | INTNTGNP | ARRYFDWLLGAFDI (24) | SSDVGDYNY (62) | DVS (63) | SSYAGNYNVV |
| | (22) | (23) | | | | (65) |
| IAPB65 | GGTFSSYA | ISAIFGTA | ARHLHNAIHLDY (39) | QSVSNF (66) | GAS (50) | QQGKHWPWT |
| | (25) | (26) | | | | (67) |

In some embodiments are provided an IL1RAP-specific antibody, or an antigen-binding fragment thereof, comprising a heavy chain comprising a CDR1, a CDR2, and a CDR3 of any one of the antibodies described in Table 1. In some embodiments are provided an IL1RAP-specific antibody, or an antigen-binding fragment thereof, comprising a heavy chain comprising a CDR1, a CDR2, and a CDR3 of any one of the antibodies described in Table 1 and a light chain comprising a CDR1, a CDR2, and a CDR3 of any one of the antibodies described in Table 1. In some embodiments described herein, the IL1RAP-specific antibody or antigen-binding fragment thereof competes for binding to IL1RAP with an antibody or antigen-binding comprising a heavy chain comprising a CDR1, a CDR2, and a CDR3 of any one of the antibodies described in Table 1 and a light chain comprising a CDR1, a CDR2, and a CDR3 of any one of the antibodies described in Table 1.

The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcgRs) on the surface of immune effector cells such as natural killers and macrophages, leading to the phagocytosis or lysis of the targeted cells. In CDC, the antibodies kill the targeted cells by triggering the complement cascade at the cell surface. The antibodies described herein include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcgRs or the complement factors. The binding of IgG to the activating (FcgRI, FcgRIIa, FcgRIIIa and FcgRIIIb) and inhibitory (FcgRIIb) FcgRs or the first component of complement (Clq) depends on residues located in the hinge region and the CH2 domain. Mutations have been introduced in IgG1, IgG2 and IgG4 to reduce or silence Fc functionalities. The antibodies described herein may include these modifications.

In one embodiment, the antibody comprises an Fc region with one or more of the following properties: (a) reduced effector function when compared to the parent Fc; (b) reduced affinity to Fcg RI, Fcg RIIa, Fcg RIIb, Fcg RIIIb and/or Fcg RIIIa, (c) reduced affinity to FcgRI (d) reduced affinity to FcgRIIa (e) reduced affinity to FcgRIIb, (f) reduced affinity to Fcg RIIIb or (g) reduced affinity to FcgRIIIa.

In some embodiments, the antibodies or antigen-binding fragments are IgG, or derivatives thereof, e.g., IgG1, IgG2, IgG3, and IgG4 isotypes. In some embodiments wherein the antibody has an IgG1 isotype, the antibody contains L234A, L235A, and/or K409R substitution(s) in its Fc region. In some embodiments wherein the antibody has an IgG4 isotype, the antibody contains S228P, L234A, and L235A substitutions in its Fc region. The antibodies described herein may include these modifications.

In some embodiments the described antibodies are capable of binding to IL1RAP with a dissociation constant of 50 nM or less as measured by surface plasmon resonance (SPR). In some embodiments, the antibodies comprise the CDRs of the antibodies presented in Table 1 above. Assays for measuring affinity include assays performed using a BIAcore 3000 machine, where the assay is performed at room temperature (e.g. at or near 25° C.), wherein the antibody capable of binding to IL1RAP is captured on the BIAcore sensor chip by an anti-Fc antibody (e.g. goat anti-human IgG Fc specific antibody Jackson ImmunoResearch laboratories Prod #109-005-098) to a level around 75 RUs, followed by the collection of association and dissociation data at a flow rate of 40 μL/min.

In addition to the described IL1RAP-specific antibodies and antigen-binding fragments, also provided are polynucleotide sequences capable of encoding the described antibodies and antigen-binding fragments. Vectors comprising the described polynucleotides are also provided, as are cells expressing the IL1RAP-specific antibodies or antigen-binding fragments provided herein. Also described are cells capable of expressing the disclosed vectors. These cells may be mammalian cells (such as HEK-293F cells, CHO-K1 cells), insect cells (such as Sf7 cells), yeast cells, plant cells, or bacteria cells (such as E. coli). The described antibodies may also be produced by hybridoma cells.

Methods of Using IL1RAP-Specific Antibodies

Methods of using the described IL1RAP-specific antibodies or antigen-binding fragments are also disclosed. Particular antibodies for use in the methods discussed in this section include those with the set of CDRs described for antibodies in Table 1. For example, these antibodies or antigen-binding fragments may be useful in treating cancer, by 1) interfering with IL1RAP-receptor interactions, 2) where the antibody is conjugated to a toxin, so targeting the toxin to the IL1RAP-expressing cancer, or 3) redirecting the body's immune cells to the site of the IL1RAP-expressing cancer (ADCC, T cell redirection). Further, these antibodies or antigen-binding fragments may be useful for detecting the presence of IL1RAP in a biological sample, such as blood or serum; for quantifying the amount of IL1RAP in a biological sample, such as blood or serum; for diagnosing IL1RAP-expressing cancer; determining a method of treating a subject afflicted with cancer; or monitoring the progression of IL1RAP-expressing cancer in a subject. In some embodiments, IL1RAP-expressing cancer may be a hematological cancer, such as acute myeloid leukemia (AML), myelodysplastic syndrome (MDS, low, intermediate, or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas. The described methods may be carried out before the subject receives treatment for IL1RAP-expressing cancer, such as treatment with a multispecific antibody against IL1RAP and CD3. Furthermore, the described methods may be carried out after the subject receives treatment for IL1RAP-expressing cancer, such as treatment with a multispecific antibody against IL1RAP and CD3 described herein.

The described methods of detecting IL1RAP in a biological sample include exposing the biological sample to one or more of the IL1RAP-specific antibodies or antigen-binding fragments described herein.

The described methods of diagnosing IL1RAP-expressing cancer in a subject also involve exposing the biological sample to one or more of the IL1RAP-specific antibodies or antigen-binding fragments described herein; however, the methods also include quantifying the amount of IL1RAP present in the sample; comparing the amount of IL1RAP present in the sample to a known standard or reference sample; and determining whether the subject's IL1RAP levels fall within the levels of IL1RAP associated with cancer.

Also described herein are methods of monitoring IL1RAP-expressing cancer in a subject. The described methods include exposing the biological sample to one or more of the IL1RAP-specific antibodies or antigen-binding fragments described herein; quantifying the amount of IL1RAP present in the sample that is bound by the antibody, or antigen-binding fragment thereof; comparing the amount of IL1RAP present in the sample to either a known standard or reference sample or the amount of IL1RAP in a similar sample previously obtained from the subject; and determining whether the subject's IL1RAP levels are indicative of cancer progression, regression or stable disease based on the difference in the amount of IL1RAP in the compared samples.

The samples obtained, or derived from, subjects are biological samples such as urine, blood, serum, plasma, saliva, ascites, circulating cells, circulating tumor cells, cells that are not tissue associated, tissues, surgically resected tumor tissue, biopsies, fine needle aspiration samples, or histological preparations.

The described IL1RAP-specific antibodies or antigen-binding fragments may be labeled for use with the described methods, or other methods known to those skilled in the art. For example, the antibodies described herein, or antigen-binding fragments thereof, may be labeled with a radiolabel, a fluorescent label, an epitope tag, biotin, a chromophore label, an ECL label, an enzyme, ruthenium, ¹¹¹In-DOTA, diethylenetriaminepentaacetic acid (DTPA), horseradish peroxidase, alkaline phosphatase and beta-galactosidase, or poly-histidine or similar such labels known in the art.

IL1RAP-Specific Antibody Kits

Described herein are kits including the disclosed IL1RAP-specific antibodies or antigen-binding fragments thereof. The described kits may be used to carry out the methods of using the IL1RAP-specific antibodies or antigen-binding fragments provided herein, or other methods known to those skilled in the art. In some embodiments the described kits may include the antibodies or antigen-binding fragments described herein and reagents for use in detecting the presence of IL1RAP in a biological sample. Accordingly, the described kits may include one or more of the antibodies, or an antigen-binding fragment(s) thereof, described herein and a vessel for containing the antibody or fragment when not in use, instructions for use of the antibody or fragment, the antibody or fragment affixed to a solid support, and/or detectably labeled forms of the antibody or fragment, as described herein.

IL1RAP x CD3-Multispecific Antibodies

The redirection of T-lymphocytes to IL1RAP-expressing cancer cells via the TCR/CD3 complex represents an attractive alternative approach. The TCR/CD3 complex of T-lymphocytes consists of either a TCR alpha (α)/beta (β) or TCR gamma (γ)/delta (δ) heterodimer coexpressed at the cell surface with the invariant subunits of CD3 labeled gamma (γ), delta (δ), epsilon (ε), zeta (ζ), and eta (η). Human CD3c is described under UniProt P07766 (CD3E HUMAN). An anti-CD3c antibody described in the state of the art is SP34 (Yang S J, The Journal of Immunology (1986) 137; 1097-1100). SP34 reacts with both primate and human CD3. SP34 is available from Pharmingen. A further anti-CD3 antibody described in the state of the art is UCHT-1 (see WO2000041474). A further anti-CD3 antibody described in the state of the art is BC-3 (Fred Hutchinson Cancer Research Institute; used in Phase I/II trials of GvHD, Anasetti et al., Transplantation 54: 844 (1992)). SP34 differs from UCHT-1 and BC-3 in that SP-34 recognizes an epitope present on solely the c chain of CD3 (see Salmeron et al., (1991) J. Immunol. 147: 3047) whereas UCHT-1 and BC-3 recognize an epitope contributed by both the and γ chains. The sequence of an antibody with the same sequence as of antibody SP34 is mentioned in WO2008119565, WO2008119566, WO2008119567, WO2010037836, WO2010037837 and WO2010037838. A sequence which is 96% identical to VH of antibody SP34 is mentioned in U.S. Pat. No. 8,236,308 (WO2007042261).

Described herein are recombinant multispecific antibodies that bind IL1RAP and CD3 (“IL1RAP x CD3 multispecific antibodies”) and multispecific antigen-binding fragments thereof. In some embodiments a recombinant antibody, or an antigen-binding fragment thereof, that binds specifically to IL1RAP is provided.

In some embodiments, the IL1RAP-specific arm of the multispecific antibody binds human IL1RAP and/or cynomolgus monkey IL1RAP. In some embodiments, the IL1RAP-specific arm of the IL1RAP x CD3-multispecific antibodies or antigen-binding fragments binds the extracellular domain of human IL1RAP. In preferred embodiments, the IL1RAP x CD3 multispecific antibody or antigen-binding fragment is a bispecific antibody or antigen-binding fragment. In some embodiments, a recombinant IL1RAP x CD3 bispecific antibody comprising: a) a first heavy chain (HC1); b) a second heavy chain (HC2); c) a first light chain (LC1); and d) a second light chain (LC2), wherein the HC1 and the LC1 pair to form a first antigen-binding site that specifically binds IL1RAP, and the HC2 and the LC2 pair to form a second antigen-binding site that specifically binds CD3, or an IL1RAP x CD3-bispecific binding fragment thereof is provided. In another embodiment, a recombinant cell expressing the antibody or bispecific binding fragment is provided. In some embodiments, the IL1RAP-binding arm (or “IL1RAP-specific arm”) of the IL1RAP x CD3 multispecific antibody is derived from an IL1RAP antibody described herein (for example, from an antibody having the CDR sequences listed in Table 1).

In some embodiments, the IL1RAP-specific arm of the IL1RAP x CD3-multispecific antibodies or antigen-binding fragments are IgG, or derivatives thereof. In some embodiments the described IL1RAP x CD3-multispecific antibodies are capable of binding to IL1RAP with a dissociation constant of 30 nM or less as measured by surface plasmon resonance. In some embodiments the described IL1RAP x CD3-multispecific antibody is not an agonist. In some embodiments the described IL1RAP x CD3-multispecific antibody inhibits IL-1β-mediated activation of AP-1 and NF-κB activation at concentrations above 6.7 nM.

In some embodiments, the CD3-binding arm (or “CD3-specific arm”) of the IL1RAP x CD3 multispecific antibody is derived from the mouse monoclonal antibody SP34, a mouse IgG3/lambda isotype. (K. R. Abhinandan and A. C. Martin, 2008. Mol. Immunol. 45, 3832-3839). In some embodiments, the CD3-binding arm of the IL1RAP x CD3 multispecific antibody comprises one VH domain and one VL domain selected from Table 2.

TABLE 2
Heavy chains and light chains of the CD3-specific antibodies and
antigen-binding fragments. CDRs, as defined by Kabat are underlined.

| VH | VL |
|---|---|
| CD3B220 (SEQ ID NO: 92): | CD3B220 (SEQ ID NO: 93): |
| EVQLVESGGGLVQPGGSLKLSCAASGFTFNT | QAVVTQEPSLTVSPGGTVTLTCRSSTGAVTTSNYA |
| YAMNWVRQASGKGLEWVGRIRSKYNAYATY | NWVQQKPGQAPRGLIGGTNKRAPGTPARFSGSLL |
| YAASVKGRFTISRDDSKNTAYLQMNSLKTED | GGKAALTLSGAQPEDEAEYYCALWYSNLWVFGG |
| TAVYYCTRHGNFGNSYVSWFAYWGQGTLVT | GTKLTVLGQPKAAPSVTLFPPSSEELQANKATLVC |
| VSSASTKGPSVFPLAPCSRSTSESTAALGCL | LISDFYPGAVTVAWKADSSPVKAGVETTTPSKQSN |
| VKDYFPEPVTVSWNSGALTSGVHTFPAVLQS | NKYAASSYLSLTPEQWKSHRSYSCQVTHEGSTVE |
| SGLYSLSSVVTVPSSSLGTKTYTCNVDHKPS | KTVAPTECS |
| NTKVDKRVESKYGPPCPPCPAPEAAGGPSV | |
| FLFPPKPKDTLMISRTPEVTCVVVDVSQEDPE | |
| VQFNWYVDGVEVHNAKTKPREEQFNSTYRV | |
| VSVLTVLHQDWLNGKEYKCKVSNKGLPSSIE | |
| KTISKAKGQPREPQVYTLPPSQEEMTKNQVS | |
| LTCLVKGFYPSDIAVEWESNGQPENNYKTTP | |
| PVLDSDGSFLLYSKLTVDKSRWQEGNVFSCS | |
| VMHEALHNHYTQKSLSLSLGK | |
| CD3B219 (SEQ ID NO: 94): | CD3B219 (SEQ ID NO: 95): |
| EVQLVESGGGLVQPGGSLRLSCAASGFTFN | QTVVTQEPSLTVSPGGTVTLTCRSSTGAVTTSNYA |
| TYAMNWVRQAPGKGLEWVARIRSKYNNYAT | NWVQQKPGQAPRGLIGGTNKRAPGTPARFSGSLL |
| YYAASVKGRFTISRDDSKNSLYLQMNSLKTE | GGKAALTLSGVQPEDEAEYYCALWYSNLVVVFGG |
| DTAVYYCARHGNFGNSYVSWFAYWGQGTL | GTKLTVLGQPKAAPSVTLFPPSSEELQANKATLVC |
| VTVSSASTKGPSVFPLAPCSRSTSESTAALG | LISDFYPGAVTVAWKADSSPVKAGVETTTPSKQSN |
| CLVKDYFPEPVTVSWNSGALTSGVHTFPAVL | NKYAASSYLSLTPEQWKSHRSYSCQVTHEGSTVE |
| QSSGLYSLSSVVTVPSSSLGTKTYTCNVDHK | KTVAPTECS |
| PSNTKVDKRVESKYGPPCPPCPAPEAAGGP | |
| SVFLFPPKPKDTLMISRTPEVTCVVVDVSQED | |
| PEVQFNWYVDGVEVHNAKTKPREEQFNSTY | |
| RVVSVLTVLHQDWLNGKEYKCKVSNKGLPS | |
| SIEKTISKAKGQPREPQVYTLPPSQEEMTKN | |
| QVSLTCLVKGFYPSDIAVEWESNGQPENNYK | |
| TTPPVLDSDGSFLLYSKLTVDKSRWQEGNVF | |
| SCSVMHEALHNHYTQKSLSLSLGK | |

The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcgRs) on the surface of immune effector cells such as natural killers and macrophages, leading to the phagocytosis or lysis of the targeted cells. In CDC, the antibodies kill the targeted cells by triggering the complement cascade at the cell surface.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcgRs or the complement factors. The binding of IgG to the activating (FcgRI, FcgRlla, FcgRIIIa and FcgRIIIb) and inhibitory (FcgRIIb) FcgRs or the first component of complement (Clq) depends on residues located in the hinge region and the CH2 domain. Mutations have been introduced in IgG1, IgG2 and IgG4 to reduce or silence Fc functionalities.

In one embodiment, the antibody comprises an Fc region with one or more of the following properties: (a) reduced effector function when compared to the parent Fc; (b) reduced affinity to Fcg RI, Fcg RIIa, Fcg RIIb, Fcg RIIIb and/or Fcg RIIIa, (c) reduced affinity to FcgRI (d) reduced affinity to FcgRIIa (e) reduced affinity to FcgRIIb, (f) reduced affinity to Fcg RIIIb or (g) reduced affinity to FcgRIIIa.

In some embodiments, the CD3-specific antibody or antigen-binding fragment from which the CD3-specific arm of the multispecific antibody is derived is IgG, or a derivative thereof. In some embodiments, the CD3-specific antibody or antigen-binding fragment from which the CD3-specific arm of the multispecific antibody is derived is IgG1, or a derivative thereof. In some embodiments, for example, the Fc region of the CD3-specific IgG1 antibody from which the CD3-binding arm is derived comprises L234A, L235A, and F405L substitutions in its Fc region. In some embodiments, the CD3-specific antibody or antigen-binding fragment from which the CD3-specific arm of the multispecific antibody is derived is IgG4, or a derivative thereof. In some embodiments, for example, the Fc region of the CD3-specific IgG4 antibody from which the CD3-binding arm is derived comprises S228P, L234A, L235A, F405L, and R409K substitutions in its Fc region. In some embodiments, the CD3-specific antibody or antigen-binding fragment from which the CD3-specific arm of the multispecific antibody is derived binds CD3c on primary human T cells and/or primary cynomolgus T cells. In some embodiments, the CD3-specific antibody or antigen-binding fragment from which the CD3-specific arm of the multispecific antibody is derived activates primary human CD4+ T cells and/or primary cynomolgus CD4+ T cells.

In addition to the described IL1RAP x CD3-multispecific antibodies, also provided are polynucleotide sequences capable of encoding the described IL1RAP x CD3-multispecific antibodies. In some embodiments, an isolated synthetic polynucleotide encoding the HC1, the HC2, the LC1 or the LC2 of the IL1RAP x CD3 bispecific antibody or bispecific binding fragment is provided. Vectors comprising the described polynucleotides are also provided, as are cells expressing the IL1RAP x CD3-multispecific antibodies provided herein. Also described are cells capable of expressing the disclosed vectors. These cells may be mammalian cells (such as HEK-293F cells, CHO-K1 cells), insect cells (such as Sf7 cells), yeast cells, plant cells, or bacteria cells (such as E. coli). The described antibodies may also be produced by hybridoma cells. In some embodiments, methods for generating the IL1RAP x CD3 bispecific antibody or bispecific binding fragment by culturing cells is provided.

Further provided herein are pharmaceutical compositions comprising the IL1RAP x CD3 multispecific antibodies or antigen-binding fragments and a pharmaceutically acceptable carrier.

Methods of Using IL1RAP x CD3-Multispecific Antibodies

Methods of using the described IL1RAP x CD3-multispecific antibodies and multispecific antigen-binding fragments thereof are also disclosed. For example, the IL1RAP x CD3-multispecific antibodies and multispecific antigen-binding fragments thereof may be useful in the treatment of an IL1RAP-expressing cancer in a subject in need thereof. In some embodiments, the IL1RAP-expressing cancer is a hematological cancer, such as acute myeloid leukemia (AML) myelodysplastic syndrome (MDS, low, intermediate, or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas.

The described methods of treating IL1RAP-expressing cancer in a subject in need thereof include administering to the subject a therapeutically effective amount of a described IL1RAP x CD3-multispecific antibody or multispecific antigen-binding fragment thereof. In some embodiments, the subject is a mammal, preferably a human. In preferred embodiments are provided methods for treating a subject having cancer by administering a therapeutically effective amount of the IL1RAP x CD3 bispecific antibody or bispecific antigen-binding fragment to a patient in need thereof for a time sufficient to treat the cancer.

Further provided herein are methods for inhibiting growth or proliferation of cancer cells by administering a therapeutically effective amount of the IL1RAP x CD3 bispecific antibody or bispecific binding fragment to inhibit the growth or proliferation of cancer cells.

Also provided herein are methods of redirecting a T cell to an IL1RAP-expressing cancer cell by administering a therapeutically effective amount of the IL1RAP x CD3 bispecific antibody or bispecific binding fragment to redirect a T cell to a cancer.

IL1RAP x CD3-Specific Antibody Kits

Described herein are kits including the disclosed IL1RAP x CD3-multispecific antibodies. The described kits may be used to carry out the methods of using the IL1RAP x CD3-multispecific antibodies provided herein, or other methods known to those skilled in the art. In some embodiments the described kits may include the antibodies described herein and reagents for use in treating an IL1RAP-expressing cancer. Accordingly, the described kits may include one or more of the multispecific antibodies, or a multispecific antigen-binding fragment(s) thereof, described herein and a vessel for containing the antibody or fragment when not in use, and/or instructions for use of the antibody or fragment, the antibody or fragment affixed to a solid support, and/or detectably labeled forms of the antibody or fragment, as described herein.

### BRIEF DESCRIPTION OF THE DRAWINGS

FIG. 1. pDisplay vector used for cloning IL1RAP extracellular domains.

FIGS. 2A, 2B, 2C, 2D, 2E and 2F. Supernatants resulting from the IL1RAP phage display and OMT-1 hybridomas were screened for agonist or antagonist activity (addition of exogenous recombinant human IL-1β) in HEK-Blue™ IL-1 reporter cells. Values are presented as raw optical density (OD @ 650 nm) units of an average of three reads per sample.

FIGS. 3A, 3B, 3C and 3D. IAPB57 epitope location and interactions between IL1RAP and IAPB57. (FIG. 3A) Overview of the epitope location. IAPB57 binds to the D2 and D3 domains of IL1RAP (black regions). (FIG. 3B) 2D Interaction map between IL1RAP and IAPB57. Residues from all CDRs except CDR-L1 and -L2 contact IL1RAP. Van der Waals interactions are shown as dashed lines, H-bonds are solid lines with arrows indicating backbone H bonds and pointing to the backbone atoms. IL1RAP, LC and HC residues are in gray boxes, white boxes and ovals, respectively. A distance cut-off of 4 Å was used to identify the contact residues. (C, D) Close view of IL1RAP main interactions with the Fab Light (FIG. 3C) and Heavy (FIG. 4D) Chains. H-bonds are shown as dashed lines.

FIG. 4. Epitope and paratope residues of IAPB57. The epitope residues are underlined in the IL1RAP isoforms with differences in sequences shown as shaded regions. Only the extracellular region of isoforms 1 and 4 is shown. The paratope residues are shaded and the CDR regions are underlined (Kabat definition).

FIG. 5. Competition profiles for epitope groups: Members of any one epitope group have the same competition profile. In the Venn diagram, if epitope groups overlap, they compete. Otherwise, they do not compete for human IL1RAP.

FIGS. 6A and 6B. A representative data set for the IL1RAP x CD3 bispecific antibody mediated T-cell killing assays using MV4-11 AML cells: (6A) for the first nine IL1RAPxCD3 bispecific antibodies, and for the remaining 6 bispecific IL1RAP x CD3 bispecific antibodies. IL1RAP negative/low cell line was (SU-DHL-10) and control data was also obtained (not shown). The assay was run with pan human T-cells (donor D103) at an E:T ratio of 5:1 with increasing concentrations of antibody.

FIGS. 7A and 7B. The NF-κB signaling assessment: (7A) IC3B18, IC3B19, and respective null arm bispecific control antibodies (IAPB100, IAPB101, and CNTO 7008) were analyzed for antagonist activity in the presence of exogenous recombinant human IL-1β in HEK-Blue™ IL-1 reporter cells. (7B) IC3B18, IC3B19, and respective null arm bispecific control antibodies (IAPB100, IAPB101, and CNTO 7008) were analyzed for agonistic activity in the absence of exogenous recombinant human IL-1β (0.1 ng/mL) in HEK-Blue™ IL-1 reporter cells. All data are presented as percent of control from an average of 3 reads per sample.

FIGS. 8A, 8B, 8C, 8D and 8E. IL1RAPxCD3 T-cell mediated cytotoxicity assays. IL1RAP x CD3 bispecific antibodies using anti-CD3 arm CD3B219 were incubated with human pan T cells and either an IL1RAP+AML cell line (8A, 8B, 8C and 8D) or an IL1RAP negative/low B cell lymphoma cell line (8E) line acquired from cell banking services. After 48 hours at 37° C., 5% CO2, total tumor cell cytotoxicity was measured by flow cytometry.

FIG. 9. Summary of the EC₅₀ values for four cell lines examined.

FIG. 10. Ex vivo assessment of IC3B18- and IC3B19-mediated cytotoxicity of isolated autologous normal healthy human CD14⁺ monocytes and CD3⁺ T-cells. The graph shows the percent of CD14⁺ monocytes cytotoxicity of IC3B18, IC3B19, CNTO 7008 (Null x CD3), IAPB100 (IAPB63xB23B49), and IAPB101 (IAPB57xB23B49) bispecific antibodies.

FIGS. 11A and 11B. Ex vivo assessment of IC3B18 and IC3B19 cytotoxicity of SKNO-1 cells exogenously added to normal healthy human whole blood (Donor 27067): percent of cytotoxicity SKNO-1 cells using IC3B18 and IC3B19 (IL1RAP x CD3) and CNTO 7008 (Null x CD3) bispecific antibodies at 24 hours (11A) and 48 hours (11B) time points.

FIGS. 12A, 12B, 12C, 12D and 12E. Ex vivo assessment of IC3B18 and IC3B19 cytotoxicity of blasts and T-cell activation in fresh AML donor whole blood: (12A) shows the percent of total cell cytotoxicity of AML cells using IC3B18 and IC3B19, CNTO 7008 (Null x CD3), and IAPB100 or IAPB101 (IL1RAP x Null) bispecific antibodies; (12B) shows T-cell activation induced by IC3B18 and IC3B19, CNTO 7008 and IAPB100 and IAPB101 bispecific antibodies. No Fc blocker was added. (12C) IC3B19 elicits IL1RAP⁺ specific cell cytotoxicity of primary AML IL1RAP⁺ blasts. Control antibodies IAPB101 (12D) and CNTO 7008 (12E) do not induce cytotoxicity.

FIGS. 13A and 13B. IC3B19 Mediated Cytotoxicity of OCI-AML5 Cells in Normal Healthy Human Whole Blood.

FIGS. 14A, 14B, 14C, 14D and 14E. Representative data for IL1RAPxCD3 bispecific antibodies IC3B18 and IC3B19 were tested for binding to (13A) HEK-293F parental, (13B) HEK-293F Human HE2, (13C) HEK-293F Cyno CB8, (13D) HEK-293F Mouse clone 5, and (13E) HEK-293F Rat clone 1 IL1RAP FL ECD cell lines. Values are presented as MSD light units from an average of duplicate reads per sample tested.

FIG. 15. Tumorigenesis Prevention of OCI-AML5 Human AML Xenografts Treated with IC3B19 in PBMC-Humanized NSG Mice. NSG mice were intravenously engrafted with human PBMCs, seven days later subcutaneously inoculated with OCI-AML5 cells and intravenously dosed with IC3B19 at 0.0005 mg/kg, 0.005 mg/kg, 0.05 mg/kg, and 0.5 mg/kg on Days 0, 3, 5, 7 and 10 (indicated by the arrows). SC tumors were measured twice weekly and the results presented as the average tumor volume, expressed in mm3±standard error of the mean (SEM), of each group.

FIG. 16. Tumorigenesis Prevention of MOLM-13 Human AML Xenografts Treated with IC3B19 in PBMC-Humanized NSG Mice. NSG mice were intravenously engrafted with human PBMCs, seven days later subcutaneously inoculated with MOLM-13 cells then dosed intravenously with IC3B19 at 0.0005 mg/kg, 0.005 mg/kg, 0.05 mg/kg, and 0.5 mg/kg on Days 0, 2, 5, 7, and 9 (indicated by arrows). SC tumors were measured twice weekly and the results presented as the average tumor volume, expressed in mm3±standard error of the mean (SEM), of each group.

FIG. 17. Tumorigenesis Prevention of MOLM-13 Human AML Xenografts Treated with IC3B18 and IC3B19 in PBMC-Humanized NSG Mice. NSG mice were intravenously engrafted with human PBMCs then seven days later subcutaneously inoculated with MOLM-13 cells then dosed intravenously with IC3B18 or IC3B19 at 0.005 mg/kg, 0.05 mg/kg, and 0.5 mg/kg on Days 0, 2, 4, 7, and 9 (indicated by arrows). SC tumors were measured twice weekly and the results presented as the average tumor volume, expressed in mm3±standard error of the mean (SEM), of each group.

FIG. 18. Anti-Tumor Efficacy IC3B19 in OCI-AML5 Human AML Xenografts in PBMC Humanized NSG Mice. NSG mice were subcutaneously inoculated with OCI-AML5 cells, and then intravenously engrafted with human PBMCs when tumors were established (mean tumor volume=93.7 mm³). Mice were then intravenously dosed with IC3B19 at 0.0005 mg/kg, 0.005 mg/kg, 0.05 m/kg, and 0.5 mg/kg on Days 28, 31, 33, 35, and 38 (indicated by black arrows) or IC3B19 at 0.05 mg/kg and 0.5 mg/kg on Days 31, 33, 35, 38, 40, 47, and 54 (indicated by gray arrows). SC tumors were measured twice weekly and the results presented as the average tumor volume, expressed in mm3±standard error of the mean (SEM), of each group.

FIG. 19. Anti-Tumor Efficacy IC3B18 and IC3B19 in OCI-AML5 Human AML Xenografts in PBMC-Humanized NSG Mice Comparing Treatment Initiated on Day 31 versus Day 35. NSG mice were subcutaneously inoculated with OCI-AML5 cells, and then intravenously engrafted with human PBMCs when tumors were established (mean tumor volume=111.5 mm³). On Day 31, seven groups were intravenously dosed with PBS, IC3B18, or IC3B19 at 0.05 mg/kg, 0.5 mg/kg, and 1 mg/kg on Days 31, 33, 35, 38, and 40 (indicated by black arrows). Additionally, on Day 35, four groups were intravenously dosed with IC3B18 or IC3B19 at 0.5 mg/kg and 1 mg/kg on Days 35, 38, 41, 42 and 46 (indicated by gray arrows). SC tumors were measured twice weekly and the results presented as the average tumor volume, expressed in mm3±standard error of the mean (SEM), of each group.

FIG. 20 Anti-Tumor Efficacy IC3B19 in SKNO-1 Xenografts in PBMC-Humanized NSG Mice. NSG mice were subcutaneously inoculated with SKNO-1 tumor fragments via trocar implantation and when tumors were established (mean tumor volume=135.0 mm³) randomized into treatment groups and intravenously inoculated with human PBMCs. On Day 57, animals were intravenously dosed with PBS or IC3B19 at 0.5 mg/kg, administered on Days 57, 60, 62, 64, and 67 post-tumor implantation (indicated by arrows). SC tumors were measured twice weekly and the results presented as the average tumor volume, expressed in mm3±(SEM), of each group.

FIGS. 21A, 21B, 21C, 21D and 21E. Binding competition to the human Fc ligands FcγRI, FcγRIIa, FcγRIIb, FcγRIIIa, and FcRn measured for IC3B18 and IC3B19 relative to wild type hIgG1, hIgG4 PAA isotype, and a collection of related IgG4 PAA parental (bivalent) and null-arm (monovalent) control antibodies as determined by the AlphaScreen™ assay described in Example 23. FIG. 20A) FcγRI competition. FIG. 20B) FcγRIIa competition. FIG. 20C. FcγRIIb competition. FIG. 20D) FcγRIIIa competition. FIG. 1E) FcRn competition.

FIG. 22. Anti-Tumor Efficacy of IC3B19 in SKNO-1 Human AML Xenografts in T Cell Humanized NSG Mice. NSG mice were sc inoculated with SKNO-1 AML tumor fragments on Day 0, and then ip engrafted with human T cells on Day 34. Mice were iv dosed with IC3B19 at 0.5 or 1 mg/kg on Days 35, 37, 39, 41, 43, 46, 48, 50, 53, 55 (arrows). Sc tumors were measured twice weekly and the results presented as the average tumor volume, expressed in mm³±(SEM), of each group. Only data through Day 60 post-implantation is graphically represented due to subsequent loss of multiple animals per group, due to reaching maximal tumor size limits. Key: AML=acute myeloid leukemia; NSG=NOD scid gamma (NOD.Cg-Prkdc^{scid} Il2rg^{tm1/Wjl}/SzJ); PBS phosphate buffered saline; iv=intravenous, sc=subcutaneous; ip=intraperitoneal; SEM=standard error of the mean

FIG. 23. Efficacy of IC3B19 in Disseminated MOLM-13 Luciferase Human AML Model in T Cell Humanized NSG Mice. Note: NSG mice were iv inoculated with MOLM-13 luciferase AML cells on Day 0, and then ip engrafted with human T cells on Day 3. Mice were ip dosed with IC3B19 at 0.05, 0.5 or 1 mg/kg q3d-q4d on Days 4, 8, 11, 14, 17, 21, 24, 28, 31, 35, and 38 for a total of 11 doses. Animals were euthanized due to hind limb paralysis, morbidity or excessive palpable tumor burden and survival proportions were plotted. Only data through Day 46 post-implantation is graphically represented due to subsequent loss of animals from GvHD-related morbidity. Key: AML=acute myeloid leukemia; NSG=NOD scid gamma (NOD.Cg-Prkdc^{scid} Il2rg^{tm1Wjl}/SzJ); iv=intravenous; ip=intraperitoneal; GvHD=graft vs. host disease

FIG. 24. Boxplots summarizing the transformed distribution of RNA Expression for IL1RAP. The top boxplot for each histology represent solid tissue normal and the bottom boxplot represents expression values in the tumor.

FIGS. 25A, 25B, 25C, 25D, 25E, 25F and 25G. IC3B19 stimulates a T-cell directed apoptotic response characterized by an increase in caspase activity in solid tumor lines shown here (A, B, D-G), but not in (C). The following solid tumor cancer types are represented: (A) NSCLC-Adenocarcinoma, (B) NSCLC-Squamous Cell Carcinoma, (C) NSCLC-Squamous Cell Carcinoma (D) Small Cell Lung Cancer, (E) Colon Cancer, (F) Pancreatic Cancer, (G) Prostate Cancer. Each point (n=8)±SEM for area under the curve calculated in Graphpad Prism 6.02 based on raw values at 72 hours for total green object area (μm²/well) metric with the T-cells excluded by size within the IncuCyte™ imager processing definition. Each curve represents Donor#M6807, LS-11-53847A in FIGS. 24 A, C, E, F, and G, while Donor#M7267, Lot#LS-11-53072B is shown in FIGS. 24 B, D.

FIGS. 26A, 26B and 26C. (A) IL1RAP Bispecific Abs IC3B19 elicit IL1RAP⁺ specific cell cytotoxicity of CML cell lines. Control antibodies IAPB101 (B) and CNTO 7008 (C) do not induce cytotoxicity.

FIGS. 27A, 27B and 27C. (A) IL1RAP Bispecific Abs IC3B19 elicit IL1RAP⁺ specific cell cytotoxicity of T-cell leukemia and lymphoma cell lines. Control antibodies IAPB101 (B) and CNTO 7008 (C) do not induce cytotoxicity.

FIGS. 28A, 28B and 28C. (A) IL1RAP Bispecific Abs IC3B19 elicit IL1RAP⁺ specific cell cytotoxicity of DLBCL cell line U-2940. Control antibodies IAPB101 (B) and CNTO 7008 (C) do not induce cytotoxicity.

FIG. 29. Anti-tumor efficacy of IC3B19 in H1975 human non-small cell lung carcinoma xenografts in T cell humanized NSG mice. NSG mice were sc inoculated with 1e6 H1975 human non-small cell lung carcinoma cells on Day 0, and then ip engrafted with human T cells on Day 13. Mice were ip dosed with IC3B19 at 0.5 mg/kg, 1 mg/kg or 2.5 mg/kg on days 14, 17, 20, 23, 27, 30, 35, and 38 for a total of 8 doses (arrows). Sc tumors were measured twice weekly and the results presented as the average tumor volume, expressed in mm³±(SEM), of each group. Only data through Day 30 post-implantation is graphically represented due to subsequent loss of multiple animals per group, due to reaching maximal tumor size limits. Key: AML=acute myeloid leukemia; NSG=NOD scid gamma (NOD.Cg-Prkdc^{scid} Il2rg^{tm1Wjl}/SzJ); PBS phosphate buffered saline; iv=intravenous, sc=subcutaneous; ip=intraperitoneal; SEM=standard error of the mean

FIG. 30. Ex-vivo assay IL1RAP x CD3 mediated depletion of mMDSC: Fresh Whole blood non-small cell lung cancer (NSCLC)/Prostate Cancer (PC).

FIGS. 31A, 31B, 31C, 31D and 31E. In-house MDSC gating strategy and quantification of MDSC population Fresh Whole blood. Evaluation of MDSCs population in primary Fresh Whole blood non-small cell lung cancer (NSCLC)/Prostate Cancer (PC). Representative plots showing gating strategy for MDSCs population: (A) Total nucleated cells which are viable (B) HLA-DR low/lineage markers negative (C) CD33+/CD11b+/CD15+/CD14+ MDSC population (D) CD33+/CD11b+/CD14+IL1RAP+M-MDSC (E) CD33+/CD11b+/CD15+IL1RAP+G-MDSC. All gated MDSC express IL1RAP as shown in the representative plots.

FIGS. 32A and 32B. MDSC levels variable in donor blood samples across tumors. (A) Evaluation of MDSCs population prevalence in primary Fresh Whole blood non-small cell lung cancer (NSCLC)/Prostate Cancer (PC) and (B) quantifying MDSC+IL1RAP+ receptor density comparing to healthy normal.

FIG. 33. Number of tubular networks per unit of area as a function of time in response to pro-angiogenic and anti-angiogenic treatments. Fluorescently labeled HUVEC cells were cultured on glass in the presence of VEGF to stimulate tubular elongation and branching. Suramin was added to over-ride the effect of VEGF and to prevent network expansion. The data represent the mean±SEM of three technical replicates from one experiment. Images from the first 24 hours are missing for technical reasons.

FIGS. 34A and 34B. Number of tubular networks per unit of area as a function of time in response to co-culture with healthy donor T cells (M2550), cancer cells, H1975 (A) and OCI-AML5 (B), or a combination of T cells and cancer cells. Fluorescently labeled HUVEC cells were cultured on glass in the presence of VEGF to stimulate tubular elongation and branching. The data represent the mean±SEM of three technical replicates from one experiment. Images from the first 24 hours are missing for technical reasons.

FIGS. 35A, 35B and 35C. T cells isolated from healthy volunteers (A), and H1975 (B) and OCI-AML5 (C) cell lines were stained from IL1RAP (gray line) or corresponding isotype (black line) and analyzed by flow cytometry. Percent IL1RAP-positive cells is indicated on the plots.

FIG. 36. HUVEC cultured on glass in the presence of NHDF and the indicated treatment conditions showed some expression of IL1RAP.

FIGS. 37A and 37B. Number of tubular networks per unit of area as a function of time in response to co-culture with healthy donor T cells (M2550), cancer cells, H1975 (A) and OCI-AML5 (B) in the presence of 10 nM IL1RAPxCD3 (red circles), 10 nM NullxCD3 (green triangles) or vehicle PBS (blue squares). Fluorescently labeled HUVEC cells were cultured on glass in the presence of VEGF to stimulate tubular elongation and branching. Subsequently, the cultured cells were subjected to the pharmacological treatments (indicated by the dashed lines) and network density was measured over the next 4 days. Only 10 nM dose treatment is shown. The data represent the mean±SEM of three technical replicates from one experiment. Images from the first 24 hours are missing for technical reasons.

FIG. 38. The effect of IL1RAPxCD3 on the tubular network in the presence of H1975 tumor cells and T cells, 72 hours post antibody treatment. Vehicle control (A), NullxCD3 (B) and IL1RAPxCD3 (C) treatment conditions are shown. The corresponding network masks (D, E and F) were generated by the IncuCyte™ ZOOM software. Images from one well of three technical replicates are shown. Scale bar is 500 μm.

FIGS. 39A, 39B, 39C and 39D. The effect of IL1RAPxCD3 on T cell activation the presence of cancer cells and HUVEC culture. T cells were cultured with HUVEC and H1975 tumor cells (A and B) or OCI-AML5 cells (C and D) for 4 days and analyzed by flow for CD25 expression (A and C) or IL1RAP expression (B and D). IL1RAPxCD3 bispecific antibody and NullxCD3 control were used for comparative analysis. Select conditions are shown to convey the general pattern of activation and IL1RAP expression on T cells.

FIGS. 40A, 40B, 40C and 40D. The effect of IL1RAPxCD3 on T cell surface marker expression in the presence of cancer cells and HUVEC culture. T cells were cultured with HUVEC and H1975 tumor cells (A and B) or OCI-AML5 cells (C and D) for 4 days and analyzed by flow for CD25 expression and IL1RAP expression. IL1RAPxCD3 bispecific antibody (A and C) and NullxCD3 control (B and D) were used for comparative analysis. Select conditions are shown to convey the general pattern of activation and IL1RAP expression on T cells.

FIG. 41. Cell surface expression of IL1RAP on AML and MDS blast cells were evaluated by flow cytometry on Day 0 of treatment. Cells were gated on a leukemic blasts and the expression of IL1RAP (light gray) was compared to an isotype control (dark gray).

FIGS. 42A, 42B, 42C and 42D. Ex vivo assessment of IL1RAP x CD3 mediated T cell activation and blasts depletion in primary AML sample (MT0034) in co-culture system with a human stroma cell line HS-5. T cell activation and depletion of blasts were measured by flow cytometry. (A) Graph shows percent of CD8+ T cells within population of CD45+ cells with and without IL1RAP x CD3 treatment. (B) Percent of CD4+ T cells within population of CD45+ cells. (C) Plots show activation of CD8+ and CD4+ T cells in sample treated with IL1RAP x CD3 antibody. Activation is demonstrated by expression of CD25 marker on both T cell populations. (D) Graph demonstrates depletion of AML blasts induced by IL1RAP x CD3 treatment by comparing percent of blasts within CD45+ population of cells.

FIGS. 43A, 43B, 43C, 43D, 43E, 43F, 43G and 4311. Ex vivo assessment of IL1RAP x CD3 mediated T cell activation and blast depletion of primary MDS samples (MDS_4332 and MDS_4954) in co-culture system with a human stroma cells line HS-5. T cell activation and depletion of blasts were measured by flow cytometry. (A) and (E) Graphs show percent of CD8+ T cells within population of CD45+ cells with and without IL1RAP x CD3 treatment in MDS samples 4332 and 4954 respectively. (B) and (F) Percent of CD4+ T cells within population of CD45+ cells in MDS samples 4332 and 4954. (C) and (G) Plots show activation of CD8+ and CD4+ T cells in sample treated with IL1RAP x CD3 Ab. Activation is demonstrated by expression of CD25 marker on both T cell populations. (D) and (H) Graphs demonstrate depletion of MDS blasts induced by IL1RAP x CD3 treatment by comparing percent of blasts within CD45+ population of cells.

FIGS. 44A, 44B, 44C and 44D. Ex vivo assessment of IL1RAP x CD3 mediated T cell activation and blasts depletion in primary AML sample AML_5503 in co-culture system with a human stroma cells line HS-5. T cell activation and depletion of blasts were measured by flow cytometry. (A) Graph shows decrease in percent of CD8+ T cells within population of CD45+ cells during the culture in all treatment groups. (B) Percent of CD4+ T cells within population of CD45+ cells. (C) Plots show activation of CD8+ and CD4+ T cells in the sample treated with IL1RAP x CD3 Ab; however, the number of CD8+ cells is very low and there are no CD4+ cells present in the culture. Activation is demonstrated by expression of CD25 on both T cell populations. (D) Graph demonstrates lack of depletion of AML blasts induced by IL1RAP x CD3 treatment by comparing percent of blasts within CD45+ population of cells.

FIGS. 45A, 45B, 45C, 45D and 45E. Evaluation of MDSCs population in primary AML and MDS samples. (A) Representative plots showing gating strategy for MDSCs population: HLA-DR low/lineage markers negative/CD33+/CD11b+/CD15+/CD14−. All gated MDSC express IL1RAP as shown in the representative plot on the right. (B) In samples responsive to the treatment, IL1RAP x CD3 treated samples have a significantly lower level of MDSCs comparing to the samples treated with control Ab or untreated cells. AML 5503 was a non-responsive sample that had a relatively low level of MDSCs and equal in all treatment groups.

### DETAILED DESCRIPTION OF ILLUSTRATIVE EMBODIMENTS

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to IL1RAP is substantially free of antibodies that specifically bind antigens other than IL1RAP). An isolated antibody that specifically binds to an epitope, isoform or variant of IL1RAP may, however, have cross-reactivity to other related antigens, for instance from other species (such as IL1RAP species homologs).

The term “recombinant antibody” is used to describe an antibody produced by any process involving the use of recombinant DNA technology, including any analogs of natural immunoglobulins or their fragments.

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The meaning of “substantially the same” can differ depending on the context in which the term is used. Because of the natural sequence variation likely to exist among heavy and light chains and the genes encoding them, one would expect to find some level of variation within the amino acid sequences or the genes encoding the antibodies or antigen-binding fragments described herein, with little or no impact on their unique binding properties (e.g., specificity and affinity). Such an expectation is due in part to the degeneracy of the genetic code, as well as to the evolutionary success of conservative amino acid sequence variations, which do not appreciably alter the nature of the encoded protein. Accordingly, in the context of nucleic acid sequences, “substantially the same” means at least 65% identity between two or more sequences. Preferably, the term refers to at least 70% identity between two or more sequences, more preferably at least 75% identity, more preferably at least 80% identity, more preferably at least 85% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, and more preferably at least 99% or greater identity. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

The degree of variation that may occur within the amino acid sequence of a protein without having a substantial effect on protein function is much lower than that of a nucleic acid sequence, since the same degeneracy principles do not apply to amino acid sequences. Accordingly, in the context of an antibody or antigen-binding fragment, “substantially the same” means antibodies or antigen-binding fragments having 90%, 91%, 92%, 93%, 94%, 95%, 96%, 97%, 98%, or 99% identity to the antibodies or antigen-binding fragments described. Other embodiments include IL1RAP specific antibodies, or antigen-binding fragments, that have framework, scaffold, or other non-binding regions that do not share significant identity with the antibodies and antigen-binding fragments described herein, but do incorporate one or more CDRs or other sequences needed to confer binding that are 90%, 91%, 92%, 93%, 94%, 95%, 96%, 97%, 98%, or 99% identical to such sequences described herein. A “vector” is a replicon, such as plasmid, phage, cosmid, or virus in which another nucleic acid segment may be operably inserted so as to bring about the replication or expression of the segment.

A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression or production of an antibody or antigen-binding fragment thereof may be within the cytoplasm of the cell, or into the extracellular milieu such as the growth medium of a cell culture.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

An “effective amount” or “therapeutically effective amount” refers to an amount effective, at dosages and for periods of time necessary, to achieve a desired therapeutic result. A therapeutically effective amount of an IL1RAP x CD3 antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

“Antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

“Antigen-binding fragments” are any proteinaceous structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment, which includes the V_{H} and C_{H1} domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 November; 21(11):484-90); camelid or nanobodies (Revets et al; Expert Opin Biol Ther. 2005 January; 5(1):111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof” may be used to denote that a given antigen-binding fragment incorporates one or more amino acid segments of the antibody referred to in the phrase. When used herein in the context of two or more antibodies or antigen-binding fragments, the term “competes with” or “cross-competes with” indicates that the two or more antibodies or antigen-binding fragments compete for binding to IL1RAP, e.g. compete for IL1RAP binding in the assay described in Example 11. For some pairs of antibodies or antigen-binding fragments, competition or blocking in the assay of the Examples is only observed when one antibody is coated on the plate and the other is used to compete, and not vice versa. Unless otherwise defined or negated by context, the terms “competes with” or “cross-competes with” when used herein is also intended to cover such pairs of antibodies or antigen-binding fragments.

The term “epitope” means a protein determinant capable of specific binding to an antibody. Epitopes usually consist of surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specific antigen binding peptide (in other words, the amino acid residue is within the footprint of the specifically antigen binding peptide).

“Specific binding” or “immunospecific binding” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell binding assay. Phrases such as “[antigen]-specific” antibody (e.g., IL1RAP-specific antibody) are meant to convey that the recited antibody specifically binds the recited antigen.

The term “k_{d}” (sec⁻¹), as used herein, refers to the dissociation rate constant of a particular antibody-antigen interaction. Said value is also referred to as the k_{off} value.

The term “kₐ” (M⁻¹ sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

The term “redirect” or “redirecting” as used herein refers to the ability of the IL1RAP x CD3 antibody to traffic the activity of T cells effectively, from its inherent cognate specificity toward reactivity against IL1RAP-expressing cells.

The term “sample” as used herein refers to a collection of similar fluids, cells, or tissues (e.g., surgically resected tumor tissue, biopsies, including fine needle aspiration), isolated from a subject, as well as fluids, cells, or tissues present within a subject. In some embodiments the sample is a biological fluid. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage and the like. Biological fluids may also include liquid solutions contacted with a subject or biological source, for example, cell and organ culture medium including cell or organ conditioned medium, lavage fluids and the like. The term “sample,” as used herein, encompasses materials removed from a subject or materials present in a subject.

A “known standard” may be a solution having a known amount or concentration of IL1RAP, where the solution may be a naturally occurring solution, such as a sample from a patient known to have early, moderate, late, progressive, or static cancer, or the solution may be a synthetic solution such as buffered water having a known amount of IL1RAP diluted therein. The known standards, described herein may include IL1RAP isolated from a subject, recombinant or purified IL1RAP protein, or a value of IL1RAP concentration associated with a disease condition.

The term “CD3” refers to the human CD3 protein multi-subunit complex. The CD3 protein multi-subunit complex is composed to 6 distinctive polypeptide chains. These include a CD3γ chain (SwissProt P09693), a CD3δ chain (SwissProt P04234), two CD3c chains (SwissProt P07766), and one CD3 ζ chain homodimer (SwissProt 20963), and which is associated with the T cell receptor a and chain. The term “CD3” includes any CD3 variant, isoform and species homolog which is naturally expressed by cells (including T cells) or can be expressed on cells transfected with genes or cDNA encoding those polypeptides, unless noted.

As used herein, the terms “interleukin-1 receptor accessory protein”, “IL1RAP” and “IL1-RAP” we specifically include the human IL1RAP protein, for example as described in GenBank Accession No. AAB84059, NCBI Reference Sequence: NP_002173.1 and UniProtKB/SwissProt Accession No. Q9NPH3-1 (see also Huang et al., 1997, Proc. Natl. Acad. Sci. USA. 94 (24), 12829-12832). IL1RAP is also known in the scientific literature as IL1 R3, C3orf13, FLJ37788, IL-1 RAcP and EG3556.

An “IL1RAP x CD3 antibody” is a multispecific antibody, optionally a bispecific antibody, which comprises two different antigen-binding regions, one of which binds specifically to the antigen IL1RAP and one of which binds specifically to CD3. A multispecific antibody can be a bispecific antibody, diabody, or similar molecule (see for instance PNAS USA 90(14), 6444-8 (1993) for a description of diabodies). The bispecific antibodies, diabodies, and the like, provided herein may bind any suitable target in addition to a portion of IL1RAP. The term “bispecific antibody” is to be understood as an antibody having two different antigen-binding regions defined by different antibody sequences. This can be understood as different target binding but includes as well binding to different epitopes in one target.

A “reference sample” is a sample that may be compared against another sample, such as a test sample, to allow for characterization of the compared sample. The reference sample will have some characterized property that serves as the basis for comparison with the test sample. For instance, a reference sample may be used as a benchmark for IL1RAP levels that are indicative of a subject having cancer. The reference sample does not necessarily have to be analyzed in parallel with the test sample, thus in some instances the reference sample may be a numerical value or range previously determined to characterize a given condition, such as IL1RAP levels that are indicative of cancer in a subject. The term also includes samples used for comparative purposes that are known to be associated with a physiologic state or disease condition, such as IL1RAP-expressing cancer, but that have an unknown amount of IL1RAP.

The term “progression,” as used in the context of progression of IL1RAP-expressing cancer, includes the change of a cancer from a less severe to a more severe state. This may include an increase in the number or severity of tumors, the degree of metastasis, the speed with which the cancer is growing or spreading, and the like. For example, “the progression of colon cancer” includes the progression of such a cancer from a less severe to a more severe state, such as the progression from stage I to stage II, from stage II to stage III, etc.

The term “regression,” as used in the context of regression of IL1RAP-expressing cancer, includes the change of a cancer from a more severe to a less severe state. This could include a decrease in the number or severity of tumors, the degree of metastasis, the speed with which the cancer is growing or spreading, and the like. For example, “the regression of colon cancer” includes the regression of such a cancer from a more severe to a less severe state, such as the progression from stage III to stage II, from stage II to stage I, etc.

The term “stable” as used in the context of stable IL1RAP-expressing cancer, is intended to describe a disease condition that is not, or has not, changed significantly enough over a clinically relevant period of time to be considered a progressing cancer or a regressing cancer.

The embodiments described herein are not limited to particular methods, reagents, compounds, compositions or biological systems, which can, of course, vary.

IL1RAP-Specific Antibodies and Antigen-Binding Fragments

Described herein are recombinant monoclonal antibodies or antigen-binding fragments that specifically bind IL1RAP. The general structure of an antibody molecule comprises an antigen binding domain, which includes heavy and light chains, and the Fc domain, which serves a variety of functions, including complement fixation and binding antibody receptors.

The described IL1RAP-specific antibodies or antigen-binding fragments include all isotypes, IgA, IgD, IgE, IgG and IgM, and synthetic multimers of the four-chain immunoglobulin structure. The described antibodies or antigen-binding fragments also include the IgY isotype generally found in hen or turkey serum and hen or turkey egg yolk.

The IL1RAP-specific antibodies and antigen-binding fragments may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to a human patient.

In some embodiments, the antibodies or antigen-binding fragments are chimeric. As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

In some embodiments, the antibodies are humanized antibodies. Humanized antibodies may be chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin.

The antibodies or antigen-binding fragments described herein can occur in a variety of forms, but will include one or more of the antibody CDRs shown in Table 1.

Described herein are recombinant antibodies and antigen-binding fragments that specifically bind to IL1RAP. In some embodiments, the IL1RAP-specific antibodies or antigen-binding fragments are human IgG, or derivatives thereof. While the IL1RAP-specific antibodies or antigen-binding fragments exemplified herein are human, the antibodies or antigen-binding fragments exemplified may be chimerized.

In some embodiments are provided an IL1RAP-specific antibody, or an antigen-binding fragment thereof, comprising a heavy chain comprising a CDR1, a CDR2, and a CDR3 of any one of the antibodies described in Table 1. In some embodiments are provided an IL1RAP-specific antibody, or an antigen-binding fragment thereof, comprising a heavy chain comprising a CDR1, a CDR2, and a CDR3 of any one of the antibodies described in Table 1 and a light chain comprising a CDR1, a CDR2, and a CDR3 of any one of the antibodies described in Table 1.

In some embodiments, the IL1RAP-specific antibodies and antigen-binding fragments comprise a heavy chain CDR1 comprising SEQ ID NO: 10, a heavy chain CDR2 comprising SEQ ID NO: 11, and a heavy chain CDR3 comprising SEQ ID NO: 12. In some embodiments, the IL1RAP-specific antibodies and antigen-binding fragments comprise a heavy chain CDR1 comprising SEQ ID NO: 10, a heavy chain CDR2 comprising SEQ ID NO: 11, a heavy chain CDR3 comprising SEQ ID NO: 12, a light chain CDR1 comprising SEQ ID NO: 40, a light chain CDR2 comprising SEQ ID NO: 41, and a light chain CDR3 comprising SEQ ID NO: 42. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 68. In some embodiments, the IL1RAP-specific antibodies and antigen-binding fragments comprise a heavy chain variable domain substantially the same as, or identical to, SEQ ID NO: 68 and a light chain variable domain substantially the same as, or identical to, SEQ ID NO: 69. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 13, a heavy chain CDR2 comprising SEQ ID NO: 14, and a heavy chain CDR3 comprising SEQ ID NO: 15. In some embodiments, the IL1RAP-specific antibodies and antigen-binding fragments comprise a heavy chain CDR1 comprising SEQ ID NO: 13, a heavy chain CDR2 comprising SEQ ID NO: 14, a heavy chain CDR3 comprising SEQ ID NO: 15, a light chain CDR1 comprising SEQ ID NO: 43, a light chain CDR2 comprising SEQ ID NO: 44, and a light chain CDR3 comprising SEQ ID NO: 45. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 70. In some embodiments, the IL1RAP-specific antibodies and antigen-binding fragments comprise a heavy chain variable domain substantially the same as, or identical to, SEQ ID NO: 70 and a light chain variable domain substantially the same as, or identical to, SEQ ID NO: 71. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 16, a heavy chain CDR2 comprising SEQ ID NO: 17, and a heavy chain CDR3 comprising SEQ ID NO: 18. In some embodiments, the IL1RAP-specific antibodies and antigen-binding fragments comprise a heavy chain CDR1 comprising SEQ ID NO: 16, a heavy chain CDR2 comprising SEQ ID NO: 17, a heavy chain CDR3 comprising SEQ ID NO: 18, a light chain CDR1 comprising SEQ ID NO: 46, a light chain CDR2 comprising SEQ ID NO: 47, and a light chain CDR3 comprising SEQ ID NO: 103. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 72. In some embodiments, the IL1RAP-specific antibodies and antigen-binding fragments comprise a heavy chain variable domain substantially the same as, or identical to, SEQ ID NO: 72 and a light chain variable domain substantially the same as, or identical to, SEQ ID NO: 73. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 19, a heavy chain CDR2 comprising SEQ ID NO: 20, and a heavy chain CDR3 comprising SEQ ID NO: 21. In some embodiments, the IL1RAP-specific antibodies and antigen-binding fragments comprise a heavy chain CDR1 comprising SEQ ID NO: 19, a heavy chain CDR2 comprising SEQ ID NO: 20, a heavy chain CDR3 comprising SEQ ID NO: 21, a light chain CDR1 comprising SEQ ID NO: 49, a light chain CDR2 comprising SEQ ID NO: 50, and a light chain CDR3 comprising SEQ ID NO: 51. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 74. In some embodiments, the IL1RAP-specific antibodies and antigen-binding fragments comprise a heavy chain variable domain substantially the same as, or identical to, SEQ ID NO: 74 and a light chain variable domain substantially the same as, or identical to, SEQ ID NO: 75. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 22, a heavy chain CDR2 comprising SEQ ID NO: 23, and a heavy chain CDR3 comprising SEQ ID NO: 24. In some embodiments, the IL1RAP-specific antibodies and antigen-binding fragments comprise a heavy chain CDR1 comprising SEQ ID NO: 22, a heavy chain CDR2 comprising SEQ ID NO: 23, a heavy chain CDR3 comprising SEQ ID NO: 24, a light chain CDR1 comprising SEQ ID NO: 52, a light chain CDR2 comprising SEQ ID NO: 47, and a light chain CDR3 comprising SEQ ID NO: 53. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 76. In some embodiments, the IL1RAP-specific antibodies and antigen-binding fragments comprise a heavy chain variable domain substantially the same as, or identical to, SEQ ID NO: 76 and a light chain variable domain substantially the same as, or identical to, SEQ ID NO: 77. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 25, a heavy chain CDR2 comprising SEQ ID NO: 26, and a heavy chain CDR3 comprising SEQ ID NO: 27. In some embodiments, the IL1RAP-specific antibodies and antigen-binding fragments comprise a heavy chain CDR1 comprising SEQ ID NO: 25, a heavy chain CDR2 comprising SEQ ID NO: 26, a heavy chain CDR3 comprising SEQ ID NO: 27, a light chain CDR1 comprising SEQ ID NO: 54, a light chain CDR2 comprising SEQ ID NO: 55, and a light chain CDR3 comprising SEQ ID NO: 56. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 78. In some embodiments, the IL1RAP-specific antibodies and antigen-binding fragments comprise a heavy chain variable domain substantially the same as, or identical to, SEQ ID NO: 78 and a light chain variable domain substantially the same as, or identical to, SEQ ID NO: 79. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 25, a heavy chain CDR2 comprising SEQ ID NO: 28, and a heavy chain CDR3 comprising SEQ ID NO: 29. In some embodiments, the IL1RAP-specific antibodies and antigen-binding fragments comprise a heavy chain CDR1 comprising SEQ ID NO: 25, a heavy chain CDR2 comprising SEQ ID NO: 28, a heavy chain CDR3 comprising SEQ ID NO: 29, a light chain CDR1 comprising SEQ ID NO: 54, a light chain CDR2 comprising SEQ ID NO: 55, and a light chain CDR3 comprising SEQ ID NO: 56. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 80. In some embodiments, the IL1RAP-specific antibodies and antigen-binding fragments comprise a heavy chain variable domain substantially the same as, or identical to, SEQ ID NO: 80 and a light chain variable domain substantially the same as, or identical to, SEQ ID NO: 79. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 30, a heavy chain CDR2 comprising SEQ ID NO: 31, and a heavy chain CDR3 comprising SEQ ID NO: 32. In some embodiments, the IL1RAP-specific antibodies and antigen-binding fragments comprise a heavy chain CDR1 comprising SEQ ID NO: 30, a heavy chain CDR2 comprising SEQ ID NO: 31, a heavy chain CDR3 comprising SEQ ID NO: 32, a light chain CDR1 comprising SEQ ID NO: 57, a light chain CDR2 comprising SEQ ID NO: 58, and a light chain CDR3 comprising SEQ ID NO: 59. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 81. In some embodiments, the IL1RAP-specific antibodies and antigen-binding fragments comprise a heavy chain variable domain substantially the same as, or identical to, SEQ ID NO: 81 and a light chain variable domain substantially the same as, or identical to, SEQ ID NO: 82. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 33, a heavy chain CDR2 comprising SEQ ID NO: 34, and a heavy chain CDR3 comprising SEQ ID NO: 35. In some embodiments, the IL1RAP-specific antibodies and antigen-binding fragments comprise a heavy chain CDR1 comprising SEQ ID NO: 33, a heavy chain CDR2 comprising SEQ ID NO: 34, a heavy chain CDR3 comprising SEQ ID NO: 35, a light chain CDR1 comprising SEQ ID NO: 60, a light chain CDR2 comprising SEQ ID NO: 47, and a light chain CDR3 comprising SEQ ID NO: 48. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 83. In some embodiments, the IL1RAP-specific antibodies and antigen-binding fragments comprise a heavy chain variable domain substantially the same as, or identical to, SEQ ID NO: 83 and a light chain variable domain substantially the same as, or identical to, SEQ ID NO: 84. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 13, a heavy chain CDR2 comprising SEQ ID NO: 34, and a heavy chain CDR3 comprising SEQ ID NO: 36. In some embodiments, the IL1RAP-specific antibodies and antigen-binding fragments comprise a heavy chain CDR1 comprising SEQ ID NO: 13, a heavy chain CDR2 comprising SEQ ID NO: 34, a heavy chain CDR3 comprising SEQ ID NO: 36, a light chain CDR1 comprising SEQ ID NO: 60, a light chain CDR2 comprising SEQ ID NO: 47, and a light chain CDR3 comprising SEQ ID NO: 48. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 85. In some embodiments, the IL1RAP-specific antibodies and antigen-binding fragments comprise a heavy chain variable domain substantially the same as, or identical to, SEQ ID NO: 85 and a light chain variable domain substantially the same as, or identical to, SEQ ID NO: 84. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 25, a heavy chain CDR2 comprising SEQ ID NO: 37, and a heavy chain CDR3 comprising SEQ ID NO: 38. In some embodiments, the IL1RAP-specific antibodies and antigen-binding fragments comprise a heavy chain CDR1 comprising SEQ ID NO: 25, a heavy chain CDR2 comprising SEQ ID NO: 37, a heavy chain CDR3 comprising SEQ ID NO: 38, a light chain CDR1 comprising SEQ ID NO: 60, a light chain CDR2 comprising SEQ ID NO: 47, and a light chain CDR3 comprising SEQ ID NO: 48. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 86. In some embodiments, the IL1RAP-specific antibodies and antigen-binding fragments comprise a heavy chain variable domain substantially the same as, or identical to, SEQ ID NO: 86 and a light chain variable domain substantially the same as, or identical to, SEQ ID NO: 84. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 19, a heavy chain CDR2 comprising SEQ ID NO: 20, and a heavy chain CDR3 comprising SEQ ID NO: 21. In some embodiments, the IL1RAP-specific antibodies and antigen-binding fragments comprise a heavy chain CDR1 comprising SEQ ID NO: 19, a heavy chain CDR2 comprising SEQ ID NO: 20, a heavy chain CDR3 comprising SEQ ID NO: 21, a light chain CDR1 comprising SEQ ID NO: 49, a light chain CDR2 comprising SEQ ID NO: 50, and a light chain CDR3 comprising SEQ ID NO: 61. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 74. In some embodiments, the IL1RAP-specific antibodies and antigen-binding fragments comprise a heavy chain variable domain substantially the same as, or identical to, SEQ ID NO: 74 and a light chain variable domain substantially the same as, or identical to, SEQ ID NO: 87. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 22, a heavy chain CDR2 comprising SEQ ID NO: 23, and a heavy chain CDR3 comprising SEQ ID NO: 24. In some embodiments, the IL1RAP-specific antibodies and antigen-binding fragments comprise a heavy chain CDR1 comprising SEQ ID NO: 22, a heavy chain CDR2 comprising SEQ ID NO: 23, a heavy chain CDR3 comprising SEQ ID NO: 24, a light chain CDR1 comprising SEQ ID NO: 62, a light chain CDR2 comprising SEQ ID NO: 63, and a light chain CDR3 comprising SEQ ID NO: 64. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 76. In some embodiments, the IL1RAP-specific antibodies and antigen-binding fragments comprise a heavy chain variable domain substantially the same as, or identical to, SEQ ID NO: 76 and a light chain variable domain substantially the same as, or identical to, SEQ ID NO: 88. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 22, a heavy chain CDR2 comprising SEQ ID NO: 23, and a heavy chain CDR3 comprising SEQ ID NO: 24. In some embodiments, the IL1RAP-specific antibodies and antigen-binding fragments comprise a heavy chain CDR1 comprising SEQ ID NO: 22, a heavy chain CDR2 comprising SEQ ID NO: 23, a heavy chain CDR3 comprising SEQ ID NO: 24, a light chain CDR1 comprising SEQ ID NO: 62, a light chain CDR2 comprising SEQ ID NO: 63, and a light chain CDR3 comprising SEQ ID NO: 65. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 76. In some embodiments, the IL1RAP-specific antibodies and antigen-binding fragments comprise a heavy chain variable domain substantially the same as, or identical to, SEQ ID NO: 76 and a light chain variable domain substantially the same as, or identical to, SEQ ID NO: 89. The heavy chain variable domain and light chain variable domain of antibodies discussed in this paragraph are suitable for inclusion in bispecific constructs in which one arm is an anti-IL1RAP arm.

In some embodiments, the IL1RAP-specific antibodies and antigen-binding fragments comprise a heavy chain CDR1 comprising SEQ ID NO: 25, a heavy chain CDR2 comprising SEQ ID NO: 26, and a heavy chain CDR3 comprising SEQ ID NO: 39. In some embodiments, the IL1RAP-specific antibodies and antigen-binding fragments comprise a heavy chain CDR1 comprising SEQ ID NO: 25, a heavy chain CDR2 comprising SEQ ID NO: 26, a heavy chain CDR3 comprising SEQ ID NO: 39, a light chain CDR1 comprising SEQ ID NO: 66, a light chain CDR2 comprising SEQ ID NO: 50, and a light chain CDR3 comprising SEQ ID NO: 67. This IL1RAP-specific antibody or antigen-binding fragment may comprise human framework sequences. This IL1RAP-specific antibody or antigen-binding fragment may bind to IL1RAP with an affinity of 50 nM or less. In some embodiments, the IL1RAP-specific antibodies and antigen-binding fragments comprise a heavy chain variable domain substantially the same as, or identical to, SEQ ID NO: 90. In some embodiments, the IL1RAP-specific antibodies and antigen-binding fragments comprise a heavy chain variable domain substantially the same as, or identical to, SEQ ID NO: 90 and a light chain variable domain substantially the same as, or identical to, SEQ ID NO: 91. The heavy chain variable domain and light chain variable domain of antibodies discussed in this paragraph are suitable for inclusion in bispecific constructs in which one arm is an anti-IL1RAP arm.

In some embodiments, the antibodies or antigen-binding fragments are IgG, or derivatives thereof, e.g., IgG1, IgG2, IgG3, and IgG4 isotypes. In some embodiments wherein the antibody has an IgG1 isotype, the antibody contains L234A, L235A, and K409R substitution(s) in its Fc region. In some embodiments wherein the antibody has an IgG4 isotype, the antibody contains S228P, L234A, and L235A substitutions in its Fc region. The specific antibodies defined by CDR and/or variable domain sequence discussed in the above paragraphs may include these modifications.

Also disclosed are recombinant polynucleotides that encode the antibodies or antigen-binding fragments that specifically bind to IL1RAP. The recombinant polynucleotides capable of encoding the variable domain segments provided herein may be included on the same, or different, vectors to produce antibodies or antigen-binding fragments.

Polynucleotides encoding recombinant antigen-binding proteins also are within the scope of the disclosure. In some embodiments, the polynucleotides described (and the peptides they encode) include a leader sequence. Any leader sequence known in the art may be employed. The leader sequence may include, but is not limited to, a restriction site or a translation start site.

The IL1RAP-specific antibodies or antigen-binding fragments described herein include variants having single or multiple amino acid substitutions, deletions, or additions that retain the biological properties (e.g., binding affinity or immune effector activity) of the described IL1RAP-specific antibodies or antigen-binding fragments. In the context of the present invention the following notations are, unless otherwise indicated, used to describe a mutation; i) substitution of an amino acid in a given position is written as e.g. S228P which means a substitution of a Serine in position 228 with a Proline; and ii) for specific variants the specific three or one letter codes are used, including the codes Xaa and X to indicate any amino acid residue. Thus, the substitution of Serine for Proline in position 228 is designated as: S228P, or the substitution of any amino acid residue for Serine in position 228 is designated as S228X. In case of deletion of Serine in position 228 it is indicated by S228*. The skilled person may produce variants having single or multiple amino acid substitutions, deletions, or additions.

These variants may include: (a) variants in which one or more amino acid residues are substituted with conservative or non-conservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies or antigen-binding fragments described herein may include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (deletions, mutations, etc.), chemical, and enzymatic techniques, are known to persons having ordinary skill in the art.

The IL1RAP-specific antibodies or antigen-binding fragments described herein may embody several antibody isotypes, such as IgM, IgD, IgG, IgA and IgE. In some embodiments the antibody isotype is IgG1, IgG2, IgG3, or IgG4 isotype, preferably IgG1 or IgG4 isotype. Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

The IL1RAP-specific antibodies or antigen-binding fragments described herein have binding affinities for IL1RAP that include a dissociation constant (K_{D}) of less than about 50 nM. The affinity of the described IL1RAP-specific antibodies, or antigen-binding fragments, may be determined by a variety of methods known in the art, such as surface plasmon resonance or ELISA-based methods. Assays for measuring affinity include assays performed using a BIAcore 3000 machine, where the assay is performed at room temperature (e.g. at or near 25° C.), wherein the antibody capable of binding to IL1RAP is captured on the BIAcore sensor chip by an anti-Fc antibody (e.g. goat anti-human IgG Fc specific antibody Jackson ImmunoResearch laboratories Prod #109-005-098) to a level around 75 RUs, followed by the collection of association and dissociation data at a flow rate of 40 μl/min.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the antibody- or antigen-binding fragment-coding sequence is placed under control of a powerful constitutive promoter, such as the promoters for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the IL1RAP-specific antibody or antigen-binding fragment thereof coding sequence is placed under control of an inducible promoter such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate IL1RAP-specific antibody or antigen-binding fragment-producing cells. Thus, another aspect features host cells transformed with vectors comprising a nucleic acid sequence encoding an antibody or antigen-binding fragment thereof that specifically binds IL1RAP, such as the antibodies or antigen-binding fragments described and exemplified herein.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the IL1RAP-specific antibodies or antigen-binding fragments described herein are preferably eukaryotic cells, more preferably cells of plant, rodent, or human origin, for example but not limited to NSO, CHO, CHO-K1, perC.6, Tk-ts13, BHK, HEK-293 cells, COS-7, T98G, CV-1/EBNA, L cells, C127, 3T3, HeLa, NS1, Sp2/0 myeloma cells, and BHK cell lines, among others. In addition, expression of antibodies may be accomplished using hybridoma cells. Methods for producing hybridomas are well established in the art.

Cells transformed with expression vectors described herein may be selected or screened for recombinant expression of the antibodies or antigen-binding fragments described herein. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

Methods of Using IL1RAP-Specific Antibodies for Treatment

Provided herein are IL1RAP-specific antibodies or antigen-binding fragments thereof for use in therapy. In particular, these antibodies or antigen-binding fragments may be useful in treating cancer, such as IL1RAP-expressing cancer. Accordingly, the invention provides a method of treating cancer comprising administering an antibody as described herein, such as IL1RAP-specific antibodies or antigen-binding fragments. For example, the use may be 1) by interfering with IL1RAP-receptor interactions, 2) where the antibody is conjugated to a toxin, so targeting the toxin to the IL1RAP-expressing cancer, or 3) use the antibody to redirect the body's immune cells to the IL1RAP-expressing cancer cells (e.g. ADCC, T cell redirection). In some embodiments IL1RAP-expressing cancer includes hematological cancer, such as acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas. The antibodies for use in these methods include those described herein above, for example an IL1RAP-specific antibody or antigen-binding fragment with the features set out in Table 1, for example the CDRs or variable domain sequences, and in the further discussion of these antibodies.

In some embodiments described herein, immune effector properties of the IL1RAP-specific antibodies may be enhanced or silenced through Fc modifications by techniques known to those skilled in the art. For example, Fc effector functions such as Clq binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. may be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of monoclonal antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved Fc.gamma.RIIIa binding without altering antigen binding or CDC activity. Such mAbs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the .alpha. 1,6-fucosyltrasferase (FUT8) gene (Mori et al., Biotechnol Bioeng 88:901-908, 2004), or coexpression of .beta.-1,4-N-acetylglucosaminyltransferase III and Golgi.alpha.-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the IL1RAP antibodies may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

Methods of Detecting IL1RAP

Provided herein are methods for detecting IL1RAP in a biological sample by contacting the sample with an antibody, or antigen-binding fragment thereof, described herein. As described herein, the sample may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In some embodiments the described methods include detecting IL1RAP in a biological sample by contacting the sample with any of the IL1RAP-specific antibodies or antigen-binding fragments thereof described herein.

In some embodiments the sample may be contacted with more than one of the IL1RAP-specific antibodies or antigen-binding fragments described in Table 1. For example, a sample may be contacted with a first IL1RAP-specific antibody, or antigen-binding fragment thereof, and then contacted with a second IL1RAP-specific antibody, or antigen-binding fragment thereof, wherein the first antibody or antigen-binding fragment and the second antibody or antigen-binding fragment are not the same antibody or antigen-binding fragment. In some embodiments, the first antibody, or antigen-binding fragment thereof, may be affixed to a surface, such as a multiwell plate, chip, or similar substrate prior to contacting the sample. In other embodiments the first antibody, or antigen-binding fragment thereof, may not be affixed, or attached, to anything at all prior to contacting the sample. In an alternative embodiment, a sample may be contacted with an IL1RAP-specific antibody and the sample-bound IL1RAP-specific antibody may then be detected by a labeled antibody or other antibody-targeted binding agent.

In some exemplary embodiments of the methods provided in this section suitable IL1RAP-specific antibodies include antibodies having the same heavy chain CDR1, CDR2, and CDR3 and light chain CDR1, CDR2, and CDR3 combinations of any one of the following antibodies, as disclosed in Table 1: IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65.

The described IL1RAP-specific antibodies and antigen-binding fragments may be detectably labeled. In some embodiments labeled antibodies and antigen-binding fragments may facilitate the detection IL1RAP via the methods described herein. Many such labels are readily known to those skilled in the art. For example, suitable labels include, but should not be considered limited to, radiolabels, fluorescent labels, epitope tags, biotin, chromophore labels, ECL labels, or enzymes. More specifically, the described labels include ruthenium, ¹¹¹In-DOTA, ^{III}In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes, Alexafluor® dyes, and the like.

The described IL1RAP-specific antibodies and antigen-binding fragments may be used in a variety of assays to detect IL1RAP in a biological sample. Some suitable assays include, but should not be considered limited to, western blot analysis, radioimmunoassay, surface plasmon resonance, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

In some embodiments described herein detection of IL1RAP-expressing cancer cells in a subject may be used to determine that the subject may be treated with a therapeutic agent directed against IL1RAP.

IL1RAP is present at detectable levels in blood and serum samples. Thus, provided herein are methods for detecting IL1RAP in a sample derived from blood, such as a serum sample, by contacting the sample with an antibody, or antigen-binding fragment thereof, which specifically binds IL1RAP. The blood sample, or a derivative thereof, may be diluted, fractionated, or otherwise processed to yield a sample upon which the described method may be performed. In some embodiments, IL1RAP may be detected in a blood sample, or a derivative thereof, by any number of assays known in the art, such as, but not limited to, western blot analysis, radioimmunoassay, surface plasmon resonance, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

Methods for Diagnosing Cancer

Provided herein are methods for diagnosing IL1RAP-expressing cancer in a subject. In some embodiments IL1RAP-expressing cancer includes hematological cancers, such as acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas. In some embodiments, as described above, detecting IL1RAP in a biological sample, such as a blood sample or a serum sample, provides the ability to diagnose cancer in the subject from whom the sample was obtained. Alternatively, in some embodiments other samples such as a histological sample, a fine needle aspirate sample, resected tumor tissue, circulating cells, circulating tumor cells, and the like, may also be used to assess whether the subject from whom the sample was obtained has cancer. In some embodiments, it may already be known that the subject from whom the sample was obtained has cancer, but the type of cancer afflicting the subject may not yet have been diagnosed or a preliminary diagnosis may be unclear, thus detecting IL1RAP in a biological sample obtained from the subject can allow for, or clarify, diagnosis of the cancer. For example, a subject may be known to have cancer, but it may not be known, or may be unclear, whether the subject's cancer is IL1RAP-expressing.

In some embodiments the described methods involve assessing whether a subject is afflicted with IL1RAP-expressing cancer by determining the amount of IL1RAP that is present in a biological sample derived from the subject; and comparing the observed amount of IL1RAP with the amount of IL1RAP in a control, or reference, sample, wherein a difference between the amount of IL1RAP in the sample derived from the subject and the amount of IL1RAP in the control, or reference, sample is an indication that the subject is afflicted with an IL1RAP-expressing cancer. In another embodiment the amount of IL1RAP observed in a biological sample obtained from a subject may be compared to levels of IL1RAP known to be associated with certain forms or stages of cancer, to determine the form or stage of the subject's cancer. In some embodiments the amount of IL1RAP in the sample derived from the subject is assessed by contacting the sample with an antibody, or an antigen-binding fragment thereof, which specifically binds IL1RAP, such as the IL1RAP-specific antibodies described herein. The sample assessed for the presence of IL1RAP may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In some embodiments IL1RAP-expressing cancer includes hematological cancer, such as acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas. In some embodiments the subject is a human.

In some embodiments the method of diagnosing an IL1RAP-expressing cancer will involve: contacting a biological sample of a subject with an IL1RAP-specific antibody, or an antigen-binding fragment thereof (such as those derivable from the antibodies and fragments provided in Table 1), quantifying the amount of IL1RAP present in the sample that is bound by the antibody or antigen-binding fragment thereof, comparing the amount of IL1RAP present in the sample to a known standard or reference sample; and determining whether the subject's IL1RAP levels fall within the levels of IL1RAP associated with cancer. In an additional embodiment, the diagnostic method can be followed with an additional step of administering or prescribing a cancer-specific treatment. In another embodiment, the diagnostic method can be followed with an additional step of transmitting the results of the determination to facilitate treatment of the cancer. In some embodiments the cancer-specific treatment may be directed against IL1RAP-expressing cancers, such as the IL1RAP x CD3 multispecific antibodies described herein.

In some embodiments the described methods involve assessing whether a subject is afflicted with IL1RAP-expressing cancer by determining the amount of IL1RAP present in a blood or serum sample obtained from the subject; and comparing the observed amount of IL1RAP with the amount of IL1RAP in a control, or reference, sample, wherein a difference between the amount of IL1RAP in the sample derived from the subject and the amount of IL1RAP in the control, or reference, sample is an indication that the subject is afflicted with an IL1RAP-expressing cancer.

In some embodiments the control, or reference, sample may be derived from a subject that is not afflicted with IL1RAP-expressing cancer. In some embodiments the control, or reference, sample may be derived from a subject that is afflicted with IL1RAP-expressing cancer. In some embodiments where the control, or reference, sample is derived from a subject that is not afflicted with IL1RAP-expressing cancer, an observed increase in the amount of IL1RAP present in the test sample, relative to that observed for the control or reference sample, is an indication that the subject being assessed is afflicted with IL1RAP-expressing cancer. In some embodiments where the control sample is derived from a subject that is not afflicted with IL1RAP-expressing cancer, an observed decrease or similarity in the amount of IL1RAP present in the test sample, relative to that observed for the control or reference sample, is an indication that the subject being assessed is not afflicted with IL1RAP-expressing cancer. In some embodiments where the control or reference sample is derived from a subject that is afflicted with IL1RAP-expressing cancer, an observed similarity in the amount of IL1RAP present in the test sample, relative to that observed for the control or reference sample, is an indication that the subject being assessed is afflicted with IL1RAP-expressing cancer. In some embodiments where the control or reference sample is derived from a subject that is afflicted with IL1RAP-expressing cancer, an observed decrease in the amount of IL1RAP present in the test sample, relative to that observed for the control or reference sample, is an indication that the subject being assessed is not afflicted with IL1RAP-expressing cancer.

In some embodiments the amount of IL1RAP in the sample derived from the subject is assessed by contacting the sample with an antibody, or an antigen-binding fragment thereof, that specifically binds IL1RAP, such as the antibodies described herein. The sample assessed for the presence of IL1RAP may be derived from a blood sample, a serum sample, circulating cells, circulating tumor cells, cells that are not tissue associated (i.e., free cells), tissues (e.g., surgically resected tumor tissue, biopsies, including fine needle aspiration), histological preparations, and the like.

In various aspects, the amount of IL1RAP is determined by contacting the sample with an antibody, or antigen-binding fragment thereof, which specifically binds IL1RAP. In some embodiments, the sample may be contacted by more than one type of antibody, or antigen-binding fragment thereof, which specifically binds IL1RAP. In some embodiments, the sample may be contacted by a first antibody, or antigen-binding fragment thereof, which specifically binds IL1RAP and then contacted by a second antibody, or antigen-binding fragment thereof, which specifically binds IL1RAP. IL1RAP-specific antibodies or antigen-binding fragments such as those described herein may be used in this capacity.

Various combinations of the IL1RAP-specific antibodies and antigen-binding fragments can be used to provide a “first” and “second” antibody or antigen-binding fragment to carry out the described diagnostic methods. In some embodiments IL1RAP-expressing cancer includes a hematological cancer, such as acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas.

In certain embodiments, the amount of IL1RAP is determined by western blot analysis, radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

In various embodiments of the described diagnostic methods a control or reference sample is used. This sample may be a positive or negative assay control that ensures the assay used is working properly; for example, an assay control of this nature might be commonly used for immunohistochemistry assays. Alternatively, the sample may be a standardized reference for the amount of IL1RAP in a biological sample from a healthy subject. In some embodiments, the observed IL1RAP levels of the tested subject may be compared with IL1RAP levels observed in samples from subjects known to have IL1RAP-expressing cancer. In some embodiments, the control subject may be afflicted with a particular cancer of interest. In some embodiments, the control subject is known to have early stage cancer, which may or may not be IL1RAP-expressing cancer. In some embodiments, the control subject is known to have intermediate stage cancer, which may or may not be IL1RAP-expressing cancer. In some embodiments, the control subject is known to have late stage, which may or may not be IL1RAP-expressing cancer.

Methods for Monitoring Cancer

Provided herein are methods for monitoring IL1RAP-expressing cancer in a subject. In some embodiments IL1RAP-expressing cancer includes a hematological cancer, such as acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas. In some embodiments the described methods involve assessing whether IL1RAP-expressing cancer is progressing, regressing, or remaining stable by determining the amount of IL1RAP that is present in a test sample derived from the subject; and comparing the observed amount of IL1RAP with the amount of IL1RAP in a biological sample obtained, in a similar manner, from the subject at an earlier point in time, wherein a difference between the amount of IL1RAP in the test sample and the earlier sample provides an indication of whether the cancer is progressing, regressing, or remaining stable. In this regard, a test sample with an increased amount of IL1RAP, relative to the amount observed for the earlier sample, may indicate progression of an IL1RAP-expressing cancer. Conversely, a test sample with a decreased amount of IL1RAP, relative to the amount observed for the earlier sample, may indicate regression of an IL1RAP-expressing cancer.

Accordingly, a test sample with an insignificant difference in the amount of IL1RAP, relative to the amount observed for the earlier sample, may indicate a state of stable disease for an IL1RAP-expressing cancer. In some embodiments the amount of IL1RAP in a biological sample derived from the subject is assessed by contacting the sample with an antibody, or an antibody fragment thereof, which specifically binds IL1RAP, such as the antibodies described herein. The sample assessed for the presence of IL1RAP may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In some embodiments the subject is a human.

In some embodiments the methods of monitoring an IL1RAP-expressing cancer will involve: contacting a biological sample of a subject with an IL1RAP-specific antibody, or antigen-binding fragment thereof (such as those derivable from the antibodies and fragments provided in Table 1), quantifying the amount of IL1RAP present in the sample, comparing the amount of IL1RAP present in the sample to the amount of IL1RAP determined to be in a biological sample obtained, in a similar manner, from the same subject at an earlier point in time; and determining whether the subject's IL1RAP level has changed over time. A test sample with an increased amount of IL1RAP, relative to the amount observed for the earlier sample, may indicate progression of cancer. Conversely, a test sample with a decreased amount of IL1RAP, relative to the amount observed for the earlier sample, may indicate regression of an IL1RAP-expressing cancer. Accordingly, a test sample with an insignificant difference in the amount of IL1RAP, relative to the amount observed for the earlier sample, may indicate a state of stable disease for an IL1RAP-expressing cancer. In some embodiments, the IL1RAP levels of the sample may be compared to a known standard or a reference sample, alone or in addition to the IL1RAP levels observed for a sample assessed at an earlier point in time. In an additional embodiment, the diagnostic method can be followed with an additional step of administering a cancer-specific treatment. In some embodiments the cancer-specific treatment may be directed against IL1RAP-expressing cancers, such as the IL1RAP x CD3 multispecific antibodies described herein.

In various aspects, the amount of IL1RAP is determined by contacting the sample with an antibody, or antigen-binding fragment thereof, which specifically binds IL1RAP. In some embodiments, the sample may be contacted by more than one type of antibody, or antigen-binding fragment thereof, which specifically binds IL1RAP. In some embodiments, the sample may be contacted by a first antibody, or antigen-binding fragment thereof, which specifically binds IL1RAP and then contacted by a second antibody, or antigen-binding fragment thereof, which specifically binds IL1RAP. Antibodies such as those described herein may be used in this capacity.

Various combinations of the antibodies and antigen-binding fragments described in Table 1 can be used to provide a “first” and “second” antibody or antigen-binding fragment to carry out the described monitoring methods. In some embodiments IL1RAP-expressing cancer includes a hematological cancer, such as acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN). In some embodiments IL1RAP-expressing cancer includes a solid tumor, such as the following: prostate, breast, lung, colorectal, melanomas, bladder, brain/CNS, cervical, esophageal, gastric, head/neck, kidney, liver, ovarian, pancreatic, and sarcomas.

In certain embodiments, the amount of IL1RAP is determined by western blot analysis, radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

Kits for Detecting IL1RAP

Provided herein are kits for detecting IL1RAP in a biological sample. These kits include one or more of the IL1RAP-specific antibodies described herein, or an antigen-binding fragment thereof, and instructions for use of the kit.

The provided IL1RAP-specific antibody, or antigen-binding fragment, may be in solution; lyophilized; affixed to a substrate, carrier, or plate; or detectably labeled.

The described kits may also include additional components useful for performing the methods described herein. By way of example, the kits may comprise means for obtaining a sample from a subject, a control or reference sample, e.g., a sample from a subject having slowly progressing cancer and/or a subject not having cancer, one or more sample compartments, and/or instructional material which describes performance of a method of the invention and tissue specific controls or standards.

The means for determining the level of IL1RAP can further include, for example, buffers or other reagents for use in an assay for determining the level of IL1RAP. The instructions can be, for example, printed instructions for performing the assay and/or instructions for evaluating the level of expression of IL1RAP.

The described kits may also include means for isolating a sample from a subject. These means can comprise one or more items of equipment or reagents that can be used to obtain a fluid or tissue from a subject. The means for obtaining a sample from a subject may also comprise means for isolating blood components, such as serum, from a blood sample. Preferably, the kit is designed for use with a human subject.

Multispecific Antibodies

The binding domains of the anti-IL1RAP antibodies described herein recognize cells expressing IL1RAP on their surface. As noted above, IL1RAP expression can be indicative of a cancerous cell. More specific targeting to particular subsets of cells can be achieved by making bispecific or multispecific molecules, such as antibodies or antibody fragments, which bind to IL1RAP and to another target. The antigen-binding regions can take any form that allows specific recognition of the target, for example the binding region may be or may include a heavy chain variable domain, an Fv (combination of a heavy chain variable domain and a light chain variable domain), a binding domain based on a fibronectin type III domain (such as from fibronectin, or based on a consensus of the type III domains from fibronectin, or from tenascin or based on a consensus of the type III domains from tenascin, such as the Centyrin molecules from Janssen Biotech, Inc., see e.g. WO2010/051274 and WO2010/093627). Accordingly, bispecific or multispecific molecules comprising two or more different antigen-binding regions which bind IL1RAP and another antigen(s), respectively, are provided.

Some of the multispecific antibodies described herein comprise two different antigen-binding regions which bind IL1RAP and CD3, respectively. In preferred embodiments, multispecific antibodies that bind IL1RAP and CD3 (IL1RAP x CD3-multispecific antibodies) and multispecific antigen-binding fragments thereof are provided. In some embodiments, the IL1RAP x CD3-multispecific antibody comprises a first heavy chain (HC1) and a first light chain (LC1) that pair to form a first antigen-binding site that specifically binds IL1RAP and a second heavy chain (HC2) and a second light chain (LC2) that pair to form a second antigen-binding site that specifically binds CD3. In preferred embodiments, the IL1RAP x CD3-multispecific antibody is a bispecific antibody comprising an IL1RAP-specific arm comprising a first heavy chain (HC1) and a first light chain (LC1) that pair to form a first antigen-binding site that specifically binds IL1RAP and a CD3-specific arm comprising second heavy chain (HC2) and a second light chain (LC2) that pair to form a second antigen-binding site that specifically binds CD3. In some embodiments, the bispecific antibodies of the invention include antibodies having a full length antibody structure. “Full length antibody” as used herein refers to an antibody having two full length antibody heavy chains and two full length antibody light chains. A full length antibody heavy chain (HC) includes heavy chain variable and constant domains VH, CHI, CH2, and CH3. A full length antibody light chain (LC) includes light chain variable and constant domains VL and CL. The full length antibody may be lacking the C-terminal lysine (K) in either one or both heavy chains. The term “Fab-arm” or “half molecule” refers to one heavy chain-light chain pair that specifically binds an antigen. In some embodiments, one of the antigen-binding domains is a non-antibody based binding domain, e.g. a binding domain of based on a fibronectin type 3 domain, e.g. Centyrin.

The IL1RAP-binding arm of the multispecific antibodies provided herein may be derived from any of the IL1RAP-specific antibodies described above. In some exemplary embodiments of such IL1RAP-binding arms, the first antigen-binding region which binds IL1RAP comprises a heavy chain CDR1, CDR2, and CDR3 derived from an antibody as described in Table 1. In some exemplary embodiments of such IL1RAP-binding arms, the first antigen-binding region which binds IL1RAP comprises heavy chain CDR1, CDR2, and CDR3 and light chain CDR1, CDR2, and CDR3 derived from an antibody as described in Table 1. In some exemplary embodiments of such IL1RAP-binding arms, the first antigen-binding region which binds IL1RAP comprises heavy chain CDR1, CDR2, and CDR3 of any one of the following IL1RAP-specific antibodies: IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65. In some exemplary embodiments of such IL1RAP-binding arms, the first antigen-binding region which binds IL1RAP comprises heavy chain CDR1, CDR2, and CDR3 and light chain CDR1, CDR2, and CDR3 of any one of the following IL1RAP-specific antibodies: IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65. In some exemplary embodiments of such IL1RAP-binding arms, the first antigen-binding region which binds IL1RAP comprises a heavy chain variable domain derived from an antibody as described in Table 1. In some exemplary embodiments of such IL1RAP-binding arms, the first antigen-binding region which binds IL1RAP comprises heavy chain variable domain and light chain variable domain derived from an antibody as described in Table 1. In some exemplary embodiments of such IL1RAP-binding arms, the first antigen-binding region which binds IL1RAP comprises heavy chain variable domain of any one of the following IL1RAP-specific antibodies: IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65. In some exemplary embodiments of such IL1RAP-binding arms, the first antigen-binding region which binds IL1RAP comprises heavy chain variable domain and light chain variable domain of any one of the following IL1RAP-specific antibodies: IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65.

In some embodiments of the bispecific antibodies, the IL1RAP-binding arm binds also binds cynomolgus IL1RAP, preferably the extracellular domain thereof.

In some embodiments, the IL1RAP-binding arm of the multispecific antibody is IgG, or a derivative thereof, e.g., IgG1, IgG2, IgG3, and IgG4 isotypes. In some embodiments wherein the IL1RAP-binding arm has an IgG1 isotype, it contains L234A, L235A, and K409R substitution(s) in its Fc region. In some embodiments wherein the IL1RAP-binding arm has an IgG4 isotype, it contains S228P, L234A, and L235A substitution(s) in its Fc region.

In some embodiments of the bispecific antibodies, the second antigen-binding arm binds human CD3. In some preferred embodiments, the CD3-specific arm of the IL1RAP x CD3 bispecific antibody is derived from a CD3-specific antibody that binds and activates human primary T cells and/or cynomolgus monkey primary T cells. In some embodiments, the CD3-binding arm binds to an epitope at the N-terminus of CD3c. In some embodiments, the CD3-binding arm contacts an epitope including the six N-terminal amino acids of CD3c. In some embodiments, the CD3-specific binding arm of the bispecific antibody is derived from the mouse monoclonal antibody SP34, a mouse IgG3/lambda isotype. In some embodiments, the CD3-binding arm comprises the CDRs of antibody SP34. Such CD3-binding arms may bind to CD3 with an affinity of 5×10⁻⁷M or less, such as 1×10⁻⁷M or less, 5×10⁻⁸M or less, 1×10⁻⁸M or less, 5×10⁻⁹M or less, or 1×10⁻⁹M or less. The CD3-specific binding arm may be a humanized version of an arm of mouse monoclonal antibody SP34. Human framework adaptation (HFA) may be used to humanize the anti-CD3 antibody from which the CD3-specific arm is derived. In some embodiments of the bispecific antibodies, the CD3-binding arm comprises a heavy chain and light chain pair selected from Table 2.

In some embodiments, the CD3-binding arm is IgG, or a derivative thereof. In some embodiments, the CD3-binding arm is IgG1, IgG2, IgG3, or IgG4. In some embodiments wherein the CD3-binding arm has an IgG1 isotype, it contains L234A, L235A, and F405L substitution(s) in its Fc region. In some embodiments wherein the CD3-binding arm has an IgG4 isotype, it contains S228P, L234A, L235A, F405L, and R409K substitution(s) in its Fc region. In some embodiments, the antibodies or antigen-binding fragments bind CD3c on primary human T cells. In some embodiments, the antibodies or antigen-binding fragments bind CD3c on primary cynomolgus T cells. In some embodiments, the antibodies or antigen-binding fragments bind CD3c on primary human and cynomolgus T cells. In some embodiments, the antibodies or antigen-binding fragments activate primary human CD4+ T cells. In some embodiments, the antibodies or antigen-binding fragments activate primary cynomolgus CD4+ T cells.

In some embodiments are provided an IL1RAP x CD3 bispecific antibody having an IL1RAP-binding arm comprising a heavy chain of any one of antibody IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65. In some embodiments are provided an IL1RAP x CD3 bispecific antibody having an IL1RAP-binding arm comprising a heavy chain and light chain of any one of antibody IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65. In some embodiments are provided an IL1RAP x CD3 bispecific antibody having a CD3-binding arm comprising a heavy chain of antibody CD3B220 or CD3B219. In some embodiments are provided an IL1RAP x CD3 bispecific antibody having a CD3-binding arm comprising a heavy chain and light chain of antibody CD3B220 or CD3B219. In some embodiments are provided an IL1RAP x CD3 bispecific antibody having an IL1RAP-binding arm comprising a heavy chain of antibody of any one of IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65 and a CD3-binding arm comprising a heavy chain of antibody CD3B220 or CD3B219. In some embodiments are provided an IL1RAP x CD3 bispecific antibody having an IL1RAP-binding arm comprising a heavy chain and light chain of any one of antibody IAPB47, IAPB38, IAPB57, IAPB61, IAPB62, IAPB3, IAPB17, IAPB23, IAPB25, IAPB29, IAPB9, IAPB55, IAPB63, IAPB64, or IAPB65 a CD3-binding arm comprising a heavy chain and light chain of antibody CD3B220 or CD3B219.

Preferred IL1RAP x CD3 bispecific antibodies are provided in Tables 10 and 15. Different formats of bispecific antibodies have been described and were recently reviewed by Kontermann (2012) MAbs (2012) 4:182-197 and Chames and Baty (2009) Curr Opin Drug Disc Dev 12: 276.

In some embodiments, the bispecific antibody of the present invention is a diabody, a cross-body, or a bispecific antibody obtained via a controlled Fab arm exchange as those described in the present invention.

In some embodiments, the bispecific antibodies include IgG-like molecules with complementary CH3 domains to force heterodimerisation; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody)(EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules include to ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)_{2-Fab} (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based and domain antibodies include but are not limited to Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies of the invention may be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on IL1RAP and an epitope on CD3.

“Homodimerization” as used herein refers to an interaction of two heavy chains having identical CH3 amin acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Inti. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S˜L368A˜Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V_K409F Y407A/T366A_K409F, or T350V_L351Y F405A Y407V/T350V_T366L_K392L_T394W as described in U.S. Pat. Publ. No. US2012/0149876 or U.S. Pat. Publ. No. US2013/0195849.

In addition to methods described above, bispecific antibodies of the invention may be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in Inti. Pat. Publ. No. WO2011/131746. In the methods, the first monospecific bivalent antibody (e.g., anti-IL1RAP antibody) and the second monospecific bivalent antibody (e.g., anti-CD3 antibody) are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing conditions. Exemplary reducing agents that may be used are 2-mercaptoethylamine (2-MEA), dithiothreitol (DTT), dithioerythritol (DTE), glutathione, tris (2-carboxyethyl)phosphine (TCEP), L-cysteine and beta-mercaptoethanol, preferably a reducing agent selected from the group consisting of: 2-mercaptoethylamine, dithiothreitol and tris (2-carboxyethyl)phosphine. For example, incubation for at least 90 minutes at a temperature of at least 20° C. in the presence of at least 25 mM 2-MEA or in the presence of at least 0.5 mM dithiothreitol at a pH from 5-8, for example at pH of 7.0 or at pH of 7.4 may be used.

In addition to the described IL1RAP x CD3-multispecific antibodies, also provided are polynucleotide sequences capable of encoding the described IL1RAP x CD3-multispecific antibodies. Vectors comprising the described polynucleotides are also provided, as are cells expressing the IL1RAP x CD3-multispecific antibodies provided herein. Also described are cells capable of expressing the disclosed vectors. These cells may be mammalian cells (such as 293F cells, CHO cells), insect cells (such as Sf7 cells), yeast cells, plant cells, or bacteria cells (such as E. coli). The described antibodies may also be produced by hybridoma cells.

Therapeutic composition and methods of treatment using multispecific antibodies and multispecific antigen-binding fragments thereof.

The IL1RAP bispecific antibodies discussed above, for example the IL1RAP x CD3 bispecific antibodies discussed above, are useful in therapy. In particular, the IL1RAP bispecific antibodies are useful in treating cancer. Also provided herein are therapeutic compositions for the treatment of a hyperproliferative disorder in a mammal which comprises a therapeutically effective amount of a multispecific antibody or multispecific antigen-binding fragment described herein and a pharmaceutically acceptable carrier. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof. In one embodiment said pharmaceutical composition is for the treatment of an IL1RAP-expressing cancer, including (but not limited to) the following: IL1RAP-expressing hematological cancers, such as acute myeloid leukemia (AML) myelodysplastic syndrome (MDS, low, intermediate, or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN); and other hematological cancers yet to be determined in which IL1RAP is expressed. In another embodiment said pharmaceutical composition is for the treatment of an IL1RAP-expressing solid tumor, including (but not limited to) the following: prostate, breast, lung, colorectal, melanomas, bladder, brain/CNS, cervical, esophageal, gastric, head/neck, kidney, liver, ovarian, pancreatic, and sarcomas; and other tumors yet to be determined in which IL1RAP is expressed. Particular bispecific antibodies that may be used to treat cancer, such as hematological cancers or solid tumors, including the specific cancers discussed above, include antibodies IC3B1, IC3B2, IC3B3, IC3B4, IC3B5, IC3B6, IC3B6, IC3B7, IC3B8, IC3B9, IC3B10, IC3B11, IC3B12, IC3B13, IC3B14, IC3B15, IC3B16, IC3B17, IC3B18, IC3B19. One example of a useful bispecific antibody for treating cancer, such as hematological cancers or solid tumors, including these specific cancers is antibody IC3B18. Another example of a useful bispecific antibody for treating cancer, such as hematological cancer or solid tumors, including these specific cancers is antibody IC3B19. In one embodiment, antibody IC3B19 may be used to treat one or more IL1RAP-expressing hematological cancers. In one embodiment of the described methods of treatment, antibody IC3B19 may be used to treat acute myeloid leukemia (AML). In one embodiment of the described methods of treatment, antibody IC3B19 may be used to treat myelodysplastic syndrome (MDS, low or high risk). In one embodiment of the described methods of treatment, antibody IC3B19 may be used to treat acute lymphocytic leukemia (ALL, including all subtypes). In one embodiment of the described methods of treatment, antibody IC3B19 may be used to treat diffuse large B-cell lymphoma (DLBCL). In one embodiment of the described methods of treatment, antibody IC3B19 may be used to treat chronic myeloid leukemia (CML). In one embodiment of the described methods of treatment, antibody IC3B19 may be used to treat blastic plasmacytoid dendritic cell neoplasm (DPDCN).

The IL1RAP bispecific antibodies described herein may be used to inhibit angiogenesis. Also provided herein are therapeutic compositions for inhibiting angiogenesis in a mammal which comprises a therapeutically effective amount of a multispecific antibody or multispecific antigen-binding fragment described herein and a pharmaceutically acceptable carrier. In some embodiments, the multispecific antibody useful for inhibiting angiogenesis is an IL1RAP x CD3-multispecific antibody as described herein, or a multispecific antigen-binding fragment thereof. In one embodiment the described IL1RAP bispecific antibodies may be used to inhibit angiogenesis associated with cancer, regardless of whether or not the cancer expresses IL1RAP, by administering one of the described IL1RAP bispecific antibodies to a subject in need of angiogenesis inhibition. In one embodiment the antibody IC3B19 may be administered to a subject to inhibit angiogenesis. In one embodiment the antibody IC3B19 may be administered to a subject to inhibit angiogenesis. In some embodiments the administration of either antibody IC3B18 or IC3B19 will inhibit angiogenesis in a subject with cancer. While a number of cancers may be treated by the administration of the bispecific antibodies described herein to inhibit angiogenesis, this sort of treatment will most commonly occur for cancer types exhibiting solid tumors, including (but not limited to) the following: prostate, breast, lung, colorectal, melanomas, bladder, brain/CNS, cervical, esophageal, gastric, head/neck, kidney, liver, ovarian, pancreatic, and sarcomas. Particular bispecific antibodies that may be used to treat cancer, by inhibiting angiogenesis, include antibodies IC3B1, IC3B2, IC3B3, IC3B4, IC3B5, IC3B6, IC3B6, IC3B7, IC3B8, IC3B9, IC3B10, IC3B11, IC3B12, IC3B13, IC3B14, IC3B15, IC3B16, IC3B17, IC3B18, IC3B19. One example of a useful bispecific antibody for inhibiting angiogenesis to treat cancer is antibody IC3B18. Another example of a useful bispecific antibody for inhibiting angiogenesis to treat cancer is antibody IC3B19.

The IL1RAP bispecific antibodies described herein may be used to deplete myeloid-derived suppressor cell (MDSC) populations. Use of the described bispecific antibodies to deplete MDSCs in a subject can enhance the subject's immune response to a given stimulus by removing the effectively negating the suppressor function of the MDSCs. In some embodiments the described bispecific antibodies could be used to deplete MDSCs in a subject having cancer, thereby allowing for the same subject's immune system to be directed to attack the subject's cancer. In some embodiments, the multispecific antibody useful for depleting MDSCs is an IL1RAP x CD3-multispecific antibody as described herein, or a multispecific antigen-binding fragment thereof. In one embodiment the described IL1RAP bispecific antibodies may be used to deplete MDSCs in a subject with cancer, regardless of whether or not the cancer expresses IL1RAP, by administering one of the described IL1RAP bispecific antibodies to a subject in need of immune system enhancement. In one embodiment the antibody IC3B19 may be administered to a subject to deplete the subject's MDSC population. In one embodiment the antibody IC3B19 may be administered to a subject to deplete the subject's MDSC population. In some embodiments the administration of either antibody IC3B18 or IC3B19 will deplete MDSCs in a subject with cancer. While a number of cancers may be treated by the administration of the bispecific antibodies described herein to deplete MDSCs, this sort of treatment will most commonly occur for cancer types exhibiting solid tumors, including (but not limited to) the following: prostate, breast, lung, colorectal, melanomas, bladder, brain/CNS, cervical, esophageal, gastric, head/neck, kidney, liver, ovarian, pancreatic, and sarcomas. Particular bispecific antibodies that may be used to treat cancer by depleting MDSCs, include antibodies IC3B1, IC3B2, IC3B3, IC3B4, IC3B5, IC3B6, IC3B6, IC3B7, IC3B8, IC3B9, IC3B10, IC3B11, IC3B12, IC3B13, IC3B14, IC3B15, IC3B16, IC3B17, IC3B18, IC3B19. One example of a useful bispecific antibody for depleting MDSCs to treat cancer is antibody IC3B18. Another example of a useful bispecific antibody for depleting MDSCs to treat cancer is antibody IC3B19. In one embodiment antibody IC3B18 could be used to deplete MDSCs in a subject having lung cancer. In one embodiment antibody IC3B18 could be used to deplete MDSCs in a subject having prostate cancer. In one embodiment antibody IC3B19 could be used to deplete MDSCs in a subject having lung cancer. In one embodiment antibody IC3B19 could be used to deplete MDSCs in a subject having prostate cancer.

In some embodiments administration of the described bispecific antibodies to a subject having cancer could simultaneously direct the subject's T-cells to target IL1RAP-positive cancer cells, while also depleting the subject's MDSCs to foster a more robust immune response against cancer cells. While a number of IL1RAP-expressing cancers may be treated in this manner by the administration of the bispecific antibodies described herein, this sort of treatment will most commonly occur for cancer types exhibiting solid tumors, including (but not limited to) the following: prostate, breast, lung, colorectal, melanomas, bladder, brain/CNS, cervical, esophageal, gastric, head/neck, kidney, liver, ovarian, pancreatic, and sarcomas. Particular bispecific antibodies that may be used to direct the subject's T-cells to target IL1RAP-positive cancer cell and deplete MDSCs, include antibodies IC3B1, IC3B2, IC3B3, IC3B4, IC3B5, IC3B6, IC3B6, IC3B7, IC3B8, IC3B9, IC3B10, IC3B11, IC3B12, IC3B13, IC3B14, IC3B15, IC3B16, IC3B17, IC3B18, IC3B19. One example of a useful bispecific antibody for directing a subject's T-cells to target IL1RAP-positive cancer cells while also depleting MDSCs to treat cancer is antibody IC3B18. Another example of a useful bispecific antibody for directing a subject's T-cells to target IL1RAP-positive cancer cells while also depleting MDSCs to treat cancer is antibody IC3B19. In one embodiment antibody IC3B18 could be used to direct a subject's T-cells to target IL1RAP-positive cancer cells while also depleting MDSCs in a subject having lung cancer. In one embodiment antibody IC3B18 could be used to direct a subject's T-cells to target IL1RAP-positive cancer cells while also depleting MDSCs in a subject having prostate cancer. In one embodiment antibody IC3B19 could be used to direct a subject's T-cells to target IL1RAP-positive cancer cells while also depleting MDSCs in a subject having lung cancer. In one embodiment antibody IC3B19 could be used to direct a subject's T-cells to target IL1RAP-positive cancer cells while also depleting MDSCs in a subject having prostate cancer.

The pharmaceutical compositions provided herein comprise: a) an effective amount of a multispecific antibody or antibody fragment of the present invention, and b) a pharmaceutically acceptable carrier, which may be inert or physiologically active. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof. As used herein, the term “pharmaceutically acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH. about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

The compositions herein may also contain a further therapeutic agent, as necessary for the particular disorder being treated. Preferably, the multispecific antibody or antibody fragment and the supplementary active compound will have complementary activities that do not adversely affect each other. In a preferred embodiment, the further therapeutic agent is cytarabine, an anthracycline, histamine dihydrochloride, or interleukin 2. In a preferred embodiment, the further therapeutic agent is a chemotherapeutic agent.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperitoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the antibody, antibody fragment or antibody conjugate of the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The multispecific antibody or antibody fragment may also be orally administered. As solid compositions for oral administration, tablets, pills, powders (gelatin capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used; they are generally between 5 mg and 1000 mg per day orally for an adult with unit doses ranging from 1 mg to 250 mg of active substance. In general, the doctor will determine the appropriate dosage depending on the age, weight and any other factors specific to the subject to be treated.

Also provided herein are methods for inducing cell cytotoxicity of an IL1RAP+ cell by administering to a patient in need thereof a multispecific antibody which binds said IL1RAP and is able to recruit T cells to induce cell cytotoxicity of said IL1RAP+ cell (i.e., T cell redirection). Any of the multispecific antibodies or antibody fragments of the invention may be used therapeutically. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof.

In a preferred embodiment, multispecific antibodies or antibody fragments of the invention are used for the treatment of a hyperproliferative disorder in a mammal. In a more preferred embodiment, one of the pharmaceutical compositions disclosed above, and which contains a multispecific antibody or antibody fragment of the invention, is used for the treatment of a hyperproliferative disorder in a mammal. In one embodiment, the disorder is a cancer. In particular, the cancer is an IL1RAP-expressing cancer, including (but not limited to) the following: IL1RAP-expressing hematological cancers, such as acute myeloid leukemia (AML) myelodysplastic syndrome (MDS, low, intermediate, or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN); and other cancers yet to be determined in which IL1RAP is expressed. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof.

Accordingly, the pharmaceutical compositions of the invention are useful in the treatment or prevention of a variety of cancers, including (but not limited to) the following: an IL1RAP-expressing cancer, including (but not limited to) the following: IL1RAP-expressing hematological cancers, such as acute myeloid leukemia (AML), myelodysplastic syndrome (MDS, low, intermediate, or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN); and other cancers yet to be determined in which IL1RAP is expressed. The pharmaceutical compositions of the invention are also useful in the treatment and prevention of IL1RAP-expressing solid tumors, including (but not limited to) the following: prostate, breast, lung, colorectal, melanomas, bladder, brain/CNS, cervical, esophageal, gastric, head/neck, kidney, liver, ovarian, pancreatic, and sarcomas; and other solid tumors yet to be determined in which IL1RAP is expressed.

Similarly, further provided herein is a method for inhibiting the growth of selected cell populations comprising contacting IL1RAP-expressing target cells, or tissue containing such target cells, with an effective amount of a multispecific antibody or antibody fragment of the present invention, either alone or in combination with other cytotoxic or therapeutic agents, in the presence of a peripheral blood mononuclear cell (PBMC). In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof. In a preferred embodiment, the further therapeutic agent is cytarabine, an anthracycline, histamine dihydrochloride, or interleukin 2. In a preferred embodiment, the further therapeutic agent is a chemotherapeutic agent. The method for inhibiting the growth of selected cell populations can be practiced in vitro, in vivo, or ex vivo.

Examples of in vitro uses include treatments of autologous bone marrow prior to their transplant into the same patient in order to kill diseased or malignant cells; treatments of bone marrow prior to its transplantation in order to kill competent T cells and prevent graft-versus-host-disease (GVHD); treatments of cell cultures in order to kill all cells except for desired variants that do not express the target antigen; or to kill variants that express undesired antigen. The conditions of non-clinical in vitro use are readily determined by one of ordinary skill in the art.

Examples of clinical ex vivo use are to remove tumor cells from bone marrow prior to autologous transplantation in cancer treatment. Treatment can be carried out as follows. Bone marrow is harvested from the patient or other individual and then incubated in medium containing serum to which is added the cytotoxic agent of the invention. Concentrations range from about 1 uM to 10 uM, for about 30 minutes to about 48 hours at about 37° C. The exact conditions of concentration and time of incubation, i.e., the dose, are readily determined by one of ordinary skill in the art. After incubation the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

For clinical in vivo use, a therapeutically effective amount of the multispecific antibody or antigen-binding fragment is administered to a subject in need thereof. For example, the IL1RAP x CD3-multispecific antibodies and multispecific antigen-binding fragments thereof may be useful in the treatment of an IL1RAP-expressing cancer in a subject in need thereof. In some embodiments, the IL1RAP-expressing cancer is a hematological cancer, such as acute myeloid leukemia (AML) myelodysplastic syndrome (MDS, low, intermediate, or high risk), acute lymphocytic leukemia (ALL, including all subtypes), diffuse large B-cell lymphoma (DLBCL), chronic myeloid leukemia (CIVIL), or blastic plasmacytoid dendritic cell neoplasm (DPDCN). In some embodiments the IL1RAP-expressing cancer is a solid tumor, including (but not limited to) the following: prostate, breast, lung, colorectal, melanomas, bladder, brain/CNS, cervical, esophageal, gastric, head/neck, kidney, liver, ovarian, pancreatic, and sarcomas; and other tumors yet to be determined in which IL1RAP is expressed. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof. In some embodiments, the subject is a mammal, preferably a human. In some embodiments, the multispecific antibody or antigen-binding fragment will be administered as a solution that has been tested for sterility.

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage.

The efficient dosages and the dosage regimens for the multispecific antibodies and fragments depend on the disease or condition to be treated and may be determined by one skilled in the art. An exemplary, non-limiting range for a therapeutically effective amount of a compound of the present invention is about 0.001-10 mg/kg, such as about 0.001-5 mg/kg, for example about 0.001-2 mg/kg, such as about 0.001-1 mg/kg, for instance about 0.001, about 0.01, about 0.1, about 1 or about 10 mg/kg.

A physician or veterinarian having ordinary skill in the art may readily determine and prescribe the effective amount of the pharmaceutical composition required. For example, the physician or veterinarian could start doses of the multispecific antibody or fragment employed in the pharmaceutical composition at levels lower than that required in order to achieve the desired therapeutic effect and gradually increase the dosage until the desired effect is achieved. In general, a suitable daily dose of a bispecific antibody of the present invention will be that amount of the compound which is the lowest dose effective to produce a therapeutic effect. Administration may e.g. be parenteral, such as intravenous, intramuscular or subcutaneous. In one embodiment, the multispecific antibody or fragment may be administered by infusion in a weekly dosage of calculated by mg/m². Such dosages can, for example, be based on the mg/kg dosages provided above according to the following: dose (mg/kg)×70:1.8. Such administration may be repeated, e.g., 1 to 8 times, such as 3 to 5 times. The administration may be performed by continuous infusion over a period of from 2 to 24 hr, such as of from 2 to 12 hr. In one embodiment, the multispecific antibody or fragment may be administered by slow continuous infusion over a long period, such as more than 24 hours, in order to reduce toxic side effects.

In one embodiment, the multispecific antibody or fragment may be administered in a weekly dosage of calculated as a fixed dose for up to eight times, such as from four to six times when given once a week. Such regimen may be repeated one or more times as necessary, for example, after six months or twelve months. Such fixed dosages can, for example, be based on the mg/kg dosages provided above, with a body weight estimate of 70 kg. The dosage may be determined or adjusted by measuring the amount of bispecific antibody of the present invention in the blood upon administration by for instance taking out a biological sample and using anti-idiotypic antibodies which target the IL1RAP antigen binding region of the multispecific antibodies of the present invention.

In one embodiment, the multispecific antibody or fragment may be administered by maintenance therapy, such as, e.g., once a week for a period of six months or more.

A multispecific antibody or fragment may also be administered prophylactically in order to reduce the risk of developing cancer, delay the onset of the occurrence of an event in cancer progression, and/or reduce the risk of recurrence when a cancer is in remission.

The multispecific antibodies and fragments thereof as described herein may also be administered in combination therapy, i.e., combined with other therapeutic agents relevant for the disease or condition to be treated. Accordingly, in one embodiment, the antibody-containing medicament is for combination with one or more further therapeutic agent, such as a chemotherapeutic agent. In some embodiments, the other therapeutic agent is cytarabine, an anthracycline, histamine dihydrochloride, or interleukin 2. Such combined administration may be simultaneous, separate or sequential, in any order. For simultaneous administration the agents may be administered as one composition or as separate compositions, as appropriate.

In one embodiment, a method for treating a disorder involving cells expressing IL1RAP in a subject, which method comprises administration of a therapeutically effective amount of a multispecific antibody or fragment, such as an IL1RAP x CD3 bispecific antibody described herein, and radiotherapy to a subject in need thereof is provided. In one embodiment is provided a method for treating or preventing cancer, which method comprises administration of a therapeutically effective amount of a multispecific antibody or fragment, such as an IL1RAP x CD3 antibody described herein, and radiotherapy to a subject in need thereof. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

Kits

Also provided herein are kits, e.g., comprising a described multispecific antibody or antigen-binding fragment thereof and instructions for the use of the antibody or fragment for cytotoxicity of particular cell types. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof. The instructions may include directions for using the multispecific antibody or antigen-binding fragment thereof in vitro, in vivo or ex vivo.

Typically, the kit will have a compartment containing the multispecific antibody or antigen-binding fragment thereof. The multispecific antibody or antigen-binding fragment thereof may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the multispecific antibody or antigen-binding fragment thereof prior to administering to a patient, and tools that aid in administering the multispecific antibody or antigen-binding fragment thereof to a patient.

Diagnostic Uses

The multispecific antibodies and fragments described herein may also be used for diagnostic purposes. Thus, also provided are diagnostic compositions, comprising a multispecific antibody or fragments as defined herein, and to its use. In preferred embodiments, the multispecific antibody is an IL1RAP x CD3-multispecific antibody as described herein, or a multispecific antigen-binding fragment thereof, and more preferably an IL1RAP x CD3-bispecific antibody as described herein, or an IL1RAP x CD3-bispecific antigen-binding fragment thereof. In one embodiment, the present invention provides a kit for diagnosis of cancer comprising a container comprising a bispecific IL1RAP x CD3 antibody, and one or more reagents for detecting binding of the antibody to IL1RAP. Reagents may include, for example, fluorescent tags, enzymatic tags, or other detectable tags. The reagents may also include secondary or tertiary antibodies or reagents for enzymatic reactions, wherein the enzymatic reactions produce a product that may be visualized. For example, the multispecific antibodies described herein, or antigen-binding fragments thereof, may be labeled with a radiolabel, a fluorescent label, an epitope tag, biotin, a chromophore label, an ECL label, an enzyme, ruthenium, ¹¹¹In-DOTA, diethylenetriaminepentaacetic acid (DTPA), horseradish peroxidase, alkaline phosphatase and beta-galactosidase, or poly-histidine or similar such labels known in the art.

### Exemplary Embodiments of the Described Subject Matter

To better and more fully describe the subject matter herein, this section provides enumerated exemplary embodiments of the subject matter presented.

Enumerated Embodiments:

1. A recombinant antibody, or an antigen-binding fragment thereof, that binds specifically to IL1RAP comprising:

a. a heavy chain CDR1 having the amino acid sequence of SEQ ID NO: 10, a heavy chain CDR2 having the amino acid sequence of SEQ ID NO: 11, and a heavy chain CDR3 having the amino acid sequence of SEQ ID NO: 12;

b. a heavy chain CDR1 having the amino acid sequence of SEQ ID NO: 13, a heavy chain CDR2 having the amino acid sequence of SEQ ID NO: 14, and a heavy chain CDR3 having the amino acid sequence of SEQ ID NO: 15;

c. a heavy chain CDR1 having the amino acid sequence of SEQ ID NO: 16, a heavy chain CDR2 having the amino acid sequence of SEQ ID NO: 17, and a heavy chain CDR3 having the amino acid sequence of SEQ ID NO: 18;

d. a heavy chain CDR1 having the amino acid sequence of SEQ ID NO: 19, a heavy chain CDR2 having the amino acid sequence of SEQ ID NO: 20, and a heavy chain CDR3 having the amino acid sequence of SEQ ID NO: 21;

e. a heavy chain CDR1 having the amino acid sequence of SEQ ID NO: 22, a heavy chain CDR2 having the amino acid sequence of SEQ ID NO: 23, and a heavy chain CDR3 having the amino acid sequence of SEQ ID NO: 24;

f. a heavy chain CDR1 having the amino acid sequence of SEQ ID NO: 25, a heavy chain CDR2 having the amino acid sequence of SEQ ID NO: 26, and a heavy chain CDR3 having the amino acid sequence of SEQ ID NO: 27;

g. a heavy chain CDR1 having the amino acid sequence of SEQ ID NO: 25, a heavy chain CDR2 having the amino acid sequence of SEQ ID NO: 28, and a heavy chain CDR3 having the amino acid sequence of SEQ ID NO: 29;

h. a heavy chain CDR1 having the amino acid sequence of SEQ ID NO: 30, a heavy chain CDR2 having the amino acid sequence of SEQ ID NO: 31, and a heavy chain CDR3 having the amino acid sequence of SEQ ID NO: 32;

i. a heavy chain CDR1 having the amino acid sequence of SEQ ID NO: 33, a heavy chain CDR2 having the amino acid sequence of SEQ ID NO: 34, and a heavy chain CDR3 having the amino acid sequence of SEQ ID NO: 35;

j. a heavy chain CDR1 having the amino acid sequence of SEQ ID NO: 13, a heavy chain CDR2 having the amino acid sequence of SEQ ID NO: 34, and a heavy chain CDR3 having the amino acid sequence of SEQ ID NO: 36;

k. a heavy chain CDR1 having the amino acid sequence of SEQ ID NO: 25, a heavy chain CDR2 having the amino acid sequence of SEQ ID NO: 37, and a heavy chain CDR3 having the amino acid sequence of SEQ ID NO: 38; or

l. a heavy chain CDR1 having the amino acid sequence of SEQ ID NO: 25, a heavy chain CDR2 having the amino acid sequence of SEQ ID NO: 26, and a heavy chain CDR3 having the amino acid sequence of SEQ ID NO: 39.

2. The antibody, or antigen-binding fragment thereof, of embodiment 1, wherein

a. said antibody comprising said heavy chain CDR1 having the amino acid sequence of SEQ ID NO: 10, said heavy chain CDR2 having the amino acid sequence of SEQ ID NO: 11, and said heavy chain CDR3 having the amino acid sequence of SEQ ID NO: 12 further comprises a light chain CDR1 having the amino acid sequence of SEQ ID NO: 40, a light chain CDR2 having the amino acid sequence of SEQ ID NO: 41, and a light chain CDR3 having the amino acid sequence of SEQ ID NO: 42;

b. said antibody comprising said heavy chain CDR1 having the amino acid sequence of SEQ ID NO: 13, said heavy chain CDR2 having the amino acid sequence of SEQ ID NO: 14, and said heavy chain CDR3 having the amino acid sequence of SEQ ID NO: 15 further comprises a light chain CDR1 having the amino acid sequence of SEQ ID NO: 43, a light chain CDR2 having the amino acid sequence of SEQ ID NO: 44, and a light chain CDR3 having the amino acid sequence of SEQ ID NO: 45;

c. said antibody comprising said heavy chain CDR1 having the amino acid sequence of SEQ ID NO: 16, said heavy chain CDR2 having the amino acid sequence of SEQ ID NO: 17, and said heavy chain CDR3 having the amino acid sequence of SEQ ID NO: 18 further comprises a light chain CDR1 having the amino acid sequence of SEQ ID NO: 46, a light chain CDR2 having the amino acid sequence of SEQ ID NO: 47, and a light chain CDR3 having the amino acid sequence of SEQ ID NO: 103;

d. said antibody comprising said heavy chain CDR1 having the amino acid sequence of SEQ ID NO: 19, said heavy chain CDR2 having the amino acid sequence of SEQ ID NO: 20, and said heavy chain CDR3 having the amino acid sequence of SEQ ID NO: 21 further comprises a light chain CDR1 having the amino acid sequence of SEQ ID NO: 49, a light chain CDR2 having the amino acid sequence of SEQ ID NO: 50, and a light chain CDR3 having the amino acid sequence of SEQ ID NO: 51;

e. said antibody comprising said heavy chain CDR1 having the amino acid sequence of SEQ ID NO: 22, said heavy chain CDR2 having the amino acid sequence of SEQ ID NO: 23, and said heavy chain CDR3 having the amino acid sequence of SEQ ID NO: 24 further comprises a light chain CDR1 having the amino acid sequence of SEQ ID NO: 52, a light chain CDR2 having the amino acid sequence of SEQ ID NO: 47, and a light chain CDR3 having the amino acid sequence of SEQ ID NO: 53;

f. said antibody comprising said heavy chain CDR1 having the amino acid sequence of SEQ ID NO: 25, said heavy chain CDR2 having the amino acid sequence of SEQ ID NO: 26, and said heavy chain CDR3 having the amino acid sequence of SEQ ID NO: 27 further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56;

g. said antibody comprising said heavy chain CDR1 having the amino acid sequence of SEQ ID NO: 25, said heavy chain CDR2 having the amino acid sequence of SEQ ID NO: 28, and said heavy chain CDR3 having the amino acid sequence of SEQ ID NO: 29 further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56;

h. said antibody comprising said heavy chain CDR1 having the amino acid sequence of SEQ ID NO: 30, said heavy chain CDR2 having the amino acid sequence of SEQ ID NO: 31, and said heavy chain CDR3 having the amino acid sequence of SEQ ID NO: 32 further comprises a light chain CDR1 having the amino acid sequence of SEQ ID NO: 57, a light chain CDR2 having the amino acid sequence of SEQ ID NO: 58, and a light chain CDR3 having the amino acid sequence of SEQ ID NO: 59;

i. said antibody comprising said heavy chain CDR1 having the amino acid sequence of SEQ ID NO: 33, said heavy chain CDR2 having the amino acid sequence of SEQ ID NO: 34, and said heavy chain CDR3 having the amino acid sequence of SEQ ID NO: 35 further comprises a light chain CDR1 having the amino acid sequence of SEQ ID NO: 60, a light chain CDR2 having the amino acid sequence of SEQ ID NO: 47, and a light chain CDR3 having the amino acid sequence of SEQ ID NO: 48;

j. said antibody comprising said heavy chain CDR1 having the amino acid sequence of SEQ ID NO: 13, said heavy chain CDR2 having the amino acid sequence of SEQ ID NO: 34, and said heavy chain CDR3 having the amino acid sequence of SEQ ID NO: 36 further comprises a light chain CDR1 having the amino acid sequence of SEQ ID NO: 60, a light chain CDR2 having the amino acid sequence of SEQ ID NO: 47, and a light chain CDR3 having the amino acid sequence of SEQ ID NO: 48;

k. said antibody comprising said heavy chain CDR1 having the amino acid sequence of SEQ ID NO: 25, said heavy chain CDR2 having the amino acid sequence of SEQ ID NO: 37, and said heavy chain CDR3 having the amino acid sequence of SEQ ID NO: 38 further comprises a light chain CDR1 having the amino acid sequence of SEQ ID NO: 60, a light chain CDR2 having the amino acid sequence of SEQ ID NO: 47, and a light chain CDR3 having the amino acid sequence of SEQ ID NO: 48;

l. said antibody comprising said heavy chain CDR1 having the amino acid sequence of SEQ ID NO: 19, said heavy chain CDR2 having the amino acid sequence of SEQ ID NO: 20, and said heavy chain CDR3 having the amino acid sequence of SEQ ID NO: 21 further comprises a light chain CDR1 having the amino acid sequence of SEQ ID NO: 49, a light chain CDR2 having the amino acid sequence of SEQ ID NO: 50, and a light chain CDR3 having the amino acid sequence of SEQ ID NO: 61;

m. said antibody comprising said heavy chain CDR1 having the amino acid sequence of SEQ ID NO: 22, said heavy chain CDR2 having the amino acid sequence of SEQ ID NO: 23, and said heavy chain CDR3 having the amino acid sequence of SEQ ID NO: 24 further comprises a light chain CDR1 having the amino acid sequence of SEQ ID NO: 62, a light chain CDR2 having the amino acid sequence of SEQ ID NO: 63, and a light chain CDR3 having the amino acid sequence of SEQ ID NO: 64;

n. said antibody comprising said heavy chain CDR1 having the amino acid sequence of SEQ ID NO: 22, said heavy chain CDR2 having the amino acid sequence of SEQ ID NO: 23, and said heavy chain CDR3 having the amino acid sequence of SEQ ID NO: 24 further comprises a light chain CDR1 having the amino acid sequence of SEQ ID NO: 62, a light chain CDR2 having the amino acid sequence of SEQ ID NO: 63, and a light chain CDR3 having the amino acid sequence of SEQ ID NO: 65; or

o. said antibody comprising said heavy chain CDR1 having the amino acid sequence of SEQ ID NO: 25, said heavy chain CDR2 having the amino acid sequence of SEQ ID NO: 26, and said heavy chain CDR3 having the amino acid sequence of SEQ ID NO: 39 further comprises a light chain CDR1 having the amino acid sequence of SEQ ID NO: 66, a light chain CDR2 having the amino acid sequence of SEQ ID NO: 50, and a light chain CDR3 having the amino acid sequence of SEQ ID NO: 67.

3. The antibody or antigen-binding fragment of embodiment 1, wherein the antibody of (a) comprises a heavy chain sequence set forth in SEQ ID NO: 68 and a light chain sequence set forth in SEQ ID NO: 69;

the antibody of (b) comprises a heavy chain sequence set forth in SEQ ID NO: 70 and a light chain sequence set forth in SEQ ID NO: 71;

the antibody of (c) comprises a heavy chain sequence set forth in SEQ ID NO: 72 and a light chain sequence set forth in SEQ ID NO: 73;

the antibody of (d) comprises a heavy chain sequence set forth in SEQ ID NO: 74 and a light chain sequence set forth in SEQ ID NO: 75;

the antibody of (e) comprises a heavy chain sequence set forth in SEQ ID NO: 76 and a light chain sequence set forth in SEQ ID NO: 77;

the antibody of (f) comprises a heavy chain sequence set forth in SEQ ID NO: 78 and a light chain sequence set forth in SEQ ID NO: 79;

the antibody of (g) comprises a heavy chain sequence set forth in SEQ ID NO: 80 and a light chain sequence set forth in SEQ ID NO: 79;

the antibody of (h) comprises a heavy chain sequence set forth in SEQ ID NO: 81 and a light chain sequence set forth in SEQ ID NO: 82;

the antibody of (i) comprises a heavy chain sequence set forth in SEQ ID NO: 83 and a light chain sequence set forth in SEQ ID NO: 84;

the antibody of (j) comprises a heavy chain sequence set forth in SEQ ID NO: 85 and a light chain sequence set forth in SEQ ID NO: 84;

the antibody of (k) comprises a heavy chain sequence set forth in SEQ ID NO: 86 and a light chain sequence set forth in SEQ ID NO: 84;

the antibody of (1) comprises a heavy chain sequence set forth in SEQ ID NO: 74 and a light chain sequence set forth in SEQ ID NO: 87;

the antibody of (m) comprises a heavy chain sequence set forth in SEQ ID NO: 76 and a light chain sequence set forth in SEQ ID NO: 88;

the antibody of (n) comprises a heavy chain sequence set forth in SEQ ID NO: 76 and a light chain sequence set forth in SEQ ID NO: 89; or the antibody of (o) comprises a heavy chain sequence set forth in SEQ ID NO: 90 and a light chain sequence set forth in SEQ ID NO: 91;

4. The antibody or antigen-binding fragment of any one of embodiments 1 to 3 wherein the antibody or antigen-binding fragment thereof binds to the extracellular domain of human IL1RAP.
5. The antibody or antigen-binding fragment of any one of embodiments 1 to 4 wherein the antibody or antigen-binding fragment is a human antibody or antigen-binding fragment.
6. The antigen binding fragment of any one of embodiments 1 to 5 wherein the antigen binding fragment is a Fab fragment, a Fab2 fragment, or a single chain antibody.
7. The antibody or antigen-binding fragment of any one of embodiments 1 to 6 wherein the antibody or antigen-binding fragment thereof specifically binds IL1RAP with a K_{D} of less than about 50 nM as measured by surface plasmon resonance.
8. The antibody or antigen-binding fragment of any one of embodiments 1 to 7 wherein the antibody or antigen-binding fragment thereof are of IgG1, IgG2, IgG3, or IgG4 isotype.
9. The antibody or antigen-binding fragment of any of embodiments 1 to 8 is IgG1 or IgG4 isotype.
10. The antibody of embodiment 9 wherein the IgG1 has a K409R substitution in its Fc region.
11. The antibody of embodiment 9 wherein the IgG1 has an F405L substitution in its Fc region.
12. The antibody of embodiment 9 wherein the IgG4 has an F405L substitution and an R409K substitution in its Fc region.
13. The antibody of any one of embodiments 10 to 12 further comprising an S228P substitution, an L234A substitution, and an L235A substitution in its Fc region.
14. The antibody or antigen-binding fragment of any one of embodiments 1 to 13 wherein the antibody or antigen-binding fragment thereof specifically binds human IL1RAP and cross reacts with cynomolgus monkey IL1RAP.
15. A recombinant cell expressing the antibody or antigen-binding fragment of any one of embodiments 1 to 14.
16. The cell of embodiment 15 wherein the cell is a hybridoma or a transfectoma.
17. The cell of embodiment 15 wherein the antibody is recombinantly produced.

18. A recombinant IL1RAP x CD3 bispecific antibody comprising:

a) a first heavy chain (HC1);

b) a second heavy chain (HC2);

c) a first light chain (LC1); and

d) a second light chain (LC2),

wherein the HC1 and the LC1 pair to form a first antigen-binding site that specifically binds CD3, and the HC2 and the LC2 pair to form a second antigen-binding site that specifically binds IL1RAP, or an IL1RAP x CD3-bispecific binding fragment thereof.
19. The IL1RAP x CD3 bispecific antibody or bispecific binding fragment of embodiment 18 wherein the antibody or bispecific binding fragment is IgG1, IgG2, IgG3, or IgG4 isotype.
20. The IL1RAP x CD3 bispecific antibody or bispecific binding fragment of any of embodiments 19 and 20 wherein the antibody or bispecific binding fragment is IgG1 or IgG4 isotype.
21. The IL1RAP x CD3 bispecific antibody or bispecific binding fragment of any one of embodiments 18 to 20 wherein HC1 comprises SEQ ID NO: 92 or SEQ ID NO: 94 and LC1 comprises SEQ ID NO: 93 or SEQ ID NO: 95.
22. The IL1RAP x CD3 bispecific antibody or bispecific binding fragment of embodiment 21 wherein HC2 comprises SEQ ID NO: 68 and LC2 comprises SEQ ID NO: 69.
23. The IL1RAP x CD3 bispecific antibody or bispecific binding fragment of embodiment 21 wherein HC2 comprises SEQ ID NO: 70 and LC2 comprises SEQ ID NO: 71.
24. The IL1RAP x CD3 bispecific antibody or bispecific binding fragment of embodiment 21 wherein HC2 comprises SEQ ID NO: 72 and LC2 comprises SEQ ID NO: 73.
25. The IL1RAP x CD3 bispecific antibody or bispecific binding fragment of embodiment 21 wherein HC2 comprises SEQ ID NO: 74 and LC2 comprises SEQ ID NO: 75.
26. The IL1RAP x CD3 bispecific antibody or bispecific binding fragment of embodiment 21 wherein HC2 comprises SEQ ID NO: 76 and LC2 comprises SEQ ID NO: 77.
27. The IL1RAP x CD3 bispecific antibody or bispecific binding fragment of embodiment 21 wherein HC2 comprises SEQ ID NO: 78 and LC2 comprises SEQ ID NO: 79.
28. The IL1RAP x CD3 bispecific antibody or bispecific binding fragment of embodiment 21 wherein HC2 comprises SEQ ID NO: 80 and LC2 comprises SEQ ID NO: 79.
29. The IL1RAP x CD3 bispecific antibody or bispecific binding fragment of embodiment 21 wherein HC2 comprises SEQ ID NO: 81 and LC2 comprises SEQ ID NO: 82.
30. The IL1RAP x CD3 bispecific antibody or bispecific binding fragment of embodiment 21 wherein HC2 comprises SEQ ID NO: 83 and LC2 comprises SEQ ID NO: 84.
31. The IL1RAP x CD3 bispecific antibody or bispecific binding fragment of embodiment 21 wherein HC2 comprises SEQ ID NO: 84 and LC2 comprises SEQ ID NO: 84.
32. The IL1RAP x CD3 bispecific antibody or bispecific binding fragment of embodiment 21 wherein HC2 comprises SEQ ID NO: 86 and LC2 comprises SEQ ID NO: 84.
33. The IL1RAP x CD3 bispecific antibody or bispecific binding fragment of embodiment 21 wherein HC2 comprises SEQ ID NO: 74 and LC2 comprises SEQ ID NO: 87.
34. The IL1RAP x CD3 bispecific antibody or bispecific binding fragment of embodiment 21 wherein HC2 comprises SEQ ID NO: 76 and LC2 comprises SEQ ID NO: 88.
35. The IL1RAP x CD3 bispecific antibody or bispecific binding fragment of embodiment 21 wherein HC2 comprises SEQ ID NO: 76 and LC2 comprises SEQ ID NO: 89.
36. The IL1RAP x CD3 bispecific antibody or bispecific binding fragment of embodiment 21 wherein HC2 comprises SEQ ID NO: 90 and LC2 comprises SEQ ID NO: 91.
37. The IL1RAP x CD3 bispecific antibody or bispecific binding fragment of embodiment 18 to 36 wherein the antibody or bispecific binding fragment specifically binds IL1RAP with a K_{D} of less than about 30 nM as measured by surface plasmon resonance.
38. The IL1RAP x CD3 bispecific antibody or bispecific binding fragment of embodiments 18 to 37 wherein the antibody or bispecific binding fragment thereof binds IL1RAP on the surface of cells selected from the group consisting of human acute myeloid leukemia cells, human lung cancer cells, human colon cancer cells, human pancreatic cancer cells, human myelodysplastic syndrome cancer cells, human chronic myeloid leukemia, human diffuse large B-Cell lymphoma cells, human acute lymphoblastic leukemia cells, and human T-cell leukemia/lymphoma cells.
39. The IL1RAP x CD3 bispecific antibody or bispecific binding fragment of embodiment 18 to 38 wherein the antibody or bispecific binding fragment inhibits IL-1β mediated signaling through AP-1 and NF-κB responsive elements at concentrations above 6.7 nM.
40. The IL1RAP x CD3 bispecific antibody or bispecific binding fragment of embodiment 18 to 39 wherein the antibody or bispecific binding fragment induces T-cell dependent cytotoxicity of IL1RAP-expressing cells in vitro with an EC₅₀ of less than about 1.3 nM.
41. A recombinant IL1RAP x CD3 bispecific antibody or an IL1RAP x CD3 bispecific binding fragment thereof comprising:

a) a first heavy chain (HC1);

b) a second heavy chain (HC2);

c) a first light chain (LC1); and

d) a second light chain (LC2),

wherein the HC1 and the LC1 pair to form a first antigen-binding site that specifically binds CD3 and comprise a heavy chain CDR1 (HCDR1) as depicted in SEQ ID NO: 96, an HCDR2 as depicted in SEQ ID NO: 102, an HCDR3 as depicted in SEQ ID NO: 98 a light chain CDR1 (LCDR1) as depicted in SEQ ID NO: 99, an LCDR2 as depicted in SEQ ID NO: 100, and an LCDR3 as depicted in SEQ ID NO: 101;
and the HC2 and the LC2 pair to form a second antigen-binding site that specifically binds IL1RAP and comprise a heavy chain CDR1 (HCDR1) as depicted in SEQ ID NO: 16 or 22, an HCDR2 as depicted in SEQ ID NO: 17 or 23, an HCDR3 as depicted in SEQ ID NO: 18 or 24 a light chain CDR1 (LCDR1) as depicted in SEQ ID NO: 46 or 62, an LCDR2 as depicted in SEQ ID NO: 47 or 63, and an LCDR3 as depicted in SEQ ID NO: 103 or 64.
42. A recombinant cell expressing the antibody or bispecific binding fragment of any one of embodiments 18 to 41.
43. The cell of embodiment 42 wherein the cell is a hybridoma.
44. The cell of embodiment 42 wherein the antibody or bispecific binding fragment is recombinantly produced.
45. A method for treating a subject having cancer, said method comprising:
administering a therapeutically effective amount of the IL1RAP x CD3 bispecific antibody or bispecific binding fragment of any one of embodiments 18 to 41 to a patient in need thereof for a time sufficient to treat the cancer.
   46. A method for inhibiting growth or proliferation of cancer cells, said method comprising:
administering a therapeutically effective amount of the IL1RAPx CD3 bispecific antibody or bispecific binding fragment of any one of embodiments 16 to 39 to inhibit the growth or proliferation of cancer cells.
   47. A method of redirecting a T cell to an IL1RAP-expressing cancer cell, said method comprising:
administering a therapeutically effective amount of the IL1RAP x CD3 bispecific antibody or bispecific binding fragment of any one of embodiments 18 to 41 to redirect a T cell to a cancer.
   48. The method of embodiment 47 wherein the cancer is an IL1RAP-expressing cancer.
   49. The method of embodiment 48 wherein the IL1RAP-expressing cancer, is acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CML), blastic plasmacytoid dendritic cell neoplasm (DPDCN), T-cell leukemia/lymphoma, prostate cancer, lung cancer, colorectal cancer, or pancreatic cancer.
   50. The method of embodiment 45 further comprising administering a second therapeutic agent.
   51. The method of embodiment 50 wherein the second therapeutic agent is a chemotherapeutic agent or a targeted anti-cancer therapy.
   52. The method of embodiment 51 wherein the chemotherapeutic agent is cytarabine, an anthracycline, histamine dihydrochloride, or interleukin 2.
   53. The method of embodiment 52 wherein the second therapeutic agent is administered to said subject simultaneously with, sequentially, or separately from the bispecific antibody.
   54. A pharmaceutical composition comprising the IL1RAP x CD3 bispecific antibody or bispecific binding fragment of any one of embodiments 18 to 41 and a pharmaceutically acceptable carrier.
   55. A method for generating the IL1RAP x CD3 bispecific antibody or bispecific binding fragment of any one of embodiments 18 to 41 by culturing the cell of any one of embodiments 42 to 45.
   56. An isolated synthetic polynucleotide encoding the HC1, the HC2, the LC1 or the LC2 of the IL1RAP x CD3 bispecific antibody or bispecific binding fragment of any one of embodiments 18 to 41.
   57. A kit comprising the IL1RAP x CD3 bispecific antibody or bispecific binding fragment of any one of embodiments 18 to 41 and instructions for use thereof 58. A method of inhibiting angiogenesis in a subject, said method comprising:
   administering to a subject in need thereof a IL1RAP x CD3 bispecific antibody or bispecific binding fragment of any one of embodiments 18 to 41.
   59. The method of embodiment 58, wherein the subject has cancer.
   60. The method of embodiment 59, wherein the cancer presents with one or more solid tumors.
   59. The method of embodiment 59 or 60 wherein the cancer is an IL1RAP-expressing cancer.
   60. The method of embodiment 59 or 60 wherein the cancer is not an IL1RAP-expressing cancer.
   61. A method of depleting MDSCs in a subject, said method comprising:
   administering to a subject in need thereof a IL1RAP x CD3 bispecific antibody or bispecific binding fragment of any one of embodiments 18 to 41.
   62. The method of embodiment 58, wherein the subject has cancer.
   63. The method of embodiment 59, wherein the cancer presents with one or more solid tumors.
   64. The method of embodiment 59 or 60 wherein the cancer is an IL1RAP-expressing cancer.
   65. The method of embodiment 59 or 60 wherein the cancer is not an IL1RAP-expressing cancer.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Materials

Generation of Soluble IL1RAP ECD Protein

The extracellular domain (ECD) of human (h) IL1RAP isoform 1 (SEQ ID NO:1), hIL1RAP isoform 2 (SEQ ID NOs: 2 and 3), and cynomolgous (cyno) IL1RAP (SEQ ID NO:4) were expressed and purified for use in binding and affinity measurements. The cDNA encoding each protein was prepared using gene synthesis techniques (U.S. Pat. Nos. 6,670,127; 6,521,427) and the plasmids for expression were prepared using standard molecular biology techniques. Furthermore, each ECD protein had 6x-His tags at either the N- or C-terminus for ease of purification. The constructs with N-terminal 6x-His tags also included a HRV3C cleavage site for removal of the tag if required. All IL1RAP ECD proteins were used for binding and affinity measurements and epitope mapping.

Additionally, recombinant hIL1RAP ECD-His tag protein (Lot # MB06N00704), (SEQ ID NO:5) was also obtained from Sino Biologicals, Inc. for use in phage panning and screening. The protein was tested for endotoxin prior to use. This material was also used for binding and affinity measurements.

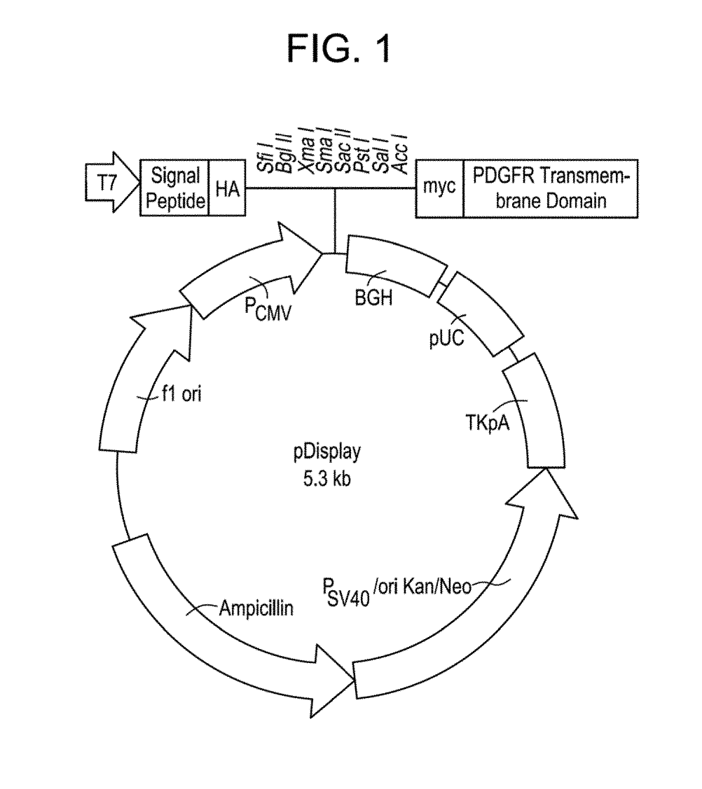

The soluble IL1RAP ECD proteins were biotinylated using the SureLink Biotinylation Kit (KPL #86-00-01) as per the manufacturer's instructions. Proteins were run on SDS/PAGE to confirm monomeric state (FIG. 1).

Generation of IL1RAP Cell Lines

A set of pDisplay™ vectors presenting human IL1RAP ECD (SEQ ID NO:6), cyno IL1RAP ECD (SEQ ID NO:7), mouse IL1RAP ECD (SEQ ID NO:8), and rat IL1RAP ECD (SEQ ID NO:9), were generated for use as screening tools to assess the anti-IL1RAP leads. A mammalian expression vector that allows display of proteins on the cell surface, pDisplay (Invitrogen) was used (FIG. 1). Proteins expressed from pDisplay™ are fused at the N-terminus to the murine Ig κ-chain leader sequence, which directs the protein to the secretory pathway, and at the C-terminus to the platelet derived growth factor receptor (PDGFR) transmembrane domain, which anchors the protein to the plasma membrane, displaying it on the extracellular side. Recombinant proteins expressed from pDisplay™ contain the hemagglutinin A and myc epitopes for detection by flow cytometry, western blot, and/or immunofluorescence. The CMV promoter drives expression of the sequences.

The vectors were transfected into HEK-293F cells using standard methods. Transfected HEK-293F adherent cells were cultured in selection media for stable plasmid integration, then single cell sorted or isolated and the IL1RAP surface receptor expression was quantified by FACS using the BangsLabs Quantum™ Simply Cellular® anti-mouse IgG (Catalog #815, Bangs Laboratories, Inc) or the BD BioSciences PE Phycoerythrin Fluorescence Quantitation Kit (cat#340495). A set of 10 single cell clones for each cell line were selected for screening, and quantified for IL1RAP ECD expression. The cell lines used for subsequent hit screening had surface expression of approximately 500,000 IL1RAP ECD copies per cell.

### Example 2: Generation of IL1RAP Antibodies Using Phage Display Technology

Solution panning of the de novo Human Fab-pIX libraries [Shi, L., et al J Mol Biol, 2010. 397(2): p. 385-396. WO 2009/085462], consisting of VH1-69, 3-23 and 5-51 heavy chain libraries paired with Vk1-39, 3-11, 3-20 and 4-1 light chain libraries, was performed using a biotinylated antigen-streptavidin magnetic bead capture method as described (Rothe et al., J. Mol. Biol. 376:1182-1200, 2008; Steidl et al., Mol. Immunol. 46: 135-144, 2008) in four subsequent rounds.

The pIX gene was excised from phagemid DNA following the fourth round of panning to generate soluble his-tagged Fab coding regions. Fabs were expressed in E. coli and screened for binding to IL1RAP in an ELISA. Briefly, 96-well Nunc Maxisorp plates (Nunc #437111) were coated with sheep anti-human Fd (The Binding Site #PC075) in PBS at 1 μg/mL overnight at 4° C. Bacterial colonies containing the Fab expression vector were grown in 450 μL of 2×YT (Carbenecillin) in deep-well culture plates until turbid (OD600≈0.6). Fab expression was induced by the addition of IPTG to a concentration of 1 mM. Cultures were grown overnight at 30° C. and then clarified by centrifugation. Anti-Fd coated Maxisorp plates were washed once with TBS, 0.5% Tween-20 (Sigma #79039-10PAK) and blocked with 200 μL PBS-Tween (0.5%)+nonfat dried milk (3%) per well for one hour at room temperature. At this step and all subsequent steps plates are washed three times with TBS, 0.5% Tween-20 (Sigma #79039-10PAK). Each well received 50 μL of Fab supernatant followed by one hour incubation at room temperature. After washing, 50 uL of biotinylated IL1RAP was added and incubated for one hour at room temperature. After washing, 50 μL of Streptavidin:HRP (Pierce #21130) was added at a 1:5000 dilution and plates were incubated for one hour at room temperature. Plates were washed and 50 uL chemiluminescent substrate, PoD (Roche #121-5829500001), was added according to manufacturer's instructions. Plates were then read for luminescence on an EnVision (Perkin Elmer) plate reader. Wells displaying signal >5-fold over background were considered hits.

Antibodies that demonstrated binding to IL1RAP were sequenced in the heavy (HC) and light chain (LC) variable regions. A total of 52 unique Fab sequences were identified via phage panning and 45 were ultimately converted to IgG1 isotype by in-fusion cloning. In-fusion cloning was performed by PCR-amplification using PCR SuperMix High Fidelity kit (Life Technologies #10790-020), of the HC and LC variable regions and cloning into Esp3I sites in vDR149 for HC and vDR157 for LC using the In-Fusion® HD Cloning Plus kit (Clontech #638909).

### Example 3: Isolation of Human ILMAP Monoclonal Antibody Expressing Hybridomas

A human immunoglobulin transgenic rat strain (OmniRat®; OMT, Inc.) was used to develop human IL1RAP monoclonal antibody expressing hybridoma cells. The OmniRat® contains a chimeric human/rat IgH locus (comprising 22 human VHs, all human D and JH segments in natural configuration linked to the rat CH locus) together with fully human IgL loci (12 Vκs linked to Jκ-Cκ and 16 Vλs linked to Jλ-Cλ). (see e.g., Osborn, et al. (2013) J Immunol 190(4): 1481-1490). Accordingly, the rats exhibit reduced expression of rat IgM or κ, and in response to immunization, the introduced human heavy and light chain transgenes undergo class switching and somatic mutation to generate high affinity human IgG monoclonal antibodies. The preparation and use of OmniRat®, and the genomic modifications carried by such rats, is described in PCT Publication WO 14/093908 to Bruggemann et al.

When immunized with recombinant human IL1RAP (rhIL1RAP), this transgenic rat produces human IgG antibodies specific to human IL1RAP.

Two immunization schemes were performed as follows: For the first scheme, four rats were immunized with rhuIL1RAP. Following a 35 day immunization regimen, spleens and lymph nodes from rat 10344 were harvested and used to generate hybridomas. Seventy-six 96-well plates of hybridoma supernatants were screened via binding ELISA, of which seventy-six hybridoma supernatants were selected. Similarly, for the second scheme, four rats were immunized with rhuIL1RAP. Following a 77 day immunization regimen, lymph nodes from rats 10428, 10424, and 10600 were harvested and used to generate hybridomas. Twenty-four 96-well plates of hybridoma supernatants were screened by ELISA to identify mAbs which exhibited binding to rhuIL1RAP. After further confirmatory screenings, hybridoma supernatants from both screens that exhibited binding specific to rhuIL1RAP and cyno IL1RAP (rcynoIL1RAP) were sequenced, cloned and expressed in small scale.

### Example 4: MSD Cell Binding to IL1RAP

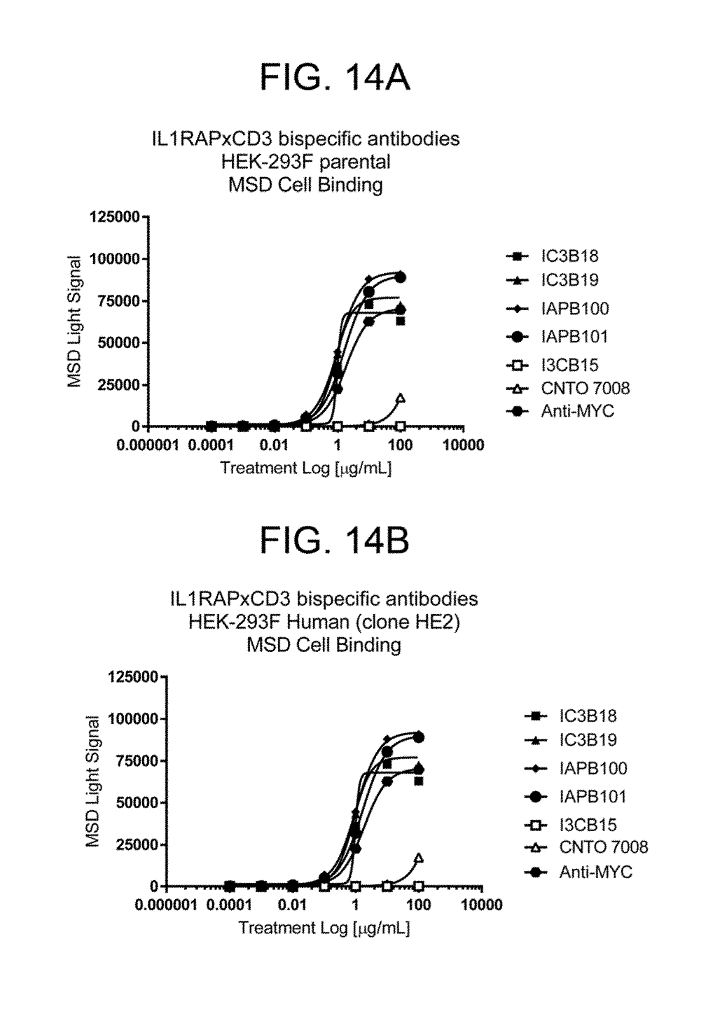

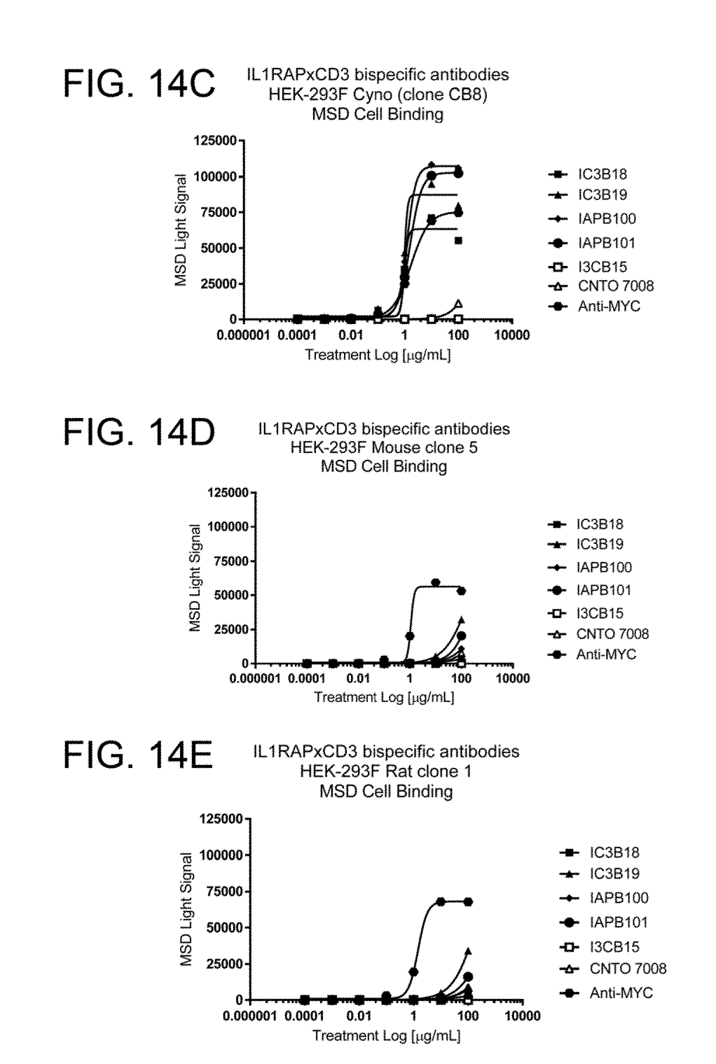

Binding of IL1RAP antibodies to engineered pDisplay cells (IL1RAP expressing HEK-293F cells) were assessed using a MSD (Mesoscale Discovery) cell binding assay. The object of the screening assay was to identify antibodies that bound to cells expressing hIL1RAP as well as cross reactivity with cells expressing cyno IL1RAP (FIG. 14).

Cells were immobilized and IL1RAP antibody samples were assayed in triplicate. Briefly, expression supernatants of purified IL1RAP antibodies were normalized to 10 μg/mL. 5000 cells per well were plated into a 384 well plate (MA6000, cat. L21XB, MSD) and allowed to adhere for 2 hr. Cells were then blocked with 20% FBS in PBS (Gibco) for 15 mins. Antibody supernatants were then added and left at RT for 1 hr. Cells were washed 3 times with PBS and a ruthenium labeled secondary antibody (Mesoscale Discovery) was then added at 2 μg/mL and incubated for 1 hour at room temperature. A further washing step was then applied and 35 μL per well of 2×MSD Read buffer T (surfactant free) was then added and incubated for 5-30 minutes for detection. Plates were then read using Sector Imager 2400 (MSD). Data was normalized to controls and graphed using GraphPad Prism Version 5. A positive binder was determined to be a hit with a signal 3× greater than parental cell line background. The assay was repeated for data consistency and top binders were selected for further development.

### Example 5: Affinity Measurements by SPR

ProteOn Affinity Measurements

The affinities of 52 [38 mAbs from phage panning, 11 mAbs from Hybridoma set 1 and three mutants produced to eliminate sequence liabilities (IAPB63, IAPB64, and IAPB65)] anti-IL1RAP candidates to recombinant human IL1RAP ECD were measured by Surface Plasmon Resonance (SPR) using a ProteOn XPR36 protein interaction array system (BioRad).

The rates of IL1RAP ECD association and dissociation were measured for each variant. The biosensor surface was prepared by covalently coupling Goat anti-Human IgG (Fc) to the surface of a GLC chip (BioRad) using the manufacturer instructions for amine-coupling chemistry. Approximately 8800 RU (response units) of Goat anti-Human IgG (Fc) antibody (Jackson ImmunoResearch laboratories Prod #109-005-098) were immobilized. The RU immobilized also included a goat anti-mouse Fc antibody that was added to capture other antibodies not included in the ones reported here. Since the mixture was 1:1 about 50% of these RU immobilized are expected to be goat anti-human Fc. The kinetic experiments were performed at 25° C. in running buffer (PBS pH 7.4, 0.005% P20, 3 mM EDTA). 4-fold (1:3) serial dilutions of human IL1RAP ECD, starting at 400 nM were prepared in running buffer. An average of 300 RU of mAb (174-600) were captured on each channel of the sensor chip. The reference spots (Goat anti-Human IgG (Fc)-modified surface) containing no candidate captured were used as a reference surface. Capture of mAb was followed by a 3 minute injection (association phase) of antigen at 40 μL/min, followed by 10 minutes of buffer flow (dissociation phase). The chip surface was regenerated by injection of 0.85% phosphoric acid at 100 μL/min. Data was processed on the instrument software. Double reference subtraction of the data was performed by subtracting the curves generated by buffer injection from the reference-subtracted curves for analyte injections. Kinetic analysis of the data was performed using 1:1 Langmuir binding model with group fit. The result for each mAb was reported in the format of Kₐ (kon or on-rate), Kd (koff or off-rate), K_{D} (Equilibrium dissociation constant) (Table 3).

The results for the phage hits are presented in Table 4. All 38 mAbs bound to human IL1RAP ECD and with affinities ranging from 1.19-30.4 nM (Table 3). It was observed that 10 mAbs (denoted with asterisk) had a poor fitting to the 1:1 binding model and their Chi^{t} values are greater than 20% Rmax. The results suggest good reproducibility (based on positive control antibody MAB676, n=4). No binding was observed for negative controls (MAB002, CNT09412, and Mock Transfection) up to 400 nM, the highest concentration tested. This suggests the antibody binding to human IL1RAP ECD is specific.

TABLE 3
Summary of kinetic affinities for Phage mAbs (unpurified) binding to human IL1RAP
(concentration range of 1.56-400 nM). The parameters reported in this table were
obtained from a 1:1 Langmuir binding model. Affinity, K_{D} = kd/ka.

| Sample | ka (1/Ms) | kd (1/s) | K_{D} (M) | K_{D} (nM) |
|---|---|---|---|---|
| anti-human/cyno IL1RAP, | 2.57E+05 | 3.67E−04 | 1.43E−09 | 1.43 |
| mouse IgG1, R&D #MAB676 | | | | |
| anti-human/cyno IL1RAP, | 2.66E+05 | 3.49E−04 | 1.31E−09 | 1.31 |
| mouse IgG1, R&D #MAB676 | | | | |
| anti-human/cyno IL1RAP, | 2.93E+05 | 3.40E−04 | 1.16E−09 | 1.16 |
| mouse IgG1, R&D #MAB676 | | | | |
| anti-human/cyno IL1RAP, | 2.76E+05 | 3.73E−04 | 1.35E−09 | 1.35 |
| mouse IgG1, R&D #MAB676 | | | | |
| Mouse IgG1 isotype control, | — | — | No Binding | No Binding |
| R&D cat #MAB002 | | | | |
| Human IgG4-PAA isotype | — | — | No Binding | No Binding |
| control | | | | |
| IAPB01 | 7.70E+04 | 3.86E−04 | 5.01E−09 | 5.01 |
| IAPB02 | 3.30E+05 | 3.83E−03 | 1.16E−08 | 11.6 |
| IAPB03 | 1.35E+05 | 3.57E−04 | 2.64E−09 | 2.64 |
| IAPB04 | 2.55E+05 | 1.44E−03 | 5.66E−09 | 5.66 |
| IAPB05 | 4.73E+05 | 2.52E−03 | 5.33E−09 | 5.33 |
| IAPB06 | 4.07E+05 | 2.27E−03 | 5.58E−09 | 5.58 |
| IAPB08 | 5.85E+05 | 6.73E−03 | 1.15E−08 | 11.5 |
| IAPB09 | 5.74E+05 | 3.79E−03 | 6.59E−09 | 6.59 |
| IAPB10 | 2.31E+05 | 3.93E−04 | 1.70E−09 | 1.7 |
| IAPB11 | 7.21E+05 | 3.83E−03 | 5.32E−09 | 5.32 |
| IAPB12 | 4.72E+05 | 5.62E−04 | 1.19E−09 | 1.19 |
| IAPB13 | 3.37E+05 | 9.03E−04 | 2.68E−09 | 2.68 |
| IAPB14 | 2.01E+05 | 5.31E−04 | 2.64E−09 | 2.64 |
| IAPB15 | 4.54E+05 | 7.67E−04 | 1.69E−09 | 1.69 |
| IAPB17 | 8.44E+05 | 7.19E−03 | 8.51E−09 | 8.51 |
| IAPB22 | 5.78E+04 | 1.75E−03 | 3.02E−08 | 30.2 |
| IAPB23 | 3.17E+05 | 1.49E−03 | 4.70E−09 | 4.7 |
| IAPB24 | 8.59E+04 | 2.61E−03 | 3.04E−08 | 30.4 |
| IAPB25 | 1.44E+06 | 4.07E−02 | 2.82E−08 | 28.2 |
| IAPB26 | 7.62E+04 | 1.06E−03 | 1.39E−08 | 13.9 |
| IAPB27 | 1.15E+05 | 2.94E−03 | 2.56E−08 | 25.6 |
| IAPB28 | 2.31E+05 | 3.31E−04 | 1.43E−09 | 1.43 |
| IAPB29 | 3.07E+05 | 1.84E−03 | 6.00E−09 | 6 |
| IAPB31 | 1.22E+05 | 1.78E−03 | 1.47E−08 | 14.7 |
| IAPB32 | 2.96E+05 | 3.56E−03 | 1.20E−08 | 12 |
| IAPB33 | 4.38E+04 | 8.10E−04 | 1.85E−08 | 18.5 |
| IAPB34 | 5.22E+05 | 4.06E−03 | 7.78E−09 | 7.78 |
| IAPB36 | 3.59E+05 | 3.05E−03 | 8.49E−09 | 8.49 |
| IAPB37 | 9.09E+04 | 3.30E−04 | 3.63E−09 | 3.63 |
| IAPB39 | 9.84E+04 | 2.60E−03 | 2.65E−08 | 26.5 |
| IAPB41 | 1.90E+05 | 2.65E−03 | 1.39E−08 | 13.9 |
| IAPB43 | 4.24E+04 | 1.25E−03 | 2.95E−08 | 29.5 |
| IAPB44 | 4.24E+05 | 1.26E−03 | 2.97E−09 | 2.97 |
| IAPB47 | 6.53E+05 | 8.11E−04 | 1.24E−09 | 1.24 |
| IAPB48 | 9.19E+04 | 5.23E−04 | 5.69E−09 | 5.69 |
| IAPB49 | 4.54E+05 | 1.53E−03 | 3.38E−09 | 3.38 |
| IAPB50 | 3.54E+05 | 1.40E−03 | 3.96E−09 | 3.96 |
| Mock Transfection | — | — | No binding | No binding |
| R7633 IAPB51 | 1.05E+05 | 4.55E−04 | 4.33E−09 | 4.33 |

The results for the hybridoma hits are presented in Table 4. The results indicated that 5 out of 11 antibodies bound to human IL1RAP ECD with affinities ranging from 0.16-49.9 nM (Table 4). Positive control (MAB676) was run twice and showed good reproducibility. As expected, the negative controls (MABOO2 and CNT07967) showed no binding up to 400 nM, the highest test concentration.

TABLE 4
Summary of kinetic affinities for Hybridoma mAbs (unpurified) binding to human
IL1RAP (concentration range of 1.56-400 nM). The parameters reported in this
table were obtained from a 1:1 Langmuir binding model. Affinity, KD = kd/ka.

| Sample | Ka | Kd | KD | KD |
|---|---|---|---|---|
| CBIS ID, Hybridoma | (1/Ms) | (1/s) | (M) | (nM) |
| anti-human/cyno IL1RAP, mouse | 2.60E+05 | 3.69E−04 | 1.42E−09 | 1.42 |
| IgG1, R&D cat #MAB676 | | | | |
| anti-human/cyno IL1RAP, mouse | 2.77E+05 | 3.36E−04 | 1.21E−09 | 1.21 |
| IgG1, R&D cat #MAB676 | | | | |
| Mouse IgG1 isotype control, R&D | | | No Binding | |
| cat #MAB002 | | | | |
| CNTO7967, Rat IgG1k isotype | | | No Binding | |
| control | | | | |
| IAPB53, 5D06 | | | Weak | |
| | | | Binding | |
| IAPB54, 17B04 | 7.50E+05 | 4.38E−04 | 5.83E−10 | 0.58 |
| IAPB55, 22A09 | 4.54E+05 | 7.47E−04 | 1.64E−09 | 1.64 |
| IAPB56, 30C11 | | | No Binding | |
| IAPB57, 5G08 | 8.07E+05 | 1.29E−04 | 1.60E−10 | 0.16 |
| 12F09 | | | Weak | |
| | | | Binding | |
| IAPB59, 19C11 | 2.81E+05 | 1.40E−02 | 4.99E−08 | 49.9 |
| IAPB60, 19F09 lambda | | | Weak | |
| | | | Binding | |
| IAPB61, 25D12 | 8.10E+05 | 1.42E−02 | 1.75E−08 | 17.5 |
| 30C12 | | | No Binding | |
| 20B11 lambda | | | Weak | |
| | | | Binding | |

Table 5 shows the data for the three mutant antibodies, which were produced to eliminate sequence liabilities. The mutants were assessed and compared to their parental antibodies. The results suggest only variant IAPB63 (IAPB54 with LC mutant C91A) retained binding affinity that is less than 2-fold different from the parent. A point of note, the affinities of purified and unpurified parent, IAPB4 (phage hit B4) were within 2-fold of each other (Table 5: 4.73 nM vs. Table 3: 5.66 nM). In contrast, the parental antibody IAPB54 (17B04 with human IgG4-PAA, Table 5) showed much tighter binding than 17B04 (Hybridoma hit with Rat IgG1, Table 4). The difference might be due to species and isotypes.

TABLE 5
Comparing the kinetic affinities of point-mutant mAbs and the parents binding
to human IL1RAP (1.2-100 nM). The parameters reported in this table were
obtained from a 1:1 Langmuir binding model. Affinity, KD = kd/ka.

| | | | | Fold Different |
|---|---|---|---|---|
| Sample | ka (1/Ms) | kd (1/s) | KD (M) | from parent |
| IAPB4, Phage | 2.95E+05 | 1.40E−03 | 4.73E−09 | 1.0 |
| IAPB65, IAPB4-HC-G103A | 3.29E+05 | 3.41E−03 | 1.04E−08 | 2.2 |
| IAPB54, Hybridoma | 9.65E+05 | 7.48E−05 | 7.75E−11 | 1.0 |
| IAPB63, IAPB54-LC-C91A | 9.00E+05 | 9.76E−05 | 1.08E−10 | 1.4 |
| IAPB64, IAPB54-LC-C91S | 6.38E+05 | 2.34E−04 | 3.67E−10 | 4.7 |

### Example 6: Neutralization Assay

HEK-Blue™ IL-1β cells from Invivogen (cat# hkb-ilb) were used to assess for agonist or antagonist activity of the IL1RAP antibodies. According to the manufacture: “HEK-Blue™ IL-1β cells allow detection of bioactive IL-1β by monitoring the activation of the NF-κB and AP-1 pathways.” “They derive from HEK-Blue™ TNF-α/IL-1β cells in which the TNF-α response has been blocked. Therefore, HEK-Blue™ IL-1β cells respond specifically to IL-1β. They express a NF-κB/AP-1-inducible SEAP reporter gene. Binding of IL-1β to its receptor IL-1R on the surface of HEK-Blue™ IL-1β cells triggers a signaling cascade leading to the activation NF-κB and the subsequent production of SEAP.” All antibody supernatants were screened at a final concentration of 10 μg/mL either alone or in the presence of 1 ng/mL of recombinant human IL-1β.

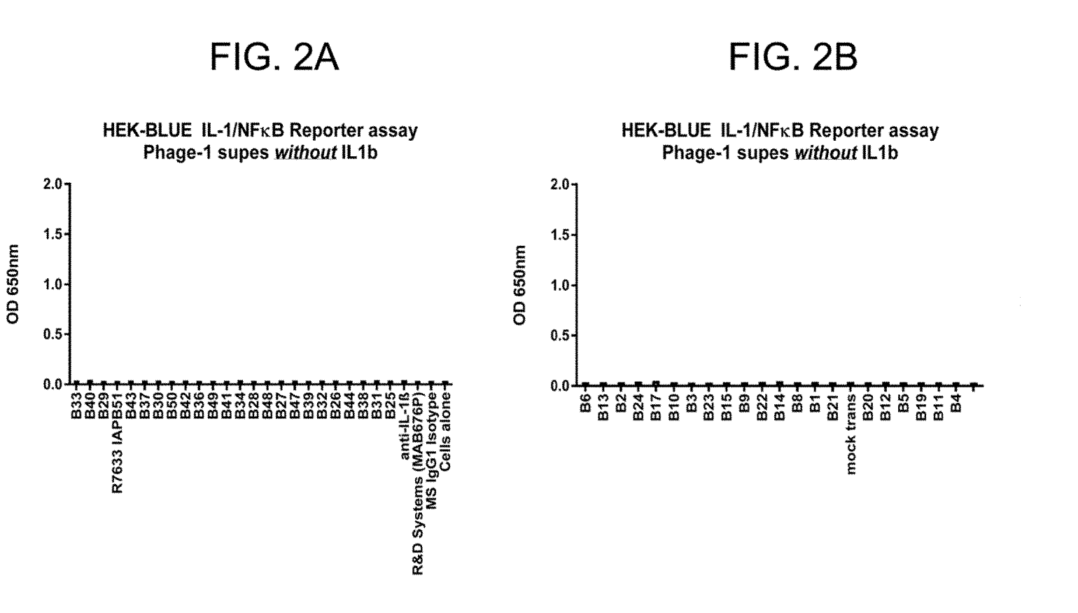

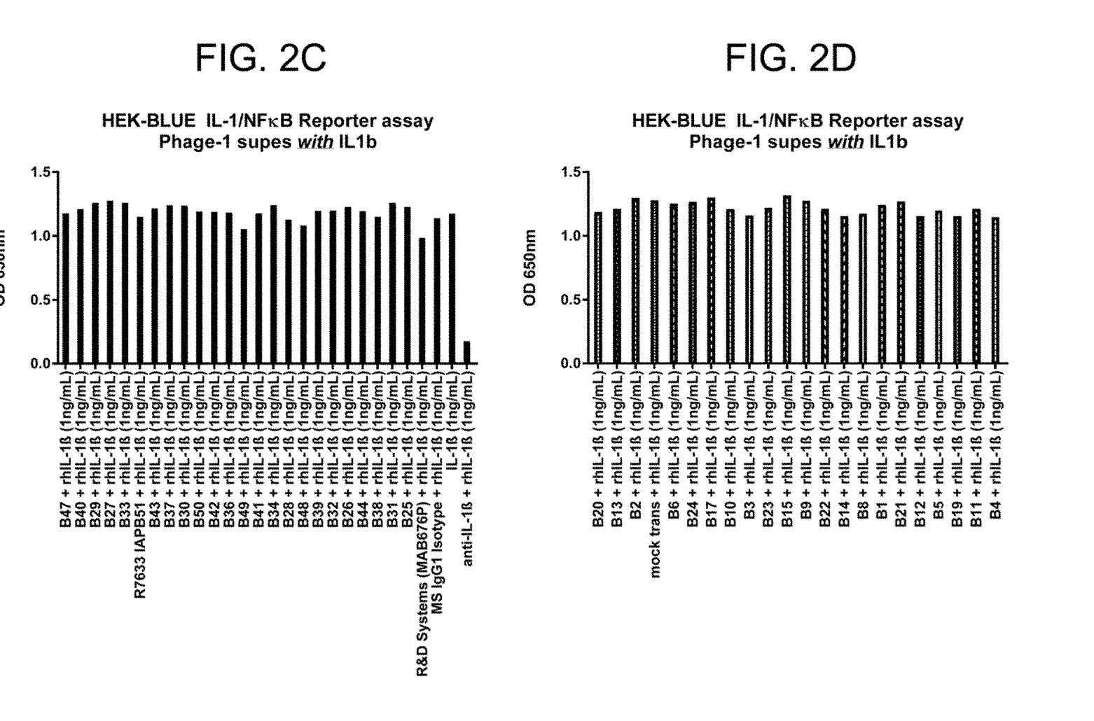

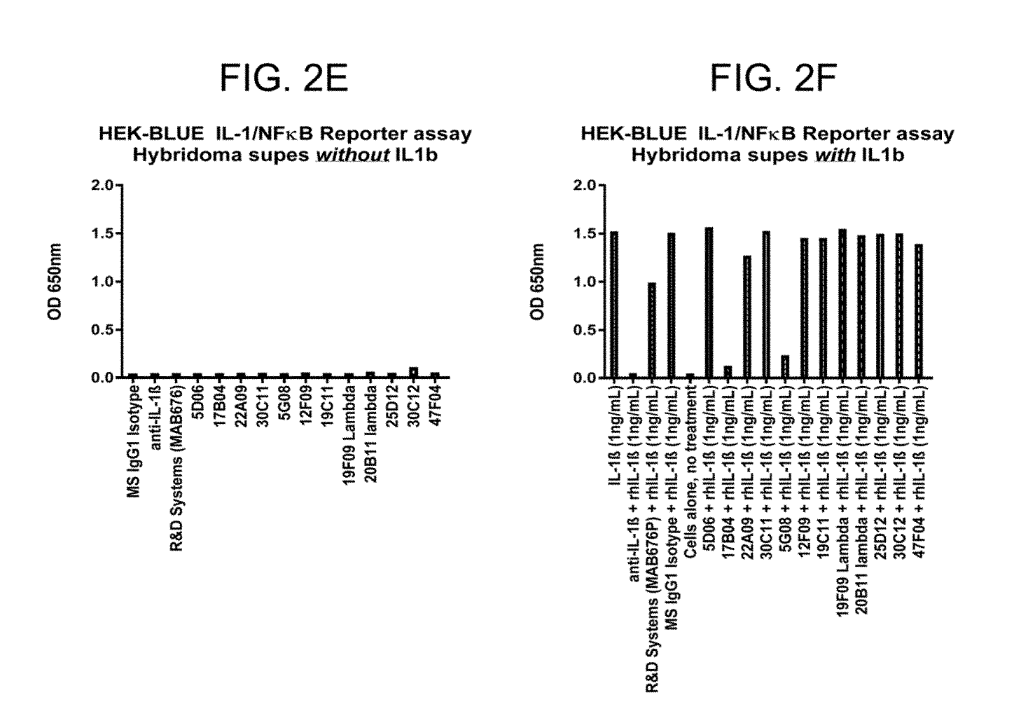

The results for the assessment of the phage hits are shown in FIG. 2. Phage supernatants were analyzed for agonist (without IL-1β) or antagonist activity (in the presence of IL-1β) in the HEK-Blue™ NFκB reporter cell line. Among the supernatants analyzed, none displayed agonist activity. However, IAPB54 and IAPB57 (hybridoma supernatants) displayed antagonist activity in the presence of recombinant human IL-1β (FIG. 2).

### Example 7: Hit Evaluation and Selection

All of the phage and hybridoma hits that were found to be cross-reactive with cynomolgus monkey and had measureable affinity via the Proteon assessment were collated together. From this list, six candidates were selected based on their characteristics and their cross reactivity with only primates and not mouse or rat (highlighted in gray in Table 6). The two hybridoma hits that showed antagonistic activity were also included (highlighted in gray in Table 6). IAPB4 and IAPB54 were not selected due to sequence liabilities; however, mutants of these parentals were made for further analysis. The mutants IAPB63 and IAPB64 are mutants of IAPB54, while IAPB65 is a mutant of IAPB4. Additionally, there was a potential desire to have surrogate molecules for investigating additional biology questions. Therefore, an additional four primate/murine cross-reactive antibodies were selected for testing as well (highlighted in gray in Table 6).

TABLE 6
Summary of initial anti-human IL1RAP Antibody Production.

| | | | | | | Mouse | Rat | Afinity |
|---|---|---|---|---|---|---|---|---|
| CBIS | | Human Binder | | Cyno Binder | | Binder | Binder | (nM) |
| Protein ID | Construct | Rec | Cells | Rec | Cells | Cells | Cells | Rec |
| IAPB01 | IgG4-PAA | + | + | + | + | + | + | 5.01 |
| IAPB02 | IgG4-PAA | + | + | + | + | + | + | 11.6 |
| IAPB03 | IgG4-PAA | + | + | + | + | − | − | 2.64 |
| IAPB04^{d} | IgG4-PAA | + | + | + | + | − | − | 5.66 |
| IAPB05 | IgG4-PAA | + | + | + | + | + | + | 5.33 |
| IAPB06 | IgG4-PAA | + | + | + | + | + | + | 5.58 |
| IAPB07 | IgG4-PAA | + | + | + | + | + | + | 11.5 |
| IAPB08 | IgG4-PAA | + | + | + | + | + | + | |
| IAPB09 | IgG4-PAA | + | + | + | + | + | + | 6.59 |
| IAPB10 | IgG4-PAA | + | + | + | + | + | + | 1.7 |
| IAPB11 | IgG4-PAA | + | + | + | + | + | + | 5.32 |
| IAPB12 | IgG4-PAA | + | + | + | + | + | + | 1.19 |
| IAPB13 | IgG4-PAA | + | + | + | + | + | + | 2.68 |
| IAPB14 | IgG4-PAA | + | + | + | + | + | + | 2.64 |
| IAPB15 | IgG4-PAA | + | + | + | + | + | + | 1.69 |
| IAPB16 | IgG4-PAA | + | + | + | + | + | + | |
| IAPB17 | IgG4-PAA | + | + | + | + | + | + | 8.51 |
| IAPB18 | IgG4-PAA | + | + | + | + | + | + | |
| IAPB21 | IgG4-PAA | + | + | + | + | + | + | |
| IAPB22 | IgG4-PAA | + | + | + | + | + | + | 30.2 |
| IAPB23 | IgG4-PAA | + | + | + | + | − | − | 4.7 |
| IAPB24 | IgG4-PAA | + | + | + | + | + | + | 30.4 |
| IAPB25 | IgG4-PAA | − | + | − | + | − | − | 28.2 |
| IAPB26 | IgG4-PAA | + | + | + | + | + | + | 13.9 |
| IAPB27 | IgG4-PAA | + | + | + | + | + | + | 25.6 |
| IAPB28 | IgG4-PAA | + | + | + | + | + | + | 1.43 |
| IAPB29 | IgG4-PAA | + | + | + | + | + | + | 6 |
| IAPB30 | IgG4-PAA | + | + | + | + | + | + | |
| IAPB31 | IgG4-PAA | + | + | + | + | + | + | 14.7 |
| IAPB32 | IgG4-PAA | + | + | + | + | + | + | 12 |
| IAPB33 | IgG4-PAA | + | + | + | + | + | + | 18.5 |
| IAPB34 | IgG4-PAA | + | + | + | + | + | + | 7.78 |
| IAPB35 | IgG4-PAA | + | + | + | + | + | + | |
| IAPB36 | IgG4-PAA | + | + | + | + | + | + | 8.49 |
| IAPB37 | IgG4-PAA | + | + | + | + | + | + | 3.63 |
| IAPB38^{a} | IgG4-PAA | + | + | + | + | + | + | |
| IAPB39 | IgG4-PAA | + | + | + | + | + | + | 26.5 |
| IAPB40 | IgG4-PAA | + | + | + | + | + | + | |
| IAPB41 | IgG4-PAA | + | + | + | + | + | + | 13.9 |
| IAPB42 | IgG4-PAA | + | + | + | + | + | + | |
| IAPB43 | IgG4-PAA | + | + | + | + | + | + | 29.5 |
| IAPB44 | IgG4-PAA | + | + | + | + | + | + | 2.97 |
| IAPB45 | IgG4-PAA | + | + | + | + | NA | NA | |
| IAPB46 | IgG4-PAA | + | + | + | + | + | + | |
| IAPB47^{a} | IgG4-PAA | + | + | + | + | + | + | 1.24 |
| IAPB66 | IgG1-FEA | | | | | | | |
| IAPB48 | IgG4-PAA | + | + | + | + | + | + | 5.69 |
| IAPB49 | IgG4-PAA | + | + | + | + | + | + | 3.38 |
| IAPB50 | IgG4-PAA | + | + | + | + | + | + | 3.96 |
| IAPB51 | IgG4-PAA | + | + | + | + | + | + | |
| IAPB52 | IgG4-PAA | + | + | + | + | + | + | |
| IgG4-PAA, B4 mutant | | Not Analyzed | | | | | | 10.4 |
| IAPB53 | IgG4-PAA | + | + | − | + | − | − | ND |
| IAPB57 | IgG4-PAA | + | + | + | + | − | − | 1.6 |
| IAPB68 | IgG1-FEA | | | | | | | |
| IAPB55 | IgG4-PAA | + | + | + | + | − | − | 1.64 |
| IAPB67 | IgG1-FEA | | | | | | | |
| IAPB61 | IgG4-PAA | + | + | + | + | − | − | 17.5 |
| IAPB54^{e} | IgG4-PAA | + | + | + | + | − | − | 0.58 |
| IAPB102 | RatG1 | + | + | + | + | + | + | 49.9 |
| IAPB59 | IgG4-PAA | | | | | | | |
| IAPB62 | IgG4-PAA | + | + | + | + | − | − | |

| | | | |
|---|---|---|---|
| IAPB63 | IgG4-PAA | Not Analyzed | 0.11 |
| IAPB81 | IgG1-FEA | | |
| IAPB64 | IgG4-PAA | Not Analyzed | 0.38 |

*Contaminated supe, ND = not determined, NA = not analyzed.
^{a,b,c}These hybridomas contained the same antibody.
^{d}NA = Not analyzed, ND = Not determined.
^{d}Analyzed the mutant of this parental in a bispecific format (IAPB65).
^{e}Analyzed the mutants of this parental in a bispecific format (IAPB63, and IPAB64).

Thus, in total a panel of 15 IL1RAP parentals (five hits from hybridoma screening and eight hits from phage panning) as well as three mutants (IAPB63, IAPB64, IAPB65)—all depicted in Table 7—were expressed and purified for the purpose of making a small-scale IL1RAP x CD3 bispecific panel.

TABLE 7
CDR sequences of the 15 IL1RAP mAb candidates selected for generation of
IL1RAP × CD3 bispecific panel (relevant SEQ ID NO: shown in parenthesis)

| ID | HC-CDR1 | HC-CDR2 | HC-CDR3 | LC-CDR1 | LC-CDR2 | LC-CDR3 |
|---|---|---|---|---|---|---|
| IAPB47 | GYSFTSYW | IYPSDSYT | ARRNSAENYADLDY (12) | QSISND (40) | YAS (41) | QQSFTAPLT |
| | (10) | (11) | | | | (42) |
| IAPB38 | GFTFSNYA | INYGGGSK | AKDYGPFALDY (15) | QSVDDW (43) | TAS (44) | QQYHHWPLT |
| | (13) | (14) | | | | (45) |
| IAPB57 | GGSISSSTYY | IYFTGST | AKEDDSSGYYSFDY (18) | QGISSY (46) | AAS (47) | QQVNSYPLT |
| | (16) | (17) | | | | (103) |
| IAPB61 | GVSISSSTYY | IYFTGNT | GSLFGDYGYFDY (21) | QFISSN (49) | GAS (50) | QQYNNWPST |
| | (19) | (20) | | | | (51) |
| IAPB62 | GYTFNTYA | INTNTGNP | ARRYFDWLLGAFDI (24) | QGISSW (52) | AAS (47) | QQANSFPLT |
| | (22) | (23) | | | | (53) |
| IAPB3 | GGTFSSYA | ISAIFGTA | ARGNSFHALWDYAFDY (27) | QSVLYSSNNKNY | WAS (55) | QQYYSTPLT |
| | (25) | (26) | | (54) | | (56) |
| IAPB17 | GGTFSSYA | IIPIFGNA | ARTIIYLDYVHILDY (29) | QSVLYSSNNKNY | WAS (55) | QQYYSTPLT |
| | (25) | (28) | | (54) | | (56) |
| IAPB23 | GFTFSNYW | IRYDGGSK | AKDAYPPYSFDY (32) | QSVSSY (57) | DAS (58) | QQRSNWPLT |
| | (30) | (31) | | | | (59) |
| IAPB25 | GFTFSSYA | ISGSGGST | AKGDEYYYPDPLDY (35) | QSISSY (60) | AAS (47) | QQSYSTPLT |
| | (33) | (34) | | | | (48) |
| IAPB29 | GFTFSNYA | ISGSGGST | AKEWSSYFGLDY (36) | QSISSY (60) | AAS (47) | QQSYSTPLT |
| | (13) | (34) | | | | (48) |
| IAPB9 | GGTFSSYA | ISPIFGTA | ARRYDNFARSGDLDY (38) | QSISSY (60) | AAS (47) | QQSYSTPLT |
| | (25) | (37) | | | | (48) |
| IAPB55 | GVSISSSTYY | IYFTGNT | GSLFGDYGYFDY (21) | QFISSN (49) | GAS (50) | QQYNNWPFT |
| | (19) | (20) | | | | (61) |
| IAPB63 | GYTFNTYA | INTNTGNP | ARRYFDWLLGAFDI (24) | SSDVGDYNY (62) | DVS (63) | ASYAGNYNVV |
| | (22) | (23) | | | | (64) |
| IAPB64 | GYTFNTYA | INTNTGNP | ARRYFDWLLGAFDI (24) | SSDVGDYNY (62) | DVS (63) | SSYAGNYNVV |
| | (22) | (23) | | | | (65) |
| IAPB65 | GGTFSSYA | ISAIFGTA | ARHLHNAIHLDY (39) | QSVSNF (66) | GAS (50) | QQGKHWPWT |
| | (25) | (26) | | | | (67) |

VH and VL of the 15 IL1RAP mAbs are shown below in Table 8.

TABLE 8
V_{H} and V_{L} sequences of the 15 IL1RAP mAb candidates
selected for generation of IL1RAP × CD3 bispecific panel

| | | | | SEQ |
|---|---|---|---|---|
| mAb | | SEQ ID | | ID |
| AA ID | VH Amino Acid Sequence | NO: | VL Amino Acid Sequence | NO |
| IAPB47 | EVQLVQSGAEVKKPGESLK | 68 | EIVLTQSPGTLSLSPGERA | 69 |
| | ISCKGSGYSFTSYWIGWVR | | TLSCRASQSISNDLNWYQ | |
| | QMPGKGLEWMGIIYPSDSY | | QKPGKAPKLLIYYASSLQ | |
| | TRYSPSFQGQVTISADKSIST | | SGVPSRFSGSGSGTDFTLT | |
| | AYLQWSSLKASDTAMYYC | | INSLQPEDFATYYCQQSFT | |
| | ARRNSAENYADLDYWGQG | | APLTFGQGTKVEIKRTVA | |
| | TLVTVSSASTKGPSVFPLAP | | APSVFIFPPSDEQLKSGTA | |
| | CSRSTSESTAALGCLVKDYF | | SVVCLLNNFYPREAKVQ | |
| | PEPVTVSWNSGALTSGVHT | | WKVDNALQSGNSQESVT | |
| | FPAVLQSSGLYSLSSVVTVP | | EQDSKDSTYSLSSTLTLSK | |
| | SSSLGTKTYTCNVDHKPSN | | ADYEKHKVYACEVTHQG | |
| | TKVDKRVESKYGPPCPPCP | | LSSPVTKSFNRGEC | |
| | APEAAGGPSVFLFPPKPKDT | | | |
| | LMISRTPEVTCVVVDVSQE | | | |
| | DPEVQFNWYVDGVEVHNA | | | |
| | KTKPREEQFNSTYRVVSVL | | | |
| | TVLHQDWLNGKEYKCKVS | | | |
| | NKGLPSSIEKTISKAKGQPR | | | |
| | EPQVYTLPPSQEEMTKNQV | | | |
| | SLTCLVKGFYPSDIAVEWES | | | |
| | NGQPENNYKTTPPVLDSDG | | | |
| | SFFLYSRLTVDKSRWQEGN | | | |
| | VFSCSVMHEALHNHYTQKS | | | |
| | LSLSLGK | | | |
| IAPB38 | EVQLLESGGGLVQPGGSLR | 70 | EIVLTQSPATLSLSPGERA | 71 |
| | LSCAASGFTFSNYAMNWV | | TLSCRASQSVDDWLAWY | |
| | RQAPGKGLEWVSGINYGG | | QQKPGQAPRLLIYTASNR | |
| | GSKYYADSVKGRFTISRDN | | ATGIPARFSGSGSGTDFTL | |
| | SKNTLYLQMNSLRAEDTAV | | TISSLEPEDFAVYYCQQY | |
| | YYCAKDYGPFALDYWGQG | | HHWPLTFGQGTKVEIKRT | |
| | TLVTVSSASTKGPSVFPLAP | | VAAPSVFIFPPSDEQLKSG | |
| | CSRSTSESTAALGCLVKDYF | | TASVVCLLNNFYPREAKV | |
| | PEPVTVSWNSGALTSGVHT | | QWKVDNALQSGNSQESV | |
| | FPAVLQSSGLYSLSSVVTVP | | TEQDSKDSTYSLSSTLTLS | |
| | SSSLGTKTYTCNVDHKPSN | | KADYEKHKVYACEVTHQ | |
| | TKVDKRVESKYGPPCPPCP | | GLSSPVTKSFNRGEC | |
| | APEAAGGPSVFLFPPKPKDT | | | |
| | LMISRTPEVTCVVVDVSQE | | | |
| | DPEVQFNWYVDGVEVHNA | | | |
| | KTKPREEQFNSTYRVVSVL | | | |
| | TVLHQDWLNGKEYKCKVS | | | |
| | NKGLPSSIEKTISKAKGQPR | | | |
| | EPQVYTLPPSQEEMTKNQV | | | |
| | SLTCLVKGFYPSDIAVEWES | | | |
| | NGQPENNYKTTPPVLDSDG | | | |
| | SFFLYSRLTVDKSRWQEGN | | | |
| | VFSCSVMHEALHNHYTQKS | | | |
| | LSLSLGK | | | |
| IAPB57 | QLQLQESGPGLVKPSETLSL | 72 | DIQLTQSPSFLSASVGDRV | 73 |
| | TCTVSGGSISSSTYYWGWIR | | TITCRASQGISSYLAWYQ | |
| | QPPGKGLEWIGSIYFTGSTD | | QKPGKAPKLLIYAASTLQ | |
| | YNPSLKSRVSISVDTSKNQF | | SGVPSRFSGSGSGTEFTLT | |
| | SLKLSSVTAADTAVYYCAK | | ISSLQPEDFATYYCQQVN | |
| | EDDSSGYYSFDYWGQGNL | | SYPLTFGGGTKVEIKRTV | |
| | VTVSSASTKGPSVFPLAPCS | | AAPSVFIFPPSDEQLKSGT | |
| | RSTSESTAALGCLVKDYFPE | | ASVVCLLNNFYPREAKVQ | |
| | PVTVSWNSGALTSGVHTFP | | WKVDNALQSGNSQESVT | |
| | AVLQSSGLYSLSSVVTVPSS | | EQDSKDSTYSLSSTLTLSK | |
| | SLGTKTYTCNVDHKPSNTK | | ADYEKHKVYACEVTHQG | |
| | VDKRVESKYGPPCPPCPAPE | | LSSPVTKSFNRGEC | |
| | AAGGPSVFLEPPKPKDTLMI | | | |
| | SRTPEVTCVVVDVSQEDPE | | | |
| | VQFNWYVDGVEVHNAKTK | | | |
| | PREEQFNSTYRVVSVLTVL | | | |
| | HQDWLNGKEYKCKVSNKG | | | |
| | LPSSIEKTISKAKGQPREPQV | | | |
| | YTLPPSQEEMTKNQVSLTC | | | |
| | LVKGFYPSDIAVEWESNGQ | | | |
| | PENNYKTTPPVLDSDGSFFL | | | |
| | YSRLTVDKSRWQEGNVFSC | | | |
| | SVMHEALHNHYTQKSLSLS | | | |
| | LGK | | | |
| IAPB61 | QLQLQESGPGLVKPSETLSL | 74 | EIVMTQSPATLSVPPGERA | 75 |
| | TCTVSGVSISSSTYYWGWL | | TLSCRASQFISSNLAWYQ | |
| | RQPPGMGLEWTGSIYFTGN | | QKPGQAPRLLIYGASTRA | |
| | TYYNPSLKSRVTISVDTSRN | | TGIPARFSGSGSGTDFTLTI | |
| | QFSLKLSSVTAADTAVYYC | | SSLQSEDFAVYYCQQYNN | |
| | GSLFGDYGYFDYWGQGTL | | WPSTFGPGTKVDIKRTVA | |
| | VTVSSASTKGPSVFPLAPCS | | APSVFIFPPSDEQLKSGTA | |
| | RSTSESTAALGCLVKDYFPE | | SVVCLLNNFYPREAKVQ | |
| | PVTVSWNSGALTSGVHTFP | | WKVDNALQSGNSQESVT | |
| | AVLQSSGLYSLSSVVTVPSS | | EQDSKDSTYSLSSTLTLSK | |
| | SLGTKTYTCNVDHKPSNTK | | ADYEKHKVYACEVTHQG | |
| | VDKRVESKYGPPCPPCPAPE | | LSSPVTKSFNRGEC | |
| | AAGGPSVFLFPPKPKDTLMI | | | |
| | SRTPEVTCVVVDVSQEDPE | | | |
| | VQFNWYVDGVEVHNAKTK | | | |
| | PREEQFNSTYRVVSVLTVL | | | |
| | HQDWLNGKEYKCKVSNKG | | | |
| | LPSSIEKTISKAKGQPREPQV | | | |
| | YTLPPSQEEMTKNQVSLTC | | | |
| | LVKGFYPSDIAVEWESNGQ | | | |
| | PENNYKTTPPVLDSDGSFFL | | | |
| | YSRLTVDKSRWQEGNVFSC | | | |
| | SVMHEALHNHYTQKSLSLS | | | |
| | LGK | | | |
| IAPB62 | QVQLVQSGSELKKPGASVK | 76 | DIQMTQSPSSVSASVGDR | 77 |
| | VSCKASGYTFNTYAMNWV | | VTITCRASQGISSWLAWY | |
| | RQAPGQGLEWMGWINTNT | | QQKPGKAPKLLIYAASSL | |
| | GNPTYAQGFTGRFVFSLDT | | QSGVPSRFSGSGSGTDFTL | |
| | SVSTAYLQISSLKAEDTAVY | | TISSLQPEDFATYYCQQA | |
| | YCARRYFDWLLGAFDIWG | | NSFPLTFGGGTKVEIKRTV | |
| | QGTMVTVSSASTKGPSVFP | | AAPSVFIFPPSDEQLKSGT | |
| | LAPCSRSTSESTAALGCLVK | | ASVVCLLNNFYPREAKVQ | |
| | DYFPEPVTVSWNSGALTSG | | WKVDNALQSGNSQESVT | |
| | VHTFPAVLQSSGLYSLSSVV | | EQDSKDSTYSLSSTLTLSK | |
| | TVPSSSLGTKTYTCNVDHK | | ADYEKHKVYACEVTHQG | |
| | PSNTKVDKRVESKYGPPCP | | LSSPVTKSFNRGEC | |
| | PCPAPEAAGGPSVFLFPPKP | | | |
| | KDTLMISRTPEVTCVVVDV | | | |
| | SQEDPEVQFNWYVDGVEV | | | |
| | HNAKTKPREEQFNSTYRVV | | | |
| | SVLTVLHQDWLNGKEYKC | | | |
| | KVSNKGLPSSIEKTISKAKG | | | |
| | QPREPQVYTLPPSQEEMTK | | | |
| | NQVSLTCLVKGFYPSDIAVE | | | |
| | WESNGQPENNYKTTPPVLD | | | |
| | SDGSFFLYSRLTVDKSRWQ | | | |
| | EGNVFSCSVMHEALHNHYT | | | |
| | QKSLSLSLGK | | | |
| IAPB3 | QVQLVQSGAEVKKPGSSVK | 78 | DIVMTQSPDSLAVSLGER | 79 |
| | VSCKASGGTFSSYAISWVR | | ATINCKSSQSVLYSSNNK | |
| | QAPGQGLEWMGGISAIFGT | | NYLAWYQQKPGQPPKLLI | |
| | ANYAQKFQGRVTITADEST | | YWASTRESGVPDRFSGSG | |
| | STAYMELSSLRSEDTAVYY | | SGTDFTLTISSLQAEDVAV | |
| | CARGNSFHALWDYAFDYW | | YYCQQYYSTPLTFGQGTK | |
| | GQGTLVTVSSASTKGPSVFP | | VEIKRTVAAPSVFIFPPSD | |
| | LAPCSRSTSESTAALGCLVK | | EQLKSGTASVVCLLNNFY | |
| | DYFPEPVTVSWNSGALTSG | | PREAKVQWKVDNALQSG | |
| | VHTFPAVLQSSGLYSLSSVV | | NSQESVTEQDSKDSTYSL | |
| | TVPSSSLGTKTYTCNVDHK | | SSTLTLSKADYEKHKVYA | |
| | PSNTKVDKRVESKYGPPCP | | CEVTHQGLSSPVTKSFNR | |
| | PCPAPEAAGGPSVFLFPPKP | | GEC | |
| | KDTLMISRTPEVTCVVVDV | | | |
| | SQEDPEVQFNWYVDGVEV | | | |
| | HNAKTKPREEQFNSTYRVV | | | |
| | SVLTVLHQDWLNGKEYKC | | | |
| | KVSNKGLPSSIEKTISKAKG | | | |
| | QPREPQVYTLPPSQEEMTK | | | |
| | NQVSLTCLVKGFYPSDIAVE | | | |
| | WESNGQPENNYKTTPPVLD | | | |
| | SDGSFFLYSRLTVDKSRWQ | | | |
| | EGNVFSCSVMHEALHNHYT | | | |
| | QKSLSLSLGK | | | |
| IAPB17 | QVQLVQSGAEVKKPGSSVK | 80 | DIVMTQSPDSLAVSLGER | 79 |
| | VSCKASGGTFSSYAISWVR | | ATINCKSSQSVLYSSNNK | |
| | QAPGQGLEWMGGIIPIFGN | | NYLAWYQQKPGQPPKLLI | |
| | ANYAQKFQGRVTITADEST | | YWASTRESGVPDRFSGSG | |
| | STAYMELSSLRSEDTAVYY | | SGTDFTLTISSLQAEDVAV | |
| | CARTIIYLDYVHILDYWGQ | | YYCQQYYSTPLTFGQGTK | |
| | GTLVTVSSASTKGPSVFPLA | | VEIKRTVAAPSVFIFPPSD | |
| | PCSRSTSESTAALGCLVKDY | | EQLKSGTASVVCLLNNFY | |
| | FPEPVTVSWNSGALTSGVH | | PREAKVQWKVDNALQSG | |
| | TFPAVLQSSGLYSLSSVVTV | | NSQESVTEQDSKDSTYSL | |
| | PSSSLGTKTYTCNVDHKPS | | SSTLTLSKADYEKHKVYA | |
| | NTKVDKRVESKYGPPCPPC | | CEVTHQGLSSPVTKSFNR | |
| | PAPEAAGGPSVFLFPPKPKD | | GEC | |
| | TLMISRTPEVTCVVVDVSQ | | | |
| | EDPEVQFNWYVDGVEVHN | | | |
| | AKTKPREEQFNSTYRVVSV | | | |
| | LTVLHQDWLNGKEYKCKV | | | |
| | SNKGLPSSIEKTISKAKGQP | | | |
| | REPQVYTLPPSQEEMTKNQ | | | |
| | VSLTCLVKGFYPSDIAVEW | | | |
| | ESNGQPENNYKTTPPVLDS | | | |
| | DGSFFLYSRLTVDKSRWQE | | | |
| | GNVFSCSVMHEALHNHYT | | | |
| | QKSLSLSLGK | | | |
| IAPB23 | EVQLLESGGGLVQPGGSLR | 81 | EIVLTQSPATLSLSPGERA | 82 |
| | LSCAASGFTFSNYWMNWV | | TLSCRASQSVSSYLAWYQ | |
| | RQAPGKGLEWVSAIRYDGG | | QKPGQAPRLLIYDASNRA | |
| | SKYYADSVKGRFTISRDNS | | TGIPARFSGSGSGTDFTLTI | |
| | KNTLYLQMNSLRAEDTAV | | SSLEPEDFAVYYCQQRSN | |
| | YYCAKDAYPPYSFDYWGQ | | WPLTFGQGTKVEIKRTVA | |
| | GTLVTVSSASTKGPSVFPLA | | APSVFIFPPSDEQLKSGTA | |
| | PCSRSTSESTAALGCLVKDY | | SVVCLLNNFYPREAKVQ | |
| | FPEPVTVSWNSGALTSGVH | | WKVDNALQSGNSQESVT | |
| | TFPAVLQSSGLYSLSSVVTV | | EQDSKDSTYSLSSTLTLSK | |
| | PSSSLGTKTYTCNVDHKPS | | ADYEKHKVYACEVTHQG | |
| | NTKVDKRVESKYGPPCPPC | | LSSPVTKSFNRGEC | |
| | PAPEAAGGPSVFLFPPKPKD | | | |
| | TLMISRTPEVTCVVVDVSQ | | | |
| | EDPEVQFNWYVDGVEVHN | | | |
| | AKTKPREEQFNSTYRVVSV | | | |
| | LTVLHQDWLNGKEYKCKV | | | |
| | SNKGLPSSIEKTISKAKGQP | | | |
| | REPQVYTLPPSQEEMTKNQ | | | |
| | VSLTCLVKGFYPSDIAVEW | | | |
| | ESNGQPENNYKTTPPVLDS | | | |
| | DGSFFLYSRLTVDKSRWQE | | | |
| | GNVFSCSVMHEALHNHYT | | | |
| | QKSLSLSLGK | | | |
| IAPB25 | EVQLLESGGGLVQPGGSLR | 83 | DIQMTQSPSSLSASVGDR | 84 |
| | LSCAASGFTFSSYAMSWVR | | VTITCRASQSISSYLNWYQ | |
| | QAPGKGLEWVSAISGSGGS | | QKPGKAPKLLIYAASSLQ | |
| | TYYADSVKGRFTISRDNSK | | SGVPSRFSGSGSGTDFTLT | |
| | NTLYLQMNSLRAEDTAVY | | ISSLQPEDFATYYCQQSYS | |
| | YCAKGDEYYYPDPLDYWG | | TPLTFGQGTKVEIKRTVA | |
| | QGTLVTVSSASTKGPSVFPL | | APSVFIFPPSDEQLKSGTA | |
| | APCSRSTSESTAALGCLVKD | | SVVCLLNNFYPREAKVQ | |
| | YFPEPVTVSWNSGALTSGV | | WKVDNALQSGNSQESVT | |
| | HTFPAVLQSSGLYSLSSVVT | | EQDSKDSTYSLSSTLTLSK | |
| | VPSSSLGTKTYTCNVDHKP | | ADYEKHKVYACEVTHQG | |
| | SNTKVDKRVESKYGPPCPP | | LSSPVTKSFNRGEC | |
| | CPAPEAAGGPSVFLFPPKPK | | | |
| | DTLMISRTPEVTCVVVDVS | | | |
| | QEDPEVQFNWYVDGVEVH | | | |
| | NAKTKPREEQFNSTYRVVS | | | |
| | VLTVLHQDWLNGKEYKCK | | | |
| | VSNKGLPSSIEKTISKAKGQ | | | |
| | PREPQVYTLPPSQEEMTKN | | | |
| | QVSLTCLVKGFYPSDIAVE | | | |
| | WESNGQPENNYKTTPPVLD | | | |
| | SDGSFFLYSRLTVDKSRWQ | | | |
| | EGNVFSCSVMHEALHNHYT | | | |
| | QKSLSLSLGK | | | |
| IAPB29 | EVQLLESGGGLVQPGGSLR | 85 | DIQMTQSPSSLSASVGDR | 84 |
| | LSCAASGFTFSNYAMSWVR | | VTITCRASQSISSYLNWYQ | |
| | QAPGKGLEWVSAISGSGGS | | QKPGKAPKLLIYAASSLQ | |
| | TYYADSVKGRFTISRDNSK | | SGVPSRFSGSGSGTDFTLT | |
| | NTLYLQMNSLRAEDTAVY | | ISSLQPEDFATYYCQQSYS | |
| | YCAKEWSSYFGLDYWGQG | | TPLTFGQGTKVEIKRTVA | |
| | TLVTVSSASTKGPSVFPLAP | | APSVFIFPPSDEQLKSGTA | |
| | CSRSTSESTAALGCLVKDYF | | SVVCLLNNFYPREAKVQ | |
| | PEPVTVSWNSGALTSGVHT | | WKVDNALQSGNSQESVT | |
| | FPAVLQSSGLYSLSSVVTVP | | EQDSKDSTYSLSSTLTLSK | |
| | SSSLGTKTYTCNVDHKPSN | | ADYEKHKVYACEVTHQG | |
| | TKVDKRVESKYGPPCPPCP | | LSSPVTKSFNRGEC | |
| | APEAAGGPSVFLFPPKPKDT | | | |
| | LMISRTPEVTCVVVDVSQE | | | |
| | DPEVQFNWYVDGVEVHNA | | | |
| | KTKPREEQFNSTYRVVSVL | | | |
| | TVLHQDWLNGKEYKCKVS | | | |
| | NKGLPSSIEKTISKAKGQPR | | | |
| | EPQVYTLPPSQEEMTKNQV | | | |
| | SLTCLVKGFYPSDIAVEWES | | | |
| | NGQPENNYKTTPPVLDSDG | | | |
| | SFFLYSRLTVDKSRWQEGN | | | |
| | VFSCSVMHEALHNHYTQKS | | | |
| | LSLSLGK | | | |
| IAPB9 | QVQLVQSGAEVKKPGSSVK | 86 | DIQMTQSPSSLSASVGDR | 84 |
| | VSCKASGGTFSSYAISWVR | | VTITCRASQSISSYLNWYQ | |
| | QAPGQGLEWMGWISPIFGT | | QKPGKAPKLLIYAASSLQ | |
| | ANYAQKFQGRVTITADEST | | SGVPSRFSGSGSGTDFTLT | |
| | STAYMELSSLRSEDTAVYY | | ISSLQPEDFATYYCQQSYS | |
| | CARRYDNFARSGDLDYWG | | TPLTFGQGTKVEIKRTVA | |
| | QGTLVTVSSASTKGPSVFPL | | APSVFIFPPSDEQLKSGTA | |
| | APCSRSTSESTAALGCLVKD | | SVVCLLNNFYPREAKVQ | |
| | YFPEPVTVSWNSGALTSGV | | WKVDNALQSGNSQESVT | |
| | HTFPAVLQSSGLYSLSSVVT | | EQDSKDSTYSLSSTLTLSK | |
| | VPSSSLGTKTYTCNVDHKP | | ADYEKHKVYACEVTHQG | |
| | SNTKVDKRVESKYGPPCPP | | LSSPVTKSFNRGEC | |
| | CPAPEAAGGPSVFLFPPKPK | | | |
| | DTLMISRTPEVTCVVVDVS | | | |
| | QEDPEVQFNWYVDGVEVH | | | |
| | NAKTKPREEQFNSTYRVVS | | | |
| | VLTVLHQDWLNGKEYKCK | | | |
| | VSNKGLPSSIEKTISKAKGQ | | | |
| | PREPQVYTLPPSQEEMTKN | | | |
| | QVSLTCLVKGFYPSDIAVE | | | |
| | WESNGQPENNYKTTPPVLD | | | |
| | SDGSFFLYSRLTVDKSRWQ | | | |
| | EGNVFSCSVMHEALHNHYT | | | |
| | QKSLSLSLGK | | | |
| IAPB55 | QLQLQESGPGLVKPSETLSL | 74 | EIVMTQSPATLSVSPGERA | 87 |
| | TCTVSGVSISSSTYYWGWL | | TLSCRASQFISSNLAWYQ | |
| | RQPPGMGLEWTGSIYFTGN | | QKPGQAPRLLIYGASTRA | |
| | TYYNPSLKSRVTISVDTSRN | | TGIPARFSGSGSGTDFTLTI | |
| | QFSLKLSSVTAADTAVYYC | | SSLQSEDFAVYYCQQYNN | |
| | GSLFGDYGYFDYWGQGTL | | WPFTFGPGTKVDIKRTVA | |
| | VTVSSASTKGPSVFPLAPCS | | APSVFIFPPSDEQLKSGTA | |
| | RSTSESTAALGCLVKDYFPE | | SVVCLLNNFYPREAKVQ | |
| | PVTVSWNSGALTSGVHTFP | | WKVDNALQSGNSQESVT | |
| | AVLQSSGLYSLSSVVTVPSS | | EQDSKDSTYSLSSTLTLSK | |
| | SLGTKTYTCNVDHKPSNTK | | ADYEKHKVYACEVTHQG | |
| | VDKRVESKYGPPCPPCPAPE | | LSSPVTKSFNRGEC | |
| | AAGGPSVFLFPPKPKDTLMI | | | |
| | SRTPEVTCVVVDVSQEDPE | | | |
| | VQFNWYVDGVEVHNAKTK | | | |
| | PREEQFNSTYRVVSVLTVL | | | |
| | HQDWLNGKEYKCKVSNKG | | | |
| | LPSSIEKTISKAKGQPREPQV | | | |
| | YTLPPSQEEMTKNQVSLTC | | | |
| | LVKGFYPSDIAVEWESNGQ | | | |
| | PENNYKTTPPVLDSDGSFFL | | | |
| | YSRLTVDKSRWQEGNVFSC | | | |
| | SVMHEALHNHYTQKSLSLS | | | |
| | LGK | | | |
| IAPB63 | QVQLVQSGSELKKPGASVK | 76 | QSALTQPRSVSGSPGHSV | 88 |
| | VSCKASGYTFNTYAMNWV | | TISCTGTSSDVGDYNYVS | |
| | RQAPGQGLEWMGWINTNT | | WYQQRPGKVPKLLIYDVS | |
| | GNPTYAQGFTGRFVFSLDT | | KRPSGVPDRFSGSKSGNT | |
| | SVSTAYLQISSLKAEDTAVY | | ASLTISGLQAEDEAIYFCA | |
| | YCARRYFDWLLGAFDIWG | | SYAGNYNVVFGGGTKLT | |
| | QGTMVTVSSASTKGPSVFP | | VLGQPKAAPSVTLFPPSSE | |
| | LAPCSRSTSESTAALGCLVK | | ELQANKATLVCLISDFYP | |
| | DYFPEPVTVSWNSGALTSG | | GAVTVAWKADSSPVKAG | |
| | VHTFPAVLQSSGLYSLSSVV | | VETTTPSKQSNNKYAASS | |
| | TVPSSSLGTKTYTCNVDHK | | YLSLTPEQWKSHRSYSCQ | |
| | PSNTKVDKRVESKYGPPCP | | VTHEGSTVEKTVAPTECS | |
| | PCPAPEAAGGPSVFLFPPKP | | | |
| | KDTLMISRTPEVTCVVVDV | | | |
| | SQEDPEVQFNWYVDGVEV | | | |
| | HNAKTKPREEQFNSTYRVV | | | |
| | SVLTVLHQDWLNGKEYKC | | | |
| | KVSNKGLPSSIEKTISKAKG | | | |
| | QPREPQVYTLPPSQEEMTK | | | |
| | NQVSLTCLVKGFYPSDIAVE | | | |
| | WESNGQPENNYKTTPPVLD | | | |
| | SDGSFFLYSRLTVDKSRWQ | | | |
| | EGNVFSCSVMHEALHNHYT | | | |
| | QKSLSLSLGK | | | |
| IAPB64 | QVQLVQSGSELKKPGASVK | 76 | QSALTQPRSVSGSPGHSV | 89 |
| | VSCKASGYTFNTYAMNWV | | TISCTGTSSDVGDYNYVS | |
| | RQAPGQGLEWMGWINTNT | | WYQQRPGKVPKLLIYDVS | |
| | GNPTYAQGFTGRFVFSLDT | | KRPSGVPDRFSGSKSGNT | |
| | SVSTAYLQISSLKAEDTAVY | | ASLTISGLQAEDEAIYFCS | |
| | YCARRYFDWLLGAFDIWG | | SYAGNYNVVFGGGTKLT | |
| | QGTMVTVSSASTKGPSVFP | | VLGQPKAAPSVTLFPPSSE | |
| | LAPCSRSTSESTAALGCLVK | | ELQANKATLVCLISDFYP | |
| | DYFPEPVTVSWNSGALTSG | | GAVTVAWKADSSPVKAG | |
| | VHTFPAVLQSSGLYSLSSVV | | VETTTPSKQSNNKYAASS | |
| | TVPSSSLGTKTYTCNVDHK | | YLSLTPEQWKSHRSYSCQ | |
| | PSNTKVDKRVESKYGPPCP | | VTHEGSTVEKTVAPTECS | |
| | PCPAPEAAGGPSVFLFPPKP | | | |
| | KDTLMISRTPEVTCVVVDV | | | |
| | SQEDPEVQFNWYVDGVEV | | | |
| | HNAKTKPREEQFNSTYRVV | | | |
| | SVLTVLHQDWLNGKEYKC | | | |
| | KVSNKGLPSSIEKTISKAKG | | | |
| | QPREPQVYTLPPSQEEMTK | | | |
| | NQVSLTCLVKGFYPSDIAVE | | | |
| | WESNGQPENNYKTTPPVLD | | | |
| | SDGSFFLYSRLTVDKSRWQ | | | |
| | EGNVFSCSVMHEALHNHYT | | | |
| | QKSLSLSLGK | | | |
| IAPB65 | QVQLVQSGAEVKKPGSSVK | 90 | EIVLTQSPATLSLSPGERA | 91 |
| | VSCKASGGTFSSYAISWVR | | TLSCRASQSVSNFLAWYQ | |
| | QAPGQGLEWMGGISAIFGT | | QKPGQAPRLLIYGASNRA | |
| | ANYAQKFQGRVTITADEST | | TGIPARFSGSGSGTDFTLTI | |
| | STAYMELSSLRSEDTAVYY | | SSLEPEDFAVYYCQQGKH | |
| | CARHLHNAIHLDYWGQGT | | WPWTFGQGTKVEIKRTV | |
| | LVTVSSASTKGPSVFPLAPC | | AAPSVFIFPPSDEQLKSGT | |
| | SRSTSESTAALGCLVKDYFP | | ASVVCLLNNFYPREAKVQ | |
| | EPVTVSWNSGALTSGVHTF | | WKVDNALQSGNSQESVT | |
| | PAVLQSSGLYSLSSVVTVPS | | EQDSKDSTYSLSSTLTLSK | |
| | SSLGTKTYTCNVDHKPSNT | | ADYEKHKVYACEVTHQG | |
| | KVDKRVESKYGPPCPPCPA | | LSSPVTKSFNRGEC | |
| | PEAAGGPSVFLFPPKPKDTL | | | |
| | MISRTPEVTCVVVDVSQED | | | |
| | PEVQFNWYVDGVEVHNAK | | | |
| | TKPREEQFNSTYRVVSVLT | | | |
| | VLHQDWLNGKEYKCKVSN | | | |
| | KGLPSSIEKTISKAKGQPREP | | | |
| | QVYTLPPSQEEMTKNQVSL | | | |
| | TCLVKGFYPSDIAVEWESN | | | |
| | GQPENNYKTTPPVLDSDGS | | | |
| | FFLYSRLTVDKSRWQEGNV | | | |
| | FSCSVMHEALHNHYTQKSL | | | |
| | SLSLGK | | | |

### Example 8: Crystal Structure of an Anti-IL1RAP Fab

The crystal structure of one anti-IL1RAP antibody (IAPB57) was determined in free fab form, as well as when bound to human IL1RAP ECD, to characterize the antibody/antigen interactions in atomic details, increase our understanding of the antibody mechanism of action, and support any required antibody engineering efforts.

Materials

His-tagged IAPB57 Fab was expressed in HEK293 cells and purified using affinity and size-exclusion chromatographies. The Fab was received in 50 mM NaCl, 20 mM Tris pH 7.4.

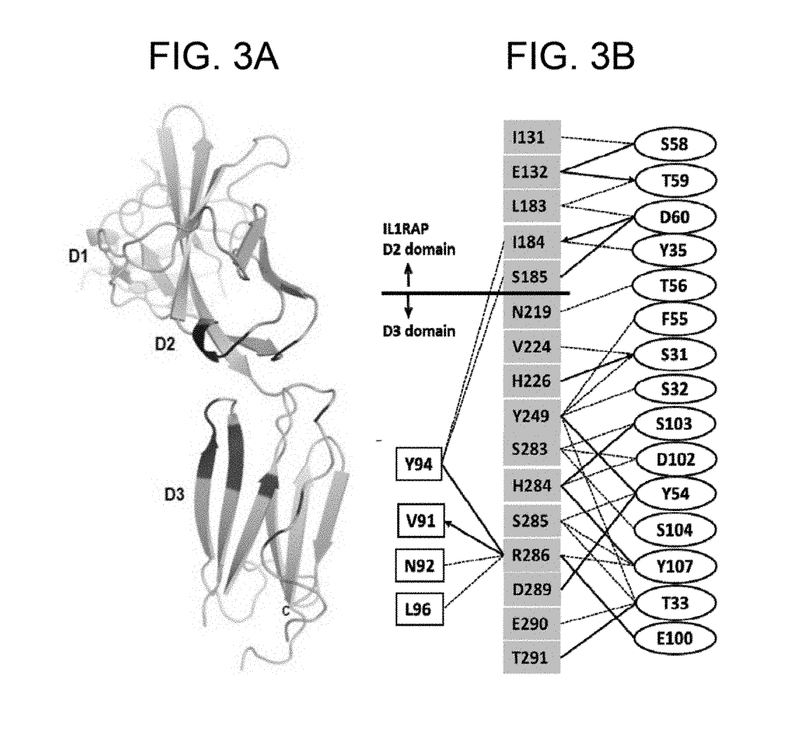

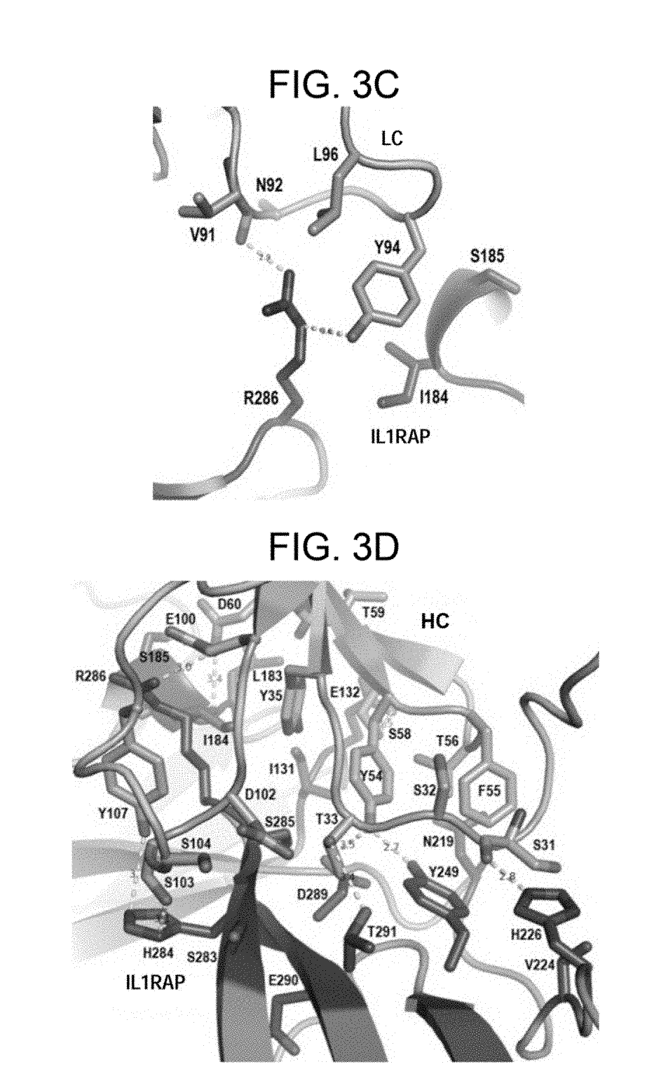

Human IL1RAP extracellular region (1-348 residues of mature isoforms 1, 2, and 4; hereafter simply IL1RAP) with a C-terminal His tag was expressed using the baculovirus system and purified by affinity and size-exclusion chromatography. The protein was received in 50 mM NaCl, 20 mM Tris pH 8 (FIGS. 3A, 3B, 3C and 3D).

Crystallization

IL1RAP/IAPB57 Fab Complex

The Fab/antigen complex was prepared by mixing IL1RAP with IAPB57 Fab at a molar ratio of 1.2:1 (excess IL1RAP) for 23 h at 4° C. while buffer exchanging to 20 mM Mes pH 6. The complex was then eluted from a monoS 5/50 column with a gradient of 16-19 mM NaCl in 20 mM Mes pH 6 and concentrated to 25 mg/mL. Crystals suitable for X-ray diffraction were obtained from 3.5 M sodium formate, 0.1 M Tris pH 8.5 using the sitting drop vapor-diffusion method at 20° C.

IAPB57 Fab

The IAPB57 Fab was concentrated to 14 mg/mL without further purification. Crystals suitable for X-ray diffraction were obtained from 25% PEG 3 kDa, 0.2 M (NH₄)₂SO₄, 0.1 M Mes pH 6.5 using the sitting drop vapor-diffusion method at 20° C.

X-Ray Data Collection and Structure Determination

For X-ray data collection, the crystals were soaked for few seconds in a cryo-protectant solution containing the corresponding mother liquor supplemented with 20% glycerol and then, flash frozen in liquid nitrogen. X-ray diffraction data were collected with a Rayonix 300HS CCD detector at beamline 22-ID of the Advanced Photon Source (APS) at Argonne National Laboratory. Diffraction data were processed with the program HKL (Otwinowski, Z. & Minor, W. (1997). Processing of X-ray diffraction data collected in oscillation mode. Methods in Enzymology 276: 307-326.).

The structures were solved by molecular replacement (MR) with Phaser (Read, R. J. (2001). Pushing the boundaries of molecular replacement with maximum likelihood. Acta Crystallogr D Biol Crystallogr 57: 1373-82). In the case of the free Fab structure, the search model for MR was the IMC-11F8 Fab (PDB code: 3B2U). In the case of the IL1RAP/Fab complex, the search models for MR were the crystal structures of IL1RAP (PDB code: 4DEP) and the IAPB57 free Fab structure. The structures were refined with PHENIX (Adams, P. D., Gopal, K., Grosse-Kunstleve, R. W., Hung, L. W., Ioerger, T. R., McCoy, A. J., Moriarty, N. W., Pai, R. K., Read, R. J., Romo, T. D., Sacchettini, J. C., Sauter, N. K., Storoni, L. C. & Terwilliger, T. C. (2004). Recent developments in the PHENIX software for automated crystallographic structure determination. J Synchrotron Radiat 11: 53-5.) and model adjustments were carried out using COOT (Emsley P. & Cowtan, K. (2004). Coot: Model building tools for molecular graphics. Acta Crystallogr. D60: 2126-2132). All other crystallographic calculations were performed with the CCP4 suite of programs (Collaborative Computational Project Number 4, 1994). All molecular graphics were generated with PyMol (DeLano, W. (2002). The PyMOL molecular graphics system. Palo Alto, Calif., USA; Delano Scientific).

The data statistics for both the IAPB57 free Fab structure and the complex are shown in Table 9.

TABLE 9
Crystallographic data for the IL1RAP ECD/IAPB57
Fab complex and free IAPB57 Fab.

| | |
|---|---|
| FAB-IL1RAP ECD | |
| Complex | Free Fab |

Crystal data
Crystallization solution

| | | |
|---|---|---|
| 0.1M Buffer | Tris pH 8.5 | Mes pH 6.5 |
| Precipitant | 3.5M Na Formate | 25% PEG 3 kDa |
| Additive | | 0.2M (NH4)₂SO4 |
| Space group | H32 | P2₁ |
| Molecules/asymmetric unit | 2 | 2 |

Unit cell

| | | |
|---|---|---|
| a, b, c (Å) | 419.6, 419.6, 92.9 | 73.9, 63.6, 100.7 |
| β (°) | 120.0 | 110.8 |
| Solvent content (%) | 73 | 47 |

X-ray data*

| | | |
|---|---|---|
| Resolution (Å) | 50.00-3.08 | 50.00-1.88 |
| Highest Resolution Shell (Å) | (3.19-3.08) | (1.95-1.88) |
| Measured reflections | 611, 321 | 261, 192 |
| Completeness (%) | 100 (100) | 99.9 (99.1) |
| Redundancy | 10.6 (3.6) | 3.7 (3.4) |
| R_{sym} (%) | 11.9 (51.7) | 5.8 (52.9) |
| <I/σ> | 18.2 (5.7) | 21.4 (2.3) |

Refinement

| | | |
|---|---|---|
| Resolution (Å) | 48.13-3.08 | 48.09-1.88 |
| Number of reflections | 57,425 | 70,151 |
| Number of all atoms | 10,465 | 6,609 |
| Number of waters | 36 | 142 |
| R_{work}/R_{free} (%) | 21.1/24.6 | 20.8/24.5 |
| Bond length RMSD (Å) | 0.014 | 0.007 |
| Bond angle RMSD (°) | 1.414 | 1.119 |
| Mean B-factor (Å²) | 71.1 | 37.3 |

MolProbity

| | | |
|---|---|---|
| Ramachandran favored (%) | 91.92 | 97.12 |
| Ramachandran allowed (%) | 7.93 | 2.65 |
| Ramachandran outliers (%) | 0.15 | 0.23 |
| Rotamer outliers (%) | 0.47 | 0.42 |
| Clash score | 6.2 | 2.7 |

The Epitope, Paratope and Interactions

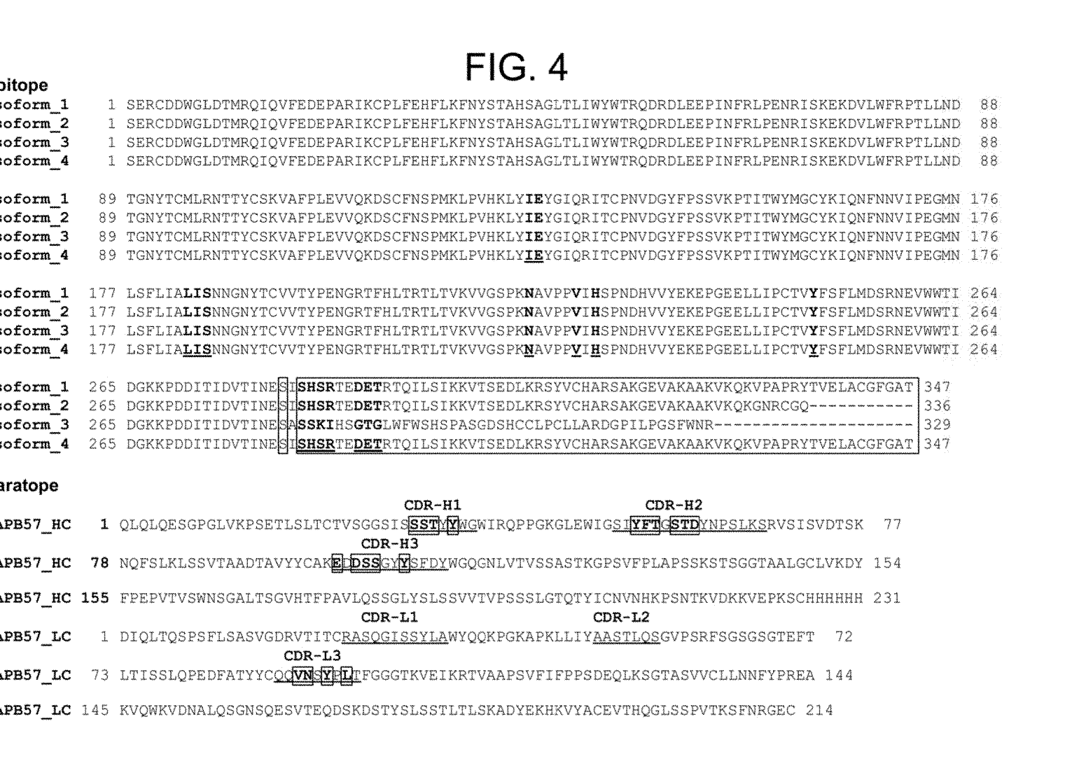

IAPB57 recognizes a conformational epitope composed of residues in the D2 (residues 1131, E132, and L183-S185) and D3 (residues N219, V224, H226, Y249, S283-R286, and D289-T291) immunoglobulin-like domains of IL1RAP as seen in FIGS. 3A, 3B, 3C, 3D and 4. The IAPB57 epitope comprises an area of about 780 Å² on IL1RAP. The majority of antibody contacts are with the D3 domain of IL1RAP; however, a number of hydrogen bond interactions involve D2 (FIG. 3A, 3B, 3C, 3D), which strengths the IAPB57 affinity for IL1RAP. Arginine 286 is a key epitope residue and it is inserted in a pocket lined by IAPB57 light and heavy chain residues V91^{L}, N92^{L}, Y94^{L}, L96^{L}, E100^{H}, and Y107^{H}. Other prevalent epitope residues are Y249 and H284, which are on opposite ends of the IL1RAP β-sheet and have extensive van der Waals and hydrogen bond interactions with the heavy chain CDRs.

The IAPB57 paratope is composed of residues from all CDRs except CDR-L1 and -L2 (FIGS. 3A, 3B, 3C, 3D and 4). The heavy chain has five-fold more contacts with IL1RAP than the light chain. The heavy chain CDRs packs onto the convex surface of IL1RAP with the CDR-H2 β-strand (S58-D60 residues) interacting with D2 residues, while the CDR-H2 loop region (Y54-T56 residues) binds D3. CDR-H3 binds only the D3 domain (S283-R286 residue range), while CDR-H1 and -L3 bind both D2 and D3.

Alternative splicing of the IL1RAP gene results in transcript variants encoding the membrane-bound isoforms 1 and 4 and the soluble isoforms 2 and 3. The extracellular region of membrane-bound isoforms 1 and 4 differs in sequence from secreted isoforms 2 and 3 (FIG. 3A, 3B, 3C, 3D). The extracellular differences are located in the D3 domain and linker region to the transmembrane domain. Six of the IAPB57 epitope residues (H284, 5285, R286, D289, E290, and T291) are located within the isoform 3 unique region. Therefore, we expect IAPB57 to bind with similar affinity to isoforms 1, 2, 4 and with lower affinity to isoform 3 due to loss of hydrogen bond interactions between the antibody and isoform 3. Specifically, the R286-Y94^{L}, R286-V91^{L}, D289-Y54^{H}, and T291-T33^{H} hydrogen bonds might be disrupted in the IAPB57/isoform 3 complex.

### Example 9: Preparation of IL1RAP and CD3 Antibodies in a Bispecific Format in IgG4 S228P, L234A, L235A

Fifteen of the monospecific IL1RAP antibodies (see table 6) were expressed as IgG4, having Fc substitutions S228P, L234A, and L235A or S228P, L234A, L235A, F405L, and R409K (CD3 arm) (numbering according to EU index). A monospecific anti-CD3 antibody CD3B220 was also generated comprising the VH and VL regions having the VH of SEQ ID NO: 92 and the VL of SEQ ID NO: 93 and IgG4 constant region with S228P, L234A, L235A, F405L, and R409K substitutions.

The monospecific antibodies were purified using standard methods using a Protein A column (HiTrap MabSelect SuRe column). After elution, the pools were dialyzed into D-PBS, pH 7.2.

Bispecific IL1RAP x CD3 antibodies were generated by combining a monospecific CD3 mAb and a monospecific IL1RAP mAb in in-vitro Fab arm exchange (as described in WO2011/131746). Briefly, at about 1-20 mg/mL at a molar ratio of 1.08:1 of anti-IL1RAP/anti-CD3 antibody in PBS, pH 7-7.4 and 75 mM 2-mercaptoethanolamine (2-MEA) was mixed together and incubated at 25-37° C. for 2-6 hours, followed by removal of the 2-MEA via dialysis, diafiltration, tangential flow filtration and/or spin cell filtration using standard methods.

Heavy and Light chains for the IL1RAP x CD3 bispecific Abs are shown below in Table 10.

TABLE 10
Heavy and Light Chain Sequences for bispecific Abs IgG4-PAA

| Ab | | Amino Acid Sequence |
|---|---|---|
| IC3B1 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | EVQLVQSGAEVKKPGESLKISCKGSGYSFTSYWIGWVRQM |
| | IAPB47 | PGKGLEWMGIIYPSDSYTRYSPSFQGQVTISADKSISTAYLQ |
| | (SEQ ID | WSSLKASDTAMYYCARRNSAENYADLDYWGQGTLVTVSS |
| | NO: 68) | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEAAGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| | Light Chain 2 | EIVLTQSPGTLSLSPGERATLSCRASQSISNDLNWYQQKPGK |
| | IAPB47 | APKLLIYYASSLQSGVPSRFSGSGSGTDFTLTINSLQPEDFAT |
| | (SEQ ID | YYCQQSFTAPLTFGQGTKVEIKRTVAAPSVFIFPPSDEQLKS |
| | NO: 69) | GTASVVCLLNNFYPREAKVQWKVDNALQSGNSQESVTEQ |
| | | DSKDSTYSLSSTLTLSKADYEKHKVYACEVTHQGLSSPVTK |
| | | SFNRGEC |
| IC3B2 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | EVQLLESGGGLVQPGGSLRLSCAASGFTFSNYAMNWVRQ |
| | IAPB38 | APGKGLEWVSGINYGGGSKYYADSVKGRFTISRDNSKNTL |
| | (SEQ ID | YLQMNSLRAEDTAVYYCAKDYGPFALDYWGQGTLVTVSS |
| | NO: 70) | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEAAGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| | Light Chain 2 | EIVLTQSPATLSLSPGERATLSCRASQSVDDWLAWYQQKP |
| | IAPB38 | GQAPRLLIYTASNRATGIPARFSGSGSGTDFTLTISSLEPEDF |
| | (SEQ ID | AVYYCQQYHHWPLTFGQGTKVEIKRTVAAPSVFIFPPSDEQ |
| | NO: 71) | LKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |
| IC3B3 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QLQLQESGPGLVKPSETLSLTCTVSGGSISSSTYYWGWIRQP |
| | IAPB57 | PGKGLEWIGSIYFTGSTDYNPSLKSRVSISVDTSKNQFSLKL |
| | (SEQ ID | SSVTAADTAVYYCAKEDDSSGYYSFDYWGQGNLVTVSSA |
| | NO: 72) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain 2 | DIQLTQSPSFLSASVGDRVTITCRASQGISSYLAWYQQKPG |
| | IAPB57 | KAPKLLIYAASTLQSGVPSRFSGSGSGTEFTLTISSLQPEDFA |
| | (SEQ ID | TYYCQQVNSYPLTFGGGTKVEIKRTVAAPSVFIFPPSDEQL |
| | NO: 73) | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| IC3B4 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QLQLQESGPGLVKPSETLSLTCTVSGVSISSSTYYWGWLRQ |
| | IAPB61 | PPGMGLEWTGSIYFTGNTYYNPSLKSRVTISVDTSRNQFSL |
| | (SEQ ID | KLSSVTAADTAVYYCGSLFGDYGYFDYWGQGTLVTVSSA |
| | NO: 74) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain 2 | EIVMTQSPATLSVPPGERATLSCRASQFISSNLAWYQQKPG |
| | IAPB61 | QAPRLLIYGASTRATGIPARFSGSGSGTDFTLTISSLQSEDFA |
| | (SEQ ID | VYYCQQYNNWPSTFGPGTKVDIKRTVAAPSVFIFPPSDEQL |
| | NO: 75) | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| IC3B5 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGSELKKPGASVKVSCKASGYTFNTYAMNWVRQ |
| | IAPB62 | APGQGLEWMGWINTNTGNPTYAQGFTGRFVFSLDTSVSTA |
| | (SEQ ID | YLQISSLKAEDTAVYYCARRYFDWLLGAFDIWGQGTMVT |
| | NO: 76) | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEAAGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLGK |
| | Light Chain 2 | DIQMTQSPSSVSASVGDRVTITCRASQGISSWLAWYQQKPG |
| | IAPB62 | KAPKLLIYAASSLQSGVPSRFSGSGSGTDFTLTISSLQPEDFA |
| | (SEQ ID | TYYCQQANSFPLTFGGGTKVEIKRTVAAPSVFIFPPSDEQLK |
| | NO: 77) | SGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| IC3B6I | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGAEVKKPGSSVKVSCKASGGTFSSYAISWVRQA |
| | IAPB3 | PGQGLEWMGGISAIFGTANYAQKFQGRVTITADESTSTAY |
| | (SEQ ID | MELSSLRSEDTAVYYCARGNSFHALWDYAFDYWGQGTLV |
| | NO: 78) | TVSSASTKGPSVFPLAPCSRSTSESTAALGCLVKDYFPEPVT |
| | | VSWNSGALTSGVHTFPAVLQSSGLYSLSSVVTVPSSSLGTK |
| | | TYTCNVDHKPSNTKVDKRVESKYGPPCPPCPAPEAAGGPS |
| | | VFLFPPKPKDTLMISRTPEVTCVVVDVSQEDPEVQFNWYV |
| | | DGVEVHNAKTKPREEQFNSTYRVVSVLTVLHQDWLNGKE |
| | | YKCKVSNKGLPSSIEKTISKAKGQPREPQVYTLPPSQEEMT |
| | | KNQVSLTCLVKGFYPSDIAVEWESNGQPENNYKTTPPVLD |
| | | SDGSFFLYSRLTVDKSRWQEGNVFSCSVMHEALHNHYTQK |
| | | SLSLSLGK |
| | Light Chain 2 | DIVMTQSPDSLAVSLGERATINCKSSQSVLYSSNNKNYLAW |
| | IAPB3(SEQ | YQQKPGQPPKLLIYWASTRESGVPDRFSGSGSGTDFTLTISS |
| | ID NO: 79) | LQAEDVAVYYCQQYYSTPLTFGQGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| IC3B7 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain | QVQLVQSGAEVKKPGSSVKVSCKASGGTFSSYAISWVRQA |
| | IAPB17 | PGQGLEWMGGIIPIFGNANYAQKFQGRVTITADESTSTAYM |
| | (SEQ ID | ELSSLRSEDTAVYYCARTIIYLDYVHILDYWGQGTLVTVSS |
| | NO: 80) | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEAAGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| | Light Chain | DIVMTQSPDSLAVSLGERATINCKSSQSVLYSSNNKNYLAW |
| | IAPB17 | YQQKPGQPPKLLIYWASTRESGVPDRFSGSGSGTDFTLTISS |
| | (SEQ ID NO: | LQAEDVAVYYCQQYYSTPLTFGQGTKVEIKRTVAAPSVFIF |
| | 79) | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| IC3B8 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain | EVQLLESGGGLVQPGGSLRLSCAASGFTFSNYWMNWVRQ |
| | IAPB23 | APGKGLEWVSAIRYDGGSKYYADSVKGRFTISRDNSKNTL |
| | (SEQ ID | YLQMNSLRAEDTAVYYCAKDAYPPYSFDYWGQGTLVTVS |
| | NO: 81) | SASTKGPSVFPLAPCSRSTSESTAALGCLVKDYFPEPVTVS |
| | | WNSGALTSGVHTFPAVLQSSGLYSLSSVVTVPSSSLGTKTY |
| | | TCNVDHKPSNTKVDKRVESKYGPPCPPCPAPEAAGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSL |
| | | SLGK |
| | Light Chain | EIVLTQSPATLSLSPGERATLSCRASQSVSSYLAWYQQKPG |
| | IAPB23 | QAPRLLIYDASNRATGIPARFSGSGSGTDFTLTISSLEPEDFA |
| | (SEQ ID | VYYCQQRSNWPLTFGQGTKVEIKRTVAAPSVFIFPPSDEQL |
| | NO: 82) | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| IC3B9 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain | EVQLLESGGGLVQPGGSLRLSCAASGFTFSSYAMSWVRQA |
| | IAPB25 | PGKGLEWVSAISGSGGSTYYADSVKGRFTISRDNSKNTLYL |
| | (SEQ ID | QMNSLRAEDTAVYYCAKGDEYYYPDPLDYWGQGTLVTV |
| | NO: 83) | SSASTKGPSVFPLAPCSRSTSESTAALGCLVKDYFPEPVTVS |
| | | WNSGALTSGVHTFPAVLQSSGLYSLSSVVTVPSSSLGTKTY |
| | | TCNVDHKPSNTKVDKRVESKYGPPCPPCPAPEAAGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSL |
| | | SLGK |
| | Light Chain | DIQMTQSPSSLSASVGDRVTITCRASQSISSYLNWYQQKPG |
| | IAPB25 | KAPKLLIYAASSLQSGVPSRFSGSGSGTDFTLTISSLQPEDFA |
| | (SEQ ID NO: | TYYCQQSYSTPLTFGQGTKVEIKRTVAAPSVFIFPPSDEQLK |
| | 84) | SGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| IC3B10 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain | EVQLLESGGGLVQPGGSLRLSCAASGFTFSNYAMSWVRQA |
| | IAPB29 | PGKGLEWVSAISGSGGSTYYADSVKGRFTISRDNSKNTLYL |
| | (SEQ ID | QMNSLRAEDTAVYYCAKEWSSYFGLDYWGQGTLVTVSSA |
| | NO: 85) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain | DIQMTQSPSSLSASVGDRVTITCRASQSISSYLNWYQQKPG |
| | IAPB29 | KAPKLLIYAASSLQSGVPSRFSGSGSGTDFTLTISSLQPEDFA |
| | (SEQ ID | TYYCQQSYSTPLTFGQGTKVEIKRTVAAPSVFIFPPSDEQLK |
| | NO: 84) | SGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| IC3B11 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGAEVKKPGSSVKVSCKASGGTFSSYAISWVRQA |
| | IAPB9 (SEQ | PGQGLEWMGWISPIFGTANYAQKFQGRVTITADESTSTAY |
| | ID NO: 86) | MELSSLRSEDTAVYYCARRYDNFARSGDLDYWGQGTLVT |
| | | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEAAGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLGK |
| | Light Chain 2 | DIQMTQSPSSLSASVGDRVTITCRASQSISSYLNWYQQKPG |
| | IAPB9 (SEQ | KAPKLLIYAASSLQSGVPSRFSGSGSGTDFTLTISSLQPEDFA |
| | ID NO: 84) | TYYCQQSYSTPLTFGQGTKVEIKRTVAAPSVFIFPPSDEQLK |
| | | SGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| IC3B12 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QLQLQESGPGLVKPSETLSLTCTVSGVSISSSTYYWGWLRQ |
| | IAPB55 | PPGMGLEWTGSIYFTGNTYYNPSLKSRVTISVDTSRNQFSL |
| | (SEQ ID | KLSSVTAADTAVYYCGSLFGDYGYFDYWGQGTLVTVSSA |
| | NO: 74) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain 2 | EIVMTQSPATLSVSPGERATLSCRASQFISSNLAWYQQKPG |
| | IAPB55 | QAPRLLIYGASTRATGIPARFSGSGSGTDFTLTISSLQSEDFA |
| | (SEQ ID | VYYCQQYNNWPFTFGPGTKVDIKRTVAAPSVFIFPPSDEQL |
| | NO: 87) | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| IC3B13 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGSELKKPGASVKVSCKASGYTFNTYAMNWVRQ |
| | IAPB63 | APGQGLEWMGWINTNTGNPTYAQGFTGRFVFSLDTSVSTA |
| | (SEQ ID | YLQISSLKAEDTAVYYCARRYFDWLLGAFDIWGQGTMVT |
| | NO: 76) | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEAAGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLGK |
| | Light Chain 2 | QSALTQPRSVSGSPGHSVTISCTGTSSDVGDYNYVSWYQQ |
| | IAPB63 | RPGKVPKLLIYDVSKRPSGVPDRFSGSKSGNTASLTISGLQA |
| | (SEQ ID | EDEAIYFCASYAGNYNVVFGGGTKLTVLGQPKAAPSVTLF |
| | NO: 88) | PPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| IC3B14 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGSELKKPGASVKVSCKASGYTFNTYAMNWVRQ |
| | IAPB64 | APGQGLEWMGWINTNTGNPTYAQGFTGRFVFSLDTSVSTA |
| | (SEQ ID | YLQISSLKAEDTAVYYCARRYFDWLLGAFDIWGQGTMVT |
| | NO: 76) | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEAAGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLGK |
| | Light Chain 2 | QSALTQPRSVSGSPGHSVTISCTGTSSDVGDYNYVSWYQQ |
| | IAPB64 | RPGKVPKLLIYDVSKRPSGVPDRFSGSKSGNTASLTISGLQA |
| | (SEQ ID | EDEAIYFCSSYAGNYNVVFGGGTKLTVLGQPKAAPSVTLFP |
| | NO: 89) | PSSEELQANKATLVCLISDFYPGAVTVAWKADSSPVKAGV |
| | | ETTTPSKQSNNKYAASSYLSLTPEQWKSHRSYSCQVTHEGS |
| | | TVEKTVAPTECS |
| IC3B15 | Heavy chain 1 | EVQLVESGGGLVQPGGSLKLSCAASGFTFNTYAMNWVRQ |
| | CD3B220 | ASGKGLEWVGRIRSKYNAYATYYAASVKGRFTISRDDSKN |
| | (SEQ ID | TAYLQMNSLKTEDTAVYYCTRHGNFGNSYVSWFAYWGQ |
| | NO: 92) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B220 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 93) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGAEVKKPGSSVKVSCKASGGTFSSYAISWVRQA |
| | IAPB65 | PGQGLEWMGGISAIFGTANYAQKFQGRVTITADESTSTAY |
| | (SEQ ID | MELSSLRSEDTAVYYCARHLHNAIHLDYWGQGTLVTVSSA |
| | NO: 90) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain 2 | EIVLTQSPATLSLSPGERATLSCRASQSVSNFLAWYQQKPG |
| | IAPB65 | QAPRLLIYGASNRATGIPARFSGSGSGTDFTLTISSLEPEDFA |
| | (SEQ ID | VYYCQQGKHWPWTFGQGTKVEIKRTVAAPSVFIFPPSDEQ |
| | NO: 91) | LKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |

### Example 10. Anti-IL1RAP Affinity Determinations on the IL1RAP x CD3 Bispecific Antibodies

Surface Plasmon Resonance (SPR) was used to measure affinity values of the 15 IL1RAPxCD3 bispecfic Abs for human and cyno IL1RAP. The protocol followed was similar to that described in Example 5. The results indicated these IL1RAP x CD3 bispecific Abs have binding affinities of 34 pM to 29.7 nM to human IL1RAP ECD (Table 11) and 86 pM to 27.8 nM binding affinities to cyno IL1RAP ECD (Table 12). However, one molecule, IC3B3, showed weak binding to both human and cyno IL1RAP ECDs. Comparing affinities of human to cyno for all good binders showed they bound within 5-fold from each other (Table 13).

TABLE 11
Summary of kinetics affinity for IL1RAP × CD3 bispecific
Abs binding to recombinant human IL1RAP ECD (1.2-100 nM).
The parameters reported in this table were obtained from
a 1:1 Langmuir binding model. Affinity, KD = kd/ka.

| | Protein | | | |
|---|---|---|---|---|
| bispecific | description | ka (1/Ms) | kd (1/s) | KD (M) |
| IC3B1 | IAPB47 × CD3B220 | 6.97E+05 | 7.59E−04 | 1.09E−09 |
| IC3B2 | IAPB38 × CD3B220 | 1.12E+05 | 8.27E−04 | 7.36E−09 |
| IC3B3 | IAPB57 × CD3B220 | 8.75E+05 | 2.98E−05 | 3.40E−11 |
| IC3B4 | IAPB61 × CD3B220 | 1.15E+06 | 1.29E−02 | 1.12E−08 |
| IC3B5 | IAPB62 × CD3B220 | | | Weak |
| | | | | binding |
| IC3B6 | IAPB3 × CD3B220 | 1.67E+05 | 3.81E−04 | 2.29E−09 |
| IC3B7 | IAPB17 × CD3B220 | 1.08E+06 | 6.59E−03 | 6.10E−09 |
| IC3B8 | IAPB23 × CD3B220 | 3.00E+05 | 2.98E−03 | 9.96E−09 |
| IC3B9 | IAPB25 × CD3B220 | 1.84E+06 | 5.47E−02 | 2.97E−08 |
| IC3B10 | IAPB29 × CD3B220 | 3.84E+05 | 1.83E−03 | 4.77E−09 |
| IC3B11 | IAPB9 × CD3B220 | 7.76E+05 | 3.54E−03 | 4.56E−09 |
| IC3B12 | IAPB55 × CD3B220 | 1.15E+06 | 3.61E−04 | 3.13E−10 |
| IC3B13 | IAPB63 × CD3B220 | 9.38E+05 | 1.14E−04 | 1.22E−10 |
| IC3B14 | IAPB64 × CD3B220 | 6.95E+05 | 1.71E−04 | 2.46E−10 |
| IC3B15 | IAPB65 × CD3B220 | 3.43E+05 | 3.95E−03 | 1.15E−08 |

TABLE 12
Summary of kinetics affinity for IL1RAPxCD3 bispecific
Abs binding to recombinant cyno IL1RAP ECD (1.2-100 nM).
The parameters reported in this table were obtained from a
1:1 Langmuir binding model. Affinity, KD = kd/ka.

| | Protein | | | |
|---|---|---|---|---|
| bispecific | Description | ka (1/Ms) | kd (1/s) | KD (M) |
| IC3B1 | IAPB47xCD3B220 | 1.11E+06 | 2.36E−04 | 2.12E−10 |
| IC3B2 | IAPB38xCD3B220 | 1.32E+05 | 2.23E−03 | 1.69E−08 |
| IC3B3 | IAPB57xCD3B220 | 9.52E+05 | 8.20E−05 | 8.61E−11 |
| IC3B4 | IAPB61xCD3B220 | 1.46E+06 | 1.48E−02 | 1.02E−08 |
| IC3B5 | IAPB62xCD3B220 | | | Weak binding |
| IC3B6 | IAPB3xCD3B220 | 1.80E+05 | 5.40E−04 | 2.99E−09 |
| IC3B7 | IAPB17xCD3B220 | 1.23E+06 | 5.83E−03 | 4.74E−09 |
| IC3B8 | IAPB23xCD3B220 | 4.48E+05 | 1.21E−03 | 2.70E−09 |
| IC3B9 | IAPB25xCD3B220 | 1.91E+06 | 5.30E−02 | 2.78E−08 |
| IC3B10 | IAPB29xCD3B220 | 2.48E+05 | 3.83E−04 | 1.54E−09 |
| IC3B11 | IAPB9xCD3B220 | 7.76E+05 | 4.09E−03 | 5.27E−09 |
| IC3B12 | IAPB55xCD3B220 | 1.52E+06 | 3.31E−04 | 2.18E−10 |
| IC3B13 | IAPB63xCD3B220 | 1.18E+06 | 5.32E−04 | 4.51E−10 |
| IC3B14 | IAPB64xCD3B220 | 8.64E+05 | 8.58E−04 | 9.93E−10 |
| IC3B15 | IAPB65xCD3B220 | 3.79E+05 | 3.44E−03 | 9.08E−09 |

TABLE 13
Comparing the Human to Cyno binding affinity of the
IL1RAPxCD3 bispecific Abs. Test human and cyno
IL1RAP at 1.2-100 nM. Affinity, KD = kd/ka.

| | | Human KD | Cyno KD | Hu/Cyno |
|---|---|---|---|---|
| bispecific | Protein Description | (M) | (M) | KD Ratio |
| IC3B1 | IAPB47xCD3B220 | 1.09E−09 | 2.12E−10 | 5.1 |
| IC3B2 | IAPB38xCD3B220 | 7.36E−09 | 1.69E−08 | 0.4 |
| IC3B3 | IAPB57xCD3B220 | 3.40E−11 | 8.61E−11 | 0.4 |
| IC3B4 | IAPB61xCD3B220 | 1.12E−08 | 1.02E−08 | 1.1 |
| IC3B5 | IAPB62xCD3B220 | Weak binding | Weak | NA |
| | | | binding | |
| IC3B6 | IAPB3xCD3B220 | 2.29E−09 | 2.99E−09 | 0.8 |
| IC3B7 | IAPB17xCD3B220 | 6.10E−09 | 4.74E−09 | 1.3 |
| IC3B8 | IAPB23xCD3B220 | 9.96E−09 | 2.70E−09 | 3.7 |
| IC3B9 | IAPB25xCD3B220 | 2.97E−08 | 2.78E−08 | 1.1 |
| IC3B10 | IAPB29xCD3B220 | 4.77E−09 | 1.54E−09 | 3.1 |
| IC3B11 | IAPB9xCD3B220 | 4.56E−09 | 5.27E−09 | 0.9 |
| IC3B12 | IAPB55xCD3B220 | 3.13E−10 | 2.18E−10 | 1.4 |
| IC3B13 | IAPB63xCD3B220 | 1.22E−10 | 4.51E−10 | 0.3 |
| IC3B14 | IAPB64xCD3B220 | 2.46E−10 | 9.93E−10 | 0.2 |
| IC3B15 | IAPB65xCD3B220 | 1.15E−08 | 9.08E−09 | 1.3 |

### Example 11: Competition Binning Assay

This assay permits assessment of the panel of the 15 produced IL1RAPxCD3 bispecific Abs individually as both capture and detection reagents with the rest of the antibodies in the panel. Antibodies forming effective capture/detection reagents with each other theoretically recognize spatially-separated epitopes on a monomeric protein, thus allowing both antibodies to bind to the target protein at the same time. Groups of antibodies exhibiting similar patterns of activity across the entire panel are hypothesized to bind to similar epitopes. Selecting clones from different groups should therefore provide antibodies recognizing different epitopes.

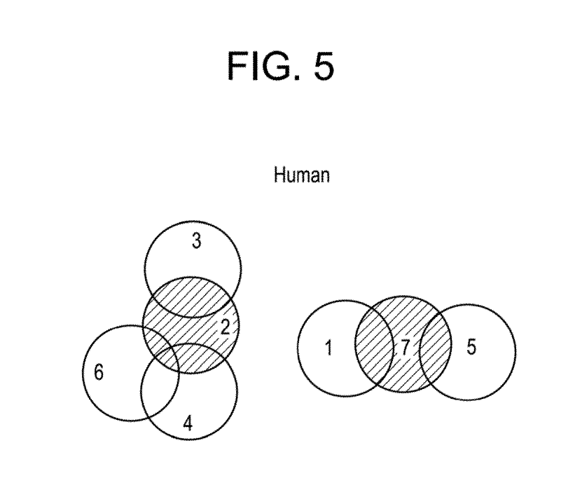

The bispecific Abs were directly immobilized on GLC sensors (BioRad). Competing samples (300 nM) were pre-incubated with 30 nM of hIL1RAP-ECD for 4 hours before injection over the chip surface for 5 minutes to allow association. Dissociation was then monitored for 5 minutes. Most of the molecules grouped into bins 1 and 2, and group members did not compete with each other (see Table 14). This indicates that there was no overlap in their binding epitopes. Bin 3 has two members, while Bins 4 to 7 have one member each. The Venn diagram shows the summary of competition profiles of epitope groups (FIG. 5). If epitope groups intersect, the antibodies compete. Otherwise, they do not compete for human IL1RAP. It should be noted that the conclusions drawn here were mostly from competition with Set1 (B1, B3, B6, B9, B12, B13) on the sensor, which gave clear results due to their strong binding affinities. Competition from Set2 (B2, B4, B8, B10, B11, B15) on the sensor were much weaker due to their weak binding affinities, Bin 7 comes from this set.

TABLE 14
Summary of epitope binning of 15 IL1RAPxCD3 bispecific Abs.
Members of any one epitope group have the same competition profiles.

| Epitope Group | |
|---|---|
| Bin # | Bispecific Abs |
| 1 | IC3B1, IC3B2, IC3B8, IC3B10 |
| 2 | IC3B4, IC3B5, IC3B12, IC3B13, IC3B14 |
| 3 | IC3B3, IC3B9 |
| 4 | IC3B6 |
| 5 | IC3B11 |
| 6 | IC3B15 |
| 7 | IC3B7 |

### Example 12: Evaluation of Bispecific Antibodies in Functional Cell Killing Assay

T-cell mediated cytotoxicity assay is a functional assay to evaluate the IL1RAP x CD3 bispecific Abs for cell lysis using T-cells from healthy donors.

The protocol of Laszlo, et al was followed (Laszlo, G., et al 2014 BLOOD 123:4, 554-561). Briefly, effector cells were harvested, counted, washed, and resuspended to 1×10⁶ cells/ml in RPMI (10% FBS) cell media. Target cells (MV4-11, SKNO-1, and OCI-AML5) were labeled with CFSE (Invitrogen #C34554) and resuspended to 2×10⁵ cells/mL in RPMI (Invitrogen #61870-036) with 10% FBS (Invitrogen #10082-147). Effectors and CFSE-labeled target cells were mixed at effector to target (E:T) ratio=5:1 in sterile 96-well round bottom plates. A 5 aliquot of each bispecific antibody was added to each well containing various concentrations. Cultures were incubated for 48 hours at 37° C. under 5% CO₂. After 48 hr, The LIVE/DEAD® Fixable Near-IR Dead Cell Stain buffer (life technologies Cat# L10119) was added to samples, and cultures were incubated for 20 minutes in the dark at RT, washed, and resuspended in 170 μL FACs buffer. The drug-induced cytotoxicity was determined using CANTO II flow cytometer (BD Biosciences) and analyzed with FlowJo Software or Dive software (BD Biosciences). The population of interest is the double positive CFSE+/live/dead+ cells.

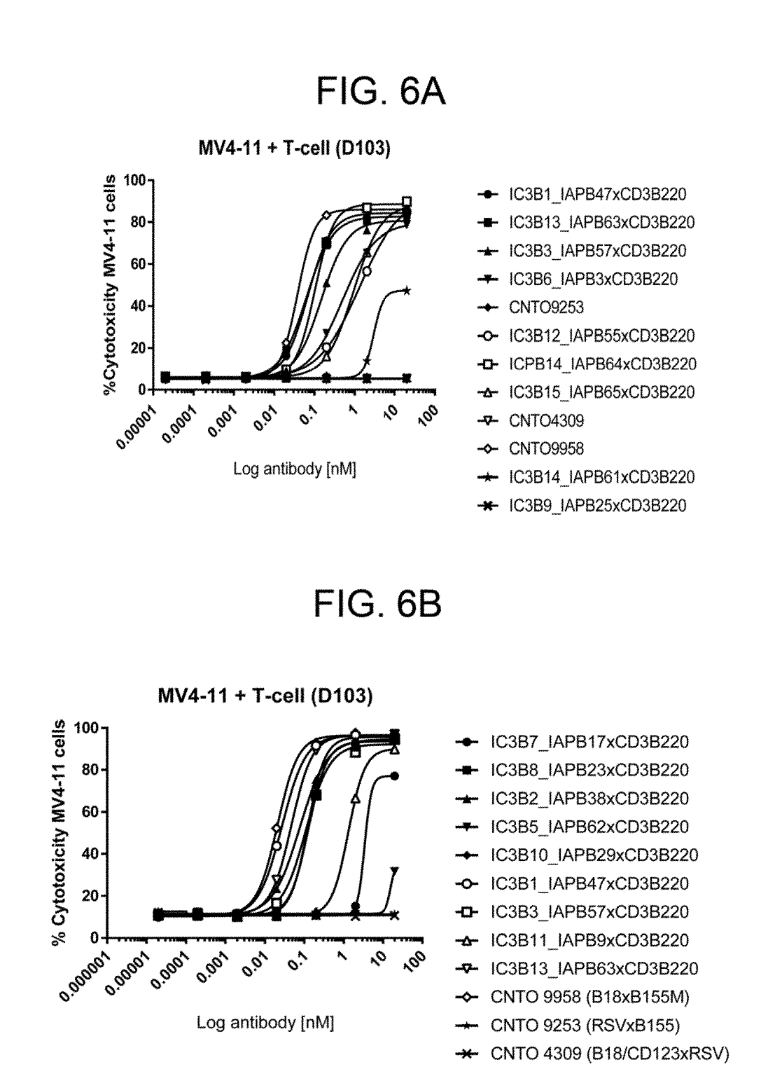

The results of the T-cell mediated cell lysis of one of the AML cell lines (MV4-11; FIGS. 6A and 6B) after 48 hour incubation at 37° C., 5% CO₂ are shown.

All of the IL1RAP antibodies, except IAPB61 and IAPB25, when combined with an anti-CD3 antibody into a bispecific format, elicit T cell redirected cell cytotoxicity of IL1RAP+MV4-11 cells at 48 hours in three different T cell donors. Table 14 summarizes the EC₅₀ values generated with the IL1RAPxCD3 multispecific antibodies.

### Example 13: Summary of Biochemical Characteristics of IL1RAPxCD3 Bispecific Abs

The results from the cell cytotoxicity and biochemical assays were collated (Table 15). A total of four bispecific antibodies: IC3B1, IC3B13, IC3B3, and IC3B12 had desirable characteristics including human/cyno-only binders. The selections spanned three different epitope bins, and all but IC3B1 had IL1RAP affinities in the sub-nM range. Additionally, two of the four bispecific Abs showed neutralization function in an antibody format.

TABLE 15
A summation of the secondary assay and screening
data for the top 15 IL1RAP x CD3 candidates.

| | Protein | Competition | Murine | | Affinity (nM) | | EC50 |
|---|---|---|---|---|---|---|---|
| bispecific | Description | Bin | Binder | Neutralizer | Human | Cyno | (nM) |
| IC3B1 | IAPB47xCD3B220 | 1 | Weak 6x | | 1.09 | 0.212 | 0.049^{b} |
| IC3B2 | IAPB38xCD3B220 | 1 | X | | 7.36 | 16.9 | 0.077 |
| IC3B8 | IAPB23xCD3B220 | 1 | | | 9.96 | 2.70 | 0.138 |
| IC3B10 | IAPB29xCD3B220 | 1 | X | | 4.77 | 1.54 | 0.124 |
| IC3B4 | IAPB61xCD3B220 | 2 | | | 11.2 | 10.2 | ND |
| IC3B5 | IAPB62xCD3B220 | 2 | | | Weak | Weak | ND |
| | | | | | binding | binding | |
| IC3B12 | IAPB55xCD3B220 | 2 | | | 0.313 | 0.218 | 1.30 |
| IC3B13 | IAPB63xCD3B220 | 2 | | X^{a} | 0.122 | 0.451 | 0.054^{b} |
| IC3B14 | IAPB64xCD3B220 | 2 | | X^{a} | 0.246 | 0.993 | 0.100 |
| IC3B3 | IAPB57xCD3B220 | 3 | | X | 0.034 | 0.086 | 0.131^{b} |
| IC3B9 | IAPB25xCD3B220 | 3 | | | 29.7 | 27.8 | ND |
| IC3B6 | IAPB3xCD3B220 | 4 | | | 2.29 | 2.99 | 0.490 |
| IC3B11 | IAPB9xCD3B220 | 5 | X | | 4.56 | 5.27 | 1.32 |
| IC3B15 | IAPB65xCD3B220 | 6 | | | 11.5 | 9.08 | 0.940 |
| IC3B7 | IAPB17xCD3B220 | 7 | X | | 6.10 | 4.74 | 3.40 |

^{a}Presumed to have the same functional activity as the IPAB54 parental.
^{b}Value is the average of two measurements.

Thus these IAPB47, IAPB55, IAPB63 and IAP57 expressed as IgG4, having Fc substitutions S228P, L234A, and L235A (numbering according to EU index) were paired with the anti-CD3 antibody CD3B219 comprising the VH and VL regions having the VH of SEQ ID NO: 94 and the VL of SEQ ID NO: 95 and IgG4 constant region with S228P, L234A, L235A, F405L, and R409K substitutions.

Similar to Example 9, the bispecific IL1RAP x CD3 antibodies were generated by combining the CD3B219 mAb and the monospecific IL1RAP mAbs in an in-vitro Fab arm exchange (as described in WO2011/131746).

Heavy and Light chains for the IL1RAP x CD3 bispecific Abs are shown below in Table 16.

TABLE 16
Heavy and Light Chain Sequences for bispecific Abs
IgG4-PAA comprising the anti-CD3 antibody CD3B219

| Ab | | Amino Acid Sequence |
|---|---|---|
| IC3B16 | Heavy chain 1 | EVQLVESGGGLVQPGGSLRLSCAASGFTFNTYAMNWVRQ |
| | CD3B219 | APGKGLEWVARIRSKYNNYATYYAASVKGRFTISRDDSKN |
| | (SEQ ID | SLYLQMNSLKTEDTAVYYCARHGNFGNSYVSWFAYWGQ |
| | NO: 94) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QTVVTQEPSLTVSPGGTVTLTCRSSTGAVTTSNYANWVQQ |
| | CD3B219 | KPGQAPRGLIGGTNKRAPGTPARFSGSLLGGKAALTLSGVQ |
| | (SEQ ID | PEDEAEYYCALWYSNLWVFGGGTKLTVLGQPKAAPSVTL |
| | NO: 95) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | EVQLVQSGAEVKKPGESLKISCKGSGYSFTSYWIGWVRQM |
| | IAPB47 | PGKGLEWMGIIYPSDSYTRYSPSFQGQVTISADKSISTAYLQ |
| | (SEQ ID | WSSLKASDTAMYYCARRNSAENYADLDYWGQGTLVTVSS |
| | NO: 68) | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEAAGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| | Light Chain 2 | EIVLTQSPGTLSLSPGERATLSCRASQSISNDLNWYQQKPGK |
| | IAPB47 | APKLLIYYASSLQSGVPSRFSGSGSGTDFTLTINSLQPEDFAT |
| | (SEQ ID | YYCQQSFTAPLTFGQGTKVEIKRTVAAPSVFIFPPSDEQLKS |
| | NO: 69) | GTASVVCLLNNFYPREAKVQWKVDNALQSGNSQESVTEQ |
| | | DSKDSTYSLSSTLTLSKADYEKHKVYACEVTHQGLSSPVTK |
| | | SFNRGEC |
| IC3B17 | Heavy chain 1 | EVQLVESGGGLVQPGGSLRLSCAASGFTFNTYAMNWVRQ |
| | CD3B219 | APGKGLEWVARIRSKYNNYATYYAASVKGRFTISRDDSKN |
| | (SEQ ID | SLYLQMNSLKTEDTAVYYCARHGNFGNSYVSWFAYWGQ |
| | NO: 94) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QTVVTQEPSLTVSPGGTVTLTCRSSTGAVTTSNYANWVQQ |
| | CD3B219 | KPGQAPRGLIGGTNKRAPGTPARFSGSLLGGKAALTLSGVQ |
| | (SEQ ID | PEDEAEYYCALWYSNLWVFGGGTKLTVLGQPKAAPSVTL |
| | NO: 95) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QLQLQESGPGLVKPSETLSLTCTVSGVSISSSTYYWGWLRQ |
| | IAPB55 | PPGMGLEWTGSIYFTGNTYYNPSLKSRVTISVDTSRNQFSL |
| | (SEQ ID | KLSSVTAADTAVYYCGSLFGDYGYFDYWGQGTLVTVSSA |
| | NO: 74) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain 2 | EIVMTQSPATLSVSPGERATLSCRASQFISSNLAWYQQKPG |
| | IAPB55 | QAPRLLIYGASTRATGIPARFSGSGSGTDFTLTISSLQSEDFA |
| | (SEQ ID NO: | VYYCQQYNNWPFTFGPGTKVDIKRTVAAPSVFIFPPSDEQL |
| | 87) | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| IC3B18 | Heavy chain 1 | EVQLVESGGGLVQPGGSLRLSCAASGFTFNTYAMNWVRQ |
| | CD3B219 | APGKGLEWVARIRSKYNNYATYYAASVKGRFTISRDDSKN |
| | (SEQ ID | SLYLQMNSLKTEDTAVYYCARHGNFGNSYVSWFAYWGQ |
| | NO: 94) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B219 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 95) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QVQLVQSGSELKKPGASVKVSCKASGYTFNTYAMNWVRQ |
| | IAPB63 | APGQGLEWMGWINTNTGNPTYAQGFTGRFVFSLDTSVSTA |
| | (SEQ ID | YLQISSLKAEDTAVYYCARRYFDWLLGAFDIWGQGTMVT |
| | NO: 76) | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEAAGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLGK |
| | Light Chain 2 | QSALTQPRSVSGSPGHSVTISCTGTSSDVGDYNYVSWYQQ |
| | IAPB63 | RPGKVPKLLIYDVSKRPSGVPDRFSGSKSGNTASLTISGLQA |
| | (SEQ ID | EDEAIYFCASYAGNYNVVFGGGTKLTVLGQPKAAPSVTLF |
| | NO: 88) | PPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| IC3B19 | Heavy chain 1 | EVQLVESGGGLVQPGGSLRLSCAASGFTFNTYAMNWVRQ |
| | CD3B219 | APGKGLEWVARIRSKYNNYATYYAASVKGRFTISRDDSKN |
| | (SEQ ID | SLYLQMNSLKTEDTAVYYCARHGNFGNSYVSWFAYWGQ |
| | NO: 94) | GTLVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEAA |
| | | GGPSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFLLYSKLTVDKSRWQEGNVFSCSVMHEALHNHYT |
| | | QKSLSLSLGK |
| | Light Chain 1 | QAVVTQEPSLTVSPGGTVTLTCRSSTGAVTTSNYANWVQQ |
| | CD3B219 | KPGQAPRGLIGGTNKRAPGTPARFSGSLLGGKAALTLSGAQ |
| | (SEQ ID | PEDEAEYYCALWYSNLWVFGGGTKLTVLGQPKAAPSVTL |
| | NO: 95) | FPPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| | Heavy chain 2 | QLQLQESGPGLVKPSETLSLTCTVSGGSISSSTYYWGWIRQP |
| | IAPB57 | PGKGLEWIGSIYFTGSTDYNPSLKSRVSISVDTSKNQFSLKL |
| | (SEQ ID | SSVTAADTAVYYCAKEDDSSGYYSFDYWGQGNLVTVSSA |
| | NO: 72) | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKV |
| | | SNKGLPSSIEKTISKAKGQPREPQVYTLPPSQEEMTKNQVSL |
| | | TCLVKGFYPSDIAVEWESNGQPENNYKTTPPVLDSDGSFFL |
| | | YSRLTVDKSRWQEGNVFSCSVMHEALHNHYTQKSLSLSLGK |
| | Light Chain 2 | DIQLTQSPSFLSASVGDRVTITCRASQGISSYLAWYQQKPG |
| | IAPB57 | KAPKLLIYAASTLQSGVPSRFSGSGSGTEFTLTISSLQPEDFA |
| | (SEQ ID | TYYCQQVNSYPLTFGGGTKVEIKRTVAAPSVFIFPPSDEQL |
| | NO: 73) | KSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |

### Example 14: IL1 Signaling by IC3B18 and IC3B19

IL1RAP x CD3 bispecific antibodies were assessed for any agonist or antagonist activity. HEK-Blue™ IL-1β cells from InvivoGen were incubated with the antibodies at a concentration of 100 μg/mL (10-fold dilutions) either in the absence or in the presence of 0.1 ng/mL of recombinant human (rh) IL-1β. “HEK-Blue™ IL-1β cells allow detection of bioactive IL-1β by monitoring the activation of the NF-κB and AP-1 pathways. They derive from HEK-Blue™ TNF-α/IL-1β cells in which the TNF-α response has been blocked. Therefore, HEK-Blue™ IL-1β cells respond specifically to IL-1β. They express a NF-κB/AP-1-inducible secreted embryonic alkaline phosphatase (SEAP) reporter gene. Binding of IL-1β to its receptor IL-1R on the surface of HEK-Blue™ IL-1β cells triggers a signaling cascade leading to the activation NF-κB and the subsequent production of SEAP.”

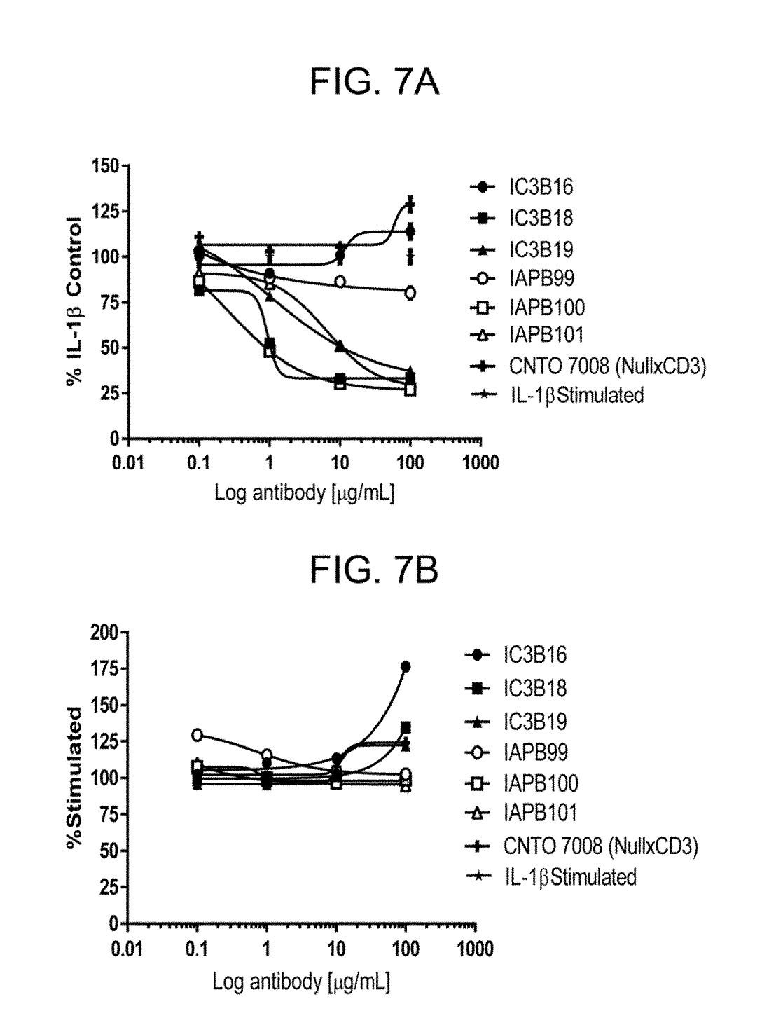

In the presence of 1 ng/mL rhIL-1β, IC3B18 and IC3B19, as well as their respective IL1RAP null arm controls IAPB100 (IAPB63 x B23B49) and IAPB101 (IAPB57 x B23B49) inhibited NF-κB reporter activity at 24 hr. The CD3 null arm control CNTO 7008 (B23B39 x CD3B219) had no antagonistic activity at any concentration tested (FIG. 7A). IC3B18, IC3B19, respective IL1RAP null arm controls IAPB100 and IAPB101, and CD3 null arm control CNTO 7008 had little-to-no agonist activity when tested in the absence of rhIL-1β (FIG. 7B). Additionally, IC3B16 and null arm control IAPB99 had no antagonistic activity at any concentration tested (FIGS. 7A and 7B).

### Example 15: Evaluation of IC3B18 and IC3B19 in Functional Cell Cytotoxicity Assay

The T-cell mediated cytotoxicity by IC3B18 and IC3B19 was evaluated using IL1RAP positive expressing AML cell lines (MOLM-13, MV4-11, SKNO-1 and OCI-AML-5) and an IL1RAP negative/low expressing Diffuse Large B-cell Lymphoma cell line (SU-DHL-10). The protocol previously described in Example 12 was followed.

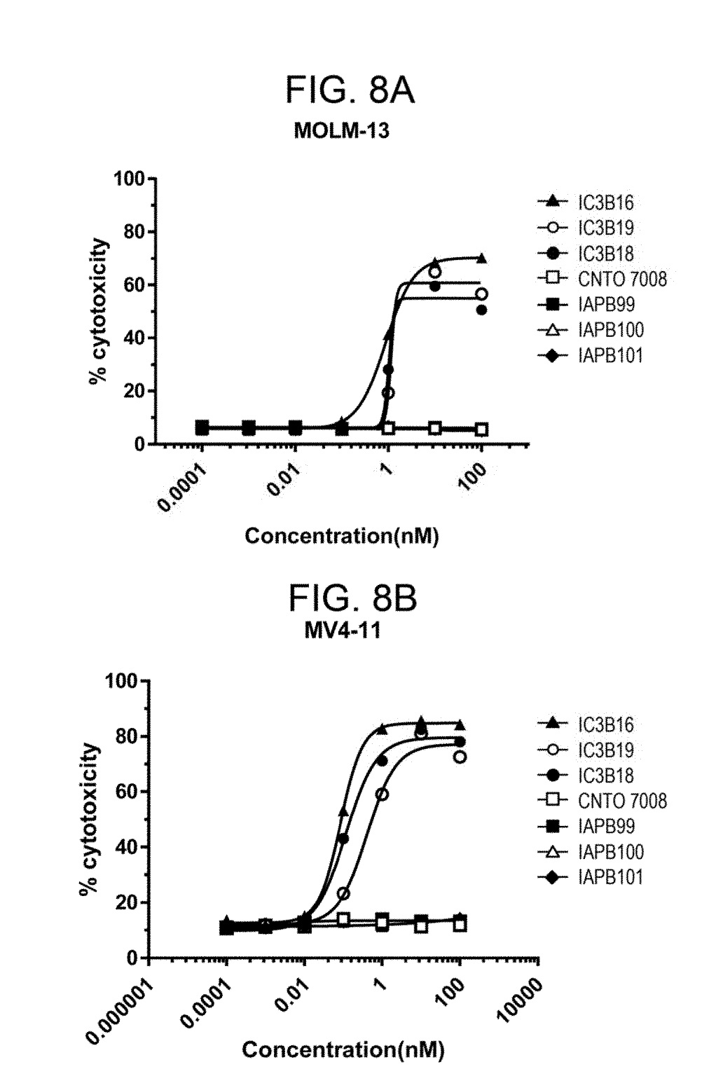

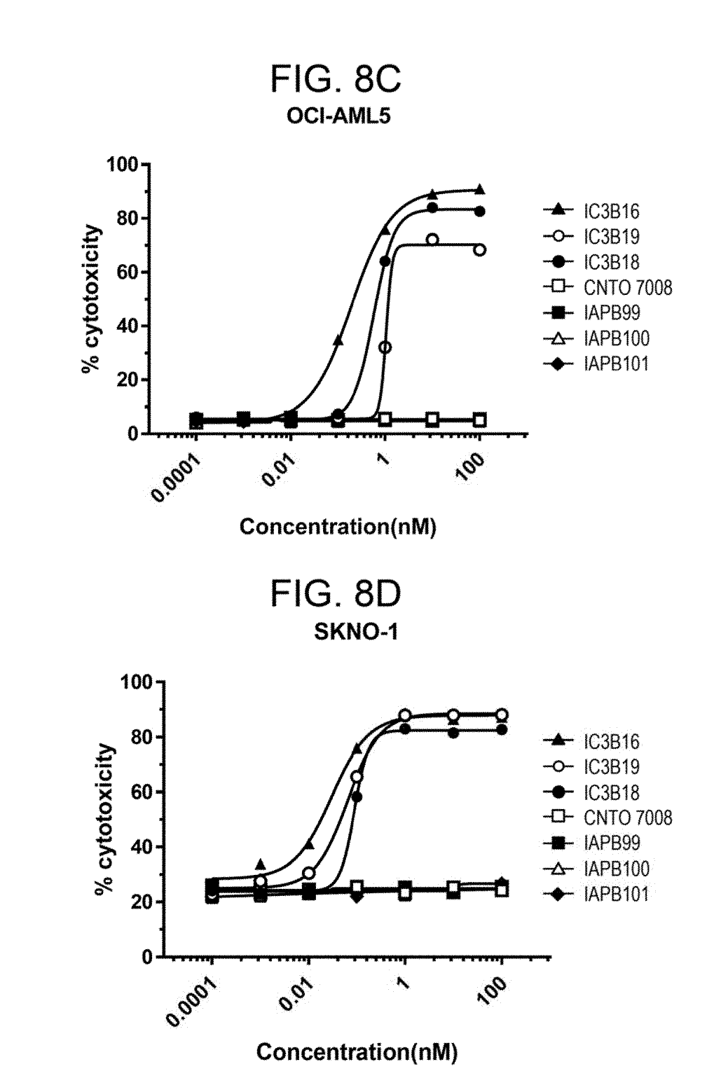

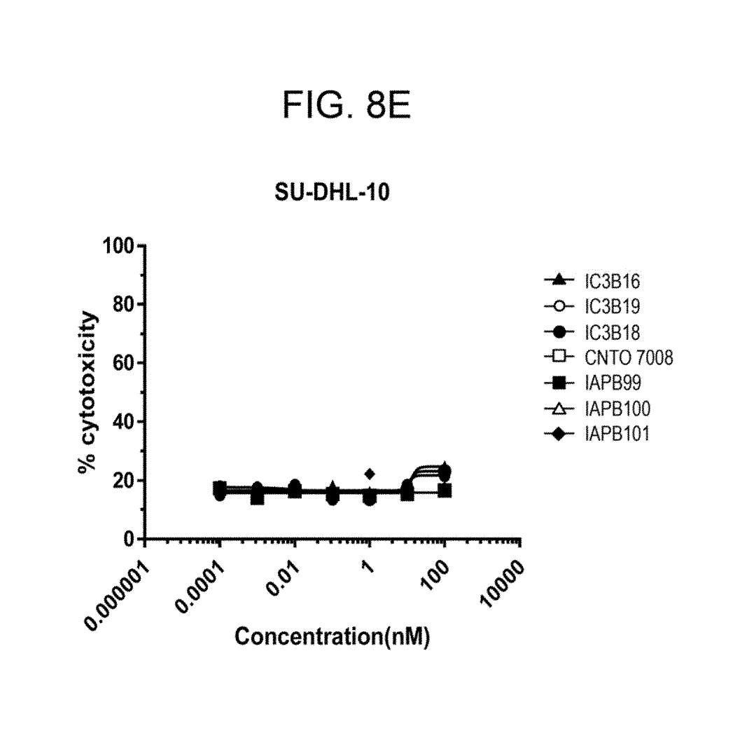

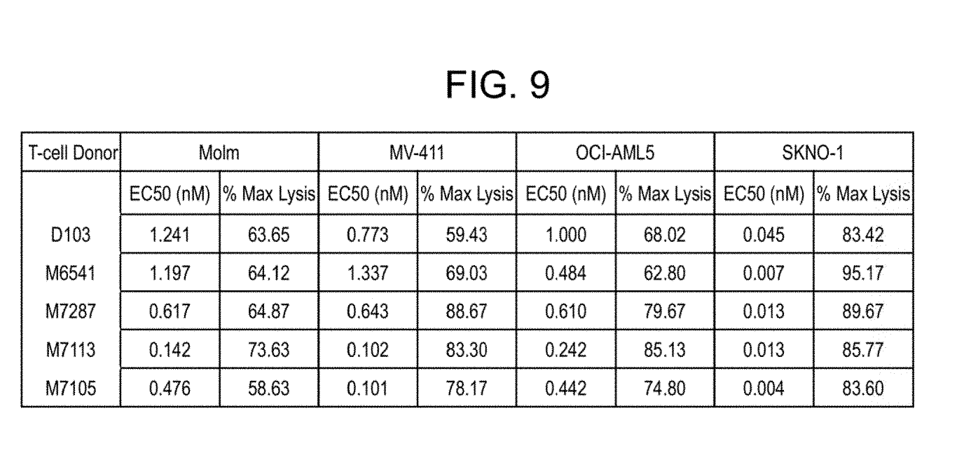

Pan T cell donor M7287 is represented (FIG. 8A, 8B, 8C, 8D, 8E and FIG. 9) as one of five pan-T cell donors that were assessed. Both IC3B18 and IC3B19 induce T-cell mediated cell cytotoxicity of IL1RAP⁺ AML cell lines Molm-13, MV4-11, SKNO-1, OCI-AML5, but not in IL1RAP negative/low expressing B-cell lymphoma line SU-DHL-10. Control antibodies (CNTO 7008, IAPB100, and IAPB101) had no overall T-cell mediated tumor cell cytotoxicity.

### Example 16: Ex Vivo Cytotoxicity by IC3B18 and IC3B19

Ex Vivo Autologous Monocyte Cytotoxicity Assay

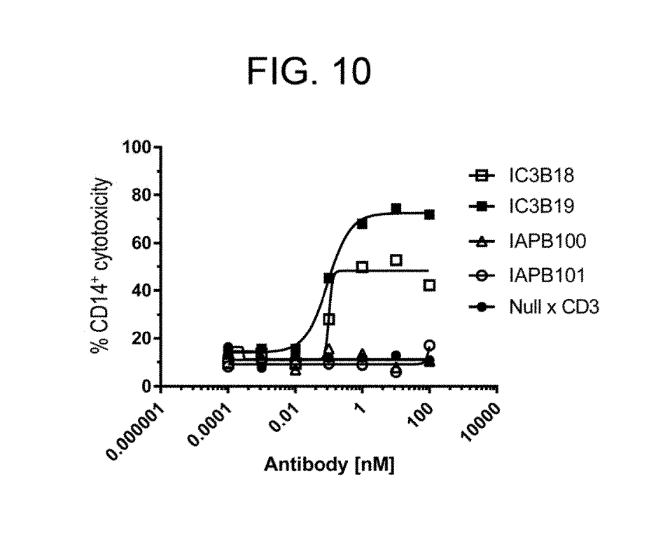

Previously, normal human monocytes (CD14⁺) were shown to have expression of IL1RAP on the surface of the cell (Jarasa, M et al. (2010) PNAS. 107: 16280-16285). To assess the cytotoxicity potential of IC3B18 and IC3B19, an ex vivo cytotoxicity assay was performed using isolated autologous (same donor) CD3⁺ T-cells and CD14⁺ monocytes at a 5:1 effector (T-cell): target (monocyte) ratio+Fc blocker to reduce potential non-specific Fc binding of the molecules. The data in FIG. 10 show that IC3B18 and IC3B19 specifically kill IL1RAP⁺ monocytes after 48 hours (depicted as % CD14⁺ cytotoxicity) but that null arm controls had little or no cytotoxicity; data are representative of two experiments performed with four individual normal human blood donors.

Ex Vivo Whole Blood SKNO-1 Cytotoxicity Assay

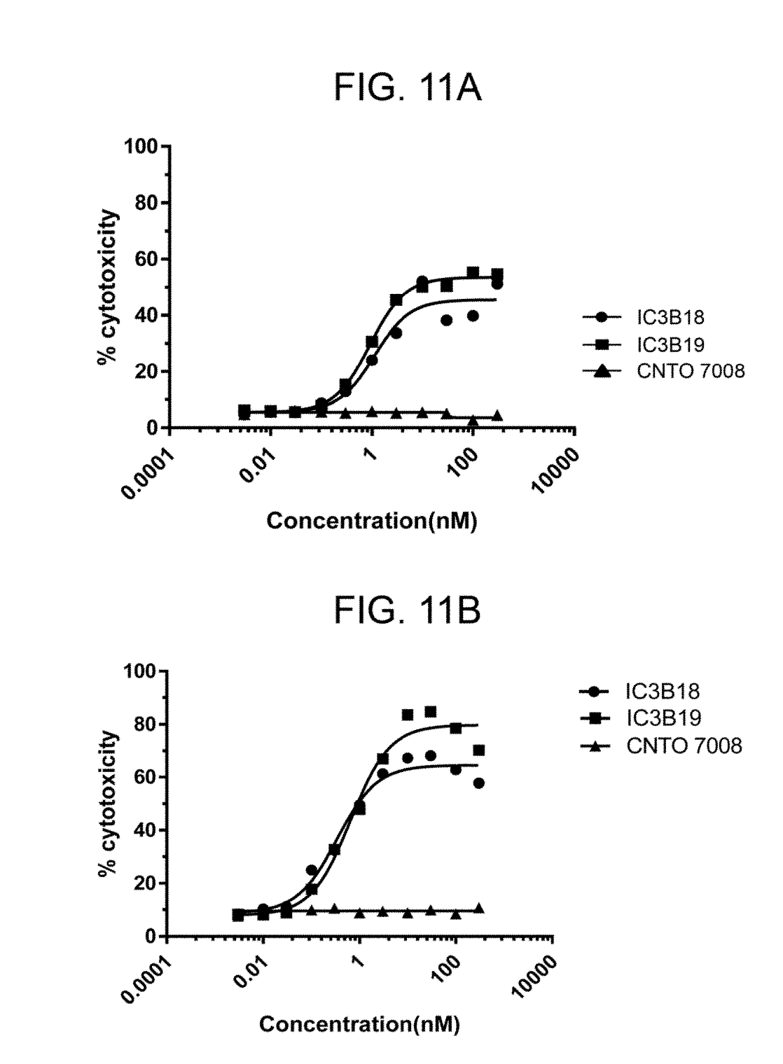

To further assess the cytotoxicity potential of IC3B18 and IC3B19 in the presence of physiological levels of soluble IL1RAP, an ex vivo cytotoxicity assay using normal healthy human whole blood with exogenously added IL1RAP⁺ AML cell line SKNO-1 was utilized. The data in FIGS. 11A and 11B indicate that both IC3B18 and IC3B19 specifically induce cell cytotoxicity of SKNO-1 cells at 24 and 48 hr. Additionally, cytotoxicity increased as well as EC₅₀ (nM) values from 24 to 48 hr. The null arm control CNTO 7008 (null x CD3) was used as a negative bispecific antibody control. The null arm control showed little-to-no cytotoxicity activity of the SKNO-1 cells. Two separate studies with a total of seven different normal healthy human donors were run on these molecules. The data in FIGS. 11A and 11B show that IC3B18 and IC3B19 specifically kill IL1RAP⁺ cell lines in vitro after 48 hours (depicted as % of cytotoxicity; data is representative of five experiments done with different T cell donors). The EC₅₀ values for each cell line and donor are shown in Table 17.

TABLE 17
EC₅₀ values for SKNO-1 cells analyzed for
cytotoxicity in each normal healthy donor blood analyzed.

| | | |
|---|---|---|
| | IC3B18 | IC3B19 |
| Whole Blood | EC₅₀ (nM) | EC₅₀ (nM) |

| Donors | 24 hour | 48 hour | 24 hour | 48 hour |
|---|---|---|---|---|
| 27067 | 1.112 | 0.337 | 0.912 | 0.647 |
| 00201 | 8.619 | 0.704 | 3.583 | 0.703 |
| 27060 | 2.500 | 0.516 | 1.878 | 1.302 |
| 00263 | 0.400 | 0.580 | 1.505 | 0.768 |
| 32782 | NA¹ | 0.650 | NA¹ | 1.621 |
| 27050 | NA | 2.035 | 1.384 | 3.361 |
| 32771 | 1.943 | NA¹ | 1.675 | NA¹ |
| Average EC₅₀ | 2.915 | 0.804 | 1.823 | 1.400 |
| (nM) | | | | |
| Standard | 3.287 | 0.616 | 0.922 | 1.035 |
| Deviation | | | | |

Ex Vivo IC3B18 and IC3B19 Mediated Reduction of Blasts and T-Cell Activation in AML Primary Sample

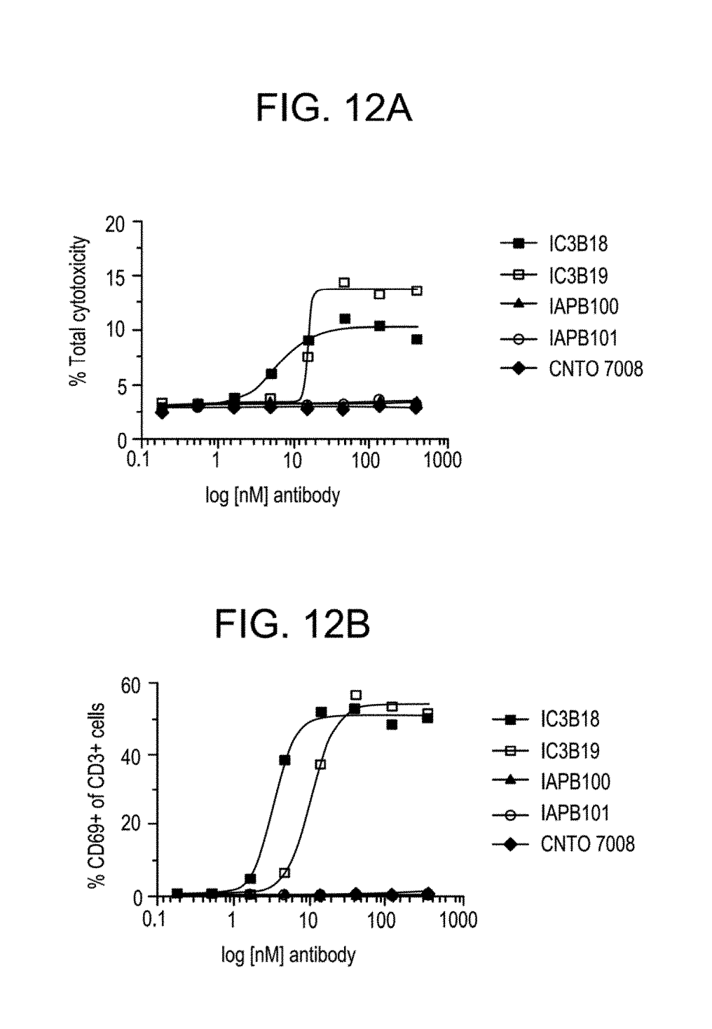

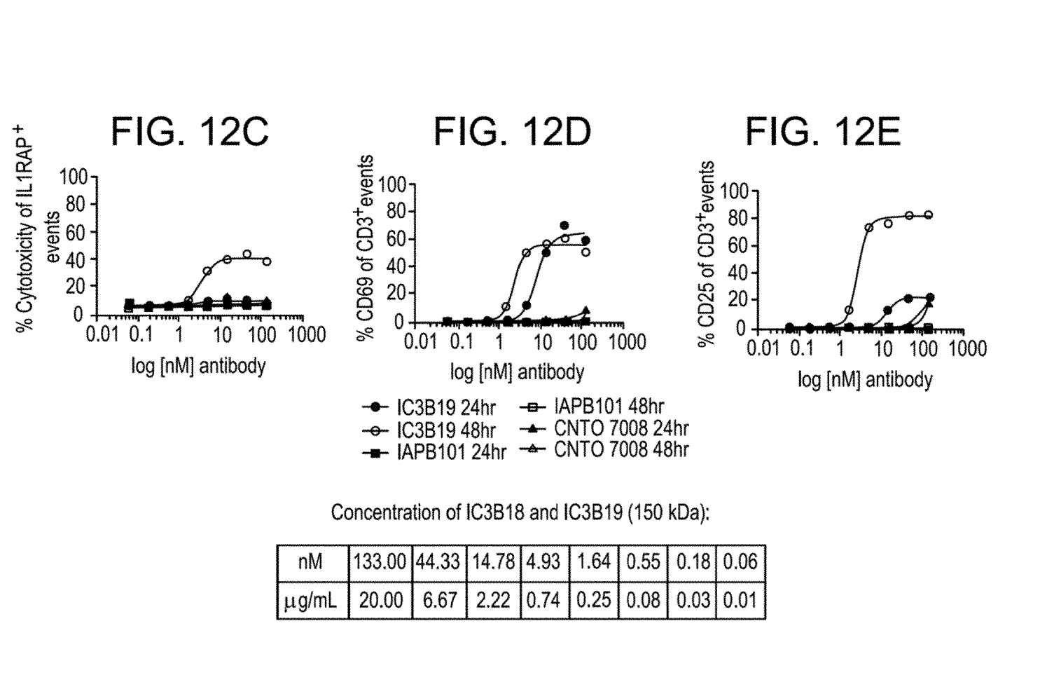

To assess the cytotoxicity potential of IC3B18 and IC3B19, an ex vivo cytotoxicity assay was performed using AML donor whole blood (FIGS. 12A, 12B, 12C, 12D and 12E). In this assay, various bispecific antibodies were added to diluted whole blood from AML donors for a period of 24 hours without providing additional T-cells, since this assay relies on the presence of autologous T-cells in the donor's blood. The extent of cytotoxicity was determined by quantifying the IL1RAP⁺ cells in the fraction in the presence of the bispecific antibodies, and expressing it as the % cytotoxicity. The T-cell activation was assessed by the expression of CD69 (shown).

As shown in FIGS. 12A, 12B, 12C, 12D and 12E, IC3B18 and IC3B19 promoted a dose-dependent reduction of total cytotoxicity that correlated with T-cell activation after 24 hr. Null arm control antibodies failed to show tumor cell cytotoxicity or T-cell activation. This result also shows that the both IC3B18 and IC3B19 antibodies work in an autologous setting. This experiment was also performed with another AML donor sample. Only the IC3B19 and null arm control antibodies were analyzed at both 24 and 48 hours IL1RAP⁺ cell cytotoxicity and showed ˜40% maximal cytotoxicity and did result in CD25 and CD69 up-regulation at 24 and 48 hours (data not shown).

Ex Vivo Whole Blood OCI-AML5 Cytotoxicity

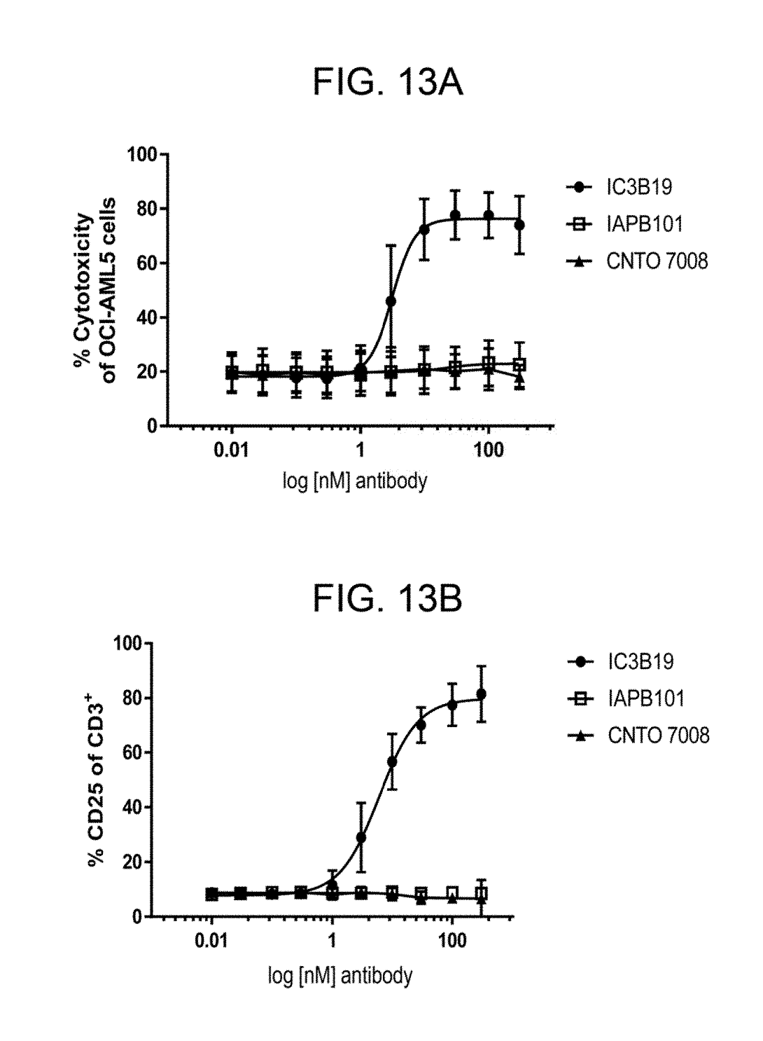

The OCI-AML5 cell line was also tested in the same ex vivo whole blood assay. FIGS. 13A and 13B shows that IC3B19 specifically kills IL1RAP⁺ OCI-AML5 cells in vitro after 48 h (depicted as % of cytotoxicity; data is representative of five experiments done with different T cell donors). The mean EC₅₀ value for cytotoxicity (FIG. 13A) in was 3.132 nM and activation (FIG. 13B) was 5.993 nM. The Null arm controls CNTO 7008 (Null x CD3) and IAPB101 (IL1RAP x Null) were used as negative control antibodies and showed little-to-no cytotoxicity activity. A total of fifteen different normal healthy human donors were run on these molecules (ELN ref: IL1RAPxCD3 bispecific-00425). These data show that when IC3B19 is added to whole blood containing exogenous OCI-AML5 cells, IC3B19 was capable of activating and redirecting T-cells to induce cytotoxicity.

### Example 17: Experimental Cross-Reactivity Assessment for IL1RAP

The MSD cell binding assay described in Example 4 was used to assess IL1RAP binding. The objective of the screening assay was to characterize whether IC3B18 and IC3B19 bound specifically to cell lines HEK-293F Human (clone HE2) and Cyno (clone CB8) IL1RAP full-length (FL) extracellular domain (ECD)-expressing cell lines as compared to HEK-293F parental control. The use of HEK-293F Mouse (Clone 5) and Rat (clone 1) cell lines were also used to identify species cross-reactivity.

The results from the binding study are shown in FIG. 14. IC3B18 and IC3B19, as well as the IL1RAP null arm controls IAPB100 (IAPB63 x B23B49) and IAPB101 (IAPB57 x B23B49) bound specifically to HEK-293F Human clone HE2 and Cyno clone CB8 IL1RAP FL-ECD cell lines. The anti-MYC positive control antibody detected expression of the construct on each cell line. The CD3 null arm CNTO 7008 (B23B39 x CD3B219) and I3CB15 (human IgG4-PAA null arm isotype control) had low binding expression. Background binding of IC3B18 and IC3B19 to the HEK-293F parental, mouse clone 5, and rat clone 1 was observed only at the highest concentrations assayed.

### Example 18: Anti-Tumor Efficacy of IC3B19 in Tumorigenesis Prevention of OCI-AML5 Human AML Xenografts in PBMC-Humanized NSG Mice

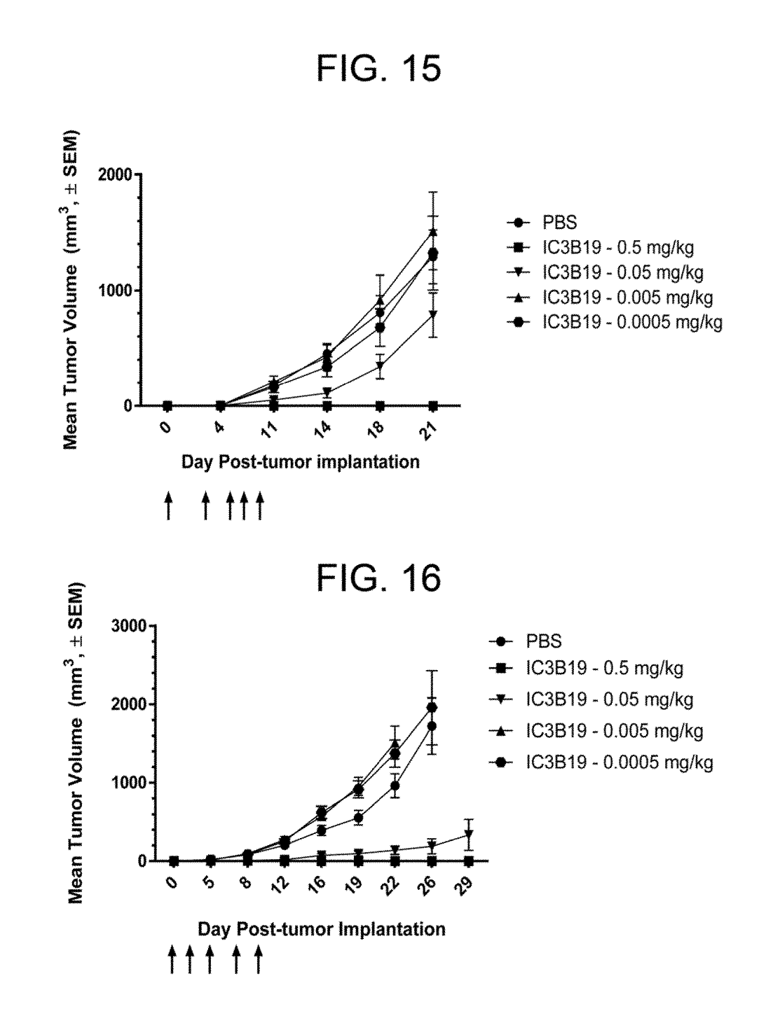

This study evaluated the efficacy of IC3B19 in preventing tumorigenesis of OCI-AML5 human AML xenografts in PBMC humanized NSG mice. Mice were intravenously injected with 1×10⁷ human PBMCs in a volume of 200 μL PBS each. On Day 7, mice were subcutaneously implanted with OCI-AML5 human AML cells (10×10⁶ cells in 200 μL PBS) on the dorsal flank, followed by intravenous administration of PBS or IC3B19 approximately every other day for five doses. There was activity of IC3B19 at 0.5 mg/kg in the presence of human effector cells as shown by the statistically significant tumor growth inhibition compared PBS treatment on Day 18 and Day 21 (p<0.0001) (FIG. 15).

### Example 19: Anti-Tumor Efficacy of IC3B19 in Tumorigenesis Prevention of MOLM-13 Human AML Xenografts in PBMC-Humanized NSG Mice

This study evaluated the efficacy of IC3B19 in preventing tumorigenesis of MOLM-13 human AML xenografts in PBMC humanized NSG mice. Mice were intravenously injected with 1×10⁷ human PBMCs in 200 μL PBS each. On Day 7, mice were subcutaneously implanted with MOLM-13 human AML cells (1×10⁶ cells in 200 μL PBS on the dorsal flank), followed by intravenous administration of PBS or IC3B19 approximately every other day for five doses. There was activity of IC3B19 0.05 mg/kg and 0.5 mg/kg in the presence of human effector cells as shown by the statistically significant tumor growth inhibition compared to PBS treatment on Day 8 (p<0.0001, p<0.0001, and p<0.0001, respectively) and Day 12 (p<0.0001, p<0.0001, and p<0.0001, respectively) (FIG. 16).

### Example 20: Anti-Tumor Efficacy of IC3B18 and IC3B19 in Tumorigenesis Prevention of MOLM-13 Human AML Xenografts in PBMC-Humanized NSG Mice

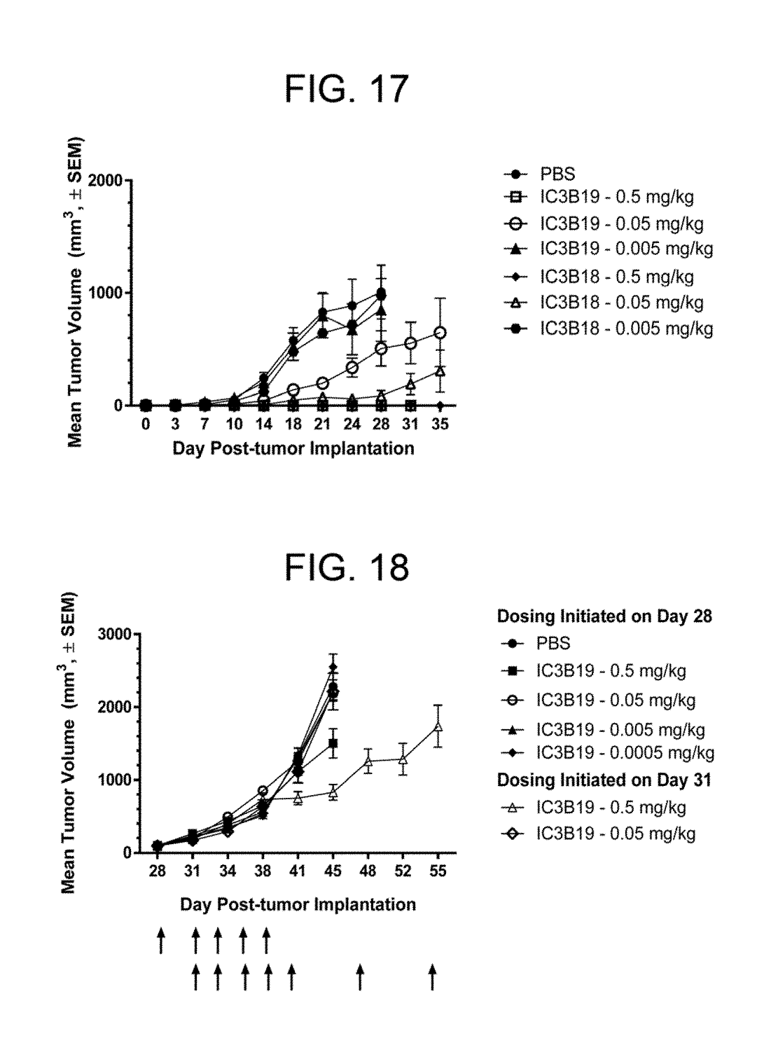

This study evaluated the efficacy of IC3B18 and IC3B19 in preventing tumorigenesis of MOLM-13 human AML xenografts in PBMC humanized NSG mice. Mice were intravenously injected with 1×10⁷ human PBMCs in 200 μL PBS each. On Day 7, mice were subcutaneously implanted with MOLM-13 human AML cells (1×10⁶ cells in 200 μL PBS on the dorsal flank), followed by intravenous administration of PBS, IC3B18, or IC3B19 approximately every other day for five doses. There was activity of IC3B19 at 0.05 mg/kg and 0.5 mg/kg in the presence of human effector cells as shown by the statistically significant tumor growth inhibition compared to PBS treatment on Day 18 (p<0.0001, p<0.0001, respectively) and Day 21 (p<0.0001, p<0.0001, respectively). Additionally, there was activity of IC3B18 at 0.5 mg/kg and 0.05 mg/kg in the presence of human effector cells show by the statistically significant tumor growth inhibition compared to PBS treatment on Day 14 (p<0.05, p<0.05, respectively), Day 18 (p<0.0001, p<0.0001, respectively) and Day 21 (p<0.0001, p<0.0001, respectively) (FIG. 17).

### Example 21: Anti-Tumor Efficacy of IC3B19 in OCI-AML5 Human AML Xenografts in PBMC Humanized NSG Comparing Treatment Initiated on Day 28 Versus Day 31

This study evaluated the efficacy of IC3B19 in established OCI-AML5 human AML xenografts in female NSG mice. Mice were each subcutaneously implanted with OCI-AML5 human AML cells (10×10⁶ cells in 200 μL PBS) on the dorsal flank. Animals were randomized by tumor volume on Day 28 at an average volume of 93.7 mm³ and received PBMC injections intravenously. On Day 28, five groups were intravenously dosed with PBS or IC3B19 approximately every other day for five doses. Additionally, on Day 35, two groups were intravenously dosed with IC3B19 approximately every other day for five doses. Animals dosed with IC3B19 at 0.5 mg/kg, on the same day as PBMC injection (Day 28), had significant tumor growth inhibition compared to PBS treatment on Day 45 (p<0.0001). Additionally, animals dosed with IC3B19 at 0.5 mg/kg, three days post PBMC injection (Day 31), had significant tumor growth inhibition compared to PBS treatment on Day 41 (p<0.0001) and Day 45 (p<0.0001) (FIG. 18).

### Example 22: Anti-Tumor Efficacy of IC3B18 and IC3B19 in OCI-AML5 Human AML Xenografts in PBMC Humanized NSG Mice Comparing Treatment Initiated on Day 31 Versus Day 35

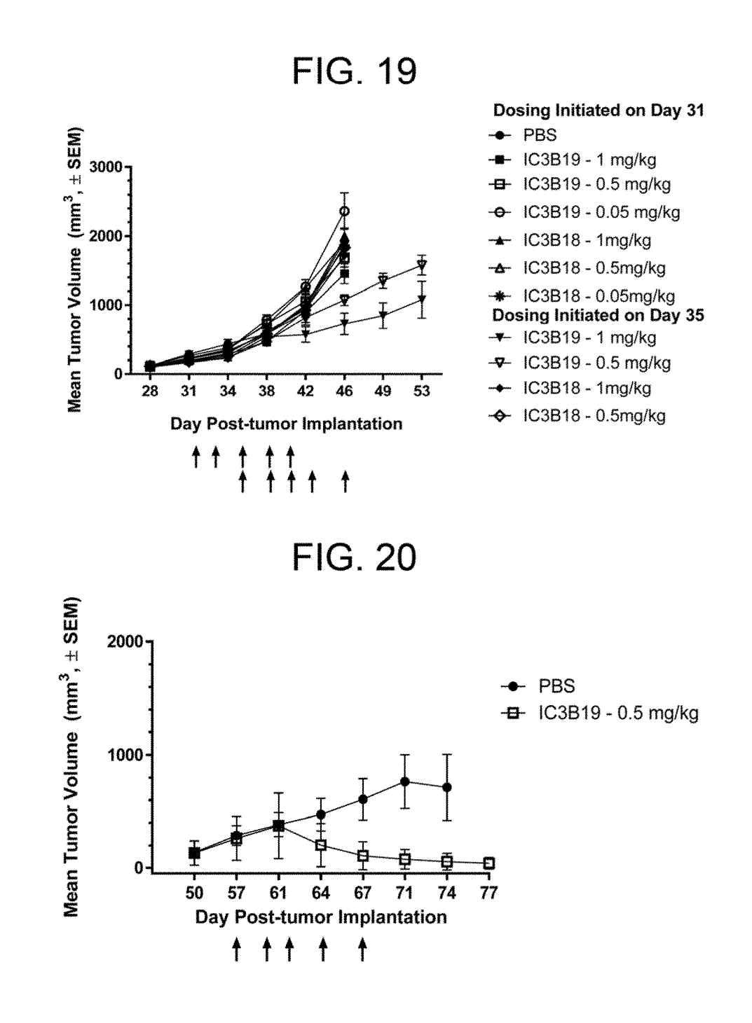

This study evaluated the efficacy of IC3B19 in established OCI-ANIL5 human AML xenografts in female NSG mice. Mice were each subcutaneously implanted with OCI-AML5 human AML cells (10×10⁶ cells in 200 μL PBS) on the dorsal flank. Animals were randomized by tumor volume on Day 28 at an average volume of 111.5 mm³ and received PBMC injections intravenously. On Day 31, seven groups were intravenously dosed with PBS, IC3B18, or IC3B19 approximately every other day for five doses. Additionally, on Day 35, four groups were intravenously dosed with IC3B18 or IC3B19 approximately every other day for five doses. There was no activity of IC3B18 in the presence of human effector cells compared to PBS treatment, regardless of dosing initiated on Day 31 or Day 35. There was activity of IC3B19 at 0.5 mg/kg, dosing initiated on Day 35, in the presence of human effector cells as shown by statistically significant tumor growth inhibition compared to PBS on Day 46 (p<0.0001). Also, there was activity of IC3B19 at 1 mg/kg, dosing initiated on Day 35, in the presence of human effector cells as shown by the statistically significant tumor growth inhibition compared to PBS treatment on Day 42 (p<0.05) and on Day 46 (p<0.0001). Additionally, there was activity of IC3B19 at 1 mg/kg, dosing initiated on Day 31, in the presence of human effector cells show by the statistically significant tumor growth inhibition compared to PBS treatment on Day 46 (p<0.01) (FIG. 19).

### Example 23: Anti-Tumor Efficacy of IC3B19 in SKNO-1 Human AML Xenografts in PBMC Humanized NSG Mice

This study evaluated the efficacy of IC3B19 in established SKNO-1 human AML xenografts in female NSG mice. On Day 0, mice were each subcutaneously implanted with SKNO-1 tumor fragments via trocar implantation bilaterally on the dorsal flank. Animals were randomized by tumor volume on Day 50 at an average volume of 135.0 mm³ and received PBMC injections intravenously. On Day 57, seven days post PBMC injection, animals were intravenously dosed with IC3B19 approximately every other day for five does. IC3B19 at 0.5 mg/kg resulted in statistically significant tumor growth inhibition compared to PBS treatment in the presence of human effector cells on Day 67 (p<0.05) and Day 71 (p<0.001) (FIG. 20).

### Example 23: Fc Ligand Binding Assays

Binding competition to the human Fc ligands FcγRT, FcγRlla, FcγRIIb, FcγRIIIa, and FcRn was measured for IC3B18 and IC3B19 relative to wild type hIgG1, hIgG4 PAA isotype, and a collection of related IgG4 PAA parental (bivalent) and null-arm (monovalent) control antibodies. Measurements were made using an AlphaScreen™ assay (Amplified Luminescent Proximity Homogeneous Assay (ALPHA), PerkinElmer, Wellesley, Mass.), a bead-based luminescent proximity assay. Laser excitation of a donor bead excites oxygen, which if sufficiently close to the acceptor bead generates a cascade of chemiluminescent events, ultimately leading to fluorescence emission at 520-620 nm. The control antibody was biotinylated by standard methods for attachment to streptavidin donor beads, and GST-tagged FcγRs and FcRn were bound to glutathione chelate acceptor beads. In the absence of competition, the IL1RAP x CD3 bispecific antibody, control or wild-type antibodies, and the human Fc ligands interact and produce a signal at 520-620 nm.

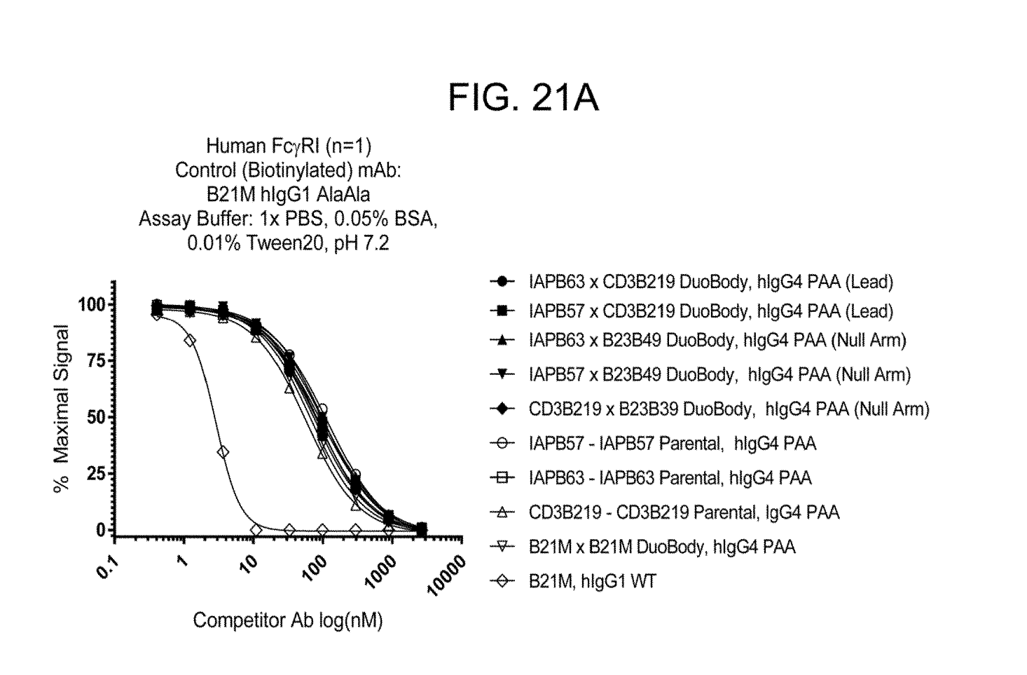

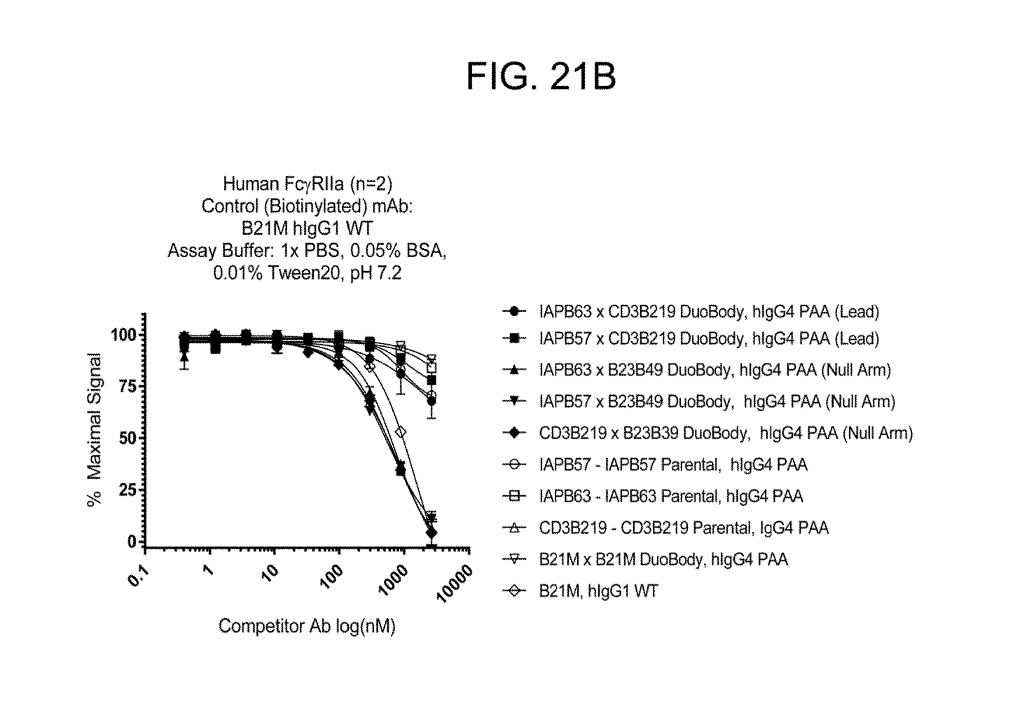

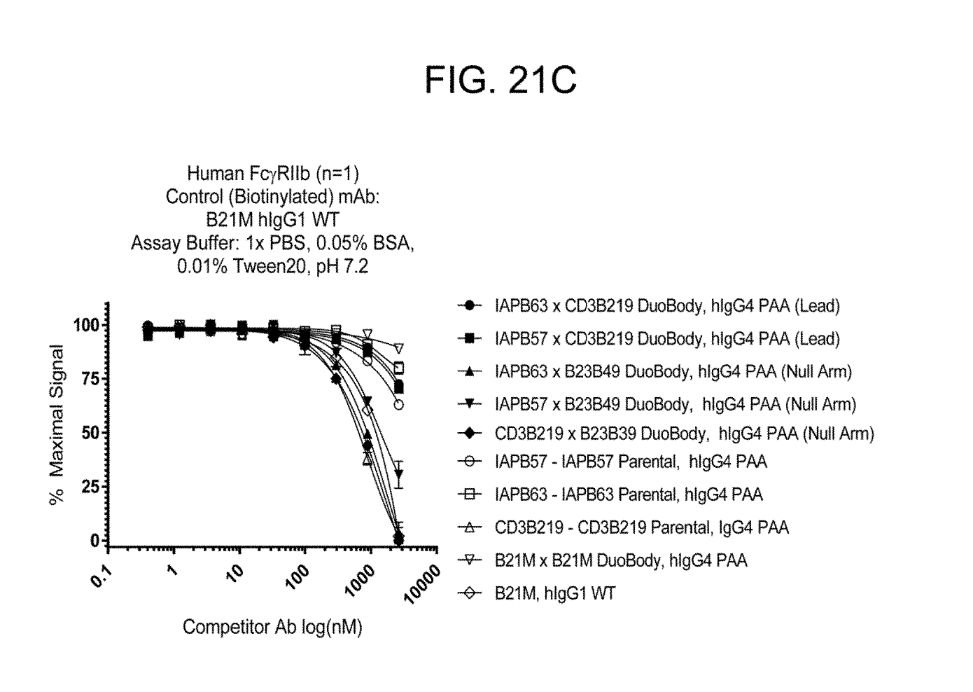

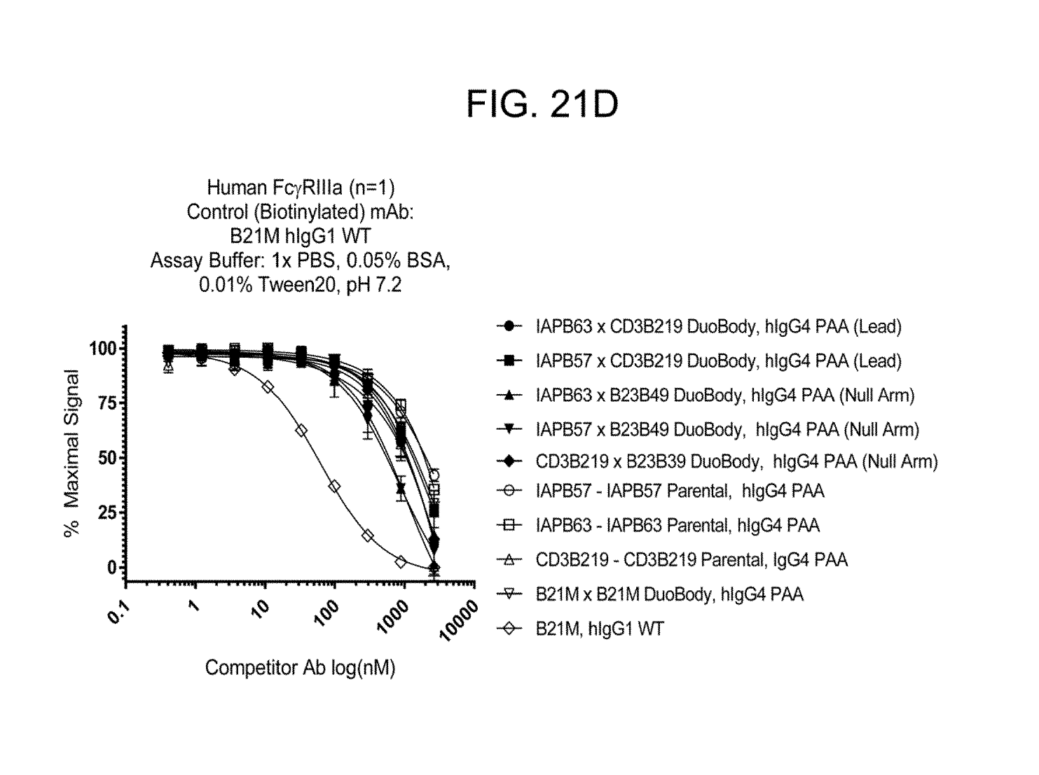

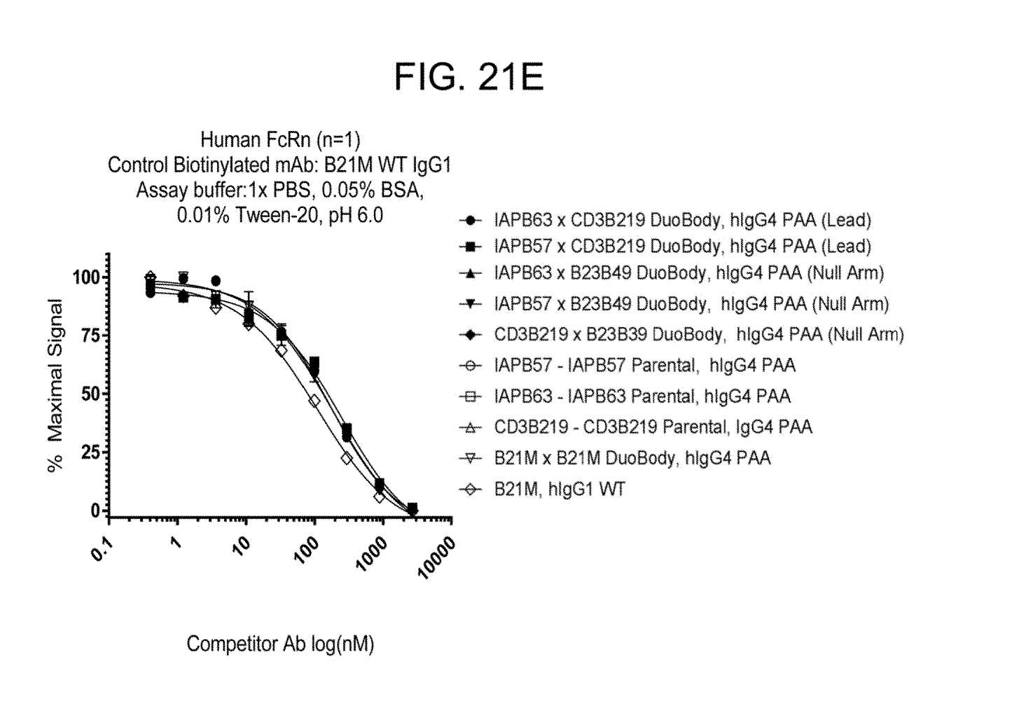

For FcγRI, IC3B18 and IC3B19 are no more competitive than hIgG4 PAA isotype control (FIG. 21A). For FcγRIIa, IC3B18 and IC3B19 are no more competitive than hIgG4 PAA isotype control (FIG. 21B). For FcγRIIb, IC3B18 and IC3B19 are no more competitive than hIgG4 PAA isotype control (FIG. 21C). For FcγRIIIa, IC3B18 and IC3B19 are no more competitive than hIgG4 PAA isotype control (FIG. 21D). IC3B18 and IC3B19 bind FcRn as efficiently as hIgG1 WT and hIgG4 PAA isotype (FIG. 21E). In summary, IC3B18 and IC3B19 bind all Fc receptors tested to essentially the same extent as matched IgG4 PAA isotype. It should be noted that on FcγRIIa and FcγRIIb, IC3B18 and IC3B19 are significantly less competitive than the CD3B219 parental and CD3B219 x B21M (null-arm) Abs (FIGS. 21B and 21C). For FcγRIIa and FcγRIIb, the IL1RAP x CD3 bispecific antibodies are also significantly less competitive than the two IL1RAP x B21M (null-arm) antibodies (FIGS. 21B and 21C).

### Example 24: Efficacy of IC3B19 in SKNO-1 Human AML Xenografts in T Cell Humanized NSG Mice

Efficacy of IC3B19 was evaluated in established SKNO-1 human AML xenografts in female NSG mice humanized with 20×10⁶ in vitro expanded and activated human T cells ip. IC3B19 at 0.5 or 1 mg/kg or PBS control was dosed q2d-q3d on Days 35, 37, 39, 41, 43, 46, 48, 50, 53, and 55 for a total of 10 doses. On day 60 post-tumor implant, which was the last date when at least six of eight animals remained in all treatment groups, tumor growth inhibition (% TGI) was calculated. Statistically significant tumor growth inhibition was observed at IC3B19 at 0.5 or 1 mg/kg with 100% TGI in both treatment groups compared to the PBS-treated controls with complete or partial regressions observed in all but one animal by day 63 (p<0.001, FIG. 22). By day 81, 6/8 tumors had completely regressed in the 0.5 mg/kg treatment group and 7/8 tumors completely regressed in the 1 mg/kg treatment group.

### Example 25: Efficacy of IC3B19 in Disseminated MOLM-13 Luciferase Human AML Model in T Cell Humanized NSG Mice

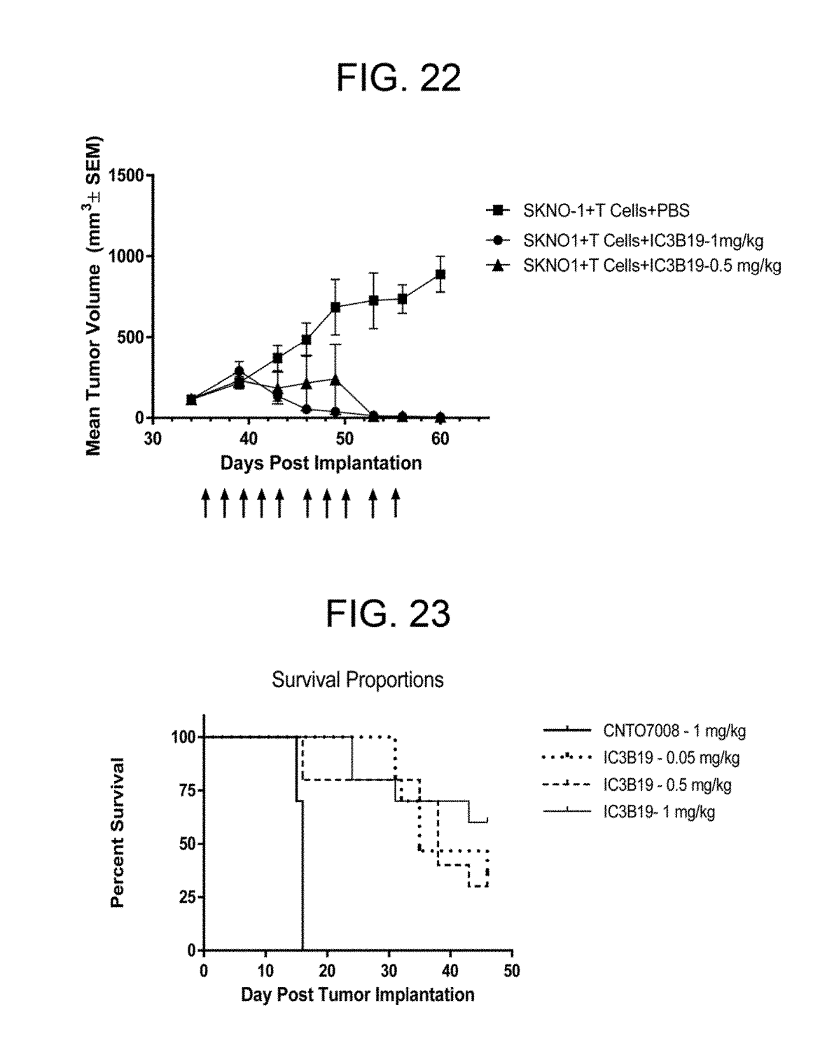

Efficacy of IC3B19 was evaluated in a luciferase transfected disseminated MOLM-13 human AML model in female NSG mice humanized with 20×10⁶ in vitro activated and expanded human T cells ip and randomized by live animal bioluminescence imaging. Treatment with IC3B19 at 0.05, 0.5 or 1 mg/kg or CD3x null control CNTO7008 at 1 mg/kg was given q3d-q4d on Days 4, 8, 11, 14, 17, 21, 24, 28, 31, 35, and 38 for a total of 11 doses. On Day 46 post-tumor implant, which was the last date before animals were euthanized due to GvHD-related morbidity, increased life span (% ILS) was calculated. IC3B19 at 0.05, 0.5 and 1 mg/kg had statistically significant increased life span of 199%, 138% and >138% respectively compared to the CD3x null control antibody (p<0.0001, p=0.0003, p<0.0001 respectively, FIG. 23). MOLM-13 luciferase cells in mice treated with CNTO7008 control honed to the hind limb and spine culminating in hind limb paralysis or morbidity by day 16. Additionally, two animals in the IC3B19 0.5 mg/kg treated group were euthanized or found dead on Day 16 due to hind limb paralysis or morbidity. Mice treated with IC3B19 showed reduced tumor burden in the spine and the hind limb at days 12 and 14 by bioluminescence. At day 46, three animals in each of the IC3B19 treatment groups (0.05, 0.5, 1 mg/kg) were tumor free as assessed by bioluminescence.

### Example 26: RNA Expression for IL1RAP in Solid Tumors

In this study, the distribution of RNA expression for IL1RAP was evaluated in a broad range of tumor types (n=14) and compared to the RNA expression of each tumor to a matched normal sample from data available in The Cancer Genome Anatomy (TCGA, http://cancergenome.nih.gov/). This study was performed to assess which solid tumor types have elevated expression of IL1RAP to help identify which patients may benefit from IL1RAP inhibition.

TCGA RNA-Seq

Data from RNASeq studies in the TCGA project were queried using an internal knowledgebase (Oncoland, TCGA_B37) provided by omicsoft (www.omicsoft.com). Derivative data is precompiled by Omicsoft using OSA aligner¹ and determination of RNA quantitation through RPKM normalization using the Genome reference library Human.B37.3 and Gene Model ‘OmicsoftGene20130723’). RNA-Seq output is evaluated by comparing tumor vs adjacent normal tissue derived from a subset of the same patients in TCGA.

Analysis Procedure

Fourteen indications with data available for both tumor and normal in solid tumors were assessed.
ID Type
ESCA Esophageal
BLCA Bladder
KIRP Renal-Papillary
UCEC Uterine
STAD Stomach
COAD Colon
HNSC Head and Neck
LUSC Lung Squamous
PRAD Prostate
THCA Thyroid-Anaplastic
LUAD Lung Adenocarcinoma
KIRC Kidney-Clear Cell
BRCA Breast
PAAD Pancreas

IL1RAP was queried in Oncoland and the number of tumors with higher expression relative to adjacent normal was tabulated and a frequency estimate calculated. Samples with elevated expression were counted when the expression value was greater than the highest expression value in the matched normal sample. Boxplots for visual evaluation of the normalized (FPKM) RNA distribution were also generated for each tumor type.

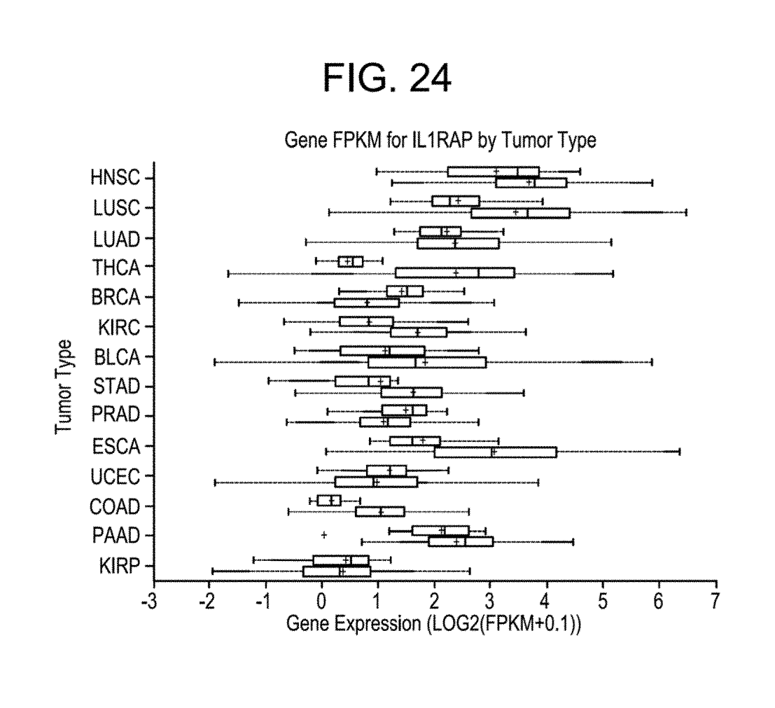

There were five tumor types identified with notable elevated expression that also had sufficient number of matched normal samples (>10) available for comparison purposes (Table 18 and FIG. 24). The tumor types with elevated expression relative to normal include Esophageal (28%), Bladder (26%), Colon (72%), Lung Squamous (29%) and Anaplastic Thyroid (70%).

TABLE 18
Table summary of IL1RAP expression in Solid Tumors.

| | | | | | Total Tumor | Tumor |
|---|---|---|---|---|---|---|
| | | Total number | | | above Normal | Percentage |
| ID | Type | of Samples | Total Normal | Total Tumor | range | High Expression |
| ESCA | Esophageal | 197 | 13 | 184 | 51 | 28 |
| BLCA | Bladder | 430 | 19 | 411 | 107 | 26 |
| KIRP | Renal-Papillary | 322 | 32 | 290 | 15 | 5 |
| UCEC | Uterine | 585 | 35 | 550 | 20 | 4 |
| STAD | Stomach | 457 | 37 | 420 | 1 | 0 |
| COAD | Colon | 512 | 41 | 471 | 337 | 72 |
| HNSC | Head and Neck | 564 | 44 | 520 | 99 | 19 |
| LUSC | Lung Squamous | 552 | 51 | 501 | 143 | 29 |
| PRAD | Prostate | 553 | 52 | 501 | 18 | 4 |
| THCA | Thyroid- | 564 | 59 | 505 | 352 | 70 |
| | Anaplastic | | | | | |
| LUAD | Lung | 587 | 59 | 528 | 13 | 2 |
| | Adenocarcinoma | | | | | |
| KIRC | Kidney- Clear | 609 | 72 | 537 | 82 | 15 |
| | Cell | | | | | |
| BRCA | Breast | 1220 | 113 | 1107 | 41 | 4 |
| PAAD | Pancreas | 182 | 4 | 178 | 56 | 31 |

### Example 27: Quantification of IL1RAP Receptors on the Surface of Solid Tumor Cell Lines

RNA Seq data from Example 26 shows the presence of IL1RAP RNA in solid tumors. In order to explore the possibilities of IL1RAP x CD3 as a solid tumor therapy, a variety of cancer tumor cell types were quantified for IL1RAP surface expression and their ability to be killed in an apoptosis cell based assay.

Lung, prostate, pancreas, and colon cell lines were cultured according to ATCC conditions and grown to 70-85% confluence. Cancer cell lines were dissociated with non-enzymatic dissociation buffer (Invitrogen, Cat#13151-004) where appropriate and washed in DPBS−/− (Invitrogen, Cat#141902-250). Cells were counted and resuspended in DPBS −/− to a concentration of 3*10\hat{ }6 cells/mL and 100 μL were plated into each well. The LIVE/DEAD® Fixable Near-IR Dead Cell Stain buffer (Invitrogen, Cat#10082-147) was added to samples for 25 min at RT. The samples were washed in 200 uL of flow cytometry stain buffer (BD Pharmigen, Cat##554657), blocked with FC block (Accurate Chemical, NB309) for 15 min at room temperature, and stained with 5 μg/mL of Isotype Control (R&D Systems, Cat# IC002P) or IL1RAP (R&D Systems, Cat#FAB676P) for 45 min at 4° C. in flow cytometry stain buffer. Stained cells evaluated on the BD FACS CANTO II™. The Geomean ratios were calculated in Flow Jo V_10 using Singlets/Live/Cells populations. Receptor densities were calculated using the Quantum™ Simply Cellular® System (Bang's Laboratories, Cat#815) and the BD Relative Linear Scale Calibration Plot macro. The IL1RAP receptor density for each cell line is summarized in Table 19 showing a wide range of surface expression in solid tumors.

TABLE 19
IL1RAP receptor density for each cell line

| | | IL1RAP receptor |
|---|---|---|
| Cell Lines | Tumor Type | #/Cell |
| A549 | Lung | 6,317 |
| Calu-3 | Lung | 70,264 |
| H1975 | Lung | 74,561^{a} |
| H2110 | Lung | 9,999 |
| H2172 | Lung | 35,127 |
| H2228 | Lung | 20,845 |
| H292 | Lung | 7,074 |
| H358 | Lung | 17,795^{b} |
| H441 | Lung | 18,299 |
| SW2171 | Lung | 71,914 |
| H82 | Lung | 1,461 |
| H146 | Lung | 4,788 |
| H196 | Lung | 73,376 |
| H226 | Lung | 101,475 |
| SKMES-1 | Lung | 12,209 |
| H1703 | Lung | 3,474 |
| SW900 | Lung | 17,567 |
| H520 | Lung | 355^{c} |
| H716 | Colon | 54,240 |
| HS675T | Colon | 24,577 |
| HT29 | Colon | <1000 |
| LS123 | Colon | 6,995 |
| SW948 | Colon | 8,837 |
| BX-PC3 | Pancreas | 23,211 |
| Capan-1 | Pancreas | 28,645 |
| Capan-2 | Pancreas | 15,975 |
| Panc0213 | Pancreas | 47,511 |
| Panc0327 | Pancreas | 72,207 |
| Panc0504 | Pancreas | 8,845 |
| 22RV1 | Prostate | 934 |
| DU145 | Prostate | 23,666 |
| H660 | Prostate | 1,068 |
| LNCAP | Prostate | 9,215 |
| PC3 | Prostate | 6,352 |
| VCAP | Prostate | 590 |

^{a}Value is an average of six measurements
^{b}Value is an average of four measurements
^{c}Value is an average of seven measurements

### Example 28: Evaluation of IL1RAP x CD3 Bispecific Antibodies in Apoptosis Assay

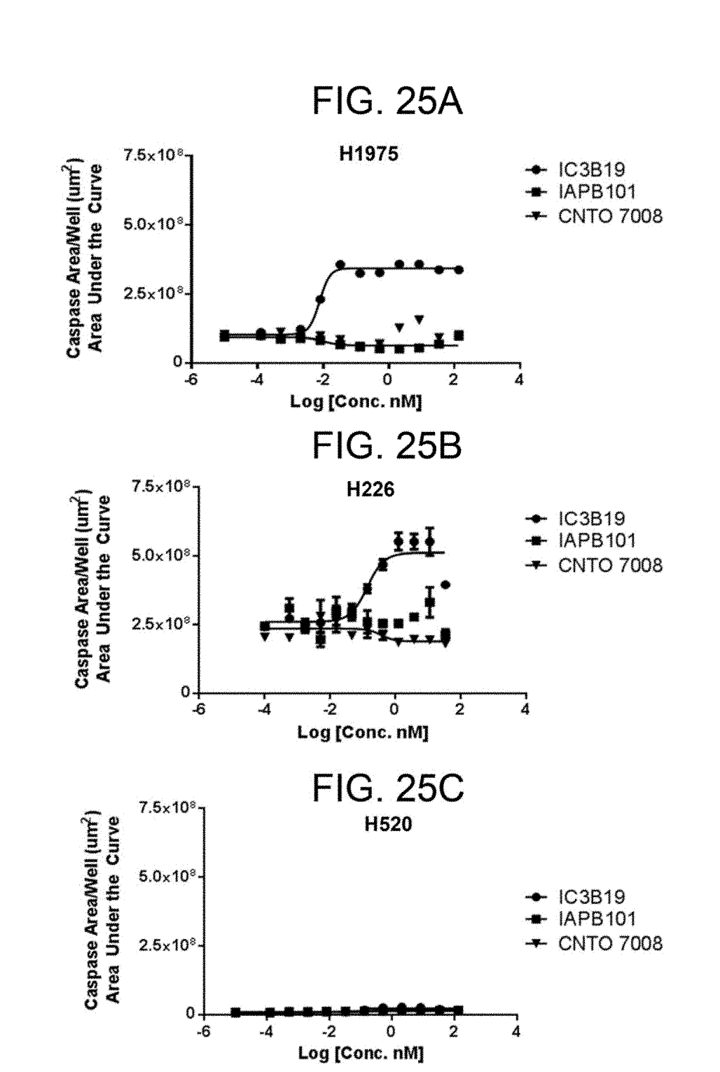

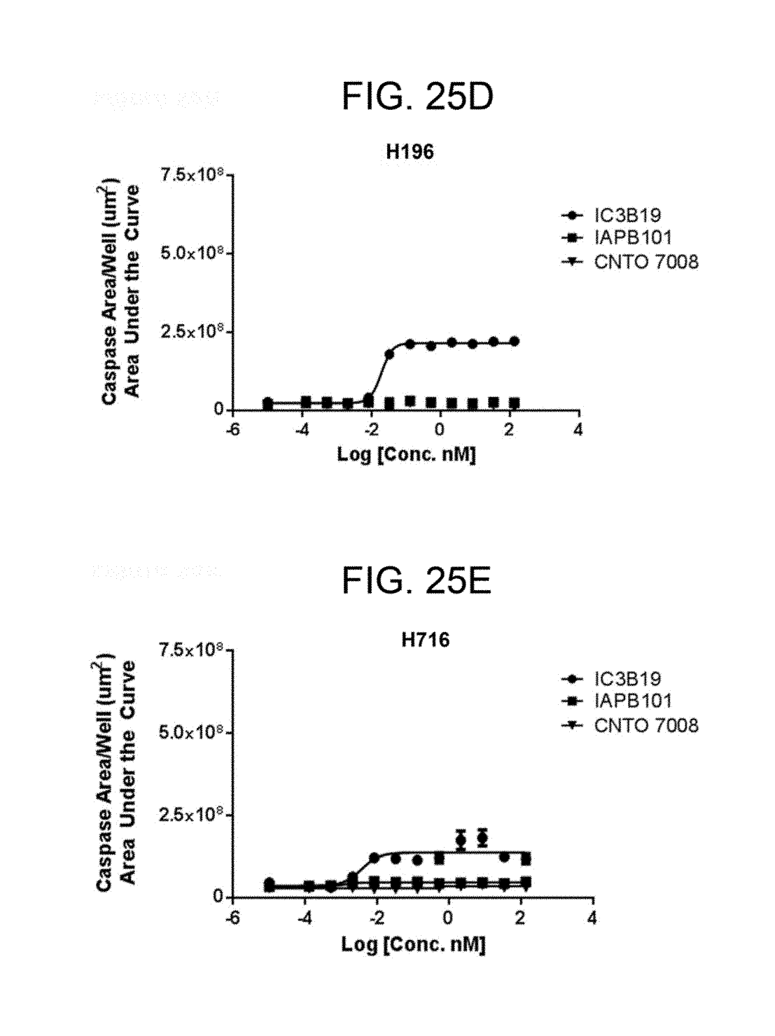

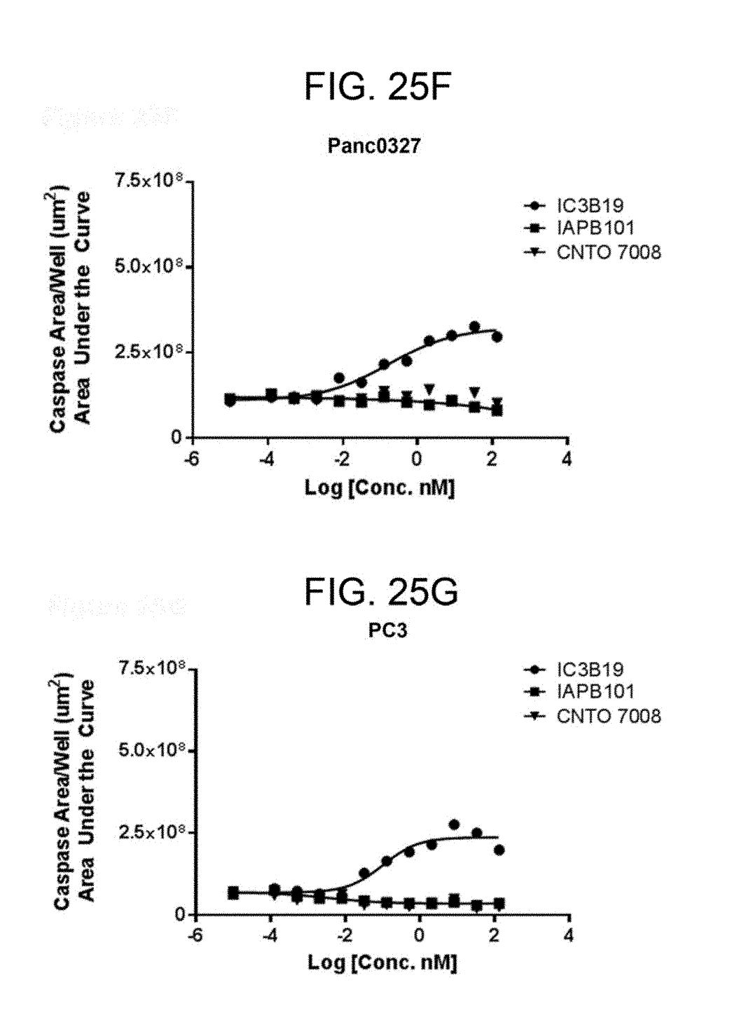

Lung, prostate, pancreas, and colon cell lines were cultured according to ATCC conditions and grown to 70-85% confluence. Target cells were dissociated with non-enzymatic dissociation buffer (Life Technologies, Cat#13151-014) where appropriate and wash in PBS. Cells were counted and resuspended in specified complete phenol-red free media to 0.4*10\hat{ }6 cells/mL. Target cells were dispensed into a sterile 96-well plate (50 μL/well) and allowed to incubate overnight at 37° C. and 5% CO₂. On the next day, Pan T-cells from healthy donors (Biological Specialties, Donors #M7412, LS-11-53108, #M6807, LS-11-53847A, or M7267, Lot#LS-11-53072B) were counted and plated at 1.0*10\hat{ }6 cells/mL in complete phenol-red free media (100 uL/well) containing 500× of Essen Bioscience's IncuCyte™ Caspase-3/7 Reagent (Cat#4440). Varying concentrations of IC3B19 (IAPB57 x CD3219) and control antibodies [CNTO 7008 (B23B39 x CD3B219) and IAPB101 (IAPB57 x B23B49]) were added to the appropriate wells. The plate was allowed to equilibrate at room temperature for 20 min and was placed in the IncuCyte™ imager maintained at 37° C. and 5% CO₂ for up to 120 hrs. Apoptosis was quantified at 72 hours using the total green object area (μm²/well) metric with the T-cells excluded by size within the IncuCyte™ imager processing definition. Area under the curve was calculated from raw values at 72 hours at each concentration in Graphpad Prism 6.02. Concentration response curves were graphed, and EC₅₀ values for IC3B19 were calculated using the non-linear regression calculation with the variable slope function. EC₅₀ values were valid if the 95% confidence interval was <log 1.5. IC3B19 stimulates a T-cell directed apoptotic response characterized by an increase in caspase activity in the majority of solid tumor cell lines tested. Control antibodies (CNTO7008 and IAPB101) did not produce measurable apoptotic responses. With the addition of IC3B19, H520 did not produce a measurable apoptotic response denoted as “No Fit” (NF). The results of the apoptosis assay are summarized in the Table 20. Representative graphs are shown in FIGS. 25A, 25B, 25C, 25D, 25E, 25F and 25G.

TABLE 20
Summary of Apoptosis Assay

| | | | Dynamic Range |
|---|---|---|---|
| | | | (Max-Min) |
| | | | Caspase |
| | | EC₅₀ value for Caspase | Area/Well Area |
| | | Area/well (nM) Area | Under the Curve |
| Cell Line | Tumor Type | Under the Curve | (×10⁸) |
| H1975 | Lung | .013 ± .009^{a} | 2.611^{a} |
| H520 | Lung | NF^{b} | ND^{b} |
| H2172 | Lung | 0.039 | 1.150 |
| H2228 | Lung | 0.043 | 1.602 |
| Calu-3 | Lung | 0.716 | 2.266 |
| SKMES-1 | Lung | 0.031 | 1.036 |
| H226 | Lung | 0.134 | 2.521 |
| SW1271 | Lung | 0.078 | 2.171 |
| H196 | Lung | 0.019 | 1.919 |
| H716 | Colon | 0.004 | 1.005 |
| Panc0213 | Pancreas | 0.192 | 1.335 |
| Panc0327 | Pancreas | 0.181 | 2.136 |
| LNCAP | Prostate | 0.039 | 0.783 |
| DU145 | Prostate | 0.445 | 1.514 |
| PC3 | Prostate | 0.102 | 1.683 |

^{a}Value is an average of seven measurements
^{b}Value is an average of three measurements
Three Healthy T-cell Donors were used; Donors #M7412, LS-11-53108 and #M6807, LS-11-53847A, and M7267, Lot#LS-11-53072B
NF = No fit is used when either Prism does not return a value (e.g., “ambiguous”) or the fit is determined to be poor (95% CI range for the log EC50 > log1.5)
ND = Not determined

In summary, IL IKAY is expressed on the surface of a variety of solid tumor cell lines including lung, colon, pancreatic, and prostate cell lines. IC3B19 stimulates a T-cell directed apoptotic response characterized by an increase in caspase activity in these IL1RAP positive solid tumor cell lines, but not in the H520s which are an IL1RAP negative cell line.

### Example 29. IL1RAP Receptor Density Levels on Hematological Malignant Cell Lines

To understand the expression of IL1RAP cell surface expression, 226 hematological cell lines were analyzed for IL1RAP cell surface receptor density level. Utilizing a commercially available phycoerythrin (PE) labeled anti-IL1RAP monoclonal antibody (R&D Systems, cat# FAB676P), receptor density levels were determined utilizing two different methods. The use of either PE-labeled beads (BD Biosciences, QuantiBRITE, cat#340768) or anti-mouse capture beads (Bang's Laboratories, Simply Cellular, cat#815) were used to capture the commercially available PE-labeled anti-IL1RAP antibody to generate standard curves. The IL1RAP geomean expression for all cell lines tested were calculated and isotype (R&D Systems, cat# IC002P) values were subtracted. Receptor density levels were generated from standard curves for both methods. Values that could not be extrapolated or were below the limit of detection were designated as not determined (ND). These data show that most hematological cell lines express IL1RAP on the cell surface at varying levels (Table 21). Among the disease indications listed, acute myeloid leukemia (AML), chronic myeloid leukemia (CIVIL), diffuse large B cell lymphoma (DLBCL), and T-cell acute lymphoblastic leukemia and T-cell leukemia's were among the disease indications that had relatively elevated IL1RAP receptor density levels.

TABLE 21
IL1RAP receptor density for each cell line as quantified by
either PE-labeled beads (QuantiBRITE) or anti-mouse
capture beads (Bangs Labs)

| | | Receptor Density | |
|---|---|---|---|
| | | (Isotype subtracted) | |
| | | | Bangs |
| Disease | Cell Line | Quantibrite | Labs |
| ALL | 697 | 10 | 19 |
| ALL | 8″E″5 | 1484 | 5388 |
| ALL | CCRF-CEM | 289 | 844 |
| | (ATCC) | | |
| ALL | CCRF-CEM | 508 | 1598 |
| | (DSMZ) | | |
| ALL | CCRF-SB | 27 | 59 |
| ALL | KASUMI-2 | 5 | 9 |
| ALL | MOLT-14 | 306 | 899 |
| ALL | MOLT-3 (ATCC) | 340 | 1014 |
| ALL | MOLT-3 (CBS) | 758 | 2515 |
| ALL | MOLT-4 (ATCC) | 139 | 368 |
| ALL | MOLT-4 (CBS) | 160 | 431 |
| ALL | P30-OHKUBO | 522 | 1650 |
| ALL | RCH-ACV | 449 | 1390 |
| ALL | RS4;11 | 744 | 2463 |
| ALL | SD-1 (DSMZ) | ND* | ND* |
| ALL | SD-1(CBS) | ND* | ND* |
| ALL | SEM | 472 | 1473 |
| ALL | SUP-B15 | 214 | 600 |
| ALL | TANOUE | 1874 | 7016 |
| AML | AML-193 | 3526 | 14360 |
| AML | AP-1060 | 3363 | 13609 |
| AML | BDCM | 70 | 169 |
| AML | CMK | 3595 | 14680 |
| AML | CTV-1 | 1460 | 5286 |
| AML | ELF-153 | 4860 | 20653 |
| AML | EOL-1 | 6521 | 28817 |
| AML | F-36p | 6196 | 27198 |
| AML | FKH-1 | 4473 | 18799 |
| AML | GF-D8 | 6264 | 27534 |
| AML | HEL | 1351 | 4843 |
| AML | HL-60 (CBS) | 1479 | 5365 |
| AML | HL-60 (DSMZ) | 2795 | 11035 |
| AML | Kasumi-1 (ATCC) | 1193 | 4206 |
| AML | Kasumi-1 (DSMZ) | 1481 | 5373 |
| AML | Kasumi-3 | 3891 | 16056 |
| AML | Kasumi-6 | 2356 | 9094 |
| AML | KG-1 (CBS) | 413 | 1266 |
| AML | KG-1 (DSMZ) | 485 | 1518 |
| AML | KG-1a | 693 | 2274 |
| AML | KMOE-2 | 2956 | 11759 |
| AML | M-07e | 2029 | 7677 |
| AML | ME-1 | 61 | 144 |
| AML | MEGAL | 369 | 1115 |
| AML | MKPL-1 | 5214 | 22368 |
| AML | ML-2 | 881 | 2984 |
| AML | MOLM-16 | 879 | 2977 |
| AML | MUTZ-8 | 2377 | 9186 |
| AML | MV4-11 (CBS) | 4632 | 19562 |
| AML | MV4-11 (DSMZ) | 5571 | 24110 |
| AML | NB-4 | 5695 | 24716 |
| AML | NOMO-1 | 1799 | 6701 |
| AML | OCI-AML2 | 4026 | 16687 |
| AML | OCI-AML3 | 4825 | 20486 |
| AML | OCI-AML4 | 663 | 2162 |
| AML | OCI-AML5 | 2277 | 8751 |
| AML | OCI-AML5 | 7396 | 33238 |
| AML | OCI-AML6 | 2387 | 9228 |
| AML | OCI-M1 | 2159 | 8236 |
| AML | OCI-M2 | 372 | 1123 |
| AML | PL-21 | 4629 | 19543 |
| AML | SH-2 | 2695 | 10590 |
| AML | SHI-1 | 4090 | 16986 |
| AML | SIG-M5 | 385 | 1168 |
| AML | SKM-1 | 1645 | 6052 |
| AML | SKNO-1 | 61688 | 367472 |
| AML | THP-1 (ATCC) | 4523 | 19037 |
| AML | THP-1 (CBS) | 4840 | 20560 |
| AML | THP-1 (DSMZ) | 1839 | 6870 |
| AML | UCSD-AML1 | 5606 | 24280 |
| AML | UT-7 | 578 | 1850 |
| B-ALL | LAZ-221 | 40 | 91 |
| B-ALL | Reh | 1346 | 4823 |
| B-ALL | ROS-50 | 578 | 1850 |
| B-ALL | VAL | ND* | ND* |
| B Cell Lymphoma | JM1 | 150 | 403 |
| B Cell Lymphoma | U-698-M | 9 | 17 |
| B-Cell Lymphoma | BC-1 | 444 | 1373 |
| B-Cell Lymphoma | BC-2 | 608 | 1959 |
| B-Cell Lymphoma | BC-3 | 371 | 1119 |
| B-Cell Lymphoma | CRO-AP2 | ND* | ND* |
| B-Cell Lymphoma | DOHH-2 | 951 | 3253 |
| B-Cell Lymphoma | Granta-519 | 275 | 799 |
| B-Cell Lymphoma | KARPAS-422 | 403 | 1230 |
| B-Cell Lymphoma | MC116 | 188 | 517 |
| B-Cell Lymphoma | OCI LY19 | 536 | 1699 |
| B-Cell Lymphoma | REC-1 | 372 | 1125 |
| B-Cell Lymphoma | SC-1 | 57 | 134 |
| B-Cell Lymphoma | U-2932 | 166 | 451 |
| B-Cell Lymphoma | ULA | 127 | 333 |
| B-Cell Lymphoma | WILL-1 | 208 | 582 |
| B-Cell Lymphoma | WILL-2 | 478 | 1492 |
| B-Cell Lymphoma | WSU-DLCL2 | 208 | 582 |
| B-Cell Lymphoma | WSU-NHL | 198 | 551 |
| B-Cell Myeloma | NCI-H929 (ATCC) | 629 | 2038 |
| B-Cell Myeloma | NCI-H929 (CBS) | 652 | 2122 |
| B-CLL | EHEB | 33 | 72 |
| B-CLL | MEC-1 | 109 | 280 |
| B-CLL | MEC-2 | 113 | 291 |
| BCP-ALL | KOPN-8 | 650 | 2114 |
| B-Lymphoblast (large | DB | 215 | 602 |
| cell lymphoma) | | | |
| B-NHL | MHH-PREB-1 | 777 | 2589 |
| B-NHL | OCI-LY1 | 57 | 134 |
| B-NHL | WSU-DLCL-2 | 358 | 1074 |
| B-NHL | WSU-FSCCL | 505 | 1587 |
| B-prolymphocytic | JVM-3 | 55 | 129 |
| leukemia | | | |
| Burkitt's lymphoma | BJAB | 50 | 115 |
| Burkitt's Lymphoma | Daudi | 266 | 768 |
| Burkitt's lymphoma | DND*-39 | 89 | 221 |
| Burkitt's lymphoma | JIYOYE | 38 | 86 |
| Burkitt's lymphoma | NAMALWA | 261 | 751 |
| Burkitt's lymphoma | P3HR-1 | 89 | 221 |
| Burkitt's Lymphoma | Raji | 265 | 765 |
| Burkitt's Lymphoma | Ramos | 1774 | 6592 |
| Chronic Neutrophilic | MOLM-20 | 547 | 1740 |
| Leukemia | | | |
| CML | BV-173 | 997 | 3432 |
| CML | CML-T1 | 427 | 1312 |
| CML | EM-2 | 6214 | 27284 |
| CML | EM-3 | 1753 | 6508 |
| CML | JURL-MK1 | 400 | 1220 |
| CML | K-562 (ATCC) | 51 | 119 |
| CML | K-562 (DSMZ) | 35 | 77 |
| CML | KU812F | 3999 | 16561 |
| CML | KYO-1 | 576 | 1843 |
| CML | LAMA-84 | 14184 | 69499 |
| CML | MEG-01 | 5587 | 24186 |
| CML | MEG-A2 | 6266 | 27544 |
| CML | MOLM-1 | 5741 | 24944 |
| CML | MOLM-6 | 2143 | 8170 |
| CML | NALM-1 (CBS) | 246 | 704 |
| CML | NALM-1 (DSMZ | 407 | 1243 |
| CML | NALM-12 (CBS) | 472 | 1473 |
| CML | NALM-6 | 1031 | 3566 |
| CML | SPI-801 | 479 | 1498 |
| CML | SPI-802 | 109 | 280 |
| CML | TMM | 53 | 124 |
| CTCL | H9 (derivative of | 169 | 459 |
| | HuT 78) | | |
| CTCL | HH | ND* | ND* |
| CTCL | HuT 78 | 59 | 139 |
| CTCL | MJ | 100 | 253 |
| DLBCL | CARNAVAL | 312 | 922 |
| DLBCL | HT | 246 | 703 |
| DLBCL | OCI LY18 | 743 | 2462 |
| DLBCL | OCI LY7 | 223 | 628 |
| DLBCL | OCI-LY10 | 287 | 838 |
| DLBCL | OCI-LY-18 | 832 | 2797 |
| DLBCL | OCI-LY19 | 244 | 698 |
| DLBCL | OCI-LY3 | 115 | 296 |
| DLBCL | Pfeiffer (ATCC) | 371 | 1120 |
| DLBCL | SU-DHL-1 | 10536 | 49625 |
| DLBCL | SU-DHL-10 | 71 | 329 |
| DLBCL | SU-DHL-10 | 126 | 171 |
| DLBCL | SU-DHL-16 | 3070 | 12273 |
| DLBCL | SU-DHL-4 | 105 | 267 |
| DLBCL | SU-DHL-5 | 156 | 420 |
| DLBCL | SU-DHL-6 | 413 | 1265 |
| DLBCL | SU-DHL-8 | 774 | 2578 |
| DLBCL | TMD-8 | 302 | 888 |
| DLBCL | TOLEDO | 362 | 1088 |
| DLBCL | U-2940 | 536 | 1701 |
| Erytholeukemia | HEL 92.1.7 | 3590 | 14653 |
| Erythroleukemia | TF-1 (ATCC) | 4361 | 18268 |
| Erythroleukemia | TF-1 (CBS) | 6451 | 28469 |
| Erythroleukemia | TF-1 (DSMZ) | 4966 | 21164 |
| Histocytic Lymphoma | JOSK-I | 3455 | 14033 |
| Histocytic Lymphoma | JOSK-M | 4134 | 17194 |
| Histocytic Lymphoma | SU-DHL-2 | 1339 | 4796 |
| Histocytic Lymphoma | U937 | 6682 | 29625 |
| Hodgkin lymphoma | HDLM-2 | 154 | 413 |
| Hodgkin lymphoma | Hs 611.T | 141 | 374 |
| Hodgkin lymphoma | HS445 | 120 | 313 |
| Hodgkin lymphoma | L-1236 | 1463 | 5302 |
| Hodgkin lymphoma | L-428 | 428 | 1318 |
| Hodgkin lymphoma | L-540 | 970 | 3329 |
| Hodgkin lymphoma | SUP-HD1 | 51 | 119 |
| Hodgkin lymphoma | TO 175.T | 555 | 1768 |
| Mantle Cell Lymphoma | JEKO-1 | 936 | 3195 |
| Mantle Cell Lymphoma | JVM-13 | 170 | 462 |
| Mantle Cell Lymphoma | JVM-2 | 18 | 37 |
| Mantle Cell Lymphoma | MAVER-1 | 668 | 2181 |
| Mantle Cell Lymphoma | MINO | 144 | 384 |
| Mantle Cell Lymphoma | Z138 | 299 | 878 |
| MCL | JVM-2 | 238 | 678 |
| MML | GDM-1 (ATCC) | 1547 | 5648 |
| Mouse Bone Marrow | FDCP-1 (CBS) | 161 | 436 |
| Multiple Myeloma | ARH77 dsRed | 184 | 506 |
| Multiple Myeloma | ARH77(ATCC) | 192 | 531 |
| Multiple Myeloma | EJM | 459 | 1426 |
| Multiple Myeloma | HuNS1 | 245 | 701 |
| Multiple Myeloma | IM-9 | 213 | 597 |
| Multiple Myeloma | KMS-11 | 1347 | 4828 |
| Multiple Myeloma | KMS-12 PE | 13 | 24 |
| Multiple Myeloma | KMS-12-BM | 35 | 77 |
| Multiple Myeloma | LP-1 | 332 | 987 |
| Multiple Myeloma | MM1R | 395 | 1204 |
| Multiple Myeloma | MM1S | 226 | 639 |
| Multiple Myeloma | MOLP-2 | 130 | 340 |
| Multiple Myeloma | MOLP-8 | 464 | 1444 |
| Multiple Myeloma | OPM-2 | 3741 | 15354 |
| Multiple Myeloma | RPMI 8226 (ATCC) | 443 | 1369 |
| Multiple Myeloma | U266 | 119 | 308 |
| Myeloma | HTK- | 2038 | 7718 |
| Myeloma | JIM-1 | 3007 | 11989 |
| Myeloma | JIM-3 | 1478 | 5363 |
| Myeloma | U266B1 | 37 | 81 |
| NHL | FARAGE | 153 | 412 |
| NHL | RL | 145 | 386 |
| Plasma Cell Leukemia | JJN-3 | 182 | 500 |
| Plasma Cell Leukemia | L-363 | 218 | 612 |
| Plasma Cell Leukemia | SK-MM-2 | 268 | 776 |
| Plasmacytoma | AMO-1 | 143 | 379 |
| T cell leukemia | TALL-1 | ND* | ND* |
| T cell lymphoma | SR-786 | 20643 | 106323 |
| T-ALL | ALL-SIL | 3008 | 11992 |
| T-ALL | CEM/C1 | 1433 | 5177 |
| T-ALL | CEM/C2 | 799 | 2673 |
| T-ALL | HPB-ALL | 371 | 1120 |
| T-ALL | Loucy | 159 | 429 |
| T-ALL | MOLT-13 | 212 | 594 |
| T-ALL | MOLT-17 | 892 | 3028 |
| T-ALL | P12-ICHIKAWA | 124 | 324 |
| T-ALL | RPMI-8402 | 176 | 482 |
| T-ALL | SUP-T11 | 255 | 734 |
| T-Cell Leukemia | Jurkat | 2523 | 9826 |
| T-Cell line from | HuT-102 | 185 | 508 |
| Lymphoma | | | |
| T-Cell Lymphoma | SUP-T1 | 848 | 2858 |
| T-CLL | MOTN-1 | 277 | 805 |

Note:
Some of the cell lines are repeated because they were obtained from different sources.
CBS = Janssen's internal cell banking service,
ATCC = American Type Culture Collection,
DSMZ = Deutsche Sammlung von Mikroorganismen und Zellkulturen (German Collection of Microorganisms and Cell Culture),
ND = not determined, levels were below the level of detection

### Example 30. Evaluation of IC3B19 in Functional Cell Cytotoxicity Assay with CML, DLBCL, T-ALL and T-Cell Leukemia Cell Lines

IC3B19 and control antibodies (CNTO 7008 and IAPB101) were tested in additional hematological indications. Chronic Myeloid Leukemia (CIVIL) target cells (LAMA-84, MEG-01, and KYO-1), Diffuse Large B-Cell Lymphoma (DLBCL) target cells (SU-DHL-16, U-2940, SU-DHL-6), and T-Acute Lymphoblastic Leukemia (ALL) and T-cell leukemia/lymphoma target cells (ALL-SIL, CEM/C1, HPB-ALL, Jurkat, and SUP-T1) were tested with three healthy control pan CD3+ T-cell donors. The protocol previously described in Example 12 was followed.

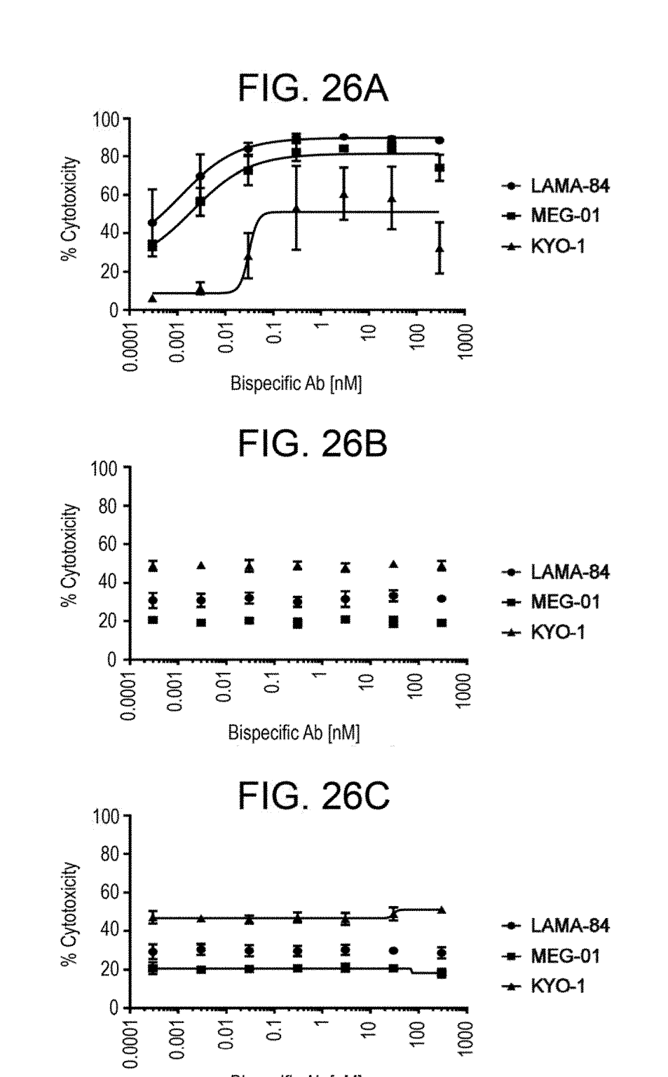

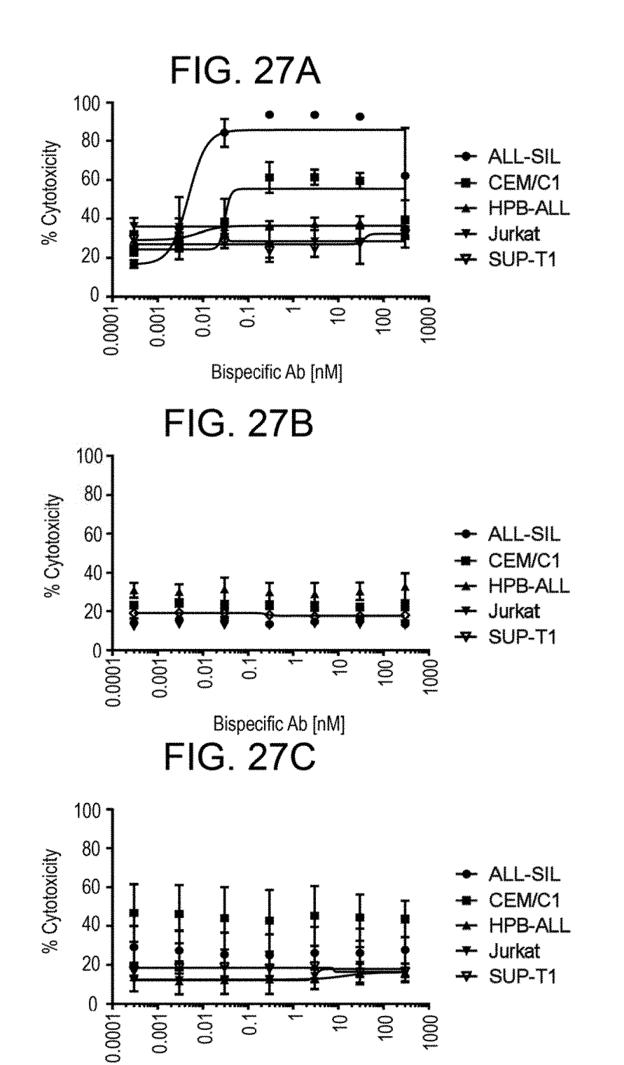

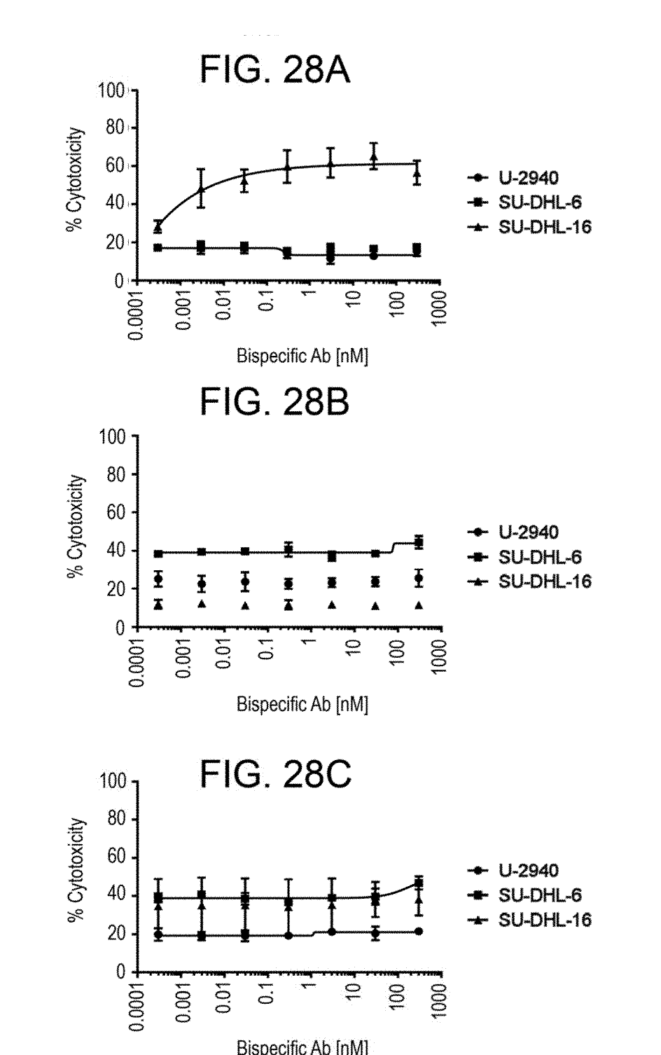

An average of the 3 healthy control pan CD3+ T-cells is represented (FIGS. 26A, 26B, 26C, 27A, 27B, 27C, 28A, 28B and 28C). IC3B19 induced cytotoxicity in CIVIL, T-ALL/T-cell leukemia/lymphoma, and DLBCL cell lines as well as T-cell mediated activation (CD25). The maximal cell cytotoxicity observed and corresponding EC₅₀ (nM) are shown in Table 22. These data show that IL1RAPxCD3 has activity in CML, T-ALL/T-cell leukemia/lymphoma and DLBCL indications but that control antibodies (CNTO 7008 and IAPB101) had no overall T-cell mediated tumor cell cytotoxicity.

TABLE 22
IC3B19 Average EC₅₀ (nM) and
Maximal Percent Cytotoxicity

| | | | Max Cytotoxicity |
|---|---|---|---|
| Cell line | Indication | EC₅₀ (nM) | (Background Subtracted) |
| LAMA-84 | CML | 0.001 | 70.5 |
| MEG-01 | CML | 0.002 | 59.3 |
| KYO-1 | CML | ND* | 54.4 |
| ALL-SIL | T-ALL | 0.004 | 77.0 |
| CEM/C1 | T-ALL | ND* | 36.5 |
| HPB-ALL | T-ALL | 0.008 | 3.4 |
| SU-DHL-16 | DLBCL | ND* | 54.7 |
| U-2940 | DLBCL | ND* | 1.2 |
| SU-DHL-6 | DLBCL | ND* | 0.9 |
| Jurkat | T-cell | ND* | 0.0 |
| | leukemia/lymphoma | | |
| SUP-T1 | T-cell | ND* | 2.0 |
| | leukemia/lymphoma | | |

Note:
*ND = Not Determined, EC50 curve was ambiguous

### Example 31. Efficacy of IC3B19 in 111975 Human Non-Small Cell Lung Carcinoma Xenografts in T Cell Humanized NSG Mice

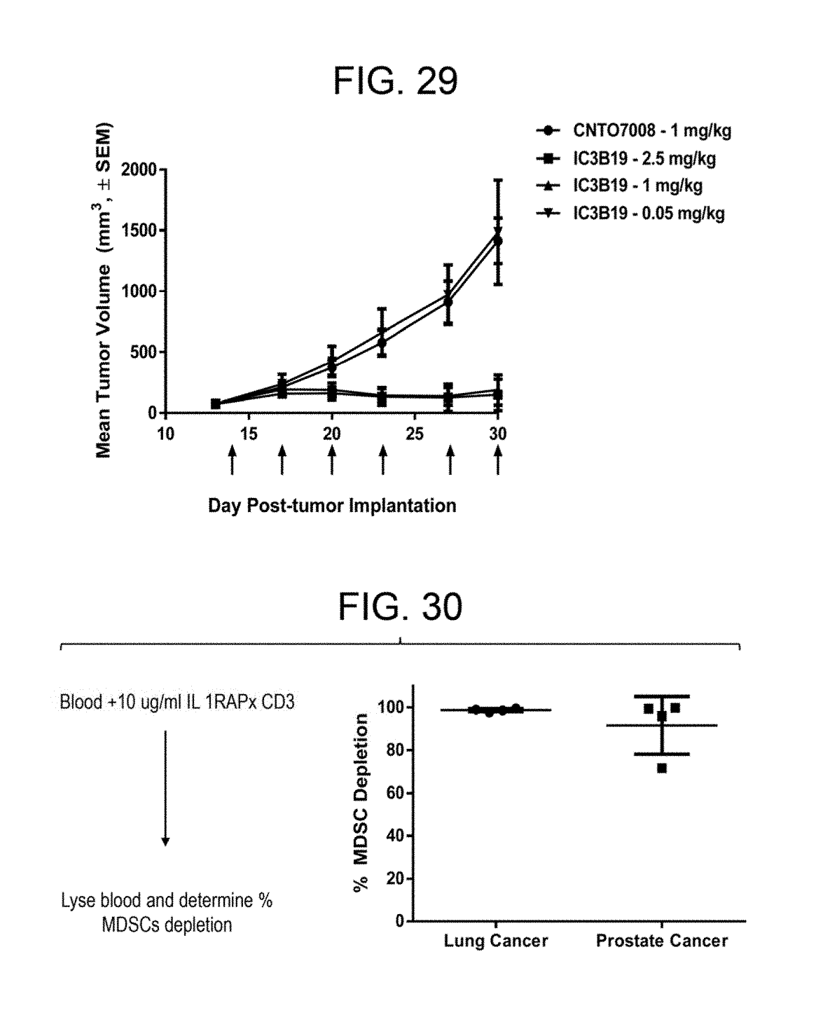

Efficacy of IC3B19 was evaluated in established H1975 human non-small cell lung carcinoma xenografts in female NSG mice humanized with 20×10⁶ in vitro expanded and activated human T cells ip. Mice were randomized by tumor volume into groups of ten animals each on day 13 post-tumor implantation at an average tumor volume of 74 mm³. IC3B19 at 0.5, 1 or 2.5 mg/kg or CNTO7008 (CD3xNull control) at 1 mg/kg were dosed ip twice weekly on days 14, 17, 20, 23, 27, 30, 35, and 38 for a total of 8 doses. On day 30 post-tumor implant, which was the last date when at least nine of ten animals remained in all treatment groups, tumor growth inhibition (% TGI) was calculated. Statistically significant tumor growth inhibition was observed at IC3B19 at 1 mg/kg and 2.5 mg/kg with 80% and 90% TGI, respectively, compared to the CNTO7008-treated controls (p<0.0001, FIG. 29). IC3B19 treatment at 2.5 mg/kg resulted in tumor stasis or regression in 4/10 mice on day 30.

### Example 32. Targeting IL1RAP⁺ Myeloid-Derived Suppressor Cells (MDSC) with IC3B19

Expansion of Tregs and MDSCs in the lung and prostate tumor microenvironment is part of the mechanism by which cancer cells escape from host immune surveillance and may limit response to checkpoint inhibitors (Peterson 2006; Dasanu 2012; Srivastava 2012, Idorn et al 2014). IL1RAP is an accessory protein for members of the IL-1 cytokine family (IL-1/IL-1R, IL-33/ST2 and IL-36/IL-1RL2) allowing cytokine signaling involved in pro-inflammatory and innate immune responses. Though IL1RAP is poorly expressed in normal tissue and normal cells, we have detected high levels of IL1RAP surface expression on myeloid-derived suppressor cells from lung and prostate cancer donor whole blood. While the biology is not fully understood, IL1RAP, IL-1, and IL-33 may enhance tumor survival/growth by suppressing immune attack and promoting angiogenesis. Because of the lack of durable outcomes in patients with both liquid and solid tumor types, IC3B19 was developed, which redirects the immune system to kill IL1RAP positive tumor cells and tumor derived MDSCs. Therefore, the depletion of this immune suppressive population with IC3B19 is hypothesized to lead to an improvement in clinical responses in solid tumors.

To test this hypothesis, an MDSC donor blood depletion ex-vivo assay was followed. Briefly, blood samples were diluted 1:1 with RPMI (10% FBS+1% penicillin/streptomycin). This served as baseline percentage of target expression (receptor density/cell) on MDSC. The MDSC panel consisted of L/D, LIN-(CD3/CD56/CD19/), HLA-DR-low, CD11b+, CD33+, CD14, CD15: Target expression on MDSC: PE IL1-RAP. Samples were stained with the above panels and incubated for 30 min at 4° C. RBCs were lysed using RBC Lysis Buffer (ebioscience cat#00-4300-54), covered for 5 min at room temperature and spun for 4 minutes at 1500 rpm to remove buffer. Lysis with buffer was performed at least 4 times. Samples were washed with DPBS (Invitrogen, Cat#141902-250), stained with Near IR L/D dye (Invitrogen, Cat#10082-147), and covered at room temperature for 10-15 minutes. A final wash was performed with PBS/FACS and samples were resuspended in FACS buffer for analysis on Fortessa. The Geometric mean ratios were calculated in Flow Jo V_10 using Singlets/Live/Cells populations followed by MDSC panel markers, and depletion (%) of MDSC population is measured (FIG. 30)

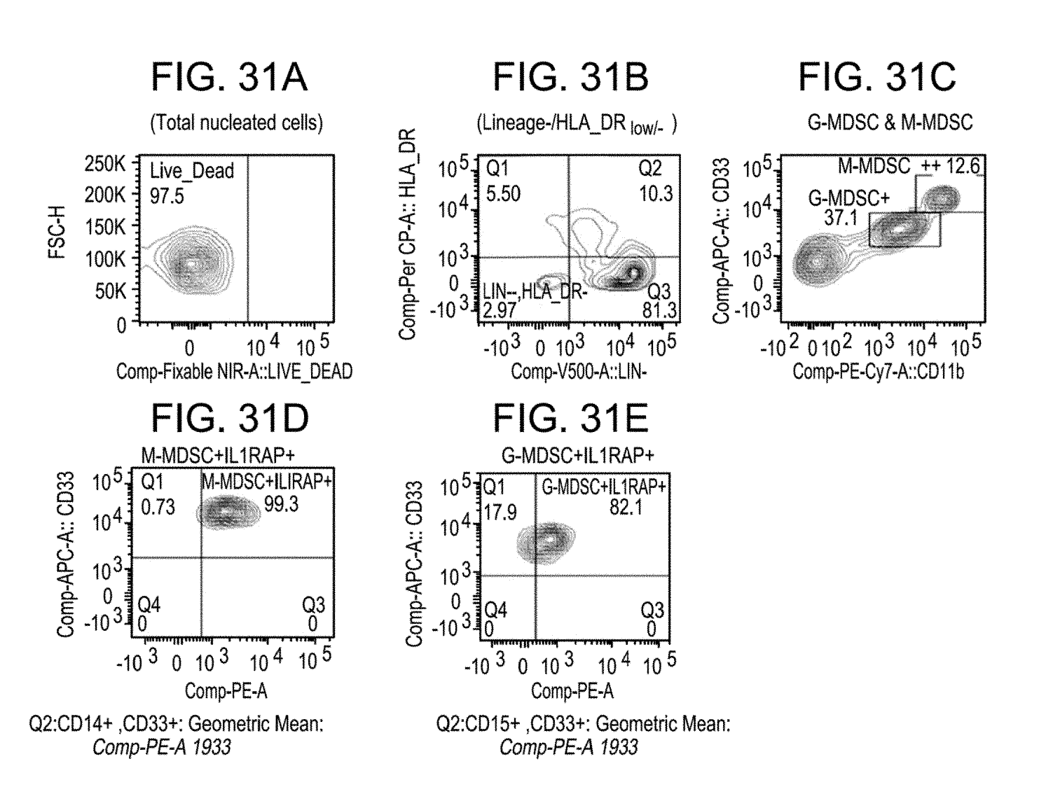

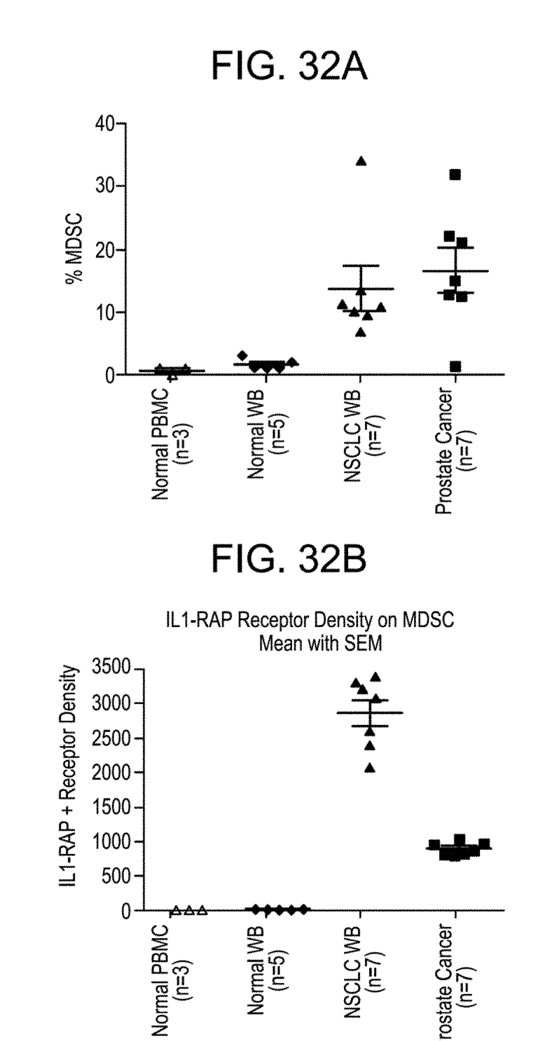

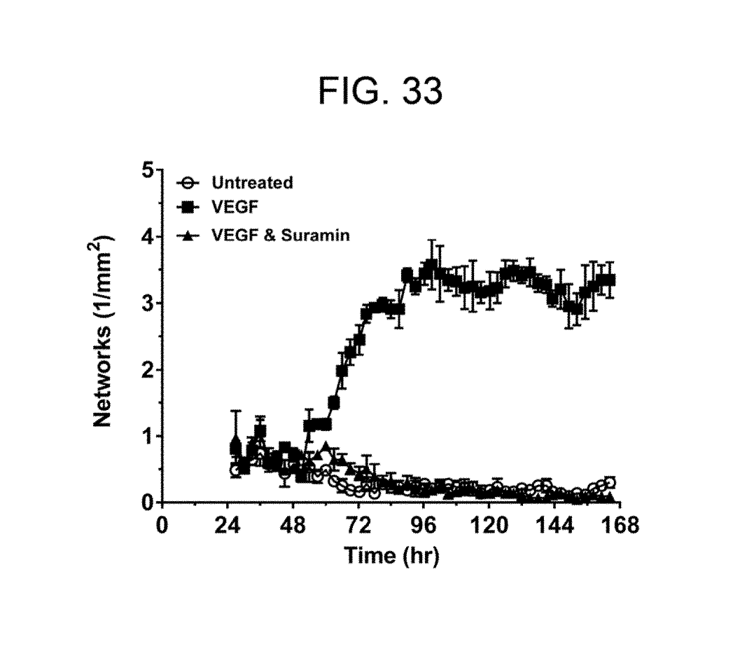

Preclinical analysis of commercially sourced peripheral blood samples from NSCLC and prostate cancer donors demonstrated significant increases in IL1RAP⁺ MDSCs in all donors tested as compared to peripheral blood from healthy subjects. Detailed analysis demonstrated elevated expression of IL1RAP on the monocytic MDSC population (FIGS. 31A, 31B, 31C, 31D and 31E) and sensitivity of these MDSCs to depletion by IL1RAPxCD3 in prostate and lung cancer donor blood in ex-vivo assay. Using the quant-brite beads quantification method, IL1RAP receptor densities range from 2500 receptors/cell for NSCLC and ˜600-800 receptors/cell for Prostate cancer in whole blood of solid tumor donors (FIGS. 32A and 32B). The depletion of the IL1RAP+ immunosuppressive cells in these blood samples leads to increased T cell activation and proliferation.

In summary, MDSC levels variable in donor blood samples across tumors ˜25% in Prostate, ˜10% in NSCLC. IL1RAP is expressed with variable receptor density seen on MDSC from patient donor samples: ˜600-800 receptors/cell for Prostate and 2500 receptors/cell for NSCLC. IL1RAPxCD3 has the ability to deplete IL1RAP⁺ MDSCs from donor blood samples.

### Example 33. Assessment of the Role of IL1RAP x CD3 Bispecific Antibody in Disrupting Nascent Tumor Vasculature

To investigate whether IL1RAPxCD3-dependent T cell redirection can disrupt and eliminate newly-established vasculature in the tumor microenvironment, the angiogenesis assay was developed, which measures relative expansion of tubular networks on 2D glass surface. To this end, a fluorescently labeled Normal Human Umbilical Vein Endothelial Cells (HUVEC) was obtained and co-cultured them with Normal Human Dermal Fibroblasts (NHDF) in the presence of VEGF stimulation (4 ng/mL). Suramin (100 μM), a general tyrosine kinase inhibitor, was supplemented to block VEGF signaling. The plates containing cultured cells were then imaged using IncuCyte™ Zoom every 3 hours. As FIG. 33 shows, VEGF stimulation induces rapid expansion of the tubular networks shortly after treatment, while addition of suramin completely negates that effect. The established networks can persist for at least 5 days in the incubator. These results demonstrate the dynamic range of the assay.

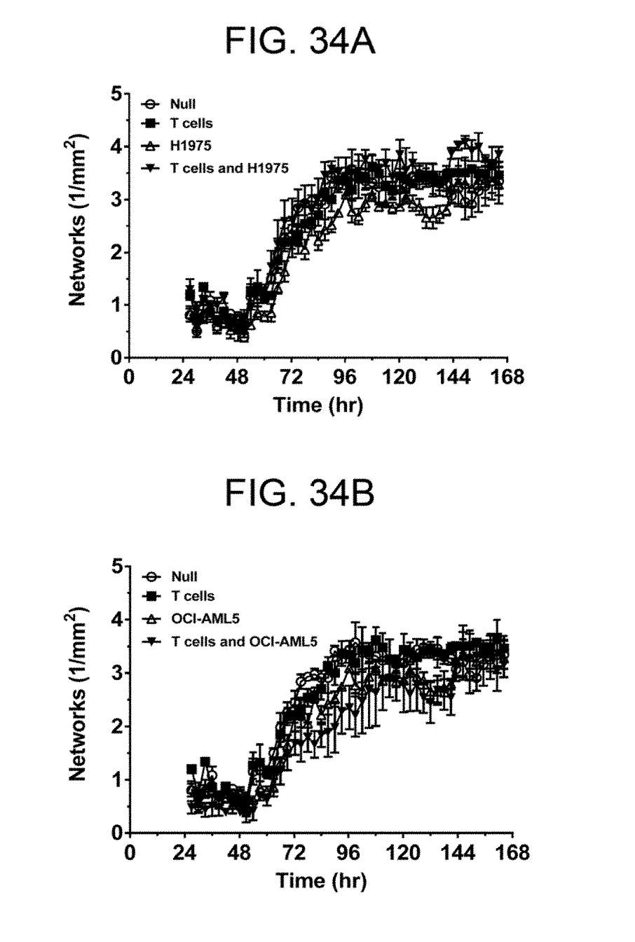

As the next step in determining the effect of IL1RAPxCD3-dependent T cell redirection, the network growth in the presence of isolated healthy donor pan-T cells and tumor cells was assessed. H1975 lung cancer cell line was used to simulate solid tumor (NSCLC) and OCI-AML5 cells were used to simulate liquid tumor (AML). FIGS. 34A and 34B shows that co-culturing HUVECs with T cells or H1975 cells does not perturb tubular network formation for the duration of the assay. Interestingly, addition of OCI-AML5 cells to HUVEC culture somewhat decelerated the network growth but did not inhibit the maximal network density, since by Day 6 of the assay (144 hours), all networks were growing comparably well.

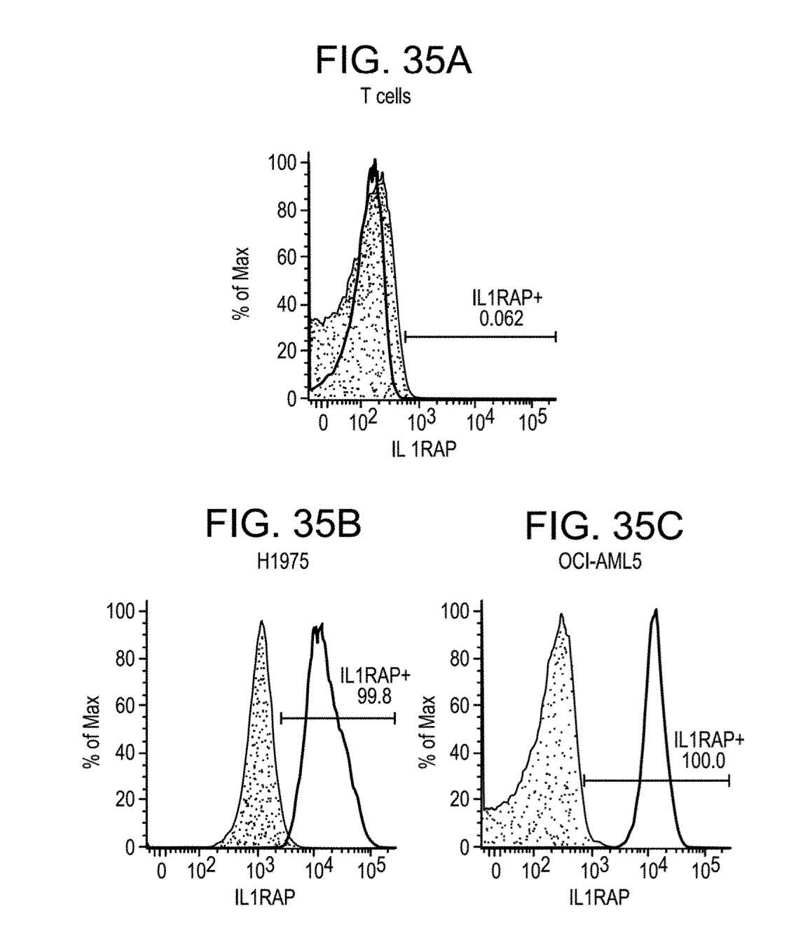

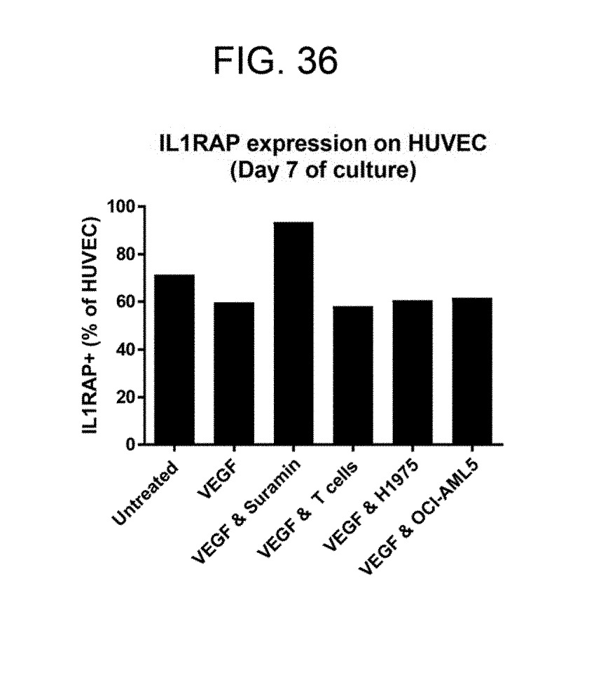

The levels of IL1RAP expression on the T cells and on the cancer cells were then assessed. In line with multiple previous observations, T cells were completely negative for IL1RAP, while H1975 and OCI-AML5 expressed high levels of the molecule on the surface (FIGS. 35A, 35B and 35C). This confirmed the intent to use these cells to model IL1RAP-positive tumor and its microenvironment in the angiogenesis assay. Having assessed IL1RAP expression levels on T cells and on cancer cells, the question came up whether HUVEC cells express IL1RAP. Flow cytometry analysis immediately after thawing revealed that IL1RAP was not present on cell surface (data not shown). However, upon culture on glass for 7 days, HUVEC showed some expression of IL1RAP, with approximately 60% of cells having protein staining above isotype (FIG. 36). The induced expression was not dependent on culture conditions but seemed to be enhanced in the presence of suramin, possibly as a mechanism to cope with stress.

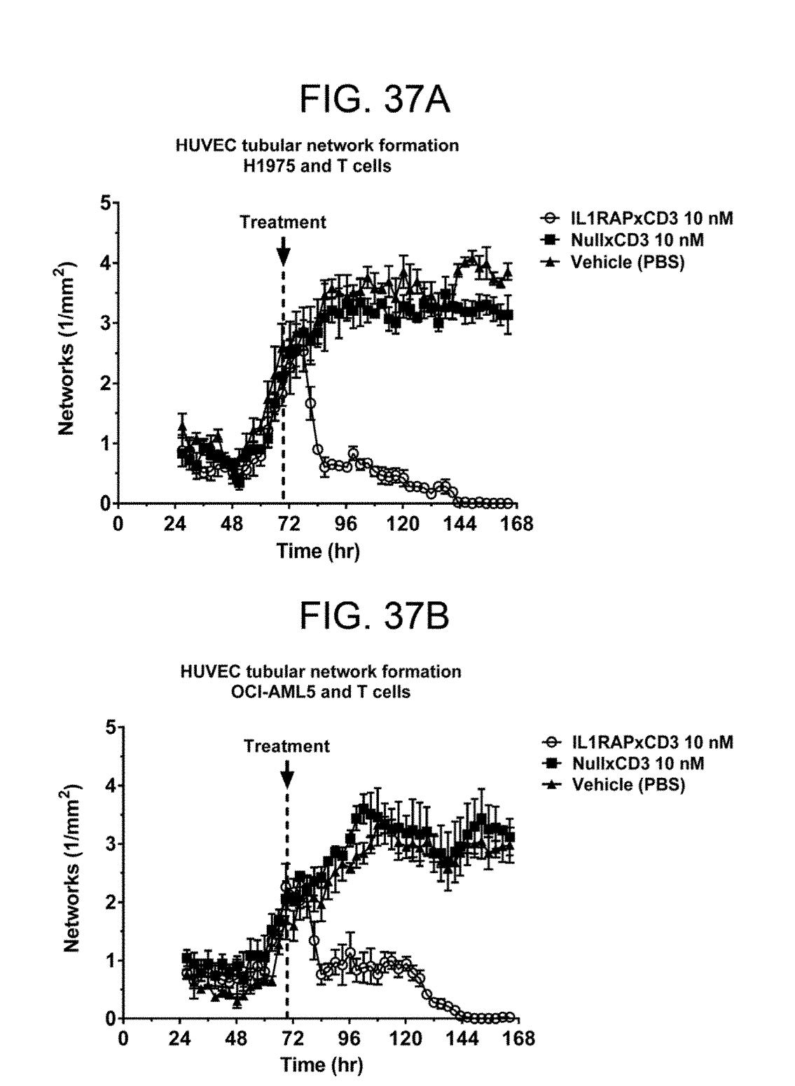

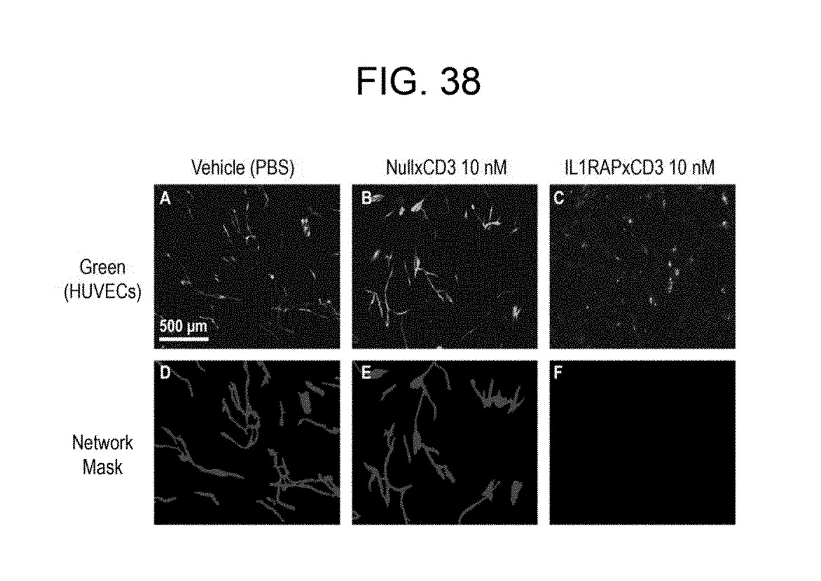

Finally, HUVEC with T cells and cancer cells were co-cultured in the presence of IL1RAP x CD3 bispecific antibody. FIGS. 37A and 37B shows that within 24 hours after treatment 10 nM IL1RAPxCD3 was sufficient to completely disrupt the tubular networks. However, treatment with the control compound (NullxCD3) or vehicle (PBS) did not alter the established network dynamics. This observation was repeated with H1975 (FIG. 37A) and OCI-AML5 (FIG. 37B) cells, indicating that the role of IL1RAPxCD3-dependent T cell redirection in tumor angiogenesis is relevant in solid and liquid tumors. Doses of 100 nM and 1 nM of IL1RAPxCD3 bispecific antibody were also tested and produced similar results. An example of representative network architecture in response to pharmacological interventions is shown in FIG. 38 where panels A, B and C show the green fluorescence from the HUVEC tubular network and D, E and F show computer-generated network masks used in the analysis.

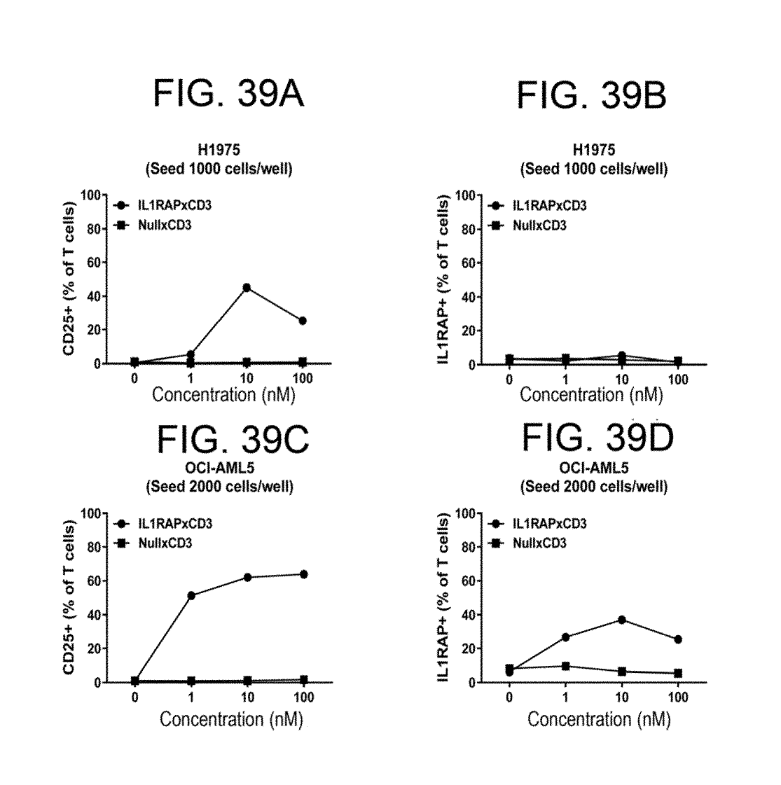

After the imaging assay was complete, the technical replicates were pooled and analyzed by flow cytometry for T cell activation marker (CD25) and IL1RAP expression on T cells. Consistent with expression of IL1RAP on HUVEC and their disruption upon treatment with IL1RAPxCD3 bispecific antobody, we saw marked increase of CD25 on T cells in an antibody dependent manner. T cells exposed to NullxCD3 DuoBody® Ab (CNTO 9253) did not upregulate CD25. This was similar between H1975 cells (FIG. 39A) and OCI-AML5 cells (FIG. 39C). Interestingly, although IL1RAP was not induced on T cells activated in the presence of H1975 (FIG. 39B), we saw substantial increase of IL1RAP on T cells activated with OCI-AML5 (FIG. 39D), suggesting that soluble factors produced by AML cell line could trigger expression of IL1RAP on T cells upon activation.

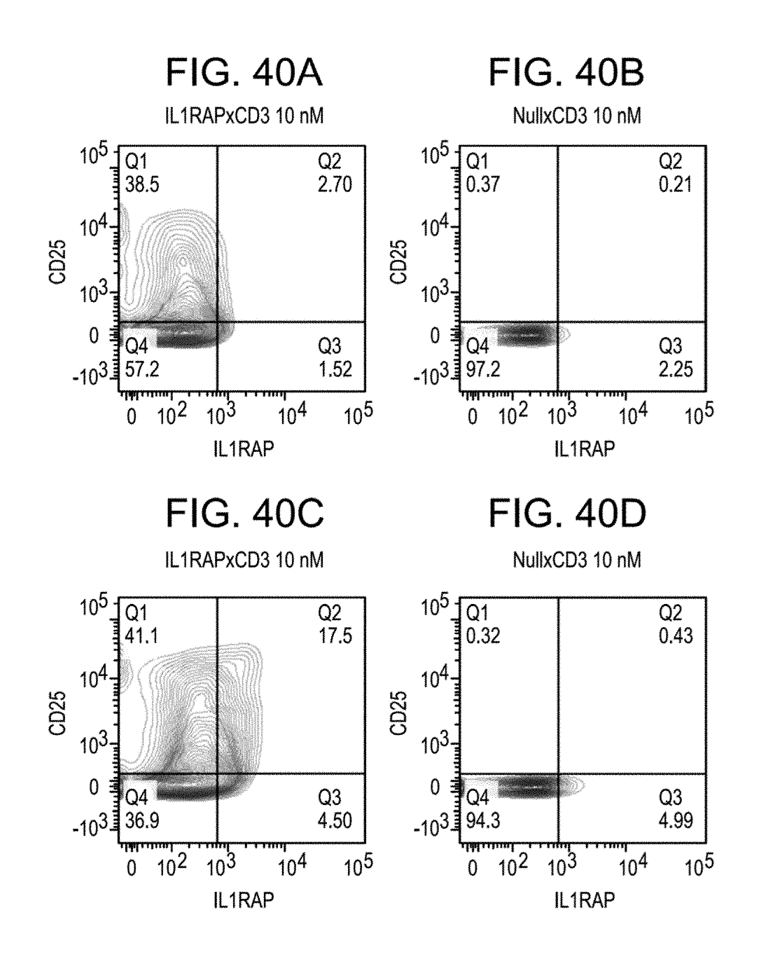

Lastly, to investigate the relationship between CD25 and IL1RAP expression on T cells, contour plots were generated and quadrant gates were set based on isotype control staining. The resulting diagrams show that in the presence of H1975 cells, 10 nM IL1RAPxCD3 induces CD25 but not IL1RAP (FIG. 40A). Activation is specific, since NullxCD3 does not produce analogous increase in CD25 (FIG. 40B). Whereas, T cells co-cultured with OCI-AML5 cells and treated with IL1RAPxCD3 increase CD25 and IL1RAP (FIG. 40C). Importantly, only a subset of activated T cells expressed IL1RAP. Furthermore, NullxCD3 does not induce CD25 or IL1RAP expression on T cells (FIG. 40D).

### Example 34. Ex-Vivo Evaluation of IL1RAP x CD3 Bispecific Antibody Effect on Primary AML and MDS Leukemic Blasts and Myeloid Derived Suppressor Cells

The purpose of this study was to investigate whether the IL1RAP x CD3 bispecific antibody can activate T cells from donors with acute myeloid leukemia (AML) and myelodysplastic syndrome (MDS) against leukemic blasts. For this reason, we established culture conditions mimicking tumor microenvironment (TME) to support growth of primary donor leukemic cells. This study was performed with the tool compound with IL1RAP binding arm (IAPB57), and CD3 binding arm (B220). Briefly, fresh mononuclear cells isolated from peripheral blood (PBMC) from two AML donor samples and cryopreserved bone marrow mononuclear (BMMC) cells from two MDS donor samples (Table 23 and Table 24 respectively) were seeded over a layer of human stroma cell line HS-5 and expanded for ten to fourteen days. Next, cell cultures were divided into three groups: untreated, treated with IL1RAP x CD3 Ab and treated with Null x CD3 Ab (both Ab at 1 μg/mL). At Day 0 and Day 14 of the treatment, cells were analyzed by flow cytometry for evaluation of IL1RAP+ blasts and myeloid derived suppressor cells (MDSC) as well as expansion/activation of T cells.

TABLE 23
AML Donor Characteristics

| | Age | | Disease | | Collection | Blast | T cell | Cytogenetic |
|---|---|---|---|---|---|---|---|---|
| Donor | (year) | Diagnosis | Phase | Material | Status | (%)² | (%)² | Abnormalities |
| AML_5503 | 63 | AML | FD | Fresh PB | De Novo | 68.9 | 9.14 | N/A |
| AML_MT0034 ¹ | 74 | AML-M7 | FD | Fresh PB | N/A | 80.3 | 7.28 | Monosomy 7 |

AML, Acute Myeloid Leukemia; M7, Megakaryoblastic; FD, First diagnosis; PB, Peripheral Blood.
¹ Donor in chemotherapy and under ongoing treatment with Dacogen ® as of June 2016. History of Myelofibrosis-grade 2 with transformation to Acute Myeloid Leukemia.
²Percent of blasts and T cells as measured by flow cytometry at Day 0 of treatment.

TABLE 24
MDS Donor Characteristics

| | | Disease | Collection | Collection | Blast | T cell | Cytogenetic |
|---|---|---|---|---|---|---|---|
| Donor | Diagnosis | Subtype | Date | Status | (%)² | (%)² | Abnormalities |
| MDS_4332¹ | MDS | RAEB-2 | Dec. 3, 2014 | De Novo | 26.6 | 1.54 | 43~45, XY, add(2)(p12), |
| | | | | | | | −3, add(4)(q31), −7, |
| | | | | | | | add(7)(q11.2), der(12)t(7; |
| | | | | | | | 12)(q11.2; p13), |
| | | | | | | | +mar[cp10]/44~46, idem, |
| | | | | | | | +add(4)(q31)[cp8]/ |
| | | | | | | | 45, idem, +8[3]/46, XY[5] |
| MDS_4594* | MDS | RAEB-2 | Aug. 6, 2014 | De Novo | 29.2 | 2.21 | 46, XY[20] |

MDS, Myelodysplastic Syndromes; RAEB-2, Refractory anemia with excess blasts-2.
¹Frozen bone marrow MNC from;
²Percent of blasts and T cells as measured by flow cytometry at Day 0 of treatment.

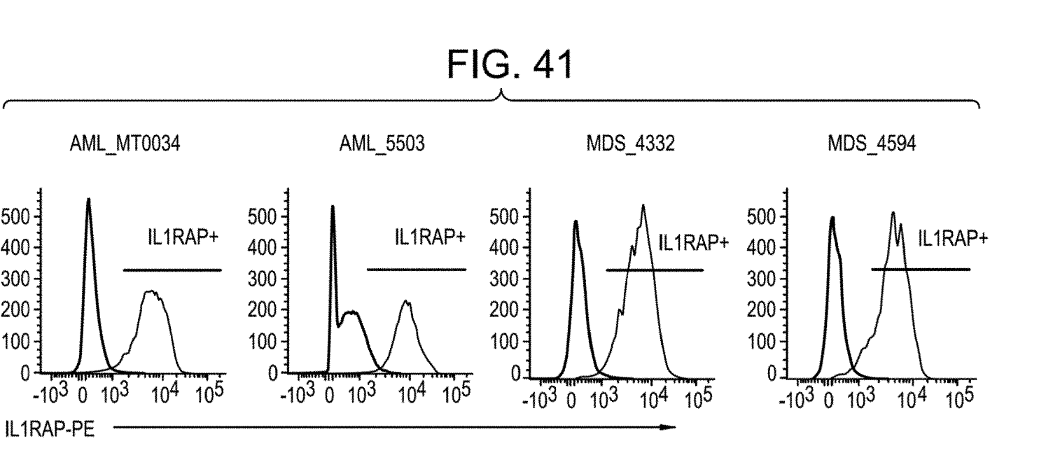

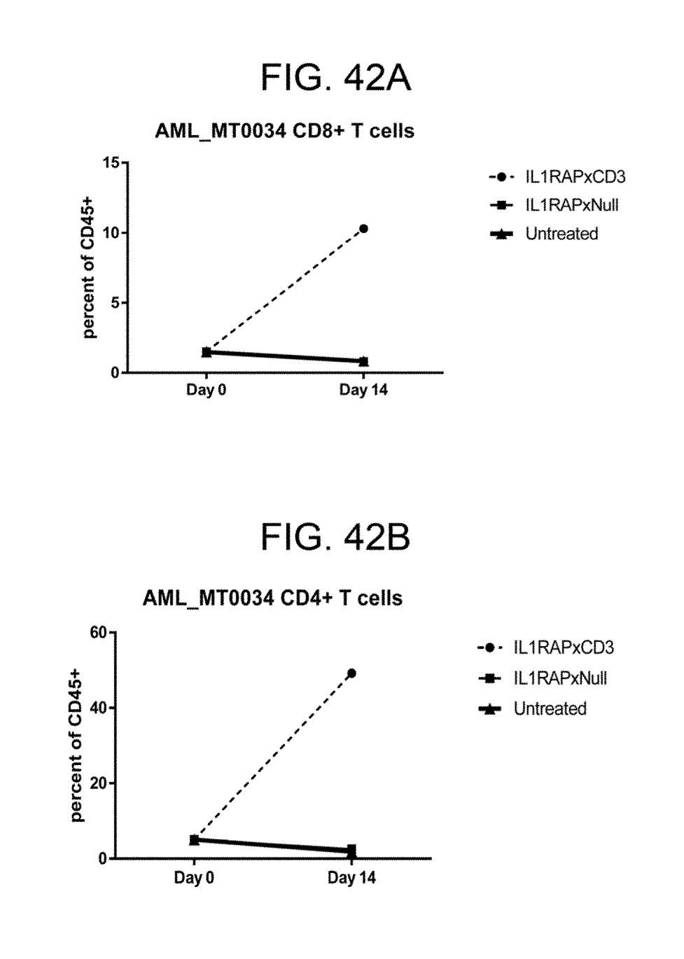

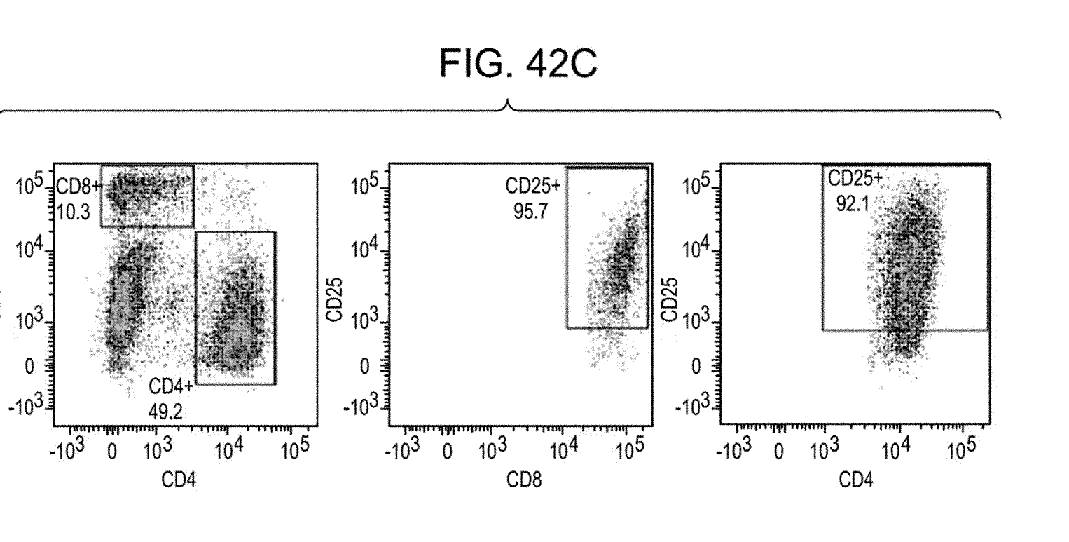

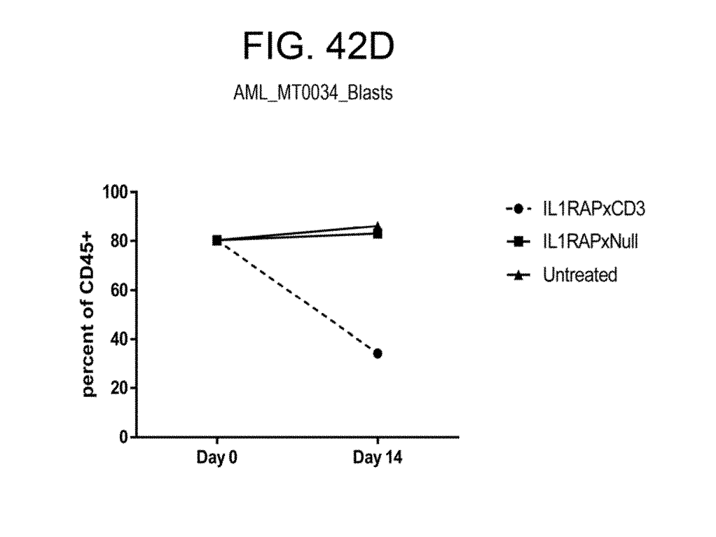

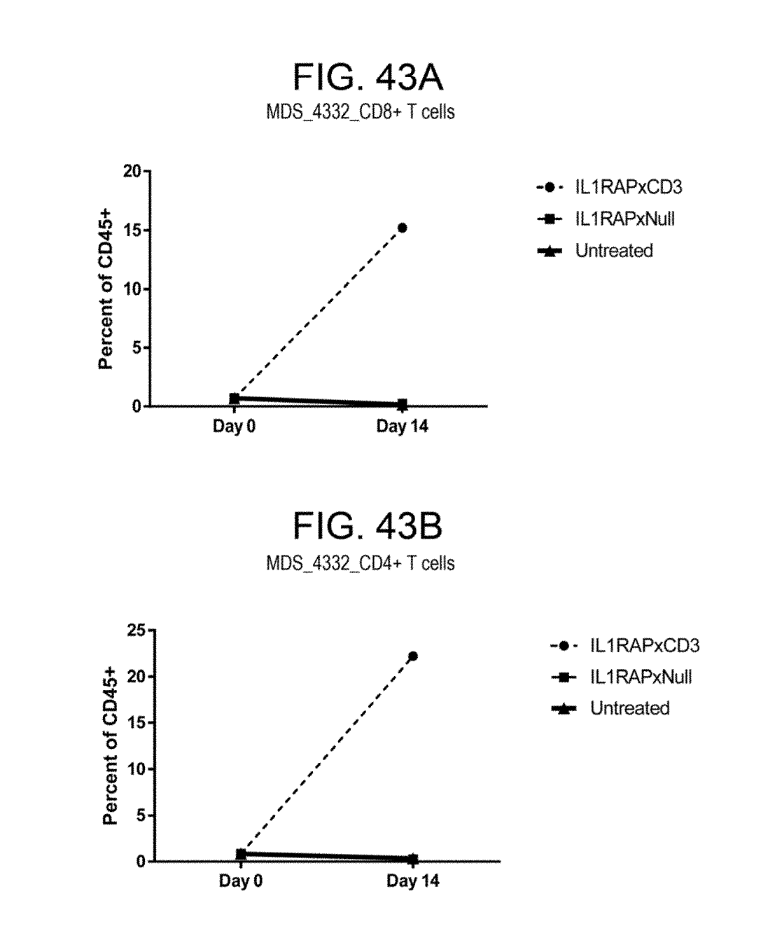

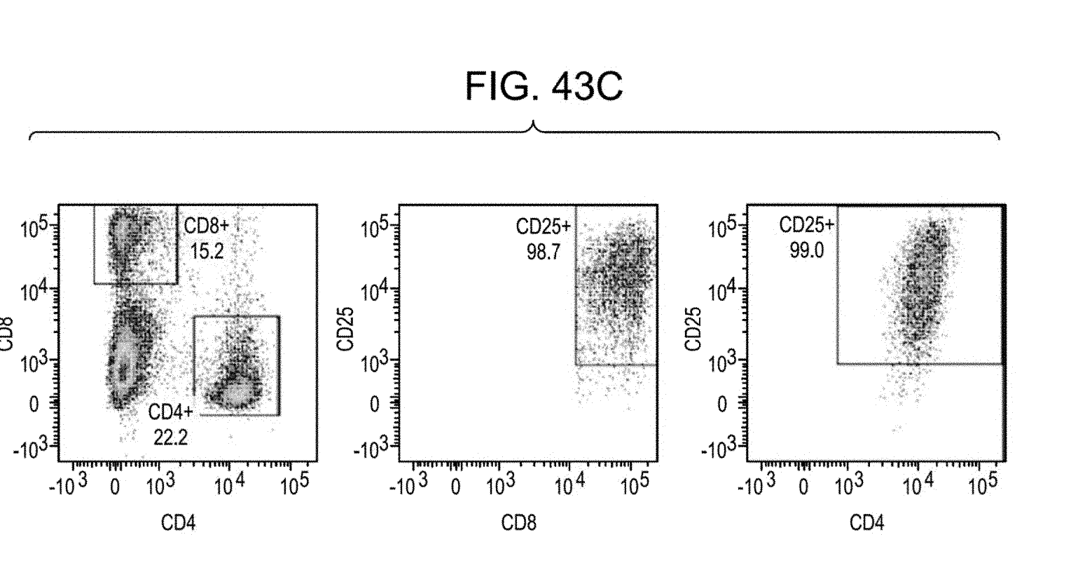

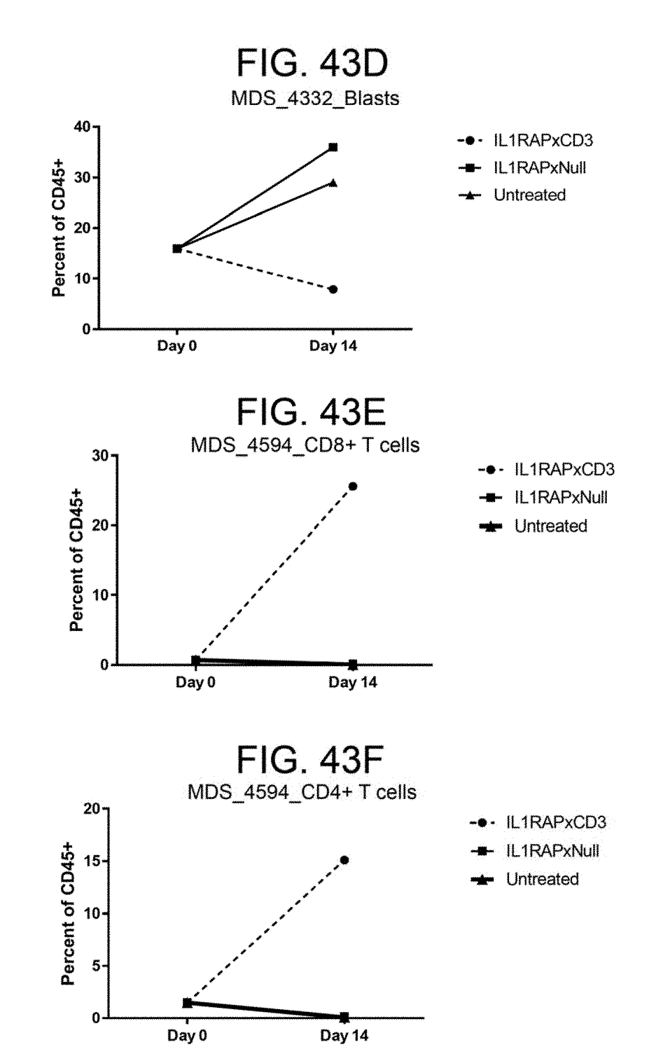

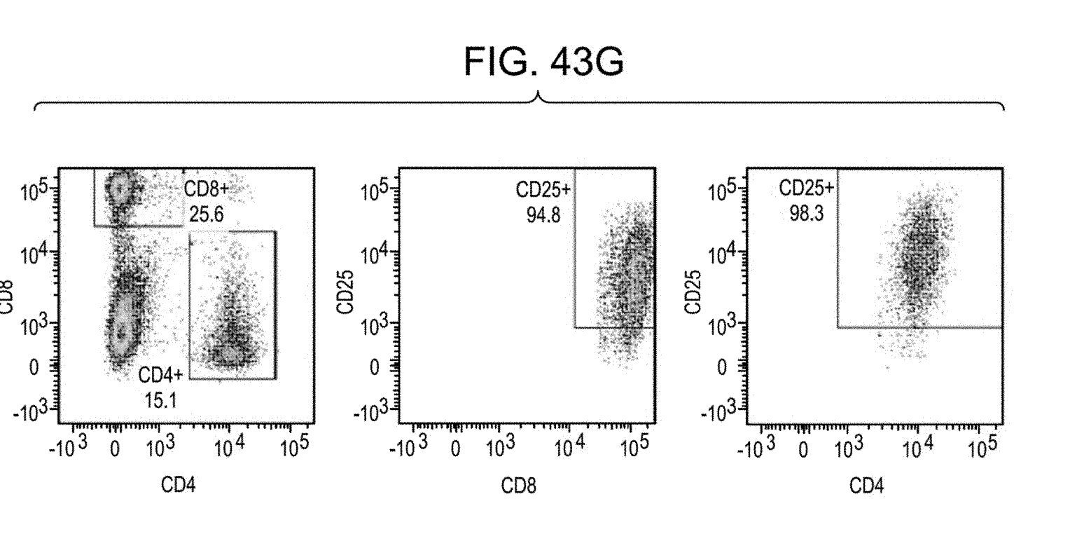

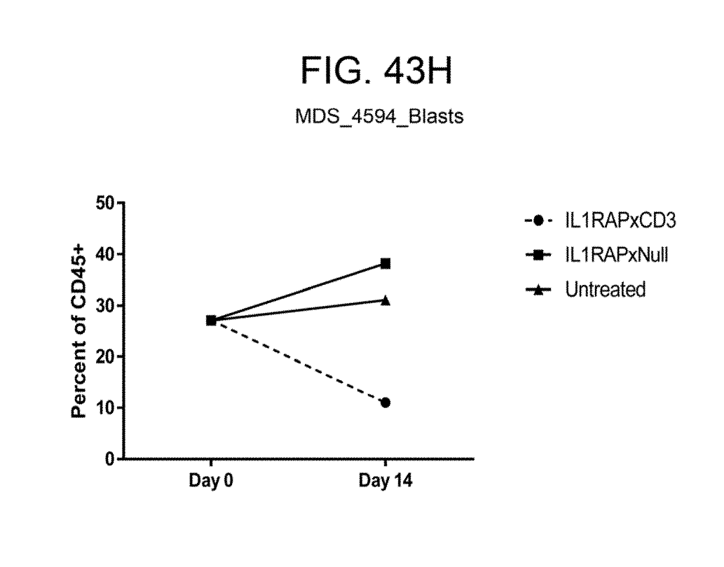

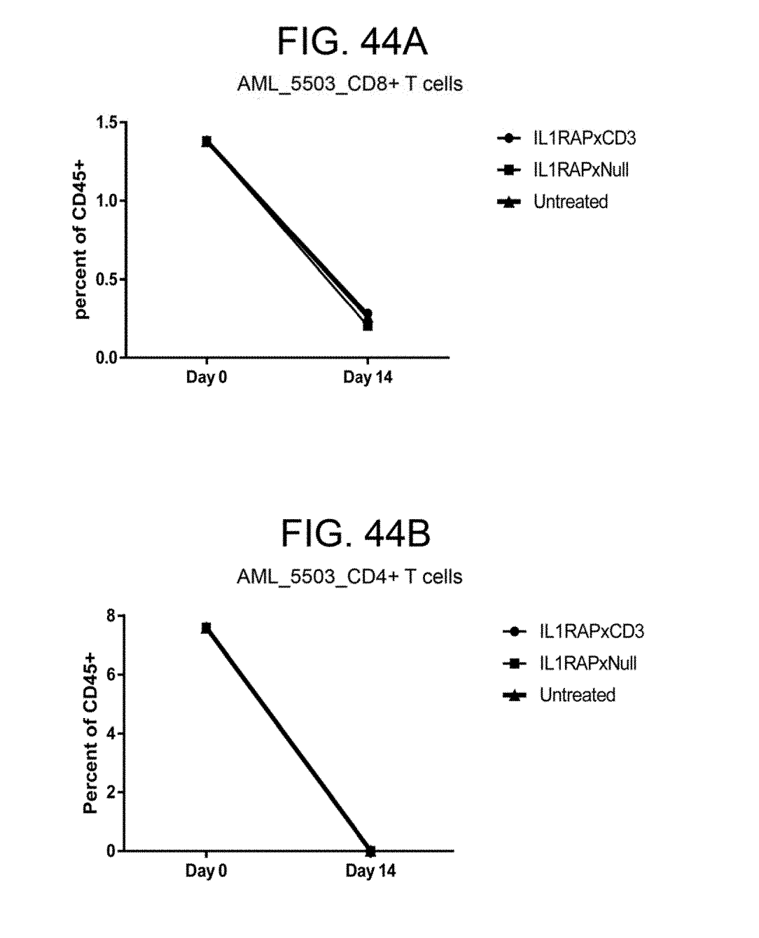

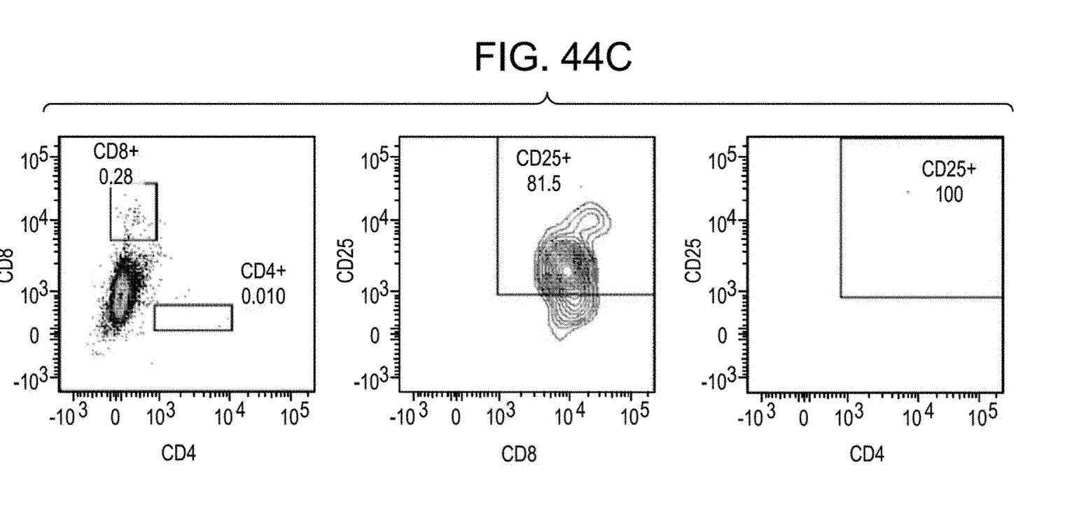

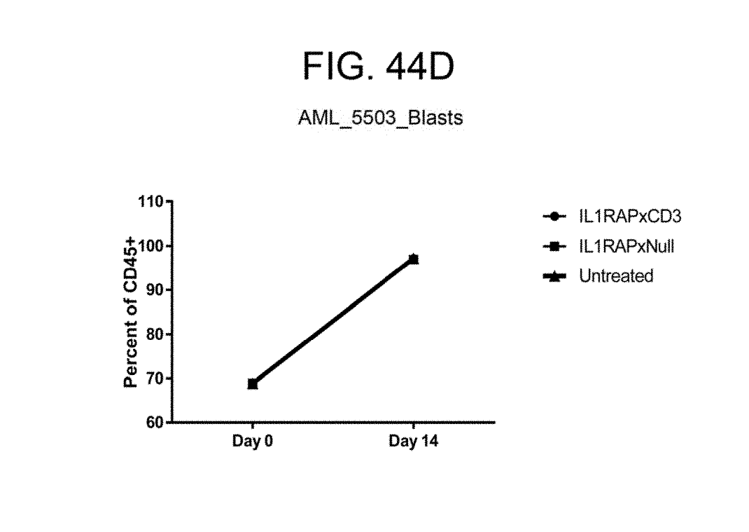

Co-culture of primary AML PBMC and MDS BMMC cells with a stroma cell line supported survival of leukemic blasts and T cells up to 28 days. In all tested samples leukemic blasts were IL1RAP positive (FIG. 41). Treatment with IL1RAP x CD3 Ab resulted in significant (40-60%) decrease in IL1RAP+ leukemic blasts in both MDS pts samples tested and one out of two AML tested samples when compared to control or Null x CD3 Ab treated cells. Decrease in IL1RAP+ cells strongly correlated with an increase in CD8+ and CD4+ T cell populations and their activation. In untreated cells or cells treated with Null x CD3 Ab, expansion of T cells was not observed (FIGS. 42A, 42B, 42C, 42D, 43A, 43B, 43C, 43D, 43E, 43F, 43G and 43H). Similar, in the non-responding AML sample, minimal CD8+ cells were present and CD4+ T cells were undetectable at Day 14 (FIGS. 44A, 44B, 44C and 44D).

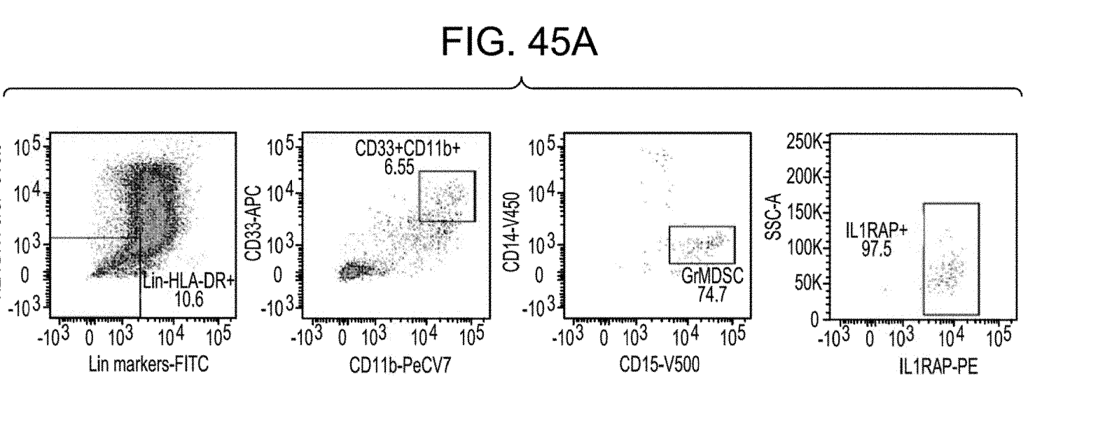

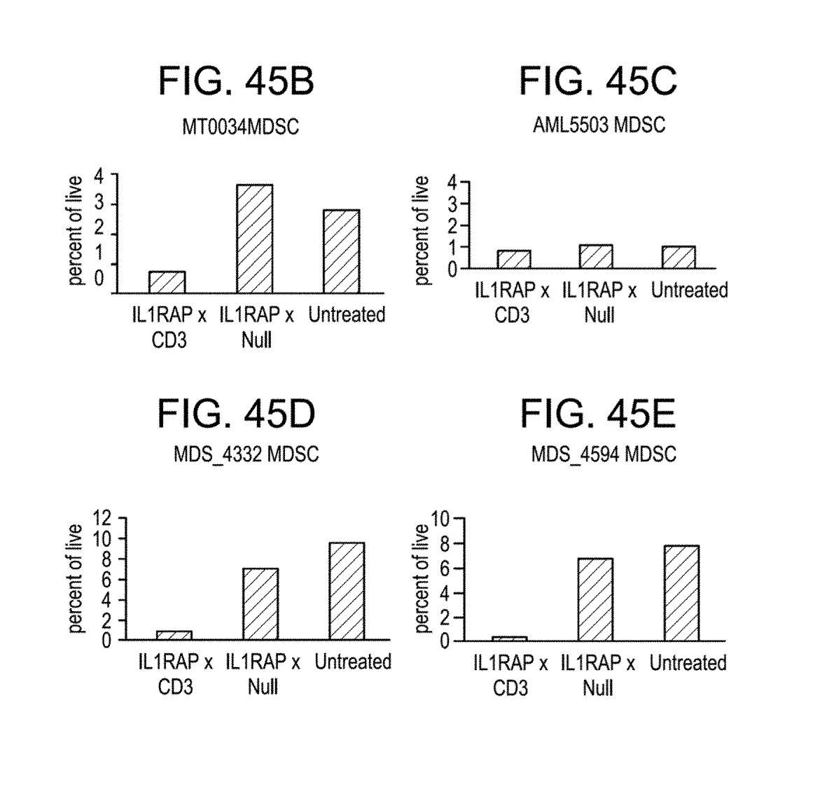

Further, in all tested samples MDSCs were generated upon activation of T cells due to the contact with stroma cells within first few days of culture. In both AML and MDS samples MDSC were IL1RAP⁺ (FIG. 45A). In responsive samples, percent of MDSCs was significantly lower after treatment with IL1RAP x CD3 in comparison to untreated control or cells treated with Null x CD3 Ab suggesting target specific killing of MDSCs. In non-responsive AML sample percent of MDSCs was the same in all three treatment groups, which correlates with lack of T cells (FIG. 45B). In responsive samples, percent of MDSCs was significantly lower after treatment with IL1RAP x CD3 in comparison to untreated control or cells treated with Null x CD3 Ab suggesting target specific killing of MDSCs. In non-responsive AML sample percent of MDSCs was the same in all three treatment groups, which correlates with lack of T cells (FIG. 45B).

### BRIEF DESCRIPTION OF THE SEQUENCE LISTING

| SEQ | | | | |
|---|---|---|---|---|
| ID | | | | |
| NO: | Type | Species | Description | Sequence |
| 1 | PRT | human | IL1RAP | SERCDDWGLDTMRQIQVFEDEPARIKC |
| | | | isoform1- | PLFEHFLKFNYSTAHSAGLTLIWYWTR |
| | | | ECD-C- | QDRDLEEPINFRLPENRISKEKDVLWFR |
| | | | terminal | PTLLNDTGNYTCMLRNTTYCSKVAFPL |
| | | | His | EVVQKDSCFNSPMKLPVHKLYIEYGIQR |
| | | | | ITCPNVDGYFPSSVKPTITWYMGCYKIQ |
| | | | | NFNNVIPEGMNLSFLIALISNNGNYTCV |
| | | | | VTYPENGRTFHLTRTLTVKVVGSPKNA |
| | | | | VPPVIHSPNDHVVYEKEPGEELLIPCTV |
| | | | | YFSFLMDSRNEVWWTIDGKKPDDITID |
| | | | | VTINESISHSRTEDETRTQILSIKKVTSED |
| | | | | LKRSYVCHARSAKGEVAKAAKVKQKV |
| | | | | PAPRYTVELACGFGATGSGSGSHHHHHH |
| 2 | PRT | human | IL1RAP | SHHHHHHGSLEVLFQGPSERCDDWGLD |
| | | | isoform2- | TMRQIQVFEDEPARIKCPLFEHFLKFNY |
| | | | ECD-N- | STAHSAGLTLIWYWTRQDRDLEEPINFR |
| | | | terminal | LPENRISKEKDVLWFRPTLLNDTGNYTC |
| | | | His | MLRNTTYCSKVAFPLEVVQKDSCFNSP |
| | | | | MKLPVHKLYIEYGIQRITCPNVDGYFPS |
| | | | | SVKPTITWYMGCYKIQNFNNVIPEGMN |
| | | | | LSFLIALISNNGNYTCVVTYPENGRTFH |
| | | | | LTRTLTVKVVGSPKNAVPPVIHSPNDHV |
| | | | | VYEKEPGEELLIPCTVYFSFLMDSRNEV |
| | | | | WWTIDGKKPDDITIDVTINESISHSRTED |
| | | | | ETRTQILSIKKVTSEDLKRSYVCHARSA |
| | | | | KGEVAKAAKVKQKGNRCGQ |
| 3 | PRT | human | IL1RAP | SERCDDWGLDTMRQIQVFEDEPARIKC |
| | | | isoform2- | PLFEHFLKFNYSTAHSAGLTLIWYWTR |
| | | | ECD-C- | QDRDLEEPINFRLPENRISKEKDVLWFR |
| | | | terminal | PTLLNDTGNYTCMLRNTTYCSKVAFPL |
| | | | His | EVVQKDSCFNSPMKLPVHKLYIEYGIQR |
| | | | | ITCPNVDGYFPSSVKPTITWYMGCYKIQ |
| | | | | NFNNVIPEGMNLSFLIALISNNGNYTCV |
| | | | | VTYPENGRTFHLTRTLTVKVVGSPKNA |
| | | | | VPPVIHSPNDHVVYEKEPGEELLIPCTV |
| | | | | YFSFLMDSRNEVWWTIDGKKPDDITID |
| | | | | VTINESISHSRTEDETRTQILSIKKVTSED |
| | | | | LKRSYVCHARSAKGEVAKAAKVKQKG |
| | | | | NRCGQGSGSGSHHHHHH |
| 4 | PRT | cyno | IL1RAP- | SERCDDWGLDTMRQIQVFEDEPARIKC |
| | | | ECD-C- | PLFEHFLKFNYSTAHSAGLTLIWYWTR |
| | | | terminal | QDRDLEEPINFRLPENRISKEKDVLWFR |
| | | | His | PTLLNDTGNYTCMLRNTTYCSKVAFPL |
| | | | | EVVQKDSCFNSPMKLPVHKLYIEYGIQR |
| | | | | ITCPNVDGYFPSSVKPTITWYMGCYKIQ |
| | | | | NFNNVIPEGMNLSFLIAFISNNGNYTCV |
| | | | | VTYPENGRTFHLTRTLTVKVVGSPKNA |
| | | | | VPPVIHSPNDHVVYEKEPGEELLIPCTV |
| | | | | YFSFLMDSRNEVWWTIDGKKPDDIPID |
| | | | | VTINESISHSRTEDETRTQILSIKKVTSED |
| | | | | LKRSYVCHARSAKGEVAKAATVKQKV |
| | | | | PAPRYTVELACGFGATGSGSGSHHHHHH |
| 5 | PRT | human | IL1RAP | SERCDDWGLDTMRQIQVFEDEPARIKC |
| | | | isoform1- | PLFEHFLKFNYSTAHSAGLTLIWYWTR |
| | | | ECD-C- | QDRDLEEPINFRLPENRISKEKDVLWFR |
| | | | terminal | PTLLNDTGNYTCMLRNTTYCSKVAFPL |
| | | | His-no | EVVQKDSCFNSPMKLPVHKLYIEYGIQR |
| | | | linker | ITCPNVDGYFPSSVKPTITWYMGCYKIQ |
| | | | | NFNNVIPEGMNLSFLIALISNNGNYTCV |
| | | | | VTYPENGRTFHLTRTLTVKVVGSPKNA |
| | | | | VPPVIHSPNDHVVYEKEPGEELLIPCTV |
| | | | | YFSFLMDSRNEVWWTIDGKKPDDITID |
| | | | | VTINESISHSRTEDETRTQILSIKKVTSED |
| | | | | LKRSYVCHARSAKGEVAKAAKVKQKV |
| | | | | PAPRYTVEAHHHHHHHHHH |
| 6 | PRT | human | IL1RAP | SERCDDWGLDTMRQIQVFEDEPARIKC |
| | | | isoform1- | PLFEHFLKFNYSTAHSAGLTLIWYWTR |
| | | | ECD | QDRDLEEPINFRLPENRISKEKDVLWFR |
| | | | | PTLLNDTGNYTCMLRNTTYCSKVAFPL |
| | | | | EVVQKDSCFNSPMKLPVHKLYIEYGIQR |
| | | | | ITCPNVDGYFPSSVKPTITWYMGCYKIQ |
| | | | | NFNNVIPEGMNLSFLIALISNNGNYTCV |
| | | | | VTYPENGRTFHLTRTLTVKVVGSPKNA |
| | | | | VPPVIHSPNDHVVYEKEPGEELLIPCTV |
| | | | | YFSFLMDSRNEVWWTIDGKKPDDITID |
| | | | | VTINESISHSRTEDETRTQILSIKKVTSED |
| | | | | LKRSYVCHARSAKGEVAKAAKVKQKV |
| | | | | PAPRYTVELACGFGAT |
| 7 | PRT | cyno | IL1RAP- | SERCDDWGLDTMRQIQVFEDEPARIKC |
| | | | ECD | PLFEHFLKFNYSTAHSAGLTLIWYWTR |
| | | | | QDRDLEEPINFRLPENRISKEKDVLWFR |
| | | | | PTLLNDTGNYTCMLRNTTYCSKVAFPL |
| | | | | EVVQKDSCFNSPMKLPVHKLYIEYGIQR |
| | | | | ITCPNVDGYFPSSVKPTITWYMGCYKIQ |
| | | | | NFNNVIPEGMNLSFLIAFISNNGNYTCV |
| | | | | VTYPENGRTFHLTRTLTVKVVGSPKNA |
| | | | | VPPVIHSPNDHVVYEKEPGEELLIPCTV |
| | | | | YFSFLMDSRNEVWWTIDGKKPDDIPID |
| | | | | VTINESISHSRTEDETRTQILSIKKVTSED |
| | | | | LKRSYVCHARSAKGEVAKAATVKQKV |
| | | | | PAPRYTVELACGFGAT |
| 8 | PRT | mouse | IL1RAP- | SERCDDWGLDTMRQIQVFEDEPARIKC |
| | | | ECD | PLFEHFLKYNYSTAHSSGLTLIWYWTR |
| | | | | QDRDLEEPINFRLPENRISKEKDVLWFR |
| | | | | PTLLNDTGNYTCMLRNTTYCSKVAFPL |
| | | | | EVVQKDSCFNSAMRFPVHKMYIEHGIH |
| | | | | KITCPNVDGYFPSSVKPSVTWYKGCTEI |
| | | | | VDFHNVLPEGMNLSFFIPLVSNNGNYTC |
| | | | | VVTYPENGRLFHLTRTVTVKVVGSPKD |
| | | | | ALPPQIYSPNDRVVYEKEPGEELVIPCK |
| | | | | VYFSFIMDSHNEVWWTIDGKKPDDVTV |
| | | | | DITINESVSYSSTEDETRTQILSIKKVTPE |
| | | | | DLRRNYVCHARNTKGEAEQAAKVKQK |
| | | | | VIPPRYTVELACGFGAT |
| 9 | PRT | rat | IL1RAP- | SERCDDWGLDTMRQIQVFEDEPARIKC |
| | | | ECD | PLFEHFLKYNYSTAHSSGLTLIWYWTR |
| | | | | QDRDLEEPINFRLPENRISKEKDVLWFR |
| | | | | PTLLNDTGNYTCMLRNTTYCSKVAFPL |
| | | | | EVVQKDSCFNSPMRLPVHRLYIEQGIHN |
| | | | | ITCPNVDGYFPSSVKPSVTWYKGCTEIV |
| | | | | NFHNVQPKGMNLSFFIPLVSNNGNYTC |
| | | | | VVTYLENGRLFHLTRTMTVKVVGSPKD |
| | | | | AVPPHIYSPNDRVVYEKEPGEELVIPCK |
| | | | | VYFSFIMDSHNEIWWTIDGKKPDDVPV |
| | | | | DITIIESVSYSSTEDETRTQILSIKKVTPE |
| | | | | DLKRNYVCHARNAEGEAEQAAKVKQK |
| | | | | VIPPRYTVELACGFGAT |
| 10 | PRT | human | IAPB47- | GYSFTSYW |
| | | | HCDR1 | |
| 11 | PRT | human | IAPB47- | IYPSDSYT |
| | | | HCDR2 | |
| 12 | PRT | human | IAPB47- | ARRNSAENYADLDY |
| | | | HCDR3 | |
| 13 | PRT | human | IAPB38, | GFTFSNYA |
| | | | and | |
| | | | IAPB29- | |
| | | | HCDR1 | |
| 14 | PRT | human | IAPB38- | INYGGGSK |
| | | | HCDR2 | |
| 15 | PRT | human | IAPB38- | AKDYGPFALDY |
| | | | HCDR3 | |
| 16 | PRT | human | IAPB57- | GGSISSSTYY |
| | | | HCDR1 | |
| 17 | PRT | human | IAPB57- | IYFTGST |
| | | | HCDR2 | |
| 18 | PRT | human | IAPB57- | AKEDDSSGYYSFDY |
| | | | HCDR3 | |
| 19 | PRT | human | IAPB61 | GVSISSSTYY |
| | | | and | |
| | | | IAPB55- | |
| | | | HCDR1 | |
| 20 | PRT | human | IAPB61 | IYFTGNT |
| | | | and | |
| | | | IAPB55- | |
| | | | HCDR2 | |
| 21 | PRT | human | IAPB61 | GSLFGDYGYFDY |
| | | | and | |
| | | | IAPB55- | |
| | | | HCDR3 | |
| 22 | PRT | human | IAPB62, | GYTFNTYA |
| | | | IAPB63 | |
| | | | and | |
| | | | IAPB64- | |
| | | | HCDR1 | |
| 23 | PRT | human | IAPB62, | INTNTGNP |
| | | | IAPB63 | |
| | | | and | |
| | | | IAPB64- | |
| | | | HCDR2 | |
| 24 | PRT | human | IAPB62, | ARRYFDWLLGAFDI |
| | | | IAPB63 | |
| | | | and | |
| | | | IAPB64- | |
| | | | HCDR3 | |
| 25 | PRT | human | IAPB3, | GGTFSSYA |
| | | | IAPB17, | |
| | | | IAPB9 and | |
| | | | IAPB65- | |
| | | | HCDR1 | |
| 26 | PRT | human | IAPB3 and | ISAIFGTA |
| | | | IAPB65- | |
| | | | HCDR2 | |
| 27 | PRT | human | IAPB3- | ARGNSFHALWDYAFDY |
| | | | HCDR3 | |
| 28 | PRT | human | IAPB17- | IIPIFGNA |
| | | | HCDR2 | |
| 29 | PRT | human | IAPB17- | ARTIIYLDYVHILDY |
| | | | HCDR3 | |
| 30 | PRT | human | IAPB23- | GFTFSNYW |
| | | | HCDR1 | |
| 31 | PRT | human | IAPB23- | IRYDGGSK |
| | | | HCDR2 | |
| 32 | PRT | human | IAPB23- | AKDAYPPYSFDY |
| | | | HCDR3 | |
| 33 | PRT | human | IAPB25- | GFTFSSYA |
| | | | HCDR1 | |
| 34 | PRT | human | IAPB25 | ISGSGGST |
| | | | and | |
| | | | IAPB29- | |
| | | | HCDR2 | |
| 35 | PRT | human | IAPB25- | AKGDEYYYPDPLDY |
| | | | HCDR3 | |
| 36 | PRT | human | IAPB29- | AKEWSSYFGLDY |
| | | | HCDR3 | |
| 37 | PRT | human | IAPB9- | ISPIFGTA |
| | | | HCDR2 | |
| 38 | PRT | human | IAPB9- | ARRYDNFARSGDLDY |
| | | | HCDR3 | |
| 39 | PRT | human | IAPB65- | ARHLHNAIHLDY |
| | | | HCDR3 | |
| 40 | PRT | human | IAPB47- | QSISND |
| | | | LCDR1 | |
| 41 | PRT | human | IAPB47- | YAS |
| | | | LCDR2 | |
| 42 | PRT | human | IAPB47- | QQSFTAPLT |
| | | | LCDR3 | |
| 43 | PRT | human | IAPB38- | QSVDDW |
| | | | LCDR1 | |
| 44 | PRT | human | IAPB38- | TAS |
| | | | LCDR2 | |
| 45 | PRT | human | IAPB38- | QQYHHWPLT |
| | | | LCDR3 | |
| 46 | PRT | human | IAPB57- | QGISSY |
| | | | LCDR1 | |
| 47 | PRT | human | IAPB57, | AAS |
| | | | IAPB62, | |
| | | | IAPB25, | |
| | | | IAPB29, | |
| | | | and | |
| | | | IAPB9- | |
| | | | LCDR2 | |
| 48 | PRT | human | IAPB25, | QQSYSTPLT |
| | | | IAPB29, | |
| | | | and | |
| | | | IAPB9- | |
| | | | LCDR3 | |
| 49 | PRT | human | IAPB61 | QFISSN |
| | | | and | |
| | | | IAPB55- | |
| | | | LCDR1 | |
| 50 | PRT | human | IAPB61, | GAS |
| | | | IAPB55 | |
| | | | and | |
| | | | IAPB65- | |
| | | | LCDR2 | |
| 51 | PRT | human | IAPB61- | QQYNNWPST |
| | | | LCDR3 | |
| 52 | PRT | human | IAPB62- | QGISSW |
| | | | LCDR1 | |
| 53 | PRT | human | IAPB62- | QQANSFPLT |
| | | | LCDR3 | |
| 54 | PRT | human | IAPB3 and | QSVLYSSNNKNY |
| | | | IAPB17- | |
| | | | LCDR1 | |
| 55 | PRT | human | IAPB3 and | WAS |
| | | | IAPB17- | |
| | | | LCDR2 | |
| 56 | PRT | human | IAPB3 and | QQYYSTPLT |
| | | | IAPB17- | |
| | | | LCDR3 | |
| 57 | PRT | human | IAPB23- | QSVSSY |
| | | | LCDR1 | |
| 58 | PRT | human | IAPB23- | DAS |
| | | | LCDR2 | |
| 59 | PRT | human | IAPB23- | QQRSNWPLT |
| | | | LCDR3 | |
| 60 | PRT | human | IAPB25, | QSISSY |
| | | | IAPB29 | |
| | | | and | |
| | | | IAPB9- | |
| | | | LCDR1 | |
| 61 | PRT | human | IAPB55- | QQYNNWPFT |
| | | | LCDR3 | |
| 62 | PRT | human | IAPB63 | SSDVGDYNY |
| | | | and | |
| | | | IAPB64- | |
| | | | LCDR1 | |
| 63 | PRT | human | IAPB63 | DVS |
| | | | and | |
| | | | IAPB64- | |
| | | | LCDR2 | |
| 64 | PRT | human | IAPB63- | ASYAGNYNVV |
| | | | LCDR3 | |
| 65 | PRT | human | IAPB64- | SSYAGNYNVV |
| | | | LCDR3 | |
| 66 | PRT | human | IAPB65- | QSVSNF |
| | | | LCDR1 | |
| 67 | PRT | human | IAPB65- | QQGKHWPWT |
| | | | LCDR3 | |
| 68 | PRT | human | IAPB47- | EVQLVQSGAEVKKPGESLKISCKGSGYS |
| | | | VH | FTSYWIGWVRQMPGKGLEWMGIIYPSD |
| | | | | SYTRYSPSFQGQVTISADKSISTAYLQW |
| | | | | SSLKASDTAMYYCARRNSAENYADLD |
| | | | | YWGQGTLVTVSSASTKGPSVFPLAPCS |
| | | | | RSTSESTAALGCLVKDYFPEPVTVSWNS |
| | | | | GALTSGVHTFPAVLQSSGLYSLSSVVTV |
| | | | | PSSSLGTKTYTCNVDHKPSNTKVDKRV |
| | | | | ESKYGPPCPPCPAPEAAGGPSVFLFPPKP |
| | | | | KDTLMISRTPEVTCVVVDVSQEDPEVQ |
| | | | | FNWYVDGVEVHNAKTKPREEQFNSTY |
| | | | | RVVSVLTVLHQDWLNGKEYKCKVSNK |
| | | | | GLPSSIEKTISKAKGQPREPQVYTLPPSQ |
| | | | | EEMTKNQVSLTCLVKGFYPSDIAVEWE |
| | | | | SNGQPENNYKTTPPVLDSDGSFFLYSRL |
| | | | | TVDKSRWQEGNVFSCSVMHEALHNHY |
| | | | | TQKSLSLSLGK |
| 69 | PRT | human | IAPB47- | EIVLTQSPGTLSLSPGERATLSCRASQSI |
| | | | VL | SNDLNWYQQKPGKAPKLLIYYASSLQS |
| | | | | GVPSRFSGSGSGTDFTLTINSLQPEDFAT |
| | | | | YYCQQSFTAPLTFGQGTKVEIKRTVAAP |
| | | | | SVFIFPPSDEQLKSGTASVVCLLNNFYP |
| | | | | REAKVQWKVDNALQSGNSQESVTEQD |
| | | | | SKDSTYSLSSTLTLSKADYEKHKVYAC |
| | | | | EVTHQGLSSPVTKSFNRGEC |
| 70 | PRT | human | IAPB38- | EVQLLESGGGLVQPGGSLRLSCAASGFT |
| | | | VH | FSNYAMNWVRQAPGKGLEWVSGINYG |
| | | | | GGSKYYADSVKGRFTISRDNSKNTLYL |
| | | | | QMNSLRAEDTAVYYCAKDYGPFALDY |
| | | | | WGQGTLVTVSSASTKGPSVFPLAPCSRS |
| | | | | TSESTAALGCLVKDYFPEPVTVSWNSG |
| | | | | ALTSGVHTFPAVLQSSGLYSLSSVVTVP |
| | | | | SSSLGTKTYTCNVDHKPSNTKVDKRVE |
| | | | | SKYGPPCPPCPAPEAAGGPSVFLFPPKP |
| | | | | KDTLMISRTPEVTCVVVDVSQEDPEVQ |
| | | | | FNWYVDGVEVHNAKTKPREEQFNSTY |
| | | | | RVVSVLTVLHQDWLNGKEYKCKVSNK |
| | | | | GLPSSIEKTISKAKGQPREPQVYTLPPSQ |
| | | | | EEMTKNQVSLTCLVKGFYPSDIAVEWE |
| | | | | SNGQPENNYKTTPPVLDSDGSFFLYSRL |
| | | | | TVDKSRWQEGNVFSCSVMHEALHNHY |
| | | | | TQKSLSLSLGK |
| 71 | PRT | human | IAPB38- | EIVLTQSPATLSLSPGERATLSCRASQSV |
| | | | VL | DDWLAWYQQKPGQAPRLLIYTASNRA |
| | | | | TGIPARFSGSGSGTDFTLTISSLEPEDFA |
| | | | | VYYCQQYHHWPLTFGQGTKVEIKRTV |
| | | | | AAPSVFIFPPSDEQLKSGTASVVCLLNN |
| | | | | FYPREAKVQWKVDNALQSGNSQESVT |
| | | | | EQDSKDSTYSLSSTLTLSKADYEKHKV |
| | | | | YACEVTHQGLSSPVTKSFNRGEC |
| 72 | PRT | human | IAPB57 | QLQLQESGPGLVKPSETLSLTCTVSGGSI |
| | | | VH | SSSTYYWGWIRQPPGKGLEWIGSIYFTG |
| | | | | STDYNPSLKSRVSISVDTSKNQFSLKLSS |
| | | | | VTAADTAVYYCAKEDDSSGYYSFDYW |
| | | | | GQGNLVTVSSASTKGPSVFPLAPCSRST |
| | | | | SESTAALGCLVKDYFPEPVTVSWNSGA |
| | | | | LTSGVHTFPAVLQSSGLYSLSSVVTVPS |
| | | | | SSLGTKTYTCNVDHKPSNTKVDKRVES |
| | | | | KYGPPCPPCPAPEAAGGPSVFLFPPKPK |
| | | | | DTLMISRTPEVTCVVVDVSQEDPEVQF |
| | | | | NWYVDGVEVHNAKTKPREEQFNSTYR |
| | | | | VVSVLTVLHQDWLNGKEYKCKVSNKG |
| | | | | LPSSIEKTISKAKGQPREPQVYTLPPSQE |
| | | | | EMTKNQVSLTCLVKGFYPSDIAVEWES |
| | | | | NGQPENNYKTTPPVLDSDGSFFLYSRLT |
| | | | | VDKSRWQEGNVFSCSVMHEALHNHYT |
| | | | | QKSLSLSLGK |
| 73 | PRT | human | IAPB57- | DIQLTQSPSFLSASVGDRVTITCRASQGI |
| | | | VL | SSYLAWYQQKPGKAPKLLIYAASTLQS |
| | | | | GVPSRFSGSGSGTEFTLTISSLQPEDFAT |
| | | | | YYCQQVNSYPLTFGGGTKVEIKRTVAA |
| | | | | PSVFIFPPSDEQLKSGTASVVCLLNNFYP |
| | | | | REAKVQWKVDNALQSGNSQESVTEQD |
| | | | | SKDSTYSLSSTLTLSKADYEKHKVYAC |
| | | | | EVTHQGLSSPVTKSFNRGEC |
| 74 | PRT | human | IAPB61 | QLQLQESGPGLVKPSETLSLTCTVSGVSI |
| | | | and | SSSTYYWGWLRQPPGMGLEWTGSIYFT |
| | | | IAPB55- | GNTYYNPSLKSRVTISVDTSRNQFSLKL |
| | | | VH | SSVTAADTAVYYCGSLFGDYGYFDYW |
| | | | | GQGTLVTVSSASTKGPSVFPLAPCSRST |
| | | | | SESTAALGCLVKDYFPEPVTVSWNSGA |
| | | | | LTSGVHTFPAVLQSSGLYSLSSVVTVPS |
| | | | | SSLGTKTYTCNVDHKPSNTKVDKRVES |
| | | | | KYGPPCPPCPAPEAAGGPSVFLFPPKPK |
| | | | | DTLMISRTPEVTCVVVDVSQEDPEVQF |
| | | | | NWYVDGVEVHNAKTKPREEQFNSTYR |
| | | | | VVSVLTVLHQDWLNGKEYKCKVSNKG |
| | | | | LPSSIEKTISKAKGQPREPQVYTLPPSQE |
| | | | | EMTKNQVSLTCLVKGFYPSDIAVEWES |
| | | | | NGQPENNYKTTPPVLDSDGSFFLYSRLT |
| | | | | VDKSRWQEGNVFSCSVMHEALHNHYT |
| | | | | QKSLSLSLGK |
| 75 | PRT | human | IAPB61- | EIVMTQSPATLSVPPGERATLSCRASQFI |
| | | | VL | SSNLAWYQQKPGQAPRLLIYGASTRAT |
| | | | | GIPARFSGSGSGTDFTLTISSLQSEDFAV |
| | | | | YYCQQYNNWPSTFGPGTKVDIKRTVAA |
| | | | | PSVFIFPPSDEQLKSGTASVVCLLNNFYP |
| | | | | REAKVQWKVDNALQSGNSQESVTEQD |
| | | | | SKDSTYSLSSTLTLSKADYEKHKVYAC |
| | | | | EVTHQGLSSPVTKSFNRGEC |
| 76 | PRT | human | IAPB62, | QVQLVQSGSELKKPGASVKVSCKASGY |
| | | | IAPB63 | TFNTYAMNWVRQAPGQGLEWMGWIN |
| | | | and | TNTGNPTYAQGFTGRFVFSLDTSVSTAY |
| | | | IAPB64- | LQISSLKAEDTAVYYCARRYFDWLLGA |
| | | | VH | FDIWGQGTMVTVSSASTKGPSVFPLAP |
| | | | | CSRSTSESTAALGCLVKDYFPEPVTVSW |
| | | | | NSGALTSGVHTFPAVLQSSGLYSLSSVV |
| | | | | TVPSSSLGTKTYTCNVDHKPSNTKVDK |
| | | | | RVESKYGPPCPPCPAPEAAGGPSVFLFP |
| | | | | PKPKDTLMISRTPEVTCVVVDVSQEDPE |
| | | | | VQFNWYVDGVEVHNAKTKPREEQFNS |
| | | | | TYRVVSVLTVLHQDWLNGKEYKCKVS |
| | | | | NKGLPSSIEKTISKAKGQPREPQVYTLPP |
| | | | | SQEEMTKNQVSLTCLVKGFYPSDIAVE |
| | | | | WESNGQPENNYKTTPPVLDSDGSFFLY |
| | | | | SRLTVDKSRWQEGNVFSCSVMHEALH |
| | | | | NHYTQKSLSLSLGK |
| 77 | PRT | human | IAPB62- | DIQMTQSPSSVSASVGDRVTITCRASQG |
| | | | VL | ISSWLAWYQQKPGKAPKLLIYAASSLQ |
| | | | | SGVPSRFSGSGSGTDFTLTISSLQPEDFA |
| | | | | TYYCQQANSFPLTFGGGTKVEIKRTVA |
| | | | | APSVFIFPPSDEQLKSGTASVVCLLNNF |
| | | | | YPREAKVQWKVDNALQSGNSQESVTE |
| | | | | QDSKDSTYSLSSTLTLSKADYEKHKVY |
| | | | | ACEVTHQGLSSPVTKSFNRGEC |
| 78 | PRT | human | IAPB3-VH | QVQLVQSGAEVKKPGSSVKVSCKASGG |
| | | | | TFSSYAISWVRQAPGQGLEWMGGISAIF |
| | | | | GTANYAQKFQGRVTITADESTSTAYME |
| | | | | LSSLRSEDTAVYYCARGNSFHALWDYA |
| | | | | FDYWGQGTLVTVSSASTKGPSVFPLAP |
| | | | | CSRSTSESTAALGCLVKDYFPEPVTVSW |
| | | | | NSGALTSGVHTFPAVLQSSGLYSLSSVV |
| | | | | TVPSSSLGTKTYTCNVDHKPSNTKVDK |
| | | | | RVESKYGPPCPPCPAPEAAGGPSVFLFP |
| | | | | PKPKDTLMISRTPEVTCVVVDVSQEDPE |
| | | | | VQFNWYVDGVEVHNAKTKPREEQFNS |
| | | | | TYRVVSVLTVLHQDWLNGKEYKCKVS |
| | | | | NKGLPSSIEKTISKAKGQPREPQVYTLPP |
| | | | | SQEEMTKNQVSLTCLVKGFYPSDIAVE |
| | | | | WESNGQPENNYKTTPPVLDSDGSFFLY |
| | | | | SRLTVDKSRWQEGNVFSCSVMHEALH |
| | | | | NHYTQKSLSLSLGK |
| 79 | PRT | human | IAPB3 and | DIVMTQSPDSLAVSLGERATINCKSSQS |
| | | | IAPB17- | VLYSSNNKNYLAWYQQKPGQPPKLLIY |
| | | | VL | WASTRESGVPDRFSGSGSGTDFTLTISS |
| | | | | LQAEDVAVYYCQQYYSTPLTFGQGTK |
| | | | | VEIKRTVAAPSVFIFPPSDEQLKSGTASV |
| | | | | VCLLNNFYPREAKVQWKVDNALQSGN |
| | | | | SQESVTEQDSKDSTYSLSSTLTLSKADY |
| | | | | EKHKVYACEVTHQGLSSPVTKSFNRGEC |
| 80 | PRT | human | IAPB17- | QVQLVQSGAEVKKPGSSVKVSCKASGG |
| | | | VH | TFSSYAISWVRQAPGQGLEWMGGIIPIF |
| | | | | GNANYAQKFQGRVTITADESTSTAYME |
| | | | | LSSLRSEDTAVYYCARTIIYLDYVHILD |
| | | | | YWGQGTLVTVSSASTKGPSVFPLAPCS |
| | | | | RSTSESTAALGCLVKDYFPEPVTVSWNS |
| | | | | GALTSGVHTFPAVLQSSGLYSLSSVVTV |
| | | | | PSSSLGTKTYTCNVDHKPSNTKVDKRV |
| | | | | ESKYGPPCPPCPAPEAAGGPSVFLFPPKP |
| | | | | KDTLMISRTPEVTCVVVDVSQEDPEVQ |
| | | | | FNWYVDGVEVHNAKTKPREEQFNSTY |
| | | | | RVVSVLTVLHQDWLNGKEYKCKVSNK |
| | | | | GLPSSIEKTISKAKGQPREPQVYTLPPSQ |
| | | | | EEMTKNQVSLTCLVKGFYPSDIAVEWE |
| | | | | SNGQPENNYKTTPPVLDSDGSFFLYSRL |
| | | | | TVDKSRWQEGNVFSCSVMHEALHNHY |
| | | | | TQKSLSLSLGK |
| 81 | PRT | human | IAPB23- | EVQLLESGGGLVQPGGSLRLSCAASGFT |
| | | | VH | FSNYWMNWVRQAPGKGLEWVSAIRYD |
| | | | | GGSKYYADSVKGRFTISRDNSKNTLYL |
| | | | | QMNSLRAEDTAVYYCAKDAYPPYSFD |
| | | | | YWGQGTLVTVSSASTKGPSVFPLAPCS |
| | | | | RSTSESTAALGCLVKDYFPEPVTVSWNS |
| | | | | GALTSGVHTFPAVLQSSGLYSLSSVVTV |
| | | | | PSSSLGTKTYTCNVDHKPSNTKVDKRV |
| | | | | ESKYGPPCPPCPAPEAAGGPSVFLFPPKP |
| | | | | KDTLMISRTPEVTCVVVDVSQEDPEVQ |
| | | | | FNWYVDGVEVHNAKTKPREEQFNSTY |
| | | | | RVVSVLTVLHQDWLNGKEYKCKVSNK |
| | | | | GLPSSIEKTISKAKGQPREPQVYTLPPSQ |
| | | | | EEMTKNQVSLTCLVKGFYPSDIAVEWE |
| | | | | SNGQPENNYKTTPPVLDSDGSFFLYSRL |
| | | | | TVDKSRWQEGNVFSCSVMHEALHNHY |
| | | | | TQKSLSLSLGK |
| 82 | PRT | human | IAPB23- | EIVLTQSPATLSLSPGERATLSCRASQSV |
| | | | VL | SSYLAWYQQKPGQAPRLLIYDASNRAT |
| | | | | GIPARFSGSGSGTDFTLTISSLEPEDFAV |
| | | | | YYCQQRSNWPLTFGQGTKVEIKRTVAA |
| | | | | PSVFIFPPSDEQLKSGTASVVCLLNNFYP |
| | | | | REAKVQWKVDNALQSGNSQESVTEQD |
| | | | | SKDSTYSLSSTLTLSKADYEKHKVYAC |
| | | | | EVTHQGLSSPVTKSFNRGEC |
| 83 | PRT | human | IAPB25- | EVQLLESGGGLVQPGGSLRLSCAASGFT |
| | | | VH | FSSYAMSWVRQAPGKGLEWVSAISGSG |
| | | | | GSTYYADSVKGRFTISRDNSKNTLYLQ |
| | | | | MNSLRAEDTAVYYCAKGDEYYYPDPL |
| | | | | DYWGQGTLVTVSSASTKGPSVFPLAPC |
| | | | | SRSTSESTAALGCLVKDYFPEPVTVSWN |
| | | | | SGALTSGVHTFPAVLQSSGLYSLSSVVT |
| | | | | VPSSSLGTKTYTCNVDHKPSNTKVDKR |
| | | | | VESKYGPPCPPCPAPEAAGGPSVFLFPP |
| | | | | KPKDTLMISRTPEVTCVVVDVSQEDPE |
| | | | | VQFNWYVDGVEVHNAKTKPREEQFNS |
| | | | | TYRVVSVLTVLHQDWLNGKEYKCKVS |
| | | | | NKGLPSSIEKTISKAKGQPREPQVYTLPP |
| | | | | SQEEMTKNQVSLTCLVKGFYPSDIAVE |
| | | | | WESNGQPENNYKTTPPVLDSDGSFFLY |
| | | | | SRLTVDKSRWQEGNVFSCSVMHEALH |
| | | | | NHYTQKSLSLSLGK |
| 84 | PRT | human | IAPB25, | DIQMTQSPSSLSASVGDRVTITCRASQSI |
| | | | IAPB29 | SSYLNWYQQKPGKAPKLLIYAASSLQS |
| | | | and | GVPSRFSGSGSGTDFTLTISSLQPEDFAT |
| | | | IAPB9-VL | YYCQQSYSTPLTFGQGTKVEIKRTVAAP |
| | | | | SVFIFPPSDEQLKSGTASVVCLLNNFYP |
| | | | | REAKVQWKVDNALQSGNSQESVTEQD |
| | | | | SKDSTYSLSSTLTLSKADYEKHKVYAC |
| | | | | EVTHQGLSSPVTKSFNRGEC |
| 85 | PRT | human | IAPB29- | EVQLLESGGGLVQPGGSLRLSCAASGFT |
| | | | VH | FSNYAMSWVRQAPGKGLEWVSAISGS |
| | | | | GGSTYYADSVKGRFTISRDNSKNTLYL |
| | | | | QMNSLRAEDTAVYYCAKEWSSYFGLD |
| | | | | YWGQGTLVTVSSASTKGPSVFPLAPCS |
| | | | | RSTSESTAALGCLVKDYFPEPVTVSWNS |
| | | | | GALTSGVHTFPAVLQSSGLYSLSSVVTV |
| | | | | PSSSLGTKTYTCNVDHKPSNTKVDKRV |
| | | | | ESKYGPPCPPCPAPEAAGGPSVFLFPPKP |
| | | | | KDTLMISRTPEVTCVVVDVSQEDPEVQ |
| | | | | FNWYVDGVEVHNAKTKPREEQFNSTY |
| | | | | RVVSVLTVLHQDWLNGKEYKCKVSNK |
| | | | | GLPSSIEKTISKAKGQPREPQVYTLPPSQ |
| | | | | EEMTKNQVSLTCLVKGFYPSDIAVEWE |
| | | | | SNGQPENNYKTTPPVLDSDGSFFLYSRL |
| | | | | TVDKSRWQEGNVFSCSVMHEALHNHY |
| | | | | TQKSLSLSLGK |
| 86 | PRT | human | IAPB9-VH | QVQLVQSGAEVKKPGSSVKVSCKASGG |
| | | | | TFSSYAISWVRQAPGQGLEWMGWISPIF |
| | | | | GTANYAQKFQGRVTITADESTSTAYME |
| | | | | LSSLRSEDTAVYYCARRYDNFARSGDL |
| | | | | DYWGQGTLVTVSSASTKGPSVFPLAPC |
| | | | | SRSTSESTAALGCLVKDYFPEPVTVSWN |
| | | | | SGALTSGVHTFPAVLQSSGLYSLSSVVT |
| | | | | VPSSSLGTKTYTCNVDHKPSNTKVDKR |
| | | | | VESKYGPPCPPCPAPEAAGGPSVFLFPP |
| | | | | KPKDTLMISRTPEVTCVVVDVSQEDPE |
| | | | | VQFNWYVDGVEVHNAKTKPREEQFNS |
| | | | | TYRVVSVLTVLHQDWLNGKEYKCKVS |
| | | | | NKGLPSSIEKTISKAKGQPREPQVYTLPP |
| | | | | SQEEMTKNQVSLTCLVKGFYPSDIAVE |
| | | | | WESNGQPENNYKTTPPVLDSDGSFFLY |
| | | | | SRLTVDKSRWQEGNVFSCSVMHEALH |
| | | | | NHYTQKSLSLSLGK |
| 87 | PRT | human | IAPB55- | EIVMTQSPATLSVSPGERATLSCRASQFI |
| | | | VL | SSNLAWYQQKPGQAPRLLIYGASTRAT |
| | | | | GIPARFSGSGSGTDFTLTISSLQSEDFAV |
| | | | | YYCQQYNNWPFTFGPGTKVDIKRTVAA |
| | | | | PSVFIFPPSDEQLKSGTASVVCLLNNFYP |
| | | | | REAKVQWKVDNALQSGNSQESVTEQD |
| | | | | SKDSTYSLSSTLTLSKADYEKHKVYAC |
| | | | | EVTHQGLSSPVTKSFNRGEC |
| 88 | PRT | human | IAPB63- | QSALTQPRSVSGSPGHSVTISCTGTSSD |
| | | | VL | VGDYNYVSWYQQRPGKVPKLLIYDVS |
| | | | | KRPSGVPDRFSGSKSGNTASLTISGLQA |
| | | | | EDEAIYFCASYAGNYNVVFGGGTKLTV |
| | | | | LGQPKAAPSVTLFPPSSEELQANKATLV |
| | | | | CLISDFYPGAVTVAWKADSSPVKAGVE |
| | | | | TTTPSKQSNNKYAASSYLSLTPEQWKS |
| | | | | HRSYSCQVTHEGSTVEKTVAPTECS |
| 89 | PRT | human | IAPB64- | QSALTQPRSVSGSPGHSVTISCTGTSSD |
| | | | VL | VGDYNYVSWYQQRPGKVPKLLIYDVS |
| | | | | KRPSGVPDRFSGSKSGNTASLTISGLQA |
| | | | | EDEAIYFCSSYAGNYNVVFGGGTKLTV |
| | | | | LGQPKAAPSVTLFPPSSEELQANKATLV |
| | | | | CLISDFYPGAVTVAWKADSSPVKAGVE |
| | | | | TTTPSKQSNNKYAASSYLSLTPEQWKS |
| | | | | HRSYSCQVTHEGSTVEKTVAPTECS |
| 90 | PRT | human | IAPB65- | QVQLVQSGAEVKKPGSSVKVSCKASGG |
| | | | VH | TFSSYAISWVRQAPGQGLEWMGGISAIF |
| | | | | GTANYAQKFQGRVTITADESTSTAYME |
| | | | | LSSLRSEDTAVYYCARHLHNAIHLDYW |
| | | | | GQGTLVTVSSASTKGPSVFPLAPCSRST |
| | | | | SESTAALGCLVKDYFPEPVTVSWNSGA |
| | | | | LTSGVHTFPAVLQSSGLYSLSSVVTVPS |
| | | | | SSLGTKTYTCNVDHKPSNTKVDKRVES |
| | | | | KYGPPCPPCPAPEAAGGPSVFLFPPKPK |
| | | | | DTLMISRTPEVTCVVVDVSQEDPEVQF |
| | | | | NWYVDGVEVHNAKTKPREEQFNSTYR |
| | | | | VVSVLTVLHQDWLNGKEYKCKVSNKG |
| | | | | LPSSIEKTISKAKGQPREPQVYTLPPSQE |
| | | | | EMTKNQVSLTCLVKGFYPSDIAVEWES |
| | | | | NGQPENNYKTTPPVLDSDGSFFLYSRLT |
| | | | | VDKSRWQEGNVFSCSVMHEALHNHYT |
| | | | | QKSLSLSLGK |
| 91 | PRT | human | IAPB65- | EIVLTQSPATLSLSPGERATLSCRASQSV |
| | | | VL | SNFLAWYQQKPGQAPRLLIYGASNRAT |
| | | | | GIPARFSGSGSGTDFTLTISSLEPEDFAV |
| | | | | YYCQQGKHWPWTFGQGTKVEIKRTVA |
| | | | | APSVFIFPPSDEQLKSGTASVVCLLNNF |
| | | | | YPREAKVQWKVDNALQSGNSQESVTE |
| | | | | QDSKDSTYSLSSTLTLSKADYEKHKVY |
| | | | | ACEVTHQGLSSPVTKSFNRGEC |
| 92 | PRT | artificial | CD3B220- | EVQLVESGGGLVQPGGSLKLSCAASGF |
| | | | VH | TFNTYAMNWVRQASGKGLEWVGRIRS |
| | | | | KYNAYATYYAASVKGRFTISRDDSKNT |
| | | | | AYLQMNSLKTEDTAVYYCTRHGNFGN |
| | | | | SYVSWFAYWGQGTLVTVSSASTKGPSV |
| | | | | FPLAPCSRSTSESTAALGCLVKDYFPEP |
| | | | | VTVSWNSGALTSGVHTFPAVLQSSGLY |
| | | | | SLSSVVTVPSSSLGTKTYTCNVDHKPSN |
| | | | | TKVDKRVESKYGPPCPPCPAPEAAGGP |
| | | | | SVFLFPPKPKDTLMISRTPEVTCVVVDV |
| | | | | SQEDPEVQFNWYVDGVEVHNAKTKPR |
| | | | | EEQFNSTYRVVSVLTVLHQDWLNGKE |
| | | | | YKCKVSNKGLPSSIEKTISKAKGQPREP |
| | | | | QVYTLPPSQEEMTKNQVSLTCLVKGFY |
| | | | | PSDIAVEWESNGQPENNYKTTPPVLDSD |
| | | | | GSFLLYSKLTVDKSRWQEGNVFSCSVM |
| | | | | HEALHNHYTQKSLSLSLGK |
| 93 | PRT | artificial | CD3B220- | QAVVTQEPSLTVSPGGTVTLTCRSSTGA |
| | | | VL | VTTSNYANWVQQKPGQAPRGLIGGTN |
| | | | | KRAPGTPARFSGSLLGGKAALTLSGAQ |
| | | | | PEDEAEYYCALWYSNLWVFGGGTKLT |
| | | | | VLGQPKAAPSVTLFPPSSEELQANKATL |
| | | | | VCLISDFYPGAVTVAWKADSSPVKAGV |
| | | | | ETTTPSKQSNNKYAASSYLSLTPEQWKS |
| | | | | HRSYSCQVTHEGSTVEKTVAPTECS |
| 94 | PRT | artificial | CD3B219- | EVQLVESGGGLVQPGGSLRLSCAASGF |
| | | | VH | TFNTYAMNWVRQAPGKGLEWVARIRS |
| | | | | KYNNYATYYAASVKGRFTISRDDSKNS |
| | | | | LYLQMNSLKTEDTAVYYCARHGNFGN |
| | | | | SYVSWFAYWGQGTLVTVSSASTKGPSV |
| | | | | FPLAPCSRSTSESTAALGCLVKDYFPEP |
| | | | | VTVSWNSGALTSGVHTFPAVLQSSGLY |
| | | | | SLSSVVTVPSSSLGTKTYTCNVDHKPSN |
| | | | | TKVDKRVESKYGPPCPPCPAPEAAGGP |
| | | | | SVFLFPPKPKDTLMISRTPEVTCVVVDV |
| | | | | SQEDPEVQFNWYVDGVEVHNAKTKPR |
| | | | | EEQFNSTYRVVSVLTVLHQDWLNGKE |
| | | | | YKCKVSNKGLPSSIEKTISKAKGQPREP |
| | | | | QVYTLPPSQEEMTKNQVSLTCLVKGFY |
| | | | | PSDIAVEWESNGQPENNYKTTPPVLDSD |
| | | | | GSFLLYSKLTVDKSRWQEGNVFSCSVM |
| | | | | HEALHNHYTQKSLSLSLGK |
| 95 | PRT | artificial | CD3B219- | QTVVTQEPSLTVSPGGTVTLTCRSSTGA |
| | | | VL | VTTSNYANWVQQKPGQAPRGLIGGTN |
| | | | | KRAPGTPARFSGSLLGGKAALTLSGVQ |
| | | | | PEDEAEYYCALWYSNLWVFGGGTKLT |
| | | | | VLGQPKAAPSVTLFPPSSEELQANKATL |
| | | | | VCLISDFYPGAVTVAWKADSSPVKAGV |
| | | | | ETTTPSKQSNNKYAASSYLSLTPEQWKS |
| | | | | HRSYSCQVTHEGSTVEKTVAPTECS |
| 96 | PRT | mouse | CD3B219 | TYAMN |
| | | | and | |
| | | | CD3B220- | |
| | | | HCDR1 | |
| 97 | PRT | mouse | CD3B220- | RIRSKYNAYATYYAASVKG |
| | | | HCDR2 | |
| 98 | PRT | mouse | CD3B219 | HGNFGNSYVSWFAY |
| | | | and | |
| | | | CD3B220- | |
| | | | HCDR3 | |
| 99 | PRT | mouse | CD3B219 | RSSTGAVTTSNYAN |
| | | | and | |
| | | | CD3B220- | |
| | | | LCDR1 | |
| 100 | PRT | mouse | CD3B219 | GTNKRAP |
| | | | and | |
| | | | CD3B220- | |
| | | | LCDR2 | |
| 101 | PRT | mouse | CD3B219 | ALWYSNLWV |
| | | | and | |
| | | | CD3B220- | |
| | | | LCDR3 | |
| 102 | PRT | artificial | CD3B219- | RIRSKYNNYATYYAASVKG |
| | | | HCDR2 | |
| 103 | PRT | Human | IAPB57- | QQVNSYPLT |
| | | | LCDR3 | |

1.-7. (canceled)

8. An isolated IL1RAP x CD3 bispecific antibody comprising:
a) a first heavy chain (HC1);
b) a second heavy chain (HC2);
c) a first light chain (LC1); and
d) a second light chain (LC2),
wherein the HC1 and the LC1 pair to form a first antigen-binding site that specifically binds CD3, and the HC2 and the LC2 pair to form a second antigen-binding site that specifically binds IL1RAP, or an IL1RAP x CD3 bispecific binding fragment thereof.

9. The IL1RAP x CD3 bispecific antibody or bispecific binding fragment of claim 8, wherein HC1 comprises SEQ ID NO: 94 LC1 comprises SEQ ID NO: 95, HC2 comprises SEQ ID NO: 72, and LC2 comprises SEQ ID NO: 73.

10. The IL1RAP x CD3 bispecific antibody or bispecific binding fragment of claim 8, wherein the antibody or bispecific binding fragment specifically binds IL1RAP with a KD of less than about 30 nM as measured by surface plasmon resonance.

11. The IL1RAP x CD3 bispecific antibody or bispecific binding fragment of claim 8, wherein the antibody or bispecific binding fragment thereof binds IL1RAP on the surface of cells selected from the group consisting of human acute myeloid leukemia cells, human lung cancer cells, human colon cancer cells, human pancreatic cancer cells, human myelodysplastic syndrome cancer cells, human chronic myeloid leukemia, human diffuse large B-Cell lymphoma cells, human acute lymphoblastic leukemia cells, and human T-cell leukemia/lymphoma cells.

12. The IL1RAP x CD3 bispecific antibody or bispecific binding fragment of claim 8, wherein the antibody or bispecific binding fragment inhibits IL-10 mediated signaling through AP-1 and NF-κB responsive elements at concentrations above 6.7 nM.

13. The IL1RAP x CD3 bispecific antibody or bispecific binding fragment of claim 8, wherein the antibody or bispecific binding fragment induces T-cell dependent cytotoxicity of IL1RAP-expressing cells in vitro with an EC50 of less than about 1.3 nM.

14. An isolated IL1RAP x CD3 bispecific antibody or an IL1RAP x CD3 bispecific binding fragment thereof comprising:
a) a first heavy chain (HC1);
b) a second heavy chain (HC2);
c) a first light chain (LC1); and
d) a second light chain (LC2),
wherein the HC1 and the LC1 pair to form a first antigen-binding site that specifically binds CD3 and comprise a heavy chain CDR1 (HCDR1) as depicted in SEQ ID NO: 96, an HCDR2 as depicted in SEQ ID NO: 102, an HCDR3 as depicted in SEQ ID NO: 98 a light chain CDR1 (LCDR1) as depicted in SEQ ID NO: 99, an LCDR2 as depicted in SEQ ID NO: 100, and an LCDR3 as depicted in SEQ ID NO: 101;
and the HC2 and the LC2 pair to form a second antigen-binding site that specifically binds IL1RAP and comprise a heavy chain CDR1 (HCDR1) as depicted in SEQ ID NO: 16 or 22, an HCDR2 as depicted in SEQ ID NO: 17 or 23, an HCDR3 as depicted in SEQ ID NO: 18 or 24 a light chain CDR1 (LCDR1) as depicted in SEQ ID NO: 46 or 62, an LCDR2 as depicted in SEQ ID NO: 47 or 63, and an LCDR3 as depicted in SEQ ID NO: 103 or 64.

15. An isolated cell expressing the antibody or bispecific binding fragment of claim 14.

16. The cell of claim 15 wherein the antibody or bispecific binding fragment is recombinantly produced.

17. A method for treating a subject having cancer, said method comprising:
administering a therapeutically effective amount of the IL1RAP x CD3 bispecific antibody or bispecific binding fragment of claim 14 to a patient in need thereof for a time sufficient to treat the cancer.

18. A method for inhibiting growth or proliferation of cancer cells, said method comprising:
administering a therapeutically effective amount of the IL1RAPx CD3 bispecific antibody or bispecific binding fragment of claim 14 to inhibit the growth or proliferation of cancer cells.

19. A method of redirecting a T cell to an IL1RAP-expressing cancer cell, said method comprising:
administering a therapeutically effective amount of the IL1RAP x CD3 bispecific antibody or bispecific binding fragment of claim 14 to redirect a T cell to a cancer.

20. The method of claim 19 wherein the cancer is an IL1RAP-expressing cancer.

21. The method of claim 20 wherein the IL1RAP-expressing cancer, is acute myeloid leukemia (AML) myelodysplastic syndrome (MDS, low or high risk), acute lymphocytic leukemia (ALL, including all subtypes), diffuse large B-cell lymphoma (DLBCL), chronic myeloid leukemia (CML), blastic plasmacytoid dendritic cell neoplasm (DPDCN), T-cell leukemia/lymphoma, prostate cancer, lung cancer, colorectal cancer, or pancreatic cancer.

22. The method of claim 19 further comprising administering a second therapeutic agent.

23. The method of claim 22 wherein the second therapeutic agent is a chemotherapeutic agent or a targeted anti-cancer therapy.

24. The method of claim 23 wherein the chemotherapeutic agent is cytarabine, an anthracycline, histamine dihydrochloride, or interleukin 2.

25. The method of claim 22 wherein the second therapeutic agent is administered to said subject simultaneously with, sequentially, or separately from the bispecific antibody.

26. A pharmaceutical composition comprising the IL1RAP x CD3 bispecific antibody or bispecific binding fragment of claim 14 and a pharmaceutically acceptable carrier.

27. An isolated synthetic polynucleotide encoding the HC1, the HC2, the LC1 or the LC2 of the IL1RAP x CD3 bispecific antibody or bispecific binding fragment of claim 14.

28. A kit comprising the IL1RAP x CD3 bispecific antibody or bispecific binding fragment of claim 14 and instructions for use thereof.

29. A method of inhibiting angiogenesis in a subject, said method comprising administering to a subject in need thereof a IL1RAP x CD3 bispecific antibody or bispecific binding fragment of claim 14, wherein the subject has cancer.

30. A method of depleting MDSCs in a subject, said method comprising administering to a subject in need thereof a IL1RAP x CD3 bispecific antibody or bispecific binding fragment of claim 14, wherein the subject has cancer.
